SEQUENCE LISTING (572 sequences)

SEQ ID NO: 1 - protein, 6 aa, synthetic construct
ENLYFQ

SEQ ID NO: 2 - protein, 18 aa, synthetic construct
LKQINVIAGVKEPIRAYG

SEQ ID NO: 3 - protein, 17 aa, synthetic construct
LKQINVIAGVKPIRAYG

SEQ ID NO: 4 - protein, 8 aa, synthetic construct
  SITE 1: note = D-Phenylalanine
  SITE 4: note = D-Tryptophan
FCFWKTET

SEQ ID NO: 5 - protein, 9 aa, synthetic construct
FCFAKTETA

SEQ ID NO: 6 - protein, 27 aa, synthetic construct
LKQINVIAGVKEPIRAYGCDSNNAANA

SEQ ID NO: 7 - protein, 26 aa, synthetic construct
LKQINVIAGVKEPIRAYGCSDNNAAA

SEQ ID NO: 8 - protein, 25 aa, synthetic construct
LKQINVIAGVKEPIRAYGCSNDAAA

SEQ ID NO: 9 - protein, 25 aa, synthetic construct
LKQINVIAGVKEPIRAYGCSAANDA

SEQ ID NO: 10 - protein, 26 aa, synthetic construct
LKQINVIAGVKEPIRAYGCSAAANDA

SEQ ID NO: 11 - protein, 27 aa, synthetic construct
LKQINVIAGVKEPIRAYGCSAAAANDA

SEQ ID NO: 12 - protein, 27 aa, synthetic construct
LKQINVIAGVKEPIRAYGAAACSANDA

SEQ ID NO: 13 - protein, 33 aa, synthetic construct
LKQINVIAGVKEPIRAYGAAACSANDACSANDA

SEQ ID NO: 14 - protein, 30 aa, synthetic construct
LKQINVIAGVKEPIRAYGAAACSACDAADA

SEQ ID NO: 15 - protein, 30 aa, synthetic construct
LKQINVIAGVKEPIRAYGAAAASACDAADA

SEQ ID NO: 16 - protein, 31 aa, synthetic construct
LKQINVIAGVKEPIRAYGAAACSAADAAADA

SEQ ID NO: 17 - protein, 9 aa, synthetic construct
AAACSANDA

SEQ ID NO: 18 - protein, 15 aa, synthetic construct
AAACSANDACSANDA

SEQ ID NO: 19 - protein, 12 aa, synthetic construct
AAACSACDAADA

SEQ ID NO: 20 - protein, 12 aa, synthetic construct
AAAASACDAADA

SEQ ID NO: 21 - protein, 12 aa, synthetic construct
AAACSAADAADA

SEQ ID NO: 22 - protein, 6 aa, synthetic construct
  SITE 1: note = D-Cysteine
CSANDA

SEQ ID NO: 23 - protein, 6 aa, synthetic construct
  SITE 5: note = D-Aspartic acid
CSANDA

SEQ ID NO: 24 - protein, 6 aa, synthetic construct
  SITE 1: note = D-Cysteine
  SITE 5: note = D-Aspartic acid
CSANDA

SEQ ID NO: 25 - protein, 6 aa, synthetic construct
DSANCA

SEQ ID NO: 26 - protein, 10 aa, synthetic construct
  VARIANT 1: note = any amino acid
  VARIANT 7: note = any amino acid
  VARIANT 8: note = any amino acid
  VARIANT 9: note = any amino acid
  VARIANT 10: note = any amino acid
XCDFRWXXXX

SEQ ID NO: 27 - protein, 7 aa, synthetic construct
  SITE 2: note = hydroxy-Cysteine
  SITE 6: note = hydroxy-Glutamic acid
GCSANEA

SEQ ID NO: 28 - protein, 7 aa, synthetic construct
  CROSSLNK 1..7: note = Circular peptide
  SITE 6: note = hydroxy-Glutamic acid
  SITE 2: note = hydroxy-Cysteine
GCSANEA

SEQ ID NO: 29 - protein, 6 aa, synthetic construct
  VARIANT 2: note = any amino acid
  VARIANT 5: note = any amino acid
EXLYXQ

SEQ ID NO: 30 - skipped

SEQ ID NO: 31 - protein, 18 aa, synthetic construct
  VARIANT 2..8: note = Each of amino acids 2-8 can be present or absent
  VARIANT 11..17: note = Each of amino acids 11-17 can be present or absent
CXXXXXXXCDXXXXXXXD

SEQ ID NO: 32 - protein, 26 aa, synthetic construct
  VARIANT 2..8: note = Each of amino acids 2-8 can be present or absent
  VARIANT 11..17: note = Each of amino acid 11-17 can be present or absent
  VARIANT 19..25: note = Each of amino acids 19-25 can be present or absent
CXXXXXXXDXXXXXXXXCXXXXXXXD

SEQ ID NO: 33 - protein, 4 aa, synthetic construct
LKQI

SEQ ID NO: 34 - protein, 5 aa, synthetic construct
LKQIN

SEQ ID NO: 35 - protein, 6 aa, synthetic construct
LKQINV

SEQ ID NO: 36 - protein, 7 aa, synthetic construct
LKQINVI

SEQ ID NO: 37 - protein, 8 aa, synthetic construct
LKQINVIA

SEQ ID NO: 38 - protein, 9 aa, synthetic construct
LKQINVIAG

SEQ ID NO: 39 - protein, 10 aa, synthetic construct
LKQINVIAGV

SEQ ID NO: 40 - protein, 11 aa, synthetic construct
LKQINVIAGVK

SEQ ID NO: 41 - protein, 12 aa, synthetic construct
LKQINVIAGVKE

SEQ ID NO: 42 - protein, 13 aa, synthetic construct
LKQINVIAGVKEP

SEQ ID NO: 43 - protein, 14 aa, synthetic construct
LKQINVIAGVKEPI

SEQ ID NO: 44 - protein, 15 aa, synthetic construct
LKQINVIAGVKEPIR

SEQ ID NO: 45 - protein, 16 aa, synthetic construct
LKQINVIAGVKEPIRA

SEQ ID NO: 46 - protein, 17 aa, synthetic construct
LKQINVIAGVKEPIRAY

SEQ ID NO: 47 - protein, 18 aa, synthetic construct
LKQINVIAGVKEPIRAYG

SEQ ID NO: 48 - protein, 19 aa, synthetic construct
LKQINVIAGVKEPIRAYGC

SEQ ID NO: 49 - protein, 20 aa, synthetic construct
LKQINVIAGVKEPIRAYGCS

SEQ ID NO: 50 - protein, 21 aa, synthetic construct
LKQINVIAGVKEPIRAYGCSA

SEQ ID NO: 51 - protein, 22 aa, synthetic construct
LKQINVIAGVKEPIRAYGCSAN

SEQ ID NO: 52 - protein, 23 aa, synthetic construct
LKQINVIAGVKEPIRAYGCSAND

SEQ ID NO: 53 - protein, 24 aa, synthetic construct
LKQINVIAGVKEPIRAYGCSANDA

SEQ ID NO: 54 - protein, 23 aa, synthetic construct
KQINVIAGVKEPIRAYGCSANDA

SEQ ID NO: 55 - protein, 22 aa, synthetic construct
QINVIAGVKEPIRAYGCSANDA

SEQ ID NO: 56 - protein, 21 aa, synthetic construct
INVIAGVKEPIRAYGCSANDA

SEQ ID NO: 57 - protein, 20 aa, synthetic construct
NVIAGVKEPIRAYGCSANDA

SEQ ID NO: 58 - protein, 19 aa, synthetic construct
VIAGVKEPIRAYGCSANDA

SEQ ID NO: 59 - protein, 18 aa, synthetic construct
IAGVKEPIRAYGCSANDA

SEQ ID NO: 60 - protein, 17 aa, synthetic construct
AGVKEPIRAYGCSANDA

SEQ ID NO: 61 - protein, 16 aa, synthetic construct
GVKEPIRAYGCSANDA

SEQ ID NO: 62 - protein, 15 aa, synthetic construct
VKEPIRAYGCSANDA

SEQ ID NO: 63 - protein, 14 aa, synthetic construct
KEPIRAYGCSANDA

SEQ ID NO: 64 - protein, 13 aa, synthetic construct
EPIRAYGCSANDA

SEQ ID NO: 65 - protein, 12 aa, synthetic construct
PIRAYGCSANDA

SEQ ID NO: 66 - protein, 11 aa, synthetic construct
IRAYGCSANDA

SEQ ID NO: 67 - protein, 10 aa, synthetic construct
RAYGCSANDA

SEQ ID NO: 68 - protein, 9 aa, synthetic construct
AYGCSANDA

SEQ ID NO: 69 - protein, 8 aa, synthetic construct
YGCSANDA

SEQ ID NO: 70 - protein, 7 aa, synthetic construct
GCSANDA

SEQ ID NO: 71 - protein, 6 aa, synthetic construct
CSANDA

SEQ ID NO: 72 - protein, 5 aa, synthetic construct
SANDA

SEQ ID NO: 73 - protein, 4 aa, synthetic construct
ANDA

SEQ ID NO: 74 - protein, 20 aa, synthetic construct
LKQINVIAGVKEPIRAYGCD

SEQ ID NO: 75 - protein, 21 aa, synthetic construct
LKQINVIAGVKEPIRAYGCDS

SEQ ID NO: 76 - protein, 22 aa, synthetic construct
LKQINVIAGVKEPIRAYGCDSN

SEQ ID NO: 77 - protein, 23 aa, synthetic construct
LKQINVIAGVKEPIRAYGCDSNN

SEQ ID NO: 78 - protein, 24 aa, synthetic construct
LKQINVIAGVKEPIRAYGCDSNNA

SEQ ID NO: 79 - protein, 25 aa, synthetic construct
LKQINVIAGVKEPIRAYGCDSNNAA

SEQ ID NO: 80 - protein, 26 aa, synthetic construct
LKQINVIAGVKEPIRAYGCDSNNAAN

SEQ ID NO: 81 - skipped

SEQ ID NO: 82 - protein, 26 aa, synthetic construct
KQINVIAGVKEPIRAYGCDSNNAANA

SEQ ID NO: 83 - protein, 25 aa, synthetic construct
QINVIAGVKEPIRAYGCDSNNAANA

SEQ ID NO: 84 - protein, 24 aa, synthetic construct
INVIAGVKEPIRAYGCDSNNAANA

SEQ ID NO: 85 - protein, 23 aa, synthetic construct
NVIAGVKEPIRAYGCDSNNAANA

SEQ ID NO: 86 - protein, 22 aa, synthetic construct
VIAGVKEPIRAYGCDSNNAANA

SEQ ID NO: 87 - protein, 21 aa, synthetic construct
IAGVKEPIRAYGCDSNNAANA

SEQ ID NO: 88 - protein, 20 aa, synthetic construct
AGVKEPIRAYGCDSNNAANA

SEQ ID NO: 89 - protein, 19 aa, synthetic construct
GVKEPIRAYGCDSNNAANA

SEQ ID NO: 90 - protein, 18 aa, synthetic construct
VKEPIRAYGCDSNNAANA

SEQ ID NO: 91 - protein, 17 aa, synthetic construct
KEPIRAYGCDSNNAANA

SEQ ID NO: 92 - protein, 16 aa, synthetic construct
EPIRAYGCDSNNAANA

SEQ ID NO: 93 - protein, 15 aa, synthetic construct
PIRAYGCDSNNAANA

SEQ ID NO: 94 - protein, 14 aa, synthetic construct
IRAYGCDSNNAANA

SEQ ID NO: 95 - protein, 13 aa, synthetic construct
RAYGCDSNNAANA

SEQ ID NO: 96 - protein, 12 aa, synthetic construct
AYGCDSNNAANA

SEQ ID NO: 97 - protein, 11 aa, synthetic construct
YGCDSNNAANA

SEQ ID NO: 98 - protein, 10 aa, synthetic construct
GCDSNNAANA

SEQ ID NO: 99 - protein, 9 aa, synthetic construct
CDSNNAANA

SEQ ID NO: 100 - protein, 8 aa, synthetic construct
DSNNAANA

SEQ ID NO: 101 - protein, 7 aa, synthetic construct
SNNAANA

SEQ ID NO: 102 - protein, 6 aa, synthetic construct
NNAANA

SEQ ID NO: 103 - protein, 5 aa, synthetic construct
NAANA

SEQ ID NO: 104 - protein, 4 aa, synthetic construct
AANA

SEQ ID NO: 105 - protein, 21 aa, synthetic construct
LKQINVIAGVKEPIRAYGCSD

SEQ ID NO: 106 - protein, 22 aa, synthetic construct
LKQINVIAGVKEPIRAYGCSDN

SEQ ID NO: 107 - protein, 23 aa, synthetic construct
LKQINVIAGVKEPIRAYGCSDNN

SEQ ID NO: 108 - protein, 24 aa, synthetic construct
LKQINVIAGVKEPIRAYGCSDNNA

SEQ ID NO: 109 - protein, 25 aa, synthetic construct
LKQINVIAGVKEPIRAYGCSDNNAA

SEQ ID NO: 110 - skipped

SEQ ID NO: 111 - protein, 25 aa, synthetic construct
KQINVIAGVKEPIRAYGCSDNNAAA

SEQ ID NO: 112 - protein, 24 aa, synthetic construct
QINVIAGVKEPIRAYGCSDNNAAA

SEQ ID NO: 113 - protein, 23 aa, synthetic construct
INVIAGVKEPIRAYGCSDNNAAA

SEQ ID NO: 114 - protein, 22 aa, synthetic construct
NVIAGVKEPIRAYGCSDNNAAA

SEQ ID NO: 115 - protein, 21 aa, synthetic construct
VIAGVKEPIRAYGCSDNNAAA

SEQ ID NO: 116 - protein, 20 aa, synthetic construct
IAGVKEPIRAYGCSDNNAAA

SEQ ID NO: 117 - protein, 19 aa, synthetic construct
AGVKEPIRAYGCSDNNAAA

SEQ ID NO: 118 - protein, 18 aa, synthetic construct
GVKEPIRAYGCSDNNAAA

SEQ ID NO: 119 - protein, 17 aa, synthetic construct
VKEPIRAYGCSDNNAAA

SEQ ID NO: 120 - protein, 16 aa, synthetic construct
KEPIRAYGCSDNNAAA

SEQ ID NO: 121 - protein, 15 aa, synthetic construct
EPIRAYGCSDNNAAA

SEQ ID NO: 122 - protein, 14 aa, synthetic construct
PIRAYGCSDNNAAA

SEQ ID NO: 123 - protein, 13 aa, synthetic construct
IRAYGCSDNNAAA

SEQ ID NO: 124 - protein, 12 aa, synthetic construct
RAYGCSDNNAAA

SEQ ID NO: 125 - protein, 11 aa, synthetic construct
AYGCSDNNAAA

SEQ ID NO: 126 - protein, 10 aa, synthetic construct
YGCSDNNAAA

SEQ ID NO: 127 - protein, 9 aa, synthetic construct
GCSDNNAAA

SEQ ID NO: 128 - protein, 8 aa, synthetic construct
CSDNNAAA

SEQ ID NO: 129 - protein, 7 aa, synthetic construct
SDNNAAA

SEQ ID NO: 130 - protein, 6 aa, synthetic construct
DNNAAA

SEQ ID NO: 131 - protein, 5 aa, synthetic construct
NNAAA

SEQ ID NO: 132 - protein, 4 aa, synthetic construct
NAAA

SEQ ID NO: 133 - protein, 21 aa, synthetic construct
LKQINVIAGVKEPIRAYGCSN

SEQ ID NO: 134 - protein, 22 aa, synthetic construct
LKQINVIAGVKEPIRAYGCSND

SEQ ID NO: 135 - protein, 23 aa, synthetic construct
LKQINVIAGVKEPIRAYGCSNDA

SEQ ID NO: 136 - protein, 24 aa, synthetic construct
LKQINVIAGVKEPIRAYGCSNDAA

SEQ ID NO: 137 - skipped

SEQ ID NO: 138 - protein, 24 aa, synthetic construct
KQINVIAGVKEPIRAYGCSNDAAA

SEQ ID NO: 139 - protein, 23 aa, synthetic construct
QINVIAGVKEPIRAYGCSNDAAA

SEQ ID NO: 140 - protein, 22 aa, synthetic construct
INVIAGVKEPIRAYGCSNDAAA

SEQ ID NO: 141 - protein, 21 aa, synthetic construct
NVIAGVKEPIRAYGCSNDAAA

SEQ ID NO: 142 - protein, 20 aa, synthetic construct
VIAGVKEPIRAYGCSNDAAA

SEQ ID NO: 143 - protein, 19 aa, synthetic construct
IAGVKEPIRAYGCSNDAAA

SEQ ID NO: 144 - protein, 18 aa, synthetic construct
AGVKEPIRAYGCSNDAAA

SEQ ID NO: 145 - protein, 17 aa, synthetic construct
GVKEPIRAYGCSNDAAA

SEQ ID NO: 146 - protein, 16 aa, synthetic construct
VKEPIRAYGCSNDAAA

SEQ ID NO: 147 - protein, 15 aa, synthetic construct
KEPIRAYGCSNDAAA

SEQ ID NO: 148 - protein, 14 aa, synthetic construct
EPIRAYGCSNDAAA

SEQ ID NO: 149 - protein, 13 aa, synthetic construct
PIRAYGCSNDAAA

SEQ ID NO: 150 - protein, 12 aa, synthetic construct
IRAYGCSNDAAA

SEQ ID NO: 151 - protein, 11 aa, synthetic construct
RAYGCSNDAAA

SEQ ID NO: 152 - protein, 10 aa, synthetic construct
AYGCSNDAAA

SEQ ID NO: 153 - protein, 9 aa, synthetic construct
YGCSNDAAA

SEQ ID NO: 154 - protein, 8 aa, synthetic construct
GCSNDAAA

SEQ ID NO: 155 - protein, 7 aa, synthetic construct
CSNDAAA

SEQ ID NO: 156 - protein, 6 aa, synthetic construct
SNDAAA

SEQ ID NO: 157 - protein, 5 aa, synthetic construct
NDAAA

SEQ ID NO: 158 - protein, 4 aa, synthetic construct
DAAA

SEQ ID NO: 159 - protein, 22 aa, synthetic construct
LKQINVIAGVKEPIRAYGCSAA

SEQ ID NO: 160 - protein, 23 aa, synthetic construct
LKQINVIAGVKEPIRAYGCSAAN

SEQ ID NO: 161 - protein, 24 aa, synthetic construct
LKQINVIAGVKEPIRAYGCSAAND

SEQ ID NO: 162 - skipped

SEQ ID NO: 163 - protein, 24 aa, synthetic construct
KQINVIAGVKEPIRAYGCSAANDA

SEQ ID NO: 164 - protein, 23 aa, synthetic construct
QINVIAGVKEPIRAYGCSAANDA

SEQ ID NO: 165 - protein, 22 aa, synthetic construct
INVIAGVKEPIRAYGCSAANDA

SEQ ID NO: 166 - protein, 21 aa, synthetic construct
NVIAGVKEPIRAYGCSAANDA

SEQ ID NO: 167 - protein, 20 aa, synthetic construct
VIAGVKEPIRAYGCSAANDA

SEQ ID NO: 168 - protein, 19 aa, synthetic construct
IAGVKEPIRAYGCSAANDA

SEQ ID NO: 169 - protein, 18 aa, synthetic construct
AGVKEPIRAYGCSAANDA

SEQ ID NO: 170 - protein, 17 aa, synthetic construct
GVKEPIRAYGCSAANDA

SEQ ID NO: 171 - protein, 16 aa, synthetic construct
VKEPIRAYGCSAANDA

SEQ ID NO: 172 - protein, 15 aa, synthetic construct
KEPIRAYGCSAANDA

SEQ ID NO: 173 - protein, 14 aa, synthetic construct
EPIRAYGCSAANDA

SEQ ID NO: 174 - protein, 13 aa, synthetic construct
PIRAYGCSAANDA

SEQ ID NO: 175 - protein, 12 aa, synthetic construct
IRAYGCSAANDA

SEQ ID NO: 176 - protein, 11 aa, synthetic construct
RAYGCSAANDA

SEQ ID NO: 177 - protein, 10 aa, synthetic construct
AYGCSAANDA

SEQ ID NO: 178 - protein, 9 aa, synthetic construct
YGCSAANDA

SEQ ID NO: 179 - protein, 8 aa, synthetic construct
GCSAANDA

SEQ ID NO: 180 - protein, 7 aa, synthetic construct
CSAANDA

SEQ ID NO: 181 - protein, 6 aa, synthetic construct
SAANDA

SEQ ID NO: 182 - protein, 5 aa, synthetic construct
AANDA

SEQ ID NO: 183 - protein, 4 aa, synthetic construct
ANDA

SEQ ID NO: 184 - protein, 23 aa, synthetic construct
LKQINVIAGVKEPIRAYGCSAAA

SEQ ID NO: 185 - protein, 24 aa, synthetic construct
LKQINVIAGVKEPIRAYGCSAAAN

SEQ ID NO: 186 - protein, 25 aa, synthetic construct
LKQINVIAGVKEPIRAYGCSAAAND

SEQ ID NO: 187 - skipped

SEQ ID NO: 188 - protein, 25 aa, synthetic construct
KQINVIAGVKEPIRAYGCSAAANDA

SEQ ID NO: 189 - protein, 24 aa, synthetic construct
QINVIAGVKEPIRAYGCSAAANDA

SEQ ID NO: 190 - protein, 23 aa, synthetic construct
INVIAGVKEPIRAYGCSAAANDA

SEQ ID NO: 191 - protein, 22 aa, synthetic construct
NVIAGVKEPIRAYGCSAAANDA

SEQ ID NO: 192 - protein, 21 aa, synthetic construct
VIAGVKEPIRAYGCSAAANDA

SEQ ID NO: 193 - protein, 20 aa, synthetic construct
IAGVKEPIRAYGCSAAANDA

SEQ ID NO: 194 - protein, 19 aa, synthetic construct
AGVKEPIRAYGCSAAANDA

SEQ ID NO: 195 - protein, 18 aa, synthetic construct
GVKEPIRAYGCSAAANDA

SEQ ID NO: 196 - protein, 17 aa, synthetic construct
VKEPIRAYGCSAAANDA

SEQ ID NO: 197 - protein, 16 aa, synthetic construct
KEPIRAYGCSAAANDA

SEQ ID NO: 198 - protein, 15 aa, synthetic construct
EPIRAYGCSAAANDA

SEQ ID NO: 199 - protein, 14 aa, synthetic construct
PIRAYGCSAAANDA

SEQ ID NO: 200 - protein, 13 aa, synthetic construct
IRAYGCSAAANDA

SEQ ID NO: 201 - protein, 12 aa, synthetic construct
RAYGCSAAANDA

SEQ ID NO: 202 - protein, 11 aa, synthetic construct
AYGCSAAANDA

SEQ ID NO: 203 - protein, 10 aa, synthetic construct
YGCSAAANDA

SEQ ID NO: 204 - protein, 9 aa, synthetic construct
GCSAAANDA

SEQ ID NO: 205 - protein, 8 aa, synthetic construct
CSAAANDA

SEQ ID NO: 206 - protein, 7 aa, synthetic construct
SAAANDA

SEQ ID NO: 207 - protein, 6 aa, synthetic construct
AAANDA

SEQ ID NO: 208 - protein, 5 aa, synthetic construct
AANDA

SEQ ID NO: 209 - protein, 4 aa, synthetic construct
ANDA

SEQ ID NO: 210 - protein, 24 aa, synthetic construct
LKQINVIAGVKEPIRAYGCSAAAA

SEQ ID NO: 211 - protein, 25 aa, synthetic construct
LKQINVIAGVKEPIRAYGCSAAAAN

SEQ ID NO: 212 - protein, 26 aa, synthetic construct
LKQINVIAGVKEPIRAYGCSAAAAND

SEQ ID NO: 213 - skipped

SEQ ID NO: 214 - protein, 26 aa, synthetic construct
KQINVIAGVKEPIRAYGCSAAAANDA

SEQ ID NO: 215 - protein, 25 aa, synthetic construct
QINVIAGVKEPIRAYGCSAAAANDA

SEQ ID NO: 216 - protein, 24 aa, synthetic construct
INVIAGVKEPIRAYGCSAAAANDA

SEQ ID NO: 217 - protein, 23 aa, synthetic construct
NVIAGVKEPIRAYGCSAAAANDA

SEQ ID NO: 218 - protein, 22 aa, synthetic construct
VIAGVKEPIRAYGCSAAAANDA

SEQ ID NO: 219 - protein, 21 aa, synthetic construct
IAGVKEPIRAYGCSAAAANDA

SEQ ID NO: 220 - protein, 20 aa, synthetic construct
AGVKEPIRAYGCSAAAANDA

SEQ ID NO: 221 - protein, 19 aa, synthetic construct
GVKEPIRAYGCSAAAANDA

SEQ ID NO: 222 - protein, 18 aa, synthetic construct
VKEPIRAYGCSAAAANDA

SEQ ID NO: 223 - protein, 17 aa, synthetic construct
KEPIRAYGCSAAAANDA

SEQ ID NO: 224 - protein, 16 aa, synthetic construct
EPIRAYGCSAAAANDA

SEQ ID NO: 225 - protein, 15 aa, synthetic construct
PIRAYGCSAAAANDA

SEQ ID NO: 226 - protein, 14 aa, synthetic construct
IRAYGCSAAAANDA

SEQ ID NO: 227 - protein, 13 aa, synthetic construct
RAYGCSAAAANDA

SEQ ID NO: 228 - protein, 12 aa, synthetic construct
AYGCSAAAANDA

SEQ ID NO: 229 - protein, 11 aa, synthetic construct
YGCSAAAANDA

SEQ ID NO: 230 - protein, 10 aa, synthetic construct
GCSAAAANDA

SEQ ID NO: 231 - protein, 9 aa, synthetic construct
CSAAAANDA

SEQ ID NO: 232 - protein, 8 aa, synthetic construct
SAAAANDA

SEQ ID NO: 233 - protein, 7 aa, synthetic construct
AAAANDA

SEQ ID NO: 234 - protein, 6 aa, synthetic construct
AAANDA

SEQ ID NO: 235 - protein, 5 aa, synthetic construct
AANDA

SEQ ID NO: 236 - protein, 4 aa, synthetic construct
ANDA

SEQ ID NO: 237 - protein, 19 aa, synthetic construct
LKQINVIAGVKEPIRAYGA

SEQ ID NO: 238 - protein, 20 aa, synthetic construct
LKQINVIAGVKEPIRAYGAA

SEQ ID NO: 239 - protein, 21 aa, synthetic construct
LKQINVIAGVKEPIRAYGAAA

SEQ ID NO: 240 - protein, 22 aa, synthetic construct
LKQINVIAGVKEPIRAYGAAAC

SEQ ID NO: 241 - protein, 23 aa, synthetic construct
LKQINVIAGVKEPIRAYGAAACS

SEQ ID NO: 242 - protein, 24 aa, synthetic construct
LKQINVIAGVKEPIRAYGAAACSA

SEQ ID NO: 243 - protein, 25 aa, synthetic construct
LKQINVIAGVKEPIRAYGAAACSAN

SEQ ID NO: 244 - protein, 26 aa, synthetic construct
LKQINVIAGVKEPIRAYGAAACSAND

SEQ ID NO: 245 - skipped

SEQ ID NO: 246 - protein, 26 aa, synthetic construct
KQINVIAGVKEPIRAYGAAACSANDA

SEQ ID NO: 247 - protein, 25 aa, synthetic construct
QINVIAGVKEPIRAYGAAACSANDA

SEQ ID NO: 248 - protein, 24 aa, synthetic construct
INVIAGVKEPIRAYGAAACSANDA

SEQ ID NO: 249 - protein, 23 aa, synthetic construct
NVIAGVKEPIRAYGAAACSANDA

SEQ ID NO: 250 - protein, 22 aa, synthetic construct
VIAGVKEPIRAYGAAACSANDA

SEQ ID NO: 251 - protein, 21 aa, synthetic construct
IAGVKEPIRAYGAAACSANDA

SEQ ID NO: 252 - protein, 20 aa, synthetic construct
AGVKEPIRAYGAAACSANDA

SEQ ID NO: 253 - protein, 19 aa, synthetic construct
GVKEPIRAYGAAACSANDA

SEQ ID NO: 254 - protein, 18 aa, synthetic construct
VKEPIRAYGAAACSANDA

SEQ ID NO: 255 - protein, 17 aa, synthetic construct
KEPIRAYGAAACSANDA

SEQ ID NO: 256 - protein, 16 aa, synthetic construct
EPIRAYGAAACSANDA

SEQ ID NO: 257 - protein, 15 aa, synthetic construct
PIRAYGAAACSANDA

SEQ ID NO: 258 - protein, 14 aa, synthetic construct
IRAYGAAACSANDA

SEQ ID NO: 259 - protein, 13 aa, synthetic construct
RAYGAAACSANDA

SEQ ID NO: 260 - protein, 12 aa, synthetic construct
AYGAAACSANDA

SEQ ID NO: 261 - protein, 11 aa, synthetic construct
YGAAACSANDA

SEQ ID NO: 262 - protein, 10 aa, synthetic construct
GAAACSANDA

SEQ ID NO: 263 - protein, 9 aa, synthetic construct
AAACSANDA

SEQ ID NO: 264 - protein, 8 aa, synthetic construct
AACSANDA

SEQ ID NO: 265 - protein, 7 aa, synthetic construct
ACSANDA

SEQ ID NO: 266 - protein, 6 aa, synthetic construct
CSANDA

SEQ ID NO: 267 - protein, 5 aa, synthetic construct
SANDA

SEQ ID NO: 268 - protein, 4 aa, synthetic construct
ANDA

SEQ ID NO: 269 - protein, 28 aa, synthetic construct
LKQINVIAGVKEPIRAYGAAACSANDAC

SEQ ID NO: 270 - protein, 29 aa, synthetic construct
LKQINVIAGVKEPIRAYGAAACSANDACS

SEQ ID NO: 271 - protein, 30 aa, synthetic construct
LKQINVIAGVKEPIRAYGAAACSANDACSA

SEQ ID NO: 272 - protein, 31 aa, synthetic construct
LKQINVIAGVKEPIRAYGAAACSANDACSAN

SEQ ID NO: 273 - protein, 32 aa, synthetic construct
LKQINVIAGVKEPIRAYGAAACSANDACSAND

SEQ ID NO: 274 - skipped

SEQ ID NO: 275 - protein, 32 aa, synthetic construct
KQINVIAGVKEPIRAYGAAACSANDACSANDA

SEQ ID NO: 276 - protein, 31 aa, synthetic construct
QINVIAGVKEPIRAYGAAACSANDACSANDA

SEQ ID NO: 277 - protein, 30 aa, synthetic construct
INVIAGVKEPIRAYGAAACSANDACSANDA

SEQ ID NO: 278 - protein, 29 aa, synthetic construct
NVIAGVKEPIRAYGAAACSANDACSANDA

SEQ ID NO: 279 - protein, 28 aa, synthetic construct
VIAGVKEPIRAYGAAACSANDACSANDA

SEQ ID NO: 280 - protein, 27 aa, synthetic construct
IAGVKEPIRAYGAAACSANDACSANDA

SEQ ID NO: 281 - protein, 26 aa, synthetic construct
AGVKEPIRAYGAAACSANDACSANDA

SEQ ID NO: 282 - protein, 25 aa, synthetic construct
GVKEPIRAYGAAACSANDACSANDA

SEQ ID NO: 283 - protein, 24 aa, synthetic construct
VKEPIRAYGAAACSANDACSANDA

SEQ ID NO: 284 - protein, 23 aa, synthetic construct
KEPIRAYGAAACSANDACSANDA

SEQ ID NO: 285 - protein, 22 aa, synthetic construct
EPIRAYGAAACSANDACSANDA

SEQ ID NO: 286 - protein, 21 aa, synthetic construct
PIRAYGAAACSANDACSANDA

SEQ ID NO: 287 - protein, 20 aa, synthetic construct
IRAYGAAACSANDACSANDA

SEQ ID NO: 288 - protein, 19 aa, synthetic construct
RAYGAAACSANDACSANDA

SEQ ID NO: 289 - protein, 18 aa, synthetic construct
AYGAAACSANDACSANDA

SEQ ID NO: 290 - protein, 17 aa, synthetic construct
YGAAACSANDACSANDA

SEQ ID NO: 291 - protein, 16 aa, synthetic construct
GAAACSANDACSANDA

SEQ ID NO: 292 - protein, 15 aa, synthetic construct
AAACSANDACSANDA

SEQ ID NO: 293 - protein, 14 aa, synthetic construct
AACSANDACSANDA

SEQ ID NO: 294 - protein, 13 aa, synthetic construct
ACSANDACSANDA

SEQ ID NO: 295 - protein, 12 aa, synthetic construct
CSANDACSANDA

SEQ ID NO: 296 - protein, 11 aa, synthetic construct
SANDACSANDA

SEQ ID NO: 297 - protein, 10 aa, synthetic construct
ANDACSANDA

SEQ ID NO: 298 - protein, 9 aa, synthetic construct
NDACSANDA

SEQ ID NO: 299 - protein, 8 aa, synthetic construct
DACSANDA

SEQ ID NO: 300 - protein, 7 aa, synthetic construct
ACSANDA

SEQ ID NO: 301 - skipped

SEQ ID NO: 302 - protein, 19 aa, synthetic construct
LKQINVIAGVKEPIRAYGA

SEQ ID NO: 303 - protein, 20 aa, synthetic construct
LKQINVIAGVKEPIRAYGAA

SEQ ID NO: 304 - protein, 21 aa, synthetic construct
LKQINVIAGVKEPIRAYGAAA

SEQ ID NO: 305 - protein, 22 aa, synthetic construct
LKQINVIAGVKEPIRAYGAAAC

SEQ ID NO: 306 - protein, 23 aa, synthetic construct
LKQINVIAGVKEPIRAYGAAACS

SEQ ID NO: 307 - protein, 24 aa, synthetic construct
LKQINVIAGVKEPIRAYGAAACSA

SEQ ID NO: 308 - protein, 25 aa, synthetic construct
LKQINVIAGVKEPIRAYGAAACSAC

SEQ ID NO: 309 - protein, 26 aa, synthetic construct
LKQINVIAGVKEPIRAYGAAACSACD

SEQ ID NO: 310 - protein, 27 aa, synthetic construct
LKQINVIAGVKEPIRAYGAAACSACDA

SEQ ID NO: 311 - protein, 28 aa, synthetic construct
LKQINVIAGVKEPIRAYGAAACSACDAA

SEQ ID NO: 312 - protein, 29 aa, synthetic construct
LKQINVIAGVKEPIRAYGAAACSACDAAD

SEQ ID NO: 313 - skipped

SEQ ID NO: 314 - protein, 29 aa, synthetic construct
KQINVIAGVKEPIRAYGAAACSACDAADA

SEQ ID NO: 315 - protein, 28 aa, synthetic construct
QINVIAGVKEPIRAYGAAACSACDAADA

SEQ ID NO: 316 - protein, 27 aa, synthetic construct
INVIAGVKEPIRAYGAAACSACDAADA

SEQ ID NO: 317 - protein, 26 aa, synthetic construct
NVIAGVKEPIRAYGAAACSACDAADA

SEQ ID NO: 318 - protein, 25 aa, synthetic construct
VIAGVKEPIRAYGAAACSACDAADA

SEQ ID NO: 319 - protein, 24 aa, synthetic construct
IAGVKEPIRAYGAAACSACDAADA

SEQ ID NO: 320 - protein, 23 aa, synthetic construct
AGVKEPIRAYGAAACSACDAADA

SEQ ID NO: 321 - protein, 22 aa, synthetic construct
GVKEPIRAYGAAACSACDAADA

SEQ ID NO: 322 - protein, 21 aa, synthetic construct
VKEPIRAYGAAACSACDAADA

SEQ ID NO: 323 - protein, 20 aa, synthetic construct
KEPIRAYGAAACSACDAADA

SEQ ID NO: 324 - protein, 19 aa, synthetic construct
EPIRAYGAAACSACDAADA

SEQ ID NO: 325 - protein, 18 aa, synthetic construct
PIRAYGAAACSACDAADA

SEQ ID NO: 326 - protein, 17 aa, synthetic construct
IRAYGAAACSACDAADA

SEQ ID NO: 327 - protein, 16 aa, synthetic construct
RAYGAAACSACDAADA

SEQ ID NO: 328 - protein, 15 aa, synthetic construct
AYGAAACSACDAADA

SEQ ID NO: 329 - protein, 14 aa, synthetic construct
YGAAACSACDAADA

SEQ ID NO: 330 - protein, 13 aa, synthetic construct
GAAACSACDAADA

SEQ ID NO: 331 - protein, 12 aa, synthetic construct
AAACSACDAADA

SEQ ID NO: 332 - protein, 11 aa, synthetic construct
AACSACDAADA

SEQ ID NO: 333 - protein, 10 aa, synthetic construct
ACSACDAADA

SEQ ID NO: 334 - protein, 9 aa, synthetic construct
CSACDAADA

SEQ ID NO: 335 - protein, 8 aa, synthetic construct
SACDAADA

SEQ ID NO: 336 - protein, 7 aa, synthetic construct
ACDAADA

SEQ ID NO: 337 - protein, 6 aa, synthetic construct
CDAADA

SEQ ID NO: 338 - protein, 5 aa, synthetic construct
DAADA

SEQ ID NO: 339 - protein, 4 aa, synthetic construct
AADA

SEQ ID NO: 340 - skipped

SEQ ID NO: 341 - protein, 22 aa, synthetic construct
LKQINVIAGVKEPIRAYGAAAA

SEQ ID NO: 342 - protein, 23 aa, synthetic construct
LKQINVIAGVKEPIRAYGAAAAS

SEQ ID NO: 343 - protein, 24 aa, synthetic construct
LKQINVIAGVKEPIRAYGAAAASA

SEQ ID NO: 344 - protein, 25 aa, synthetic construct
LKQINVIAGVKEPIRAYGAAAASAC

SEQ ID NO: 345 - protein, 26 aa, synthetic construct
LKQINVIAGVKEPIRAYGAAAASACD

SEQ ID NO: 346 - protein, 27 aa, synthetic construct
LKQINVIAGVKEPIRAYGAAAASACDA

SEQ ID NO: 347 - protein, 28 aa, synthetic construct
LKQINVIAGVKEPIRAYGAAAASACDAA

SEQ ID NO: 348 - protein, 29 aa, synthetic construct
LKQINVIAGVKEPIRAYGAAAASACDAAD

SEQ ID NO: 349 - skipped

SEQ ID NO: 350 - protein, 29 aa, synthetic construct
KQINVIAGVKEPIRAYGAAAASACDAADA

SEQ ID NO: 351 - protein, 28 aa, synthetic construct
QINVIAGVKEPIRAYGAAAASACDAADA

SEQ ID NO: 352 - protein, 27 aa, synthetic construct
INVIAGVKEPIRAYGAAAASACDAADA

SEQ ID NO: 353 - protein, 26 aa, synthetic construct
NVIAGVKEPIRAYGAAAASACDAADA

SEQ ID NO: 354 - protein, 25 aa, synthetic construct
VIAGVKEPIRAYGAAAASACDAADA

SEQ ID NO: 355 - protein, 24 aa, synthetic construct
IAGVKEPIRAYGAAAASACDAADA

SEQ ID NO: 356 - protein, 23 aa, synthetic construct
AGVKEPIRAYGAAAASACDAADA

SEQ ID NO: 357 - protein, 22 aa, synthetic construct
GVKEPIRAYGAAAASACDAADA

SEQ ID NO: 358 - protein, 21 aa, synthetic construct
VKEPIRAYGAAAASACDAADA

SEQ ID NO: 359 - protein, 20 aa, synthetic construct
KEPIRAYGAAAASACDAADA

SEQ ID NO: 360 - protein, 19 aa, synthetic construct
EPIRAYGAAAASACDAADA

SEQ ID NO: 361 - protein, 18 aa, synthetic construct
PIRAYGAAAASACDAADA

SEQ ID NO: 362 - protein, 17 aa, synthetic construct
IRAYGAAAASACDAADA

SEQ ID NO: 363 - protein, 16 aa, synthetic construct
RAYGAAAASACDAADA

SEQ ID NO: 364 - protein, 15 aa, synthetic construct
AYGAAAASACDAADA

SEQ ID NO: 365 - protein, 14 aa, synthetic construct
YGAAAASACDAADA

SEQ ID NO: 366 - protein, 13 aa, synthetic construct
GAAAASACDAADA

SEQ ID NO: 367 - protein, 12 aa, synthetic construct
AAAASACDAADA

SEQ ID NO: 368 - protein, 11 aa, synthetic construct
AAASACDAADA

SEQ ID NO: 369 - protein, 9 aa, synthetic construct
ASACDAADA

SEQ ID NO: 370 - skipped

SEQ ID NO: 371 - protein, 25 aa, synthetic construct
LKQINVIAGVKEPIRAYGAAACSAA

SEQ ID NO: 372 - protein, 26 aa, synthetic construct
LKQINVIAGVKEPIRAYGAAACSAAD

SEQ ID NO: 373 - protein, 27 aa, synthetic construct
LKQINVIAGVKEPIRAYGAAACSAADA

SEQ ID NO: 374 - protein, 28 aa, synthetic construct
LKQINVIAGVKEPIRAYGAAACSAADAA

SEQ ID NO: 375 - protein, 29 aa, synthetic construct
LKQINVIAGVKEPIRAYGAAACSAADAAD

SEQ ID NO: 376 - protein, 30 aa, synthetic construct
LKQINVIAGVKEPIRAYGAAACSAADAADA

SEQ ID NO: 377 - protein, 29 aa, synthetic construct
KQINVIAGVKEPIRAYGAAACSAADAADA

SEQ ID NO: 378 - protein, 28 aa, synthetic construct
QINVIAGVKEPIRAYGAAACSAADAADA

SEQ ID NO: 379 - protein, 27 aa, synthetic construct
INVIAGVKEPIRAYGAAACSAADAADA

SEQ ID NO: 380 - protein, 26 aa, synthetic construct
NVIAGVKEPIRAYGAAACSAADAADA

SEQ ID NO: 381 - protein, 25 aa, synthetic construct
VIAGVKEPIRAYGAAACSAADAADA

SEQ ID NO: 382 - protein, 24 aa, synthetic construct
IAGVKEPIRAYGAAACSAADAADA

SEQ ID NO: 383 - protein, 23 aa, synthetic construct
AGVKEPIRAYGAAACSAADAADA

SEQ ID NO: 384 - protein, 22 aa, synthetic construct
GVKEPIRAYGAAACSAADAADA

SEQ ID NO: 385 - protein, 21 aa, synthetic construct
VKEPIRAYGAAACSAADAADA

SEQ ID NO: 386 - protein, 20 aa, synthetic construct
KEPIRAYGAAACSAADAADA

SEQ ID NO: 387 - protein, 19 aa, synthetic construct
EPIRAYGAAACSAADAADA

SEQ ID NO: 388 - protein, 18 aa, synthetic construct
PIRAYGAAACSAADAADA

SEQ ID NO: 389 - protein, 17 aa, synthetic construct
IRAYGAAACSAADAADA

SEQ ID NO: 390 - protein, 16 aa, synthetic construct
RAYGAAACSAADAADA

SEQ ID NO: 391 - protein, 15 aa, synthetic construct
AYGAAACSAADAADA

SEQ ID NO: 392 - protein, 14 aa, synthetic construct
YGAAACSAADAADA

SEQ ID NO: 393 - protein, 13 aa, synthetic construct
GAAACSAADAADA

SEQ ID NO: 394 - protein, 12 aa, synthetic construct
AAACSAADAADA

SEQ ID NO: 395 - protein, 11 aa, synthetic construct
AACSAADAADA

SEQ ID NO: 396 - protein, 10 aa, synthetic construct
ACSAADAADA

SEQ ID NO: 397 - protein, 9 aa, synthetic construct
CSAADAADA

SEQ ID NO: 398 - skipped

SEQ ID NO: 399 - protein, 7 aa, synthetic construct
AADAADA

SEQ ID NO: 400 - protein, 6 aa, synthetic construct
ADAADA

SEQ ID NO: 401 - skipped

SEQ ID NO: 402 - protein, 4 aa, synthetic construct
GVCK

SEQ ID NO: 403 - protein, 5 aa, synthetic construct
GVCKY

SEQ ID NO: 404 - protein, 6 aa, synthetic construct
GVCKYG

SEQ ID NO: 405 - protein, 7 aa, synthetic construct
GVCKYGE

SEQ ID NO: 406 - protein, 8 aa, synthetic construct
GVCKYGEW

SEQ ID NO: 407 - protein, 9 aa, synthetic construct
GVCKYGEWC

SEQ ID NO: 408 - protein, 10 aa, synthetic construct
GVCKYGEWCE

SEQ ID NO: 409 - protein, 11 aa, synthetic construct
GVCKYGEWCEI

SEQ ID NO: 410 - protein, 12 aa, synthetic construct
GVCKYGEWCEIV

SEQ ID NO: 411 - protein, 13 aa, synthetic construct
GVCKYGEWCEIVE

SEQ ID NO: 412 - protein, 14 aa, synthetic construct
GVCKYGEWCEIVEI

SEQ ID NO: 413 - protein, 13 aa, synthetic construct
VCKYGEWCEIVEI

SEQ ID NO: 414 - protein, 12 aa, synthetic construct
CKYGEWCEIVEI

SEQ ID NO: 415 - protein, 11 aa, synthetic construct
KYGEWCEIVEI

SEQ ID NO: 416 - protein, 10 aa, synthetic construct
YGEWCEIVEI

SEQ ID NO: 417 - protein, 9 aa, synthetic construct
GEWCEIVEI

SEQ ID NO: 418 - protein, 8 aa, synthetic construct
EWCEIVEI

SEQ ID NO: 419 - protein, 7 aa, synthetic construct
WCEIVEI

SEQ ID NO: 420 - protein, 6 aa, synthetic construct
CEIVEI

SEQ ID NO: 421 - protein, 5 aa, synthetic construct
EIVEI

SEQ ID NO: 422 - protein, 4 aa, synthetic construct
IVEI

SEQ ID NO: 423 - skipped

SEQ ID NO: 424 - protein, 19 aa, synthetic construct
  SITE 19: note = D-Cysteine
LKQINVIAGVKEPIRAYGC

SEQ ID NO: 425 - protein, 20 aa, synthetic construct
  SITE 19: note = D-Cysteine
LKQINVIAGVKEPIRAYGCS

SEQ ID NO: 426 - protein, 21 aa, synthetic construct
  SITE 19: note = D-Cysteine
LKQINVIAGVKEPIRAYGCSA

SEQ ID NO: 427 - protein, 22 aa, synthetic construct
  SITE 19: note = D-Cysteine
LKQINVIAGVKEPIRAYGCSAN

SEQ ID NO: 428 - protein, 23 aa, synthetic construct
  SITE 19: note = D-Cysteine
LKQINVIAGVKEPIRAYGCSAND

SEQ ID NO: 429 - protein, 24 aa, synthetic construct
  SITE 19: note = D-Cysteine
LKQINVIAGVKEPIRAYGCSANDA

SEQ ID NO: 430 - protein, 23 aa, synthetic construct
  SITE 18: note = D-Cysteine
KQINVIAGVKEPIRAYGCSANDA

SEQ ID NO: 431 - protein, 22 aa, synthetic construct
  SITE 17: note = D-Cysteine
QINVIAGVKEPIRAYGCSANDA

SEQ ID NO: 432 - protein, 21 aa, synthetic construct
  SITE 16: note = D-Cysteine
INVIAGVKEPIRAYGCSANDA

SEQ ID NO: 433 - protein, 20 aa, synthetic construct
  SITE 15: note = D-Cysteine
NVIAGVKEPIRAYGCSANDA

SEQ ID NO: 434 - protein, 19 aa, synthetic construct
  SITE 14: note = D-Cysteine
VIAGVKEPIRAYGCSANDA

SEQ ID NO: 435 - protein, 18 aa, synthetic construct
  SITE 13: note = D-Cysteine
IAGVKEPIRAYGCSANDA

SEQ ID NO: 436 - protein, 17 aa, synthetic construct
  SITE 12: note = D-Cysteine
AGVKEPIRAYGCSANDA

SEQ ID NO: 437 - protein, 16 aa, synthetic construct
  SITE 11: note = D-Cysteine
GVKEPIRAYGCSANDA

SEQ ID NO: 438 - protein, 15 aa, synthetic construct
  SITE 10: note = D-Cysteine
VKEPIRAYGCSANDA

SEQ ID NO: 439 - protein, 14 aa, synthetic construct
  SITE 9: note = D-Cysteine
KEPIRAYGCSANDA

SEQ ID NO: 440 - protein, 13 aa, synthetic construct
  SITE 8: note = D-Cysteine
EPIRAYGCSANDA

SEQ ID NO: 441 - protein, 12 aa, synthetic construct
  SITE 7: note = D-Cysteine
PIRAYGCSANDA

SEQ ID NO: 442 - protein, 11 aa, synthetic construct
  SITE 6: note = D-Cysteine
IRAYGCSANDA

SEQ ID NO: 443 - protein, 10 aa, synthetic construct
  SITE 5: note = D-Cysteine
RAYGCSANDA

SEQ ID NO: 444 - protein, 9 aa, synthetic construct
  SITE 4: note = D-Cysteine
AYGCSANDA

SEQ ID NO: 445 - protein, 8 aa, synthetic construct
  SITE 3: note = D-Cysteine
YGCSANDA

SEQ ID NO: 446 - protein, 7 aa, synthetic construct
  SITE 2: note = D-Cysteine
GCSANDA

SEQ ID NO: 447 - protein, 6 aa, synthetic construct
  SITE 1: note = D-Cysteine
CSANDA

SEQ ID NO: 448 - skipped

SEQ ID NO: 449 - protein, 23 aa, synthetic construct
  SITE 23: note = D-Apartic acid
LKQINVIAGVKEPIRAYGCSAND

SEQ ID NO: 450 - protein, 24 aa, synthetic construct
  SITE 23: note = D-Apartic acid
LKQINVIAGVKEPIRAYGCSANDA

SEQ ID NO: 451 - protein, 23 aa, synthetic construct
  SITE 22: note = D-Apartic acid
KQINVIAGVKEPIRAYGCSANDA

SEQ ID NO: 452 - protein, 22 aa, synthetic construct
  SITE 21: note = D-Apartic acid
QINVIAGVKEPIRAYGCSANDA

SEQ ID NO: 453 - protein, 21 aa, synthetic construct
  SITE 20: note = D-Apartic acid
INVIAGVKEPIRAYGCSANDA

SEQ ID NO: 454 - protein, 20 aa, synthetic construct
  SITE 19: note = D-Apartic acid
NVIAGVKEPIRAYGCSANDA

SEQ ID NO: 455 - protein, 19 aa, synthetic construct
  SITE 18: note = D-Apartic acid
VIAGVKEPIRAYGCSANDA

SEQ ID NO: 456 - protein, 18 aa, synthetic construct
  SITE 17: note = D-Apartic acid
IAGVKEPIRAYGCSANDA

SEQ ID NO: 457 - protein, 17 aa, synthetic construct
  SITE 16: note = D-Apartic acid
AGVKEPIRAYGCSANDA

SEQ ID NO: 458 - protein, 16 aa, synthetic construct
  SITE 15: note = D-Apartic acid
GVKEPIRAYGCSANDA

SEQ ID NO: 459 - protein, 15 aa, synthetic construct
  SITE 14: note = D-Apartic acid
VKEPIRAYGCSANDA

SEQ ID NO: 460 - protein, 14 aa, synthetic construct
  SITE 13: note = D-Apartic acid
KEPIRAYGCSANDA

SEQ ID NO: 461 - protein, 13 aa, synthetic construct
  SITE 12: note = D-Apartic acid
EPIRAYGCSANDA

SEQ ID NO: 462 - protein, 12 aa, synthetic construct
  SITE 11: note = D-Apartic acid
PIRAYGCSANDA

SEQ ID NO: 463 - protein, 11 aa, synthetic construct
  SITE 10: note = D-Apartic acid
IRAYGCSANDA

SEQ ID NO: 464 - protein, 10 aa, synthetic construct
  SITE 9: note = D-Apartic acid
RAYGCSANDA

SEQ ID NO: 465 - protein, 9 aa, synthetic construct
  SITE 8: note = D-Apartic acid
AYGCSANDA

SEQ ID NO: 466 - protein, 8 aa, synthetic construct
  SITE 7: note = D-Apartic acid
YGCSANDA

SEQ ID NO: 467 - protein, 7 aa, synthetic construct
  SITE 6: note = D-Apartic acid
GCSANDA

SEQ ID NO: 468 - protein, 6 aa, synthetic construct
  SITE 5: note = D-Apartic acid
CSANDA

SEQ ID NO: 469 - protein, 5 aa, synthetic construct
  SITE 4: note = D-Apartic acid
SANDA

SEQ ID NO: 470 - protein, 4 aa, synthetic construct
  SITE 3: note = D-Apartic acid
ANDA

SEQ ID NO: 471 - skipped

SEQ ID NO: 472 - protein, 19 aa, synthetic construct
  SITE 19: note = D-Cysteine
LKQINVIAGVKEPIRAYGC

SEQ ID NO: 473 - protein, 20 aa, synthetic construct
  SITE 19: note = D-Cysteine
LKQINVIAGVKEPIRAYGCS

SEQ ID NO: 474 - protein, 21 aa, synthetic construct
  SITE 19: note = D-Cysteine
LKQINVIAGVKEPIRAYGCSA

SEQ ID NO: 475 - protein, 22 aa, synthetic construct
  SITE 19: note = D-Cysteine
LKQINVIAGVKEPIRAYGCSAN

SEQ ID NO: 476 - protein, 23 aa, synthetic construct
  SITE 19: note = D-Cysteine
  SITE 23: note = D-Aspartic acid
LKQINVIAGVKEPIRAYGCSAND

SEQ ID NO: 477 - protein, 24 aa, synthetic construct
  SITE 19: note = D-Cysteine
  SITE 23: note = D-Aspartic acid
LKQINVIAGVKEPIRAYGCSANDA

SEQ ID NO: 478 - protein, 23 aa, synthetic construct
  SITE 18: note = D-Cysteine
  SITE 22: note = D-Aspatic acid
KQINVIAGVKEPIRAYGCSANDA

SEQ ID NO: 479 - protein, 22 aa, synthetic construct
  SITE 17: note = D-Cysteine
  SITE 21: note = D-Aspatic acid
QINVIAGVKEPIRAYGCSANDA

SEQ ID NO: 480 - protein, 21 aa, synthetic construct
  SITE 16: note = D-Cysteine
  SITE 20: note = D-Aspartic acid
INVIAGVKEPIRAYGCSANDA

SEQ ID NO: 481 - protein, 20 aa, synthetic construct
  SITE 15: note = D-Cysteine
  SITE 19: note = D-Aspartic acid
NVIAGVKEPIRAYGCSANDA

SEQ ID NO: 482 - protein, 19 aa, synthetic construct
  SITE 14: note = D-Cystenine
  SITE 18: note = D-Aspartic acid
VIAGVKEPIRAYGCSANDA

SEQ ID NO: 483 - protein, 18 aa, synthetic construct
  SITE 13: note = D-Cysteine
  SITE 17: note = D-Aspartic acid
IAGVKEPIRAYGCSANDA

SEQ ID NO: 484 - protein, 17 aa, synthetic construct
  SITE 12: note = D-Cysteine
  SITE 16: note = D-Aspartic acid
AGVKEPIRAYGCSANDA

SEQ ID NO: 485 - protein, 16 aa, synthetic construct
  SITE 11: note = D-Cysteine
  SITE 15: note = D-Aspartic acid
GVKEPIRAYGCSANDA

SEQ ID NO: 486 - protein, 15 aa, synthetic construct
  SITE 10: note = D-Cysteine
  SITE 14: note = D-Aspatic acid
VKEPIRAYGCSANDA

SEQ ID NO: 487 - protein, 14 aa, synthetic construct
  SITE 9: note = D-Cysteine
  SITE 13: note = D-Aspartic acid
KEPIRAYGCSANDA

SEQ ID NO: 488 - protein, 13 aa, synthetic construct
  SITE 8: note = D-Cysteine
  SITE 12: note = D-Aspartic acid
EPIRAYGCSANDA

SEQ ID NO: 489 - protein, 12 aa, synthetic construct
  SITE 7: note = D-Cysteine
  SITE 11: note = D-Aspartic acid
PIRAYGCSANDA

SEQ ID NO: 490 - protein, 11 aa, synthetic construct
  SITE 6: note = D-Cysteine
  SITE 10: note = D-Aspartic acid
IRAYGCSANDA

SEQ ID NO: 491 - protein, 10 aa, synthetic construct
  SITE 5: note = D-Cysteine
  SITE 9: note = D-Aspartic acid
RAYGCSANDA

SEQ ID NO: 492 - protein, 9 aa, synthetic construct
  SITE 4: note = D-Cysteine
  SITE 9: note = D-Aspartic acid
AYGCSANDA

SEQ ID NO: 493 - protein, 8 aa, synthetic construct
  SITE 3: note = D-Cysteine
  SITE 7: note = D-Aspartic acid
YGCSANDA

SEQ ID NO: 494 - protein, 7 aa, synthetic construct
  SITE 2: note = D-Cysteine
  SITE 6: note = D-Aspartic acid
GCSANDA

SEQ ID NO: 495 - protein, 6 aa, synthetic construct
  SITE 1: note = D-Cysteine
  SITE 5: note = D-Aspartic acid
CSANDA

SEQ ID NO: 496 - protein, 5 aa, synthetic construct
  SITE 4: note = D-Aspartic acid
SANDA

SEQ ID NO: 497 - protein, 4 aa, synthetic construct
  SITE 3: note = D-Aspartic acid
ANDA

SEQ ID NO: 498 - skipped

SEQ ID NO: 499 - protein, 19 aa, synthetic construct
LKQINVIAGVKEPIRAYGF

SEQ ID NO: 500 - protein, 20 aa, synthetic construct
LKQINVIAGVKEPIRAYGFC

SEQ ID NO: 501 - protein, 21 aa, synthetic construct
LKQINVIAGVKEPIRAYGFCF

SEQ ID NO: 502 - protein, 22 aa, synthetic construct
LKQINVIAGVKEPIRAYGFCFA

SEQ ID NO: 503 - protein, 23 aa, synthetic construct
LKQINVIAGVKEPIRAYGFCFAK

SEQ ID NO: 504 - protein, 24 aa, synthetic construct
LKQINVIAGVKEPIRAYGFCFAKT

SEQ ID NO: 505 - protein, 25 aa, synthetic construct
LKQINVIAGVKEPIRAYGFCFAKTE

SEQ ID NO: 506 - protein, 26 aa, synthetic construct
LKQINVIAGVKEPIRAYGFCFAKTET

SEQ ID NO: 507 - protein, 27 aa, synthetic construct
LKQINVIAGVKEPIRAYGFCFAKTETA

SEQ ID NO: 508 - protein, 26 aa, synthetic construct
KQINVIAGVKEPIRAYGFCFAKTETA

SEQ ID NO: 509 - protein, 25 aa, synthetic construct
QINVIAGVKEPIRAYGFCFAKTETA

SEQ ID NO: 510 - protein, 24 aa, synthetic construct
INVIAGVKEPIRAYGFCFAKTETA

SEQ ID NO: 511 - protein, 23 aa, synthetic construct
NVIAGVKEPIRAYGFCFAKTETA

SEQ ID NO: 512 - protein, 22 aa, synthetic construct
VIAGVKEPIRAYGFCFAKTETA

SEQ ID NO: 513 - protein, 21 aa, synthetic construct
IAGVKEPIRAYGFCFAKTETA

SEQ ID NO: 514 - protein, 20 aa, synthetic construct
AGVKEPIRAYGFCFAKTETA

SEQ ID NO: 515 - protein, 19 aa, synthetic construct
GVKEPIRAYGFCFAKTETA

SEQ ID NO: 516 - protein, 18 aa, synthetic construct
VKEPIRAYGFCFAKTETA

SEQ ID NO: 517 - protein, 17 aa, synthetic construct
KEPIRAYGFCFAKTETA

SEQ ID NO: 518 - protein, 16 aa, synthetic construct
EPIRAYGFCFAKTETA

SEQ ID NO: 519 - protein, 15 aa, synthetic construct
PIRAYGFCFAKTETA

SEQ ID NO: 520 - protein, 14 aa, synthetic construct
IRAYGFCFAKTETA

SEQ ID NO: 521 - protein, 13 aa, synthetic construct
RAYGFCFAKTETA

SEQ ID NO: 522 - protein, 12 aa, synthetic construct
AYGFCFAKTETA

SEQ ID NO: 523 - protein, 11 aa, synthetic construct
YGFCFAKTETA

SEQ ID NO: 524 - protein, 10 aa, synthetic construct
GFCFAKTETA

SEQ ID NO: 525 - protein, 9 aa, synthetic construct
FCFAKTETA

SEQ ID NO: 526 - protein, 8 aa, synthetic construct
CFAKTETA

SEQ ID NO: 527 - protein, 7 aa, synthetic construct
FAKTETA

SEQ ID NO: 528 - protein, 6 aa, synthetic construct
AKTETA

SEQ ID NO: 529 - protein, 5 aa, synthetic construct
KTETA

SEQ ID NO: 530 - protein, 4 aa, synthetic construct
TETA

SEQ ID NO: 531 - protein, 4 aa, synthetic construct
TETA

SEQ ID NO: 532 - protein, 18 aa, synthetic construct
LKQINVIAGVKEPIRAYE

SEQ ID NO: 533 - protein, 19 aa, synthetic construct
LKQINVIAGVKEPIRAYEN

SEQ ID NO: 534 - protein, 20 aa, synthetic construct
LKQINVIAGVKEPIRAYENL

SEQ ID NO: 535 - protein, 21 aa, synthetic construct
LKQINVIAGVKEPIRAYENLY

SEQ ID NO: 536 - protein, 22 aa, synthetic construct
LKQINVIAGVKEPIRAYENLYF

SEQ ID NO: 537 - protein, 23 aa, synthetic construct
LKQINVIAGVKEPIRAYENLYFQ

SEQ ID NO: 538 - protein, 24 aa, synthetic construct
LKQINVIAGVKEPIRAYENLYFQF

SEQ ID NO: 539 - protein, 25 aa, synthetic construct
LKQINVIAGVKEPIRAYENLYFQFC

SEQ ID NO: 540 - protein, 26 aa, synthetic construct
LKQINVIAGVKEPIRAYENLYFQFCF

SEQ ID NO: 541 - protein, 27 aa, synthetic construct
LKQINVIAGVKEPIRAYENLYFQFCFW

SEQ ID NO: 542 - protein, 28 aa, synthetic construct
LKQINVIAGVKEPIRAYENLYFQFCFWK

SEQ ID NO: 543 - protein, 29 aa, synthetic construct
LKQINVIAGVKEPIRAYENLYFQFCFWKT

SEQ ID NO: 544 - protein, 30 aa, synthetic construct
LKQINVIAGVKEPIRAYENLYFQFCFWKTE

SEQ ID NO: 545 - protein, 31 aa, synthetic construct
LKQINVIAGVKEPIRAYENLYFQFCFWKTET

SEQ ID NO: 546 - protein, 30 aa, synthetic construct
KQINVIAGVKEPIRAYENLYFQFCFWKTET

SEQ ID NO: 547 - protein, 29 aa, synthetic construct
QINVIAGVKEPIRAYENLYFQFCFWKTET

SEQ ID NO: 548 - protein, 28 aa, synthetic construct
INVIAGVKEPIRAYENLYFQFCFWKTET

SEQ ID NO: 549 - protein, 27 aa, synthetic construct
NVIAGVKEPIRAYENLYFQFCFWKTET

SEQ ID NO: 550 - protein, 26 aa, synthetic construct
VIAGVKEPIRAYENLYFQFCFWKTET

SEQ ID NO: 551 - protein, 25 aa, synthetic construct
IAGVKEPIRAYENLYFQFCFWKTET

SEQ ID NO: 552 - protein, 24 aa, synthetic construct
AGVKEPIRAYENLYFQFCFWKTET

SEQ ID NO: 553 - protein, 23 aa, synthetic construct
GVKEPIRAYENLYFQFCFWKTET

SEQ ID NO: 554 - protein, 22 aa, synthetic construct
VKEPIRAYENLYFQFCFWKTET

SEQ ID NO: 555 - protein, 21 aa, synthetic construct
KEPIRAYENLYFQFCFWKTET

SEQ ID NO: 556 - protein, 20 aa, synthetic construct
EPIRAYENLYFQFCFWKTET

SEQ ID NO: 557 - protein, 19 aa, synthetic construct
PIRAYENLYFQFCFWKTET

SEQ ID NO: 558 - protein, 18 aa, synthetic construct
IRAYENLYFQFCFWKTET

SEQ ID NO: 559 - protein, 17 aa, synthetic construct
RAYENLYFQFCFWKTET

SEQ ID NO: 560 - protein, 16 aa, synthetic construct
AYENLYFQFCFWKTET

SEQ ID NO: 561 - protein, 15 aa, synthetic construct
YENLYFQFCFWKTET

SEQ ID NO: 562 - protein, 14 aa, synthetic construct
ENLYFQFCFWKTET

SEQ ID NO: 563 - protein, 13 aa, synthetic construct
NLYFQFCFWKTET

SEQ ID NO: 564 - protein, 12 aa, synthetic construct
LYFQFCFWKTET

SEQ ID NO: 565 - protein, 11 aa, synthetic construct
YFQFCFWKTET

SEQ ID NO: 566 - protein, 10 aa, synthetic construct
FQFCFWKTET

SEQ ID NO: 567 - protein, 9 aa, synthetic construct
QFCFWKTET

SEQ ID NO: 568 - protein, 8 aa, synthetic construct
FCFWKTET

SEQ ID NO: 569 - protein, 7 aa, synthetic construct
CFWKTET

SEQ ID NO: 570 - protein, 6 aa, synthetic construct
FWKTET

SEQ ID NO: 571 - protein, 5 aa, synthetic construct
WKTET

SEQ ID NO: 572 - protein, 4 aa, synthetic construct
KTET

(19) United States
(12) Patent Application Publication
(10) Pub. No.: US 2025/0376709 A1
(43) Pub. Date: Dec. 11, 2025

(54) PAPB AS A BIMOIETY-DEPENDENT THIOETHER INSTALLATION TOOL

(71) Applicant: UNIVERSITY OF UTAH RESEARCH FOUNDATION, Salt Lake City, UT (US)

(72) Inventors: Vahe Bandarian, Salt Lake City, UT (US); Karsten A.S. Eastman, Salt Lake City, UT (US); Andrew George Roberts, Salt Lake City, UT (US)

(21) Appl. No.: 18/855,930

(22) PCT Filed: Apr. 14, 2023
(86) PCT No.: PCT/US2023/065825

§ 371 Date: Oct. 10, 2024

Related U.S. Application Data
(60) Provisional application No. 63/331,393, filed on Apr. 15, 2022.
(60) Provisional application No. 63/337,029, filed on Apr. 29, 2022.
(60) Provisional application No. 63/393,174, filed on Jul. 28, 2022.
(60) Provisional application No. 63/446,589, filed on Feb. 17, 2023.

(51) Int. Cl. C12P 21/06; C12N 9/50

(52) U.S. Cl. CPC C12P 21/06; C12N 9/50

(57) ABSTRACT

The present disclosure is concerned with methods of chemically modifying a peptide sequence to install a thioether linkage, the method comprising reacting the peptide sequence with PapB. Also disclosed are compounds produced by such methods that may be useful in, for example, peptide therapeutic uses. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 63/446,589, filed on Feb. 17, 2023, U.S. Provisional Application No. 63/393,174, filed on Jul. 28, 2022, U.S. Provisional Application No. 63/337,029, filed on Apr. 29, 2022, and U.S. Provisional Application No. 63/331,393, filed on Apr. 15, 2022, the contents of which are incorporated herein by reference in their entireties.

### STATEMENT REGARDING FEDERALLY SPONSORED RESEARCH

This invention was made with government support under Grant No. GM126956 awarded by the National Institutes of Health. The government has certain rights in the invention.

### REFERENCE TO SEQUENCE LISTING

The Sequence Listing submitted Apr. 14, 2023 as a xml file named “21101.0436P1.xml,” created on Apr. 14, 2023, and having a size of 16,384 bytes is hereby incorporated by reference pursuant to 37 C.F.R. § 1.52 (e) (5).

### BACKGROUND

Peptide-based therapeutics are growing due to their unique structure and ability to be produced via solid phase peptide synthesis (SPPS) or by recombinant DNA. Many peptide therapeutics contain a disulfide bond in their active form. Disulfide bonds are susceptible to breakage via biological reductants such as glutathione. Additionally, many peptide therapeutics contain bulky or basic amino acid side chains which render them vulnerable to degradation by proteases. These factors contribute to their short serum half-lives. Strategies such as L-to-D amino acid swaps, derivatization of the N- and C-termini, N-to-C-terminal cyclization, the introduction of non-proteinogenic amino acids, and metal chelation have both increased peptide half-lives and diversified therapeutic targets. The extent of these modifications is limited to the chemical space afforded by organic synthesis and SPPS.

Nature can access vast chemical space through enzymatic reactions. Natural products are incredibly diverse in their structures which allow for their wide range of biological and chemical activities. Recent advances in bioinformatic filtering algorithms have uncovered previously unannotated small open reading frames (sORFs). sORFs often colocalize with maturases which further process the peptide after translation. These ribosomally synthesized and post-translationally modified peptides (RiPPs) vary significantly in peptide length, structure, and biological function. RiPP maturases include members of the radical S-adenosylmethionine (rSAM) superfamily. This superfamily has been implicated in a variety of RiPP modifications, including C—C, C—N, C—O and C—S bond formation at unactivated carbons via radical mechanisms. These molecular mechanisms are of substantial interest because they afford access to unique semi-synthetic chemical spaces for production of bioinspired peptide therapeutics. RiPP maturases have potential to offer biotechnological applications in peptide alterations such as thioether installation or peptide stapling. rSAM enzymes use a radical intermediate to complete chemical transformations involved in natural product biosynthesis as well as primary metabolism. These enzymes contain one or more iron-sulfur [Fe—S] clusters that are essential for function. The [4Fe-4S] rSAM (RS) cluster is coordinated by a canonical CxxxCxxC motif in the enzyme. In the [4Fe-4S] RS cluster, one iron coordinates the α-amino and α-carboxylate moieties of SAM. When the RS cluster is catalytically active, it transfers an electron to bound SAM. Either chemical or biological reducing systems are useful for product turnover because the RS cluster is catalytically inactive in the +2 state. Homolytic cleavage of SAM forms the reactive 5′-deoxyadenosyl radical (5′-dAdo, FIG. 1). 5′-dAdo′ acts as a radical initiator by abstracting a hydrogen atom from a specific site on the substrate, thereby forming 5′-deoxyadenosine (5′-dAdoH, FIG. 1) and a theoretical RiPP radical intermediate. The formed substrate radical is useful for substrate maturation. While only one [4Fe-4S] cluster is needed for reductive SAM cleavage, many rSAM enzymes also employ one or more auxiliary iron-sulfur clusters (ACs) for substrate turnover (FIG. 4c). These ACs are coordinated to the enzyme by cysteine-rich C-terminal extensions from the RS canonical motif (FIG. 2). Recent studies have characterized rSAM maturases with multiple [Fe—S] clusters that form intrapeptide bonds between Cα, Cβ, or Cγ on a specific residue and a cysteine thiol in the peptide substrate. Many of these thioether assembling maturases only form a single thioether in the mature peptide and are relatively slow in substrate turnover. The RS cluster in addition to at least one AC cluster is necessary for thioether formation. rSAM RiPP maturases also use a critical RiPP Recognition Element (RRE), that is responsible for binding to the leader sequence of the immature peptide (FIG. 2, left).

PapB is a RiPP maturase that catalyzes the insertion of six thioether crosslinks in the PapA polypeptide. PapB catalyzes the insertion of links between the Cys thiol and the b-carbon of the Asp, where the residues being linked are in a CX₃D motif. Prior studies have shown that the enzyme can also accept Glu at the modification site, and that PapB introduces the crosslink to the chemically analogous γ-carbon. In addition, PapB has also been shown to accept a shorter minimal substrate (msPapA), which only has a single pair of crosslinking amino acids in the CX₃D motif. PapB can catalyze both Cβ and Cγ thioether linkages, and forms six thioether linkages in the wild type PapA. PapB contains a RS cluster and two ACs (FIG. 2). Replacing Asp residue(s) to Glu residue(s) in WT-PapA still results in successful crosslinking. Both Cβ and Cγ thioether linkages were confirmed by 2D NMR.

Despite the emergence of various techniques in peptide-based therapeutics, there remains a need in the art for enzymatic systems for rapid and highly specific modification of a broad range of peptide substances to obtain natural products that are unattainable by traditional synthetic chemistry methods. These needs and others are addressed herein.

### SUMMARY

In accordance with the purpose(s) of the invention, as embodied and broadly described herein, the invention, in one aspect, relates to methods of chemically modifying a peptide sequence to install one or more thioether linkages. Additionally disclosed are compounds formed using methods of chemically modifying a peptide sequence. Also disclosed are methods of chemically modifying a modified PapA sequence, and compounds formed using methods of chemically modifying a modified PapA sequence.

Disclosed are methods of chemically modifying a compound to install a thioether linkage, the method comprising reacting the compound with PapB, wherein the compound has a structure represented by a formula:

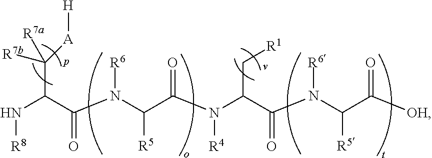

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

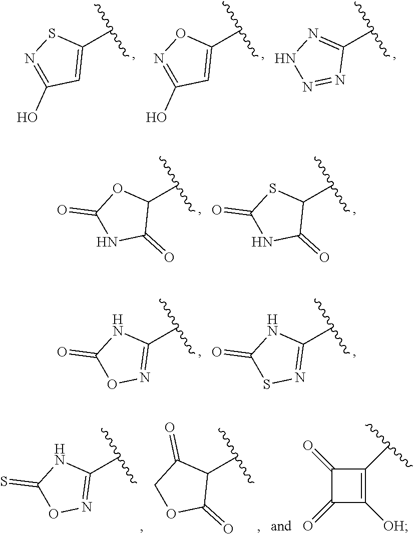

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are methods of chemically modifying a compound to install a thioether linkage, the method comprising reacting the compound with PapB, wherein the compound has a structure represented by a formula:

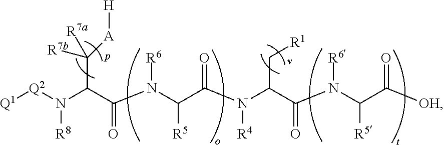

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein Q¹ is a leader sequence; wherein Q² is a cleavable moiety; wherein R¹ is selected from —CO₂H, C(O)NHOH, —SO₂NH₂, —SO₂NHC(O)CH₃, —SO₃H, —NHC(O)NHSO₂CH₃, —P(O)(OH)₂, and a structure selected from:

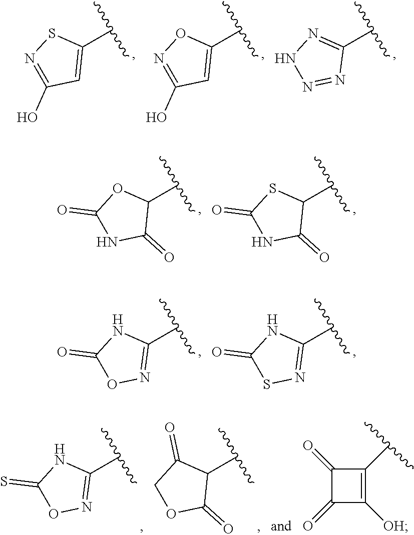

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are methods of chemically modifying a compound to install a thioether linkage, the method comprising reacting the compound with PapB, wherein the compound has a structure represented by a formula:

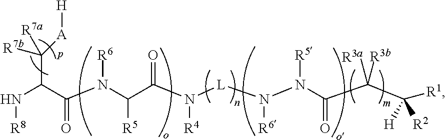

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, (C1-C4 alkyl)(OCH₂CH₂)_{q}, and a structure selected from:

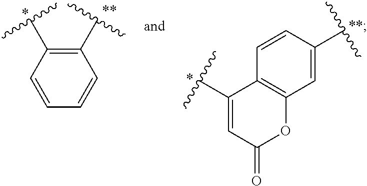

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

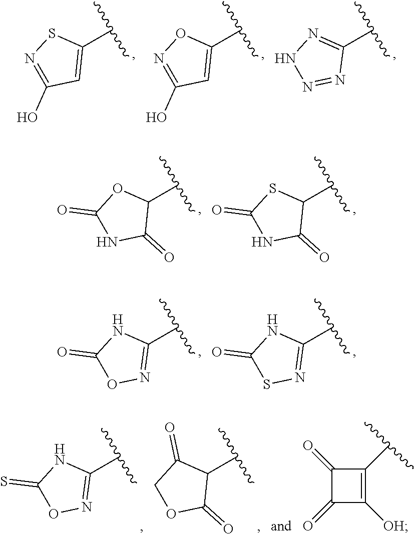

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are methods of chemically modifying a compound to install a thioether linkage, the method comprising reacting the compound with PapB, wherein the compound has a structure represented by a formula:

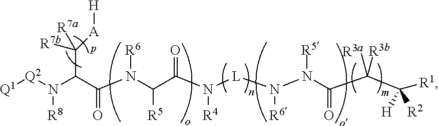

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl) (OCH₂CH₂)_{q}, and a structure selected from:

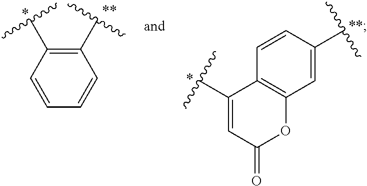

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

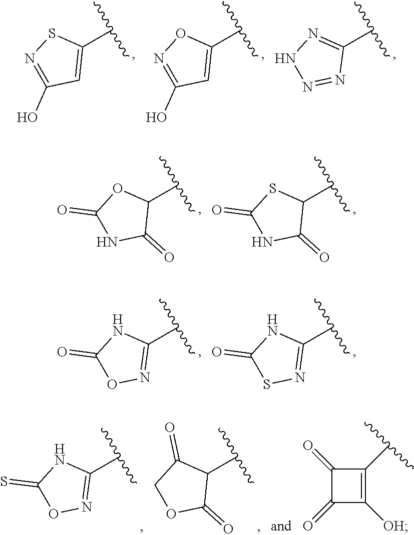

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are methods of chemically modifying a peptide sequence to install a thioether linkage, the method comprising reacting the peptide sequence with PapB, wherein the peptide sequence comprises X—Yₙ—Z, wherein n is 0, 1, 2, 3, 4, 5, 6, 7, 8, or 9; wherein X is an amino acid residue comprising a —SH or —SeH group; wherein each occurrence of Y, when present, is independently an amino acid residue; and wherein Z is an amino acid residue that is carboxyl-functionalized or tetrazolyl-functionalized, provided that the peptide sequence is not PapA.

Also disclosed are methods of chemically modifying a peptide sequence to install a thioether linkage, the method comprising reacting the peptide sequence with PapB, wherein the peptide sequence comprises X—Yₙ—Z; wherein X is a penicillamine or an amino acid residue comprising a —SH group or an amino acid residue comprising a —SeH group; wherein Y is a series of amino acid residues where n=0, 1, 2, 3, 4, 5, 6, 7, 8, or 9; wherein Z is an aspartic acid residue, a glutamic acid residue, a hydroxy-glutamic acid residue, 2-amino-3-(2H-tetrazol-5-yl) propanoic acid, or a carboxyl-functionalized amino acid residue; and wherein the peptide sequence is not PapA.

Also disclosed are methods of chemically modifying a modified PapA sequence to install a thioether linkage, the method comprising reacting the modified PapA sequence with PapB; wherein the modified PapA sequence comprises Cys-Yₙ-Asp, wherein Y is a series of amino acid residues and n=0, 1, 2, 4, 5, 6, or 7.

Also disclosed are thioether compounds produced by a disclosed method.

Also disclosed are methods of chemically modifying a modified PapA sequence to install a thioether linkage, the method comprising reacting the modified PapA sequence with PapB, wherein the modified PapA sequence comprises Cys-Yₙ-Asp, wherein Y is a series of amino acid residues, and wherein n is 0, 1, 2, 4, 5, 6, or 7.

Also disclosed are compounds produced by a disclosed method.

Also disclosed are compounds having a structure selected from:

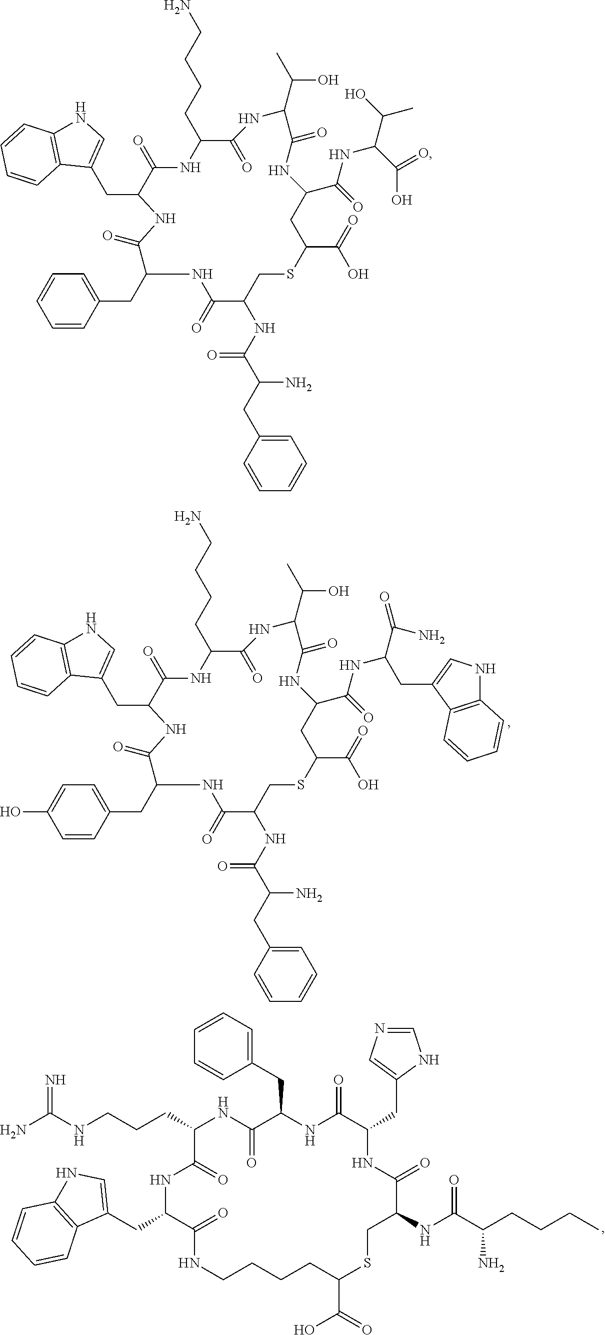

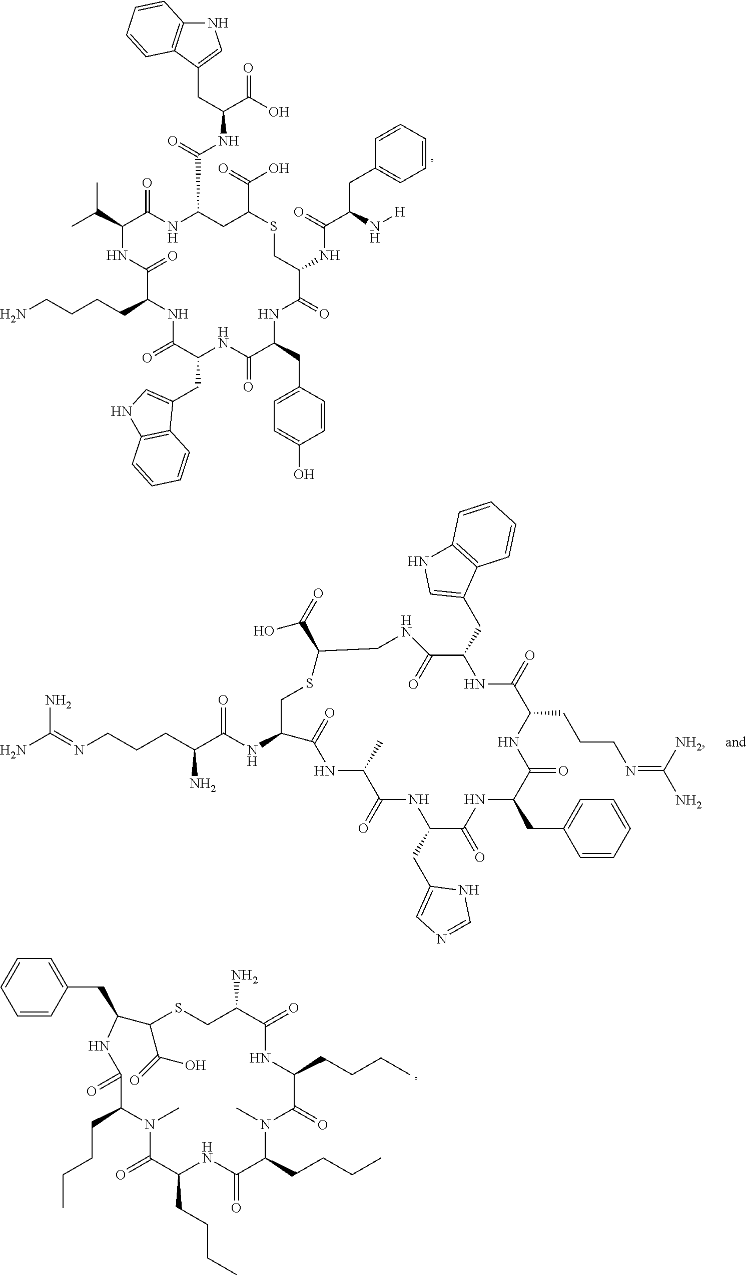 [SMILES: CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSC(C(=O)O)C[C@@H](C(=O)N[C@@H](Cc2c[nH]c3ccccc23)C(=O)O)NC1=O ; C[C@H]1NC(=O)[C@@H](NC(=O)[C@@H](N)CCCN=C(N)N)CS[C@@H](C(=O)O)CNC(=O)[C@H](Cc2c[nH]c3ccccc23)NC(=O)[C@H](CCCN=C(N)N)NC(=O)[C@@H](Cc2ccccc2)NC(=O)[C@H](Cc2cnc[nH]2)NC1=O ; CCCC[C@@H]1NC(=O)[C@@H](N)CSC(C(=O)O)[C@H](Cc2ccccc2)NC(=O)[C@H](CCCC)N(C)C(=O)[C@H](CCCC)NC(=O)[C@H](CCCC)N(C)C1=O]

or a pharmaceutically acceptable salt thereof.

Also disclosed are compounds selected from:

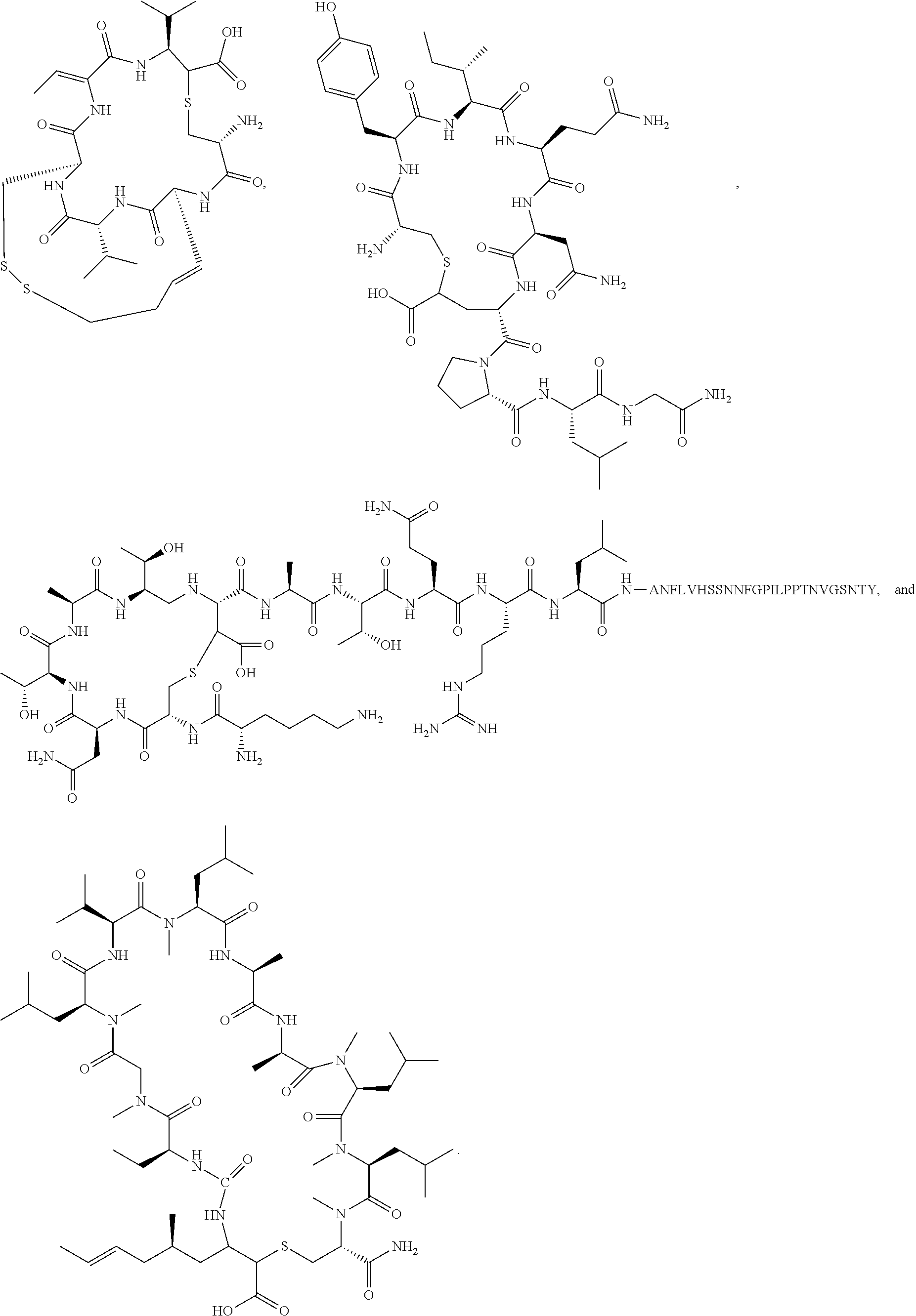

or a pharmaceutically acceptable salt thereof.

Also disclosed are pharmaceutical compositions comprising an effective amount of a disclosed compound or a pharmaceutically acceptable salt thereof and a pharmaceutically acceptable carrier.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings.

FIG. 1 is a schematic showing the proposed mechanism for beta-thioether crosslink.

FIG. 2 is a scheme showing the predicted structure of PapB.

FIG. 3 is a representative image showing SDS-PAGE analysis of reconstituted and purified PapB on a 12% crosslinked gel.

FIG. 4A and FIG. 4B show representative crosslinking data of minimal substrate PapA (msPapA) with PapB. Specifically, FIG. 4A shows representative TIC of msPapA chromatographed on C18 HPLC column (top left spectra). The peptide elutes at 8.1 min. A representative mass spectrum corresponding to the peak eluting at 8.1 min is shown at the bottom. The z=3 charge state was chosen for most peptide mass envelope comparisons. A representative mass spectrum comparison of the z=3 charge state envelopes of unreacted and reacted msPapA±PapB is shown at the top right. FIG. 4B shows the sequence of crosslinked PapB showing all of the observed b- and y-ions from tandem mass spectrometry.

FIG. 5 is a representative plot showing the comparison of activity of PapB processing Y17W msPapA with dithionite or FldA/FPR/NADPH.

FIG. 6 shows representative mass spectra demonstrating the effect of 2× and 4× enzyme concentration.

FIG. 7 shows representative mass spectra demonstrating the effect of 2× and 4× peptide concentration.

FIG. 8A-D show representative data for the Leader-C(X₀-X₆)D(Xₘ) crosslink formation. Specifically, FIG. 8A is a scheme showing the unmodified and modified peptide sequence illustrate the thioether crosslink based on the msPapA modification reported by Precord et al. FIG. 8B shows representative mass spectra for CX₀D-CX₂D PapB modification. FIG. 8C shows representative mass spectra for CX₄D-CX₆D PapB modification. FIG. 8D are schematics showing that the expected 2 Da loss is seen in each b and y fragment in the tandem mass spectrometry.

FIG. 9A-B show representative data for the iodoacetic acid treatment for CX₀D. Specifically, FIG. 9A shows representative mass spectra data for CX₀D without PapB. FIG. 9B shows shows representative mass spectra data for CX₀D with PapB.

FIG. 10A-B show representative data for the iodoacetic acid treatment for CX₁D. Specifically, FIG. 10A shows representative mass spectra data for CX₀D without PapB. FIG. 10B shows shows representative mass spectra data for CX₁D with PapB.

FIG. 11A-B show representative data for the iodoacetic acid treatment for CX₂D. Specifically, FIG. 11A shows representative mass spectra data for CX₀D without PapB. FIG. 11B shows shows representative mass spectra data for CX₂D with PapB.

FIG. 12A-B show representative data for the iodoacetic acid treatment for CX₄D. Specifically, FIG. 12A shows representative mass spectra data for CX₀D without PapB. FIG. 12B shows shows representative mass spectra data for CX₄D with PapB.

FIG. 13A-B show representative data for the iodoacetic acid treatment for CX₅D. Specifically, FIG. 13A shows representative mass spectra data for CX₀D without PapB. FIG. 13B shows shows representative mass spectra data for CX₅D with PapB.

FIG. 14A-B show representative data for the iodoacetic acid treatment for CX₆D. Specifically, FIG. 14A shows representative mass spectra data for CX₀D without PapB. FIG. 14B shows shows representative mass spectra data for CX₆D with PapB.

FIG. 15A-C show representative data for leader extensions with single, nested, and in-line crosslinks. Specifically, FIG. 15A are peptide schemes showing the apparent crosslink locations that remain consistent after distancing the thioether motifs from the leader peptide.

FIG. 15B are representative mass spectra showing the isotopic distributions of the peptides; a shift of 2 Da in the case of single thioether motifs or 4 Da with double thioether motifs upon addition of PapB. FIG. 13C are schematics showing a representation of the tandem mass spectrometry results.

FIG. 16A-B show representative data for the iodoacetic acid treatment for Leader-AAACSANDA. FIG. 16A shows representative mass spectra data for Leader-AAACSANDA without PapB. FIG. 16B shows shows representative mass spectra data for Leader-AAACSANDA with PapB.

FIG. 17A-B show representative data for the iodoacetic acid treatment for Leader-AAACSANDACSANDA. FIG. 17A shows representative mass spectra data for Leader-AAACSANDACSANDA without PapB. FIG. 17B shows shows representative mass spectra data for Leader-AAACSANDACSANDA with PapB.

FIG. 18A-B show representative data for the iodoacetic acid treatment for Leader-AAACSACDAADA. FIG. 18A shows representative mass spectra data for Leader-AAACSACDAADA without PapB. FIG. 18B shows shows representative mass spectra data for Leader-AAACSACDAADA with PapB.

FIG. 19A-B show representative data for the iodoacetic acid treatment for Leader-AAAASACDAADA. FIG. 19A shows representative mass spectra data for Leader-AAAASACDAADA without PapB. FIG. 19B shows shows representative mass spectra data for Leader-AAAASACDAADA with PapB.

FIG. 20A-B show representative data for the iodoacetic acid treatment for Leader-AAACSAADAADA. FIG. 20A shows representative mass spectra data for Leader-AAACSAADAADA without PapB. FIG. 20B shows shows representative mass spectra data for Leader-AAACSAADAADA with PapB.

FIG. 21A-C show representative data showing that PapB produces two thioether crosslinks in the AMK-1057 precursor peptide in vitro. FIG. 21A is a scheme showing that the AMK-1057 precursor peptide contains the leader peptide sequence, a TEV protease recognition sequence, and two CX₃E motifs. FIG. 21B shows representative mass spectra demonstrating that upon reaction with PapB in an in vitro assay, two crosslinks form. Additional processing with TEV protease produces the expected dicyclized peptide. FIG. 21C is a scheme demonstrating the topology of the bonds as confirmed by tandem mass spectrometry.

FIG. 22A-C show representative data for PapB crosslinking ^{D}C and ^{P}D msPapA Peptides. FIG. 22A is a scheme showing the thioether crosslink. FIG. 22B are representative mass spectra showing formation of the thioether crosslinks. FIG. 22C is a scheme demonstrating the topology of the bonds as confirmed by mass spectrometry.

FIG. 23A-B shows representative data for the iodoacetic acid treatment for Leader-^{D}CSANDA. FIG. 23A shows representative mass spectra data for Leader-^{D}CSANDA without PapB. FIG. 23B shows shows representative mass spectra data for Leader-^{D}CSANDA with PapB.

FIG. 24A-B show representative data for the iodoacetic acid treatment for Leader-CSAN^{D}DA. FIG. 24A shows representative mass spectra data for Leader-CSAN^{D}DA without PapB. FIG. 24B shows shows representative mass spectra data for Leader-CSAN^{D}DA with PapB.

FIG. 25A-B show representative data for the iodoacetic acid treatment for Leader-^{D}CSAN^{D}DA. FIG. 25A shows representative mass spectra data for Leader-^{D}CSAN^{D}DA without PapB. FIG. 25B shows shows representative mass spectra data for Leader-^{D}CSAN^{D}DA with PapB.

FIG. 26A-B show representative data for msPapA “DSANCA” peptides. FIG. 26A shows representative mass spectra data for Leader-DSANCA and Leader-^{D}DSANCA with and without PapB. FIG. 26B shows representative mass spectra data for Leader-DSAN^{D}CA and Leader-^{D}DSAN^{D}CA with and without PapB.

FIG. 27A-E show representative data for synthesis of an octreotide analog. FIG. 27A is a structure of the FDA-approved therapeutic octreotide. FIG. 27B is a schematic description of the designed peptides and the expected sites of modification upon modification with PapB. A TEV cleavage site is included in the second peptide to allow for liberation of the modified peptide sequence by PapB. FIG. 27C is representative mass spectra data showing the isotopic envelope of these peptides indicating that a mixed population of processed and unprocessed peptides are present after modification by PapB. FIG. 27D is representative mass spectra data showing that the TEV-cleaved peptide isotopic envelope reveals the anticipated 2 Da mass shift. FIG. 27E is a scheme showing the anticipated loss of 2 Da in each y fragment after the C and in each b fragment after the C-terminal E as confirmed by tandem mass spectrometry.

FIG. 28 is a structure of the synthesized thioether-linked octreotide analog.

FIG. 29 is a scheme providing a brief summary of successful PapB-mediated thioether crosslinks in tested peptide sequences.

FIG. 30 shows representative data demonstrating that the leader peptide sequence is not required for modification via PapB.

FIG. 31 shows representative mass spectrometry data for a one-to-one interpeptide crosslink as well as polymerization-like addition of X-mer subunits.

FIG. 32 shows representative mass spectrometry results for a general assay peptide before and after PapB, demonstrating the presence of interpeptide products.

FIG. 33 shows representative mass spectra data showing evidence of simple and complex mass envelopes.

FIG. 34 is a schematic showing the experimental approaches to creating modified insulin analogs using PapB.

FIG. 35 shows representative mass spectra data for the synthesized insulin analogs.

FIG. 36 shows representative mass spectra data for crosslinking in peptides containing EneA.

FIG. 37 shows representative tandem mass spectrometry data for dAdo+D24EneA msPapA adduct.

FIG. 38 shows representative data, including mass spectrometry and EXAFS, for crosslinking in selenopeptides.

FIG. 39 shows representative tandem mass spectrometry data for C19U msPapA.

FIG. 40 shows representative mass spectrometry data demonstrating that aspartic acid may be replaced with glutamic acid, and cysteine may be replaced with homocysteine. Crosslinking is observed.

FIG. 41 shows representative mass spectrometry data demonstrating that β-amino acids may be incorporated in the peptide. Crosslinking is observed.

FIG. 42 shows representative mass spectrometry data demonstrating that no crosslinking was observed when altering the position of the C and D residues.

FIG. 43 shows representative data demonstrating the effect of components in the reduction system employed.

FIG. 44 is a schematic summarizing the findings of experiments conducted using prereduced PapB.

FIG. 45 is a scatterplot showing representative data of % product as a function of time for prereduced PapB experiments.

FIG. 46 shows representative data, including photodiode array chromatography, UV-Vis, and extracted ion chromatography, for PapB with and without reductant, as well as prereduced PapB.

FIG. 47 is a concept schematic for a bioreactor setup for peptide modification via PapB.

FIG. 48A-B show representative data for C-terminal glycine sequence. FIG. 48A is a scheme showing the thioether crosslink. FIG. 48B are representative mass spectra showing formation of the thioether crosslinks.

FIG. 49A-B show representative data for deuterium labeled C-terminal glycine analogs. FIG. 49A is a scheme showing the thioether crosslink. FIG. 49B are representative mass spectra showing formation of the thioether crosslinks.

FIG. 50A-B show representative data for C-terminal glycine carboxamide sequence. FIG. 50A is the structure of the sequence. FIG. 50B are representative mass spectra showing lack of formation of the thioether crosslinks.

FIG. 51A-C show representative data for crosslinking with C-terminal β-amino acids. FIG. 50A is a scheme showing the generic thioether crosslink reaction for C-terminal β-amino acids. FIG. 50B is a scheme showing the thioether crosslink reaction with C-terminal β-alanine. FIG. 51C is the corresponding mass spectra data showing formation of the thioether crosslink.

FIG. 52A-D show representative data for the crosslinking with various C-terminal β-amino acids. FIG. 52A is a scheme showing the absence of crosslink reaction with C-terminal 2,2-dimethyl-beta-alanine. FIG. 52B is a scheme showing the absence of crosslink reaction with C-terminal (R)-3-amino-2-methylpropanoic acid. FIG. 52C is a scheme showing the crosslink reaction with C-terminal(S)-3-amino-2-methylpropanoic acid. FIG. 52D is the corresponding mass spectra data showing formation of the thioether crosslink.

FIG. 53 shows representative data for the crosslinking with common C-terminal β-amino acids.

FIG. 54A shows a schematic thioether crosslinking with a D-tryptophan β-amino acid. FIG. 54B is the corresponding mass spectra data showing formation of the thioether crosslink

FIG. 55A-D show representative structures of thioether crosslinking of N-methyl amino acids. FIG. 55A shows unsubstituted N-methylated thioether crosslinked product. FIG. 55B shows substituted N-methylated thioether crosslinked product. FIG. 55C shows a schematic thioether crosslinking with a substituted N-methylated substrate. FIG. 55D is the corresponding mass spectra data showing formation of the thioether crosslink.

FIG. 56A-D show representative data for thioether crosslinking with C-terminal L-alanine or D-alanine. FIG. 56A shows a schematic of a C-terminal L-alanine without thioether crosslink product. FIG. 58B is the corresponding mass spectra data showing lack of formation of the thioether crosslink. FIG. 56C shows a schematic of a C-terminal D-alanine with thioether crosslink product. FIG. 56D is the corresponding mass spectra data showing formation of the thioether crosslink.

FIG. 57A-B show representative data for thioether crosslinking with deuterium labeled C-terminal D-alanine. FIG. 57A shows a schematic of a deuterium labeled C-terminal D-alanine with thioether crosslink product. FIG. 57B is the corresponding mass spectra data showing formation of the thioether crosslink and loss of the deuterium labeled confirmed by mass shift and loss 3 Da.

FIG. 58A-B show representative data for thioether crosslinking with deuterium labeled C-terminal D-methionine. FIG. 58A shows a schematic of a deuterium labeled C-terminal D-methionine with thioether crosslink product. FIG. 58B is the corresponding mass spectra data showing formation of the thioether crosslink and loss of the deuterium labeled confirmed by mass shift and loss 3 Da.

FIG. 59A-B show representative data for thioether crosslinking with d2-labeled D-valine. FIG. 59A shows a structure of a deuterium labeled C-terminal D-valine. FIG. 59B is the corresponding mass spectra data showing formation of the thioether crosslink however mass shift is indicative of no loss of deuterium.

FIG. 60A-B show representative data for thioether crosslinking with d3-labeled D-valine. FIG. 60A shows a schematic of a deuterium labeled side chain C-terminal D-valine with thioether crosslink product. FIG. 60B is the corresponding mass spectra data showing formation of the thioether crosslink and loss of the deuterium labeled confirmed by mass shift and loss 3 Da.

FIG. 61A-D show representative data for thioether crosslinking with deuterium labeled C-terminal D-phenyl alanine. FIG. 61A shows a structure of a deuterium labeled Cα C-terminal D-phenyl alanine. FIG. 61B is the corresponding mass spectra data showing formation of the thioether crosslink however mass shift is indicative of no loss of deuterium. FIG. 61C shows a structure of a deuterium labeled aryl C-terminal D-phenyl alanine. FIG. 61D is the corresponding mass spectra data showing formation of the thioether crosslink however mass shift is indicative of no loss of deuterium

FIG. 62A-B show representative data for thioether crosslinking with deuterium labeled d8-C-terminal D-phenylalanine. FIG. 62A shows a schematic of a deuterium labeled d8-C-terminal D-methionine with thioether crosslink product. FIG. 62B is the corresponding mass spectra data showing formation of the thioether crosslink and loss of the deuterium labeled confirmed by mass shift.

FIG. 63 shows structures of sactipeptide thioether crosslink of corresponding D-aminoacids

FIG. 64 shows structures of ranthipeptide thioether crosslink of corresponding D-aminoacids.

FIG. 65A-B show representative data for 6-membered non-peptidic thioether crosslinking. FIG. 65A shows scheme of Leader-Cys-Gly reaction. FIG. 65B is the corresponding mass spectra data showing lack of formation of the thioether crosslink of 6-membered ring.

FIG. 66A-B show representative data for 7-membered non-peptidic thioether crosslinking. FIG. 66A shows scheme of Leader-hCys-Gly reaction. FIG. 66B is the corresponding mass spectra data showing formation of the thioether crosslink of 7-membered ring.

FIG. 67A-B show representative data for 7-membered non-peptidic thioether crosslinking. FIG. 67A shows scheme of Leader-Cys-βAla reaction. FIG. 67B is the corresponding mass spectra data showing formation of the thioether crosslink of 7-membered ring.

FIG. 68A-B show representative data for 8-membered non-peptidic thioether crosslinking. FIG. 68A shows scheme of Leader-hCys-βAla reaction. FIG. 68B is the corresponding mass spectra data showing formation of the thioether crosslink of 8-membered ring.

FIG. 69A-B show representative data for 8-membered non-peptidic thioether crosslinking. FIG. 69A shows scheme of Leader-Cys-GABA reaction. FIG. 69B is the corresponding mass spectra data showing formation of the thioether crosslink of 8-membered ring.

FIG. 70A-B show representative data for 9-membered non-peptidic thioether crosslinking. FIG. 70A shows scheme of Leader-hCys-GABA reaction. FIG. 70B is the corresponding mass spectra data showing formation of the thioether crosslink of 9-membered ring.

FIG. 71A-B show representative data for 16-membered non-peptidic thioether crosslinking. FIG. 71A shows scheme of Leader-hCys-NH-PEG₃-CO₂H reaction. FIG. 71A is the corresponding mass spectra data showing formation of the thioether crosslink of 16-membered ring.

FIG. 72A-B show representative data for 20-membered non-peptidic thioether crosslinking. FIG. 72A shows scheme of Leader-hCys-NH-PEG₄-CO₂H reaction. FIG. 72B is the corresponding mass spectra data showing formation of the thioether crosslink of 20-membered ring.

FIG. 73A-B show representative data for unusual non-peptidic thioether crosslinking. FIG. 73A shows scheme of Leader-Cys-Ser-Ala-Asn-2-(2-aminophenyl) acetic acid reaction.

FIG. 73B is the corresponding mass spectra data showing formation of the thioether crosslink of 17-membered ring.

FIG. 74A-B show representative data for unusual non-peptidic thioether crosslinking. FIG. 74A shows scheme of Leader-Cys-Ser-Ala-Asn-2-(2-(aminomethyl)phenyl) acetic acid reaction. FIG. 74B is the corresponding mass spectra data showing formation of the thioether crosslink of 18-membered ring.

FIG. 75A-B show representative data for coumarin thioether crosslinking. FIG. 75A shows scheme of Leader-Cys-coumarin reaction. FIG. 75B is the corresponding mass spectra data showing formation of the thioether crosslink of 12-membered ring.

FIG. 76A-C show representative data for the synthesis thioether peptidomimetic. FIG. 76A is a structure of Setmalanotide, an FDA approved drug. FIG. 76B shows a schematic thioether crosslinking with a modified peptide structure (e.g., an analog of Setmalanotide). FIG. 76C is the corresponding mass spectra data showing formation of the thioether crosslink.

FIG. 77A-D show representative data for the synthesis thioether peptidomimetic. FIG. 77A is a structure of a Novartis orally available peptide. FIG. 77B is a structure of the designed peptides (an analog of the therapeutic peptide from FIG. 77A) and the expected product upon modification with PapB. FIG. 77C shows a schematic thioether crosslinking with a modified peptide structure. FIG. 77D is the corresponding mass spectra data showing formation of the thioether crosslink.

FIG. 78A-D show representative therapeutic cyclic peptides that can be mimicked by a thioether crosslink peptide. FIG. 78A show the structure of a representative cyclic peptide, bremelanotide. FIG. 78B shows a representative structure of the thioether crosslinked product, an analog of bremelanotide, which contains the amino acid sequence norleucine, cysteine, D-phenylalanine, arginine, tryptophan, and epsilon-amino hexanoic acid (ACP). FIG. 78C shows a representative scheme of the Leader-XCDFRWZ XXX reaction.

FIG. 78D is the corresponding mass spectra data showing formation of the thioether crosslink of therapeutic analog.

FIG. 79A-E show representative data illustrating that PapB forms crosslinks in thiol- and carboxylate-containing extended sidechains. Specifically, FIG. 79A shows a generalized linear scenario of C19hCys msPapA in which n=CH₂ (Asp), (CH₂)₂ (Glu), or (CH₂)₃ (hGlu). FIG. 79B shows a 2 Da shift in the MS for the carboxylate-containing residue as Asp. FIG. 79C shows a 2 Da shift in the MS for the carboxylate-containing residue as Glu. FIG. 79D shows a 2 Da shift in the MS for the carboxylate-containing residue as homoGlu. FIG. 79E shows the MS for the liberated macrocyclized peptide core from the leader sequence following cleavage of the TEV protease recognition sequence with TEV protease.

FIG. 80 shows a representative proton NMR spectrum of the linear G(hC)SAN(hE)A peptide.

FIG. 81 shows a representative proton NMR spectrum of the cyclized G(hC)SAN(hE)A peptide.

FIG. 82 shows a representative ROESY spectrum of the linear G(hC)SAN(hE)A peptide.

FIG. 83 shows a representative ROESY spectrum of the cyclized G(hC)SAN(hE)A peptide.

FIG. 84A-C show representative data pertaining to a carboxylate isostere (tetrazole moiety) crosslinked by PapB. Specifically, FIG. 84A shows a schematic of the linear and cyclized peptide illustrating the putative crosslink location. FIG. 84B shows MS results illustrating a clear 2 Da loss between an assay without PapB (darker gray) and with the addition of PapB (lighter gray). FIG. 84C shows the expected tandem mass spectrometry with no fragmentation between Cys and T4Az.

FIG. 85 shows representative fragmentation of reacted D23T4Az msPapA variant.

FIG. 86 shows representative fragments of a tetrazole loss in the D23T4Az msPapA variant

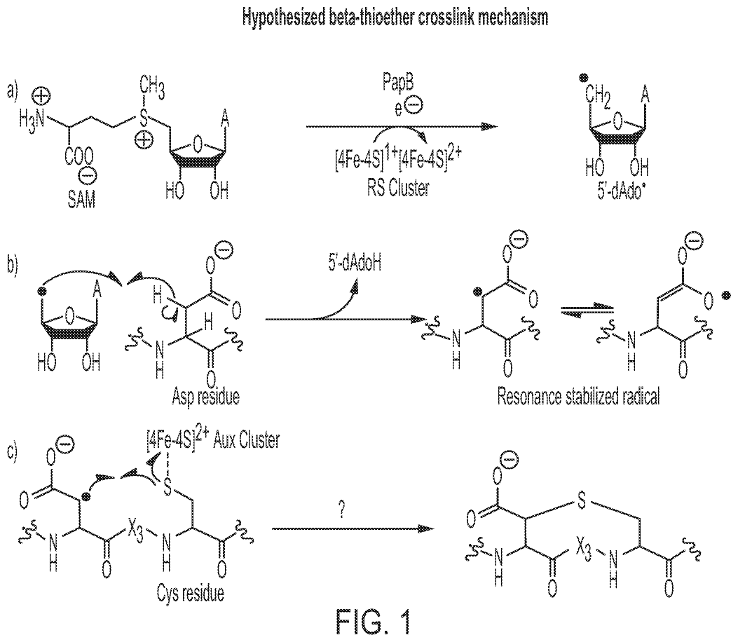

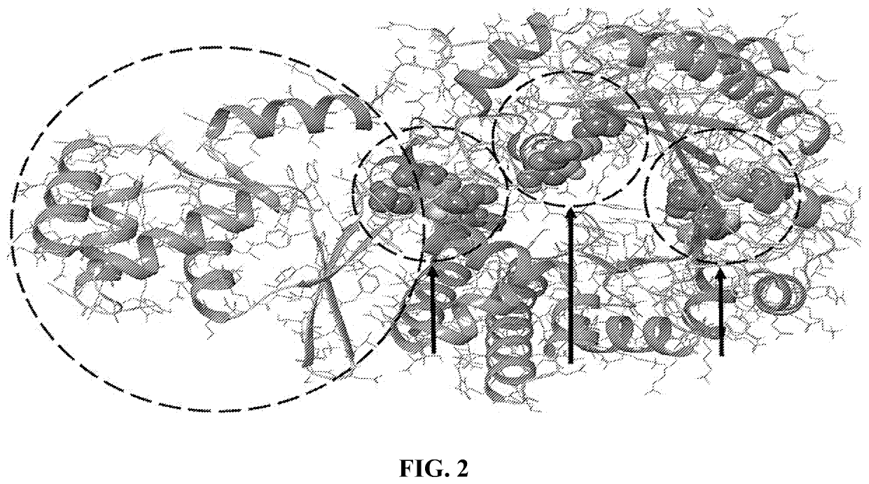

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half-maximal (50%) inhibitory concentration (IC) of a substance.

As used herein, “EC₅₀” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the term “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease, disorder, or condition. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the condition being treated and the severity of the condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-cancer and anti-neoplastic agents such as kinase inhibitors, poly ADP ribose polymerase (PARP) inhibitors and other DNA damage response modifiers, epigenetic agents such as bromodomain and extra-terminal (BET) inhibitors, histone deacetylase (HDAc) inhibitors, iron chelotors and other ribonucleotides reductase inhibitors, proteasome inhibitors and Nedd8-activating enzyme (NAE) inhibitors, mammalian target of rapamycin (mTOR) inhibitors, traditional cytotoxic agents such as paclitaxel, dox, irinotecan, and platinum compounds, immune checkpoint blockade agents such as cytotoxic T lymphocyte antigen-4 (CTLA-4) monoclonal antibody (mAB), programmed cell death protein 1 (PD-1)/programmed cell death-ligand 1 (PD-L1) mAB, cluster of differentiation 47 (CD47) mAB, toll-like receptor (TLR) agonists and other immune modifiers, cell therapeutics such as chimeric antigen receptor T-cell (CAR-T)/chimeric antigen receptor natural killer (CAR-NK) cells, and proteins such as interferons (IFNs), interleukins (ILs), and mAbs; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “sactipeptide” refers to a sulfur-to-alpha carbon thioether cross-linked peptide belonging to the ribosomally synthesized post-translationally modified peptide (RiPP) superfamily. As illustrated by the structure below, a sactipeptide contains an intramolecular thioether bond that crosslinks the sulfur atom of a cysteine residue to the α-carbon of an acceptor amino acid.

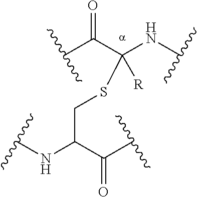

As used herein, the term “ranthipeptide” refers to a radical non-α thioether-containing peptide, which, similar to sactipeptides above, is also a member of the RiPP superfamily. For example, as illustrated below, a ranthipeptide can contain an intramolecular thioether bond that crosslinks the sulfur atom of a cysteine residue to any carbon other than the α-carbon of an acceptor amino acid.

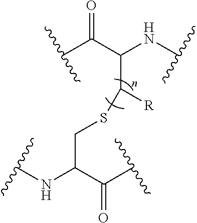

Exemplary ranthipeptide residues containing an β- or γ-carbon are shown below.

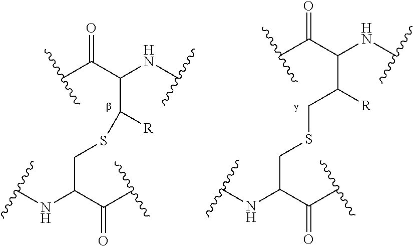

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the I clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O) H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide” as used herein can be used interchangeably and refer to F, Cl, Br, or I.

The terms “pseudohalide,” “pseudohalogen,” or “pseudohalo” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein, refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein, refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas-S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; —(CH₂)₀₋₄N(R^{◯})C(O)OR^{◯}; —N(R^{◯})N(R^{◯})C(O)R^{◯}; —N(R^{◯})N(R^{◯})C(O)NR^{◯}₂; —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)SR^{◯}; —(CH₂)₀₋₄C(O)OSiR^{◯}₃; —(CH₂)₀₋₄OC(O)R^{◯}; OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —(CH₂)₀₋₄OC(O)NR^{◯}₂; —C(O)N(OR^{◯})R^{◯}; —C(O) C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O) (OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, (CH₂)₀₋₂R^{●}, -(haloR^{●}), (CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, OSiR^{●}₃, —C(O)SR^{●}, —(C₁₋₄ straight or branched alkylene) C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, C(O)C(O)R^{†}, —C(O)CH₂C(O)R^{†}, —S(O)₂R^{†}, —S(O)₂NR^{†}₂, C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of RT, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon-containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

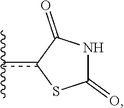

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and(S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di-, or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

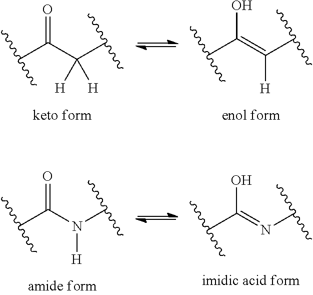 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

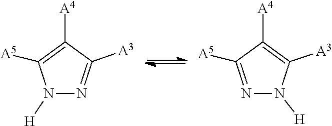

Unless stated to the contrary, the invention includes all such possible tautomers.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

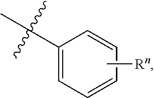

which is understood to be equivalent to a formula:

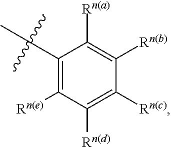

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compounds and compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. COMPOUNDS

In one aspect, disclosed are compounds having a structure represented by a formula:

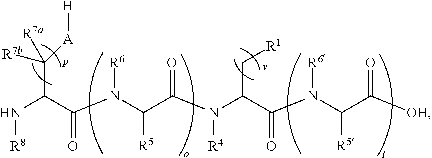

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

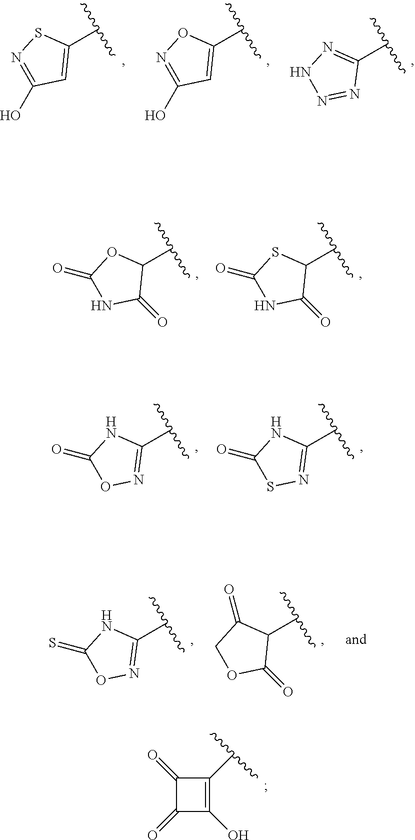

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are compounds having a structure represented by a formula:

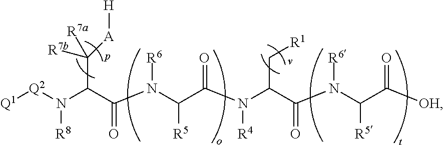

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

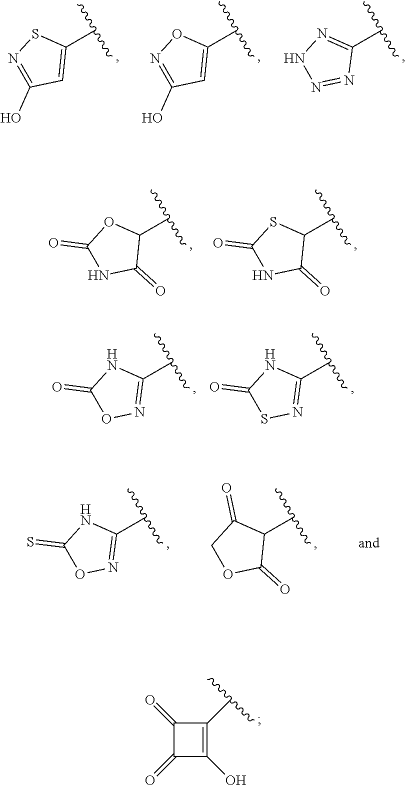

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are compounds having a structure represented by a formula:

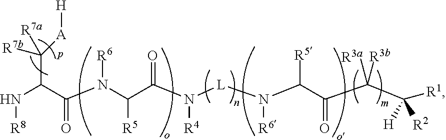

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, (C1-C4 alkyl) (OCH₂CH₂)_{q}, and a structure selected from:

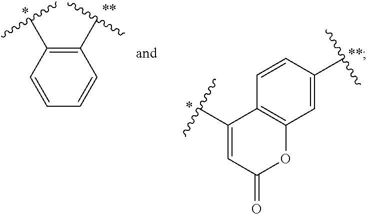

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

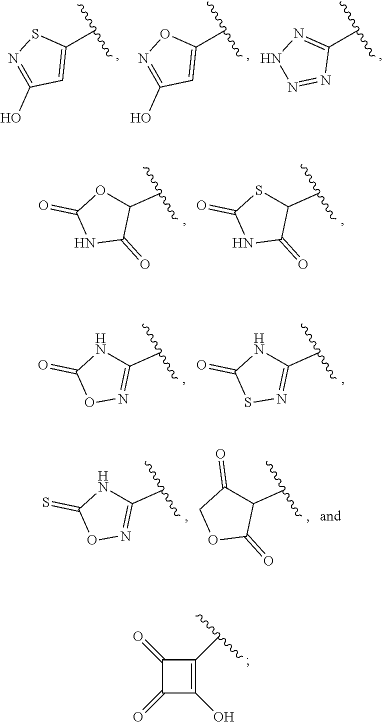

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are compounds having a structure represented by a formula:

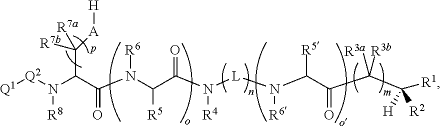

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, (C1-C4 alkyl) (OCH₂CH₂)_{q}, and a structure selected from:

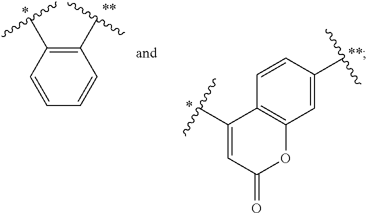

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

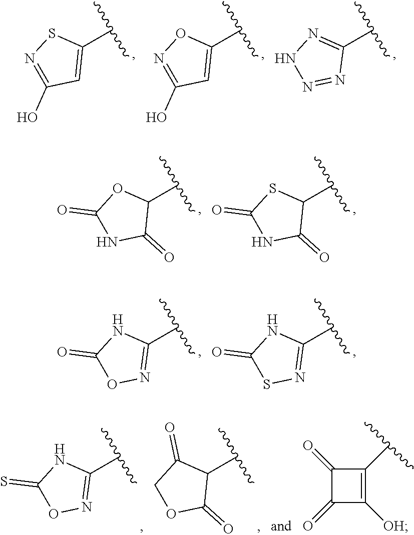

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, o is independently 0, 1, 2, 3, 4, 5, 6, or 7.

In various aspects, t is 0.

In various aspects, v is 1 or 2.

In various aspects, R¹ is —CO₂H or a structure:

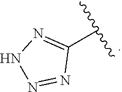 [SMILES: *c1nn[nH]n1]

In various aspects, R¹ is —CO₂H.

In various aspects, the cleavable moiety is —CO₂ (C4-C8 alkylene) OC(O)—. In a further aspect, the cleavable moiety is —CO₂CH₂CH═CHCH₂OC(O)—.

In various aspects, the cleavable moiety is a protease recognition sequence. In a further aspect, the protease recognition sequence is TEV recognition sequence.

In various aspects, the compound comprises one or more D-amino acid residues. In a further aspect, the compound comprises one or more β-amino acid residues. In a still further aspect, the compound comprises one or more N-methylated amino acid residues.

In various aspects, PapB installs a single thioether linkage in the compound. In a further aspect, PapB installs two or more thioether linkages in the compound.

In various aspects, the compound has a structure represented by a formula:

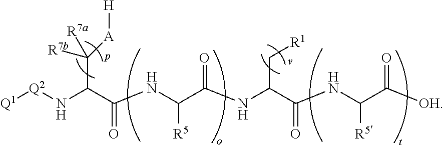

In various aspects, the compound has a structure represented by a formula:

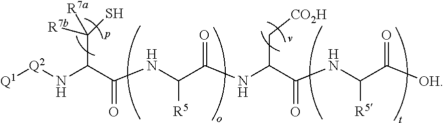

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, A is S. In a further aspect, A is Se.

In various aspects, L is C2-C4 alkyl. In a further aspect, L is —(C1-C4 alkyl) (OCH₂CH₂) q. In a still further aspect, L is a structure selected from:

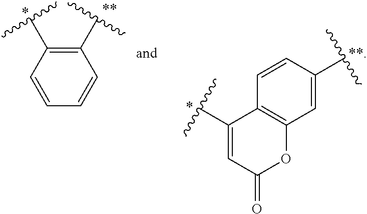 [SMILES: *c1ccccc1* ; *c1ccc2c(*)cc(=O)oc2c1]

In various aspects, the cleavable moiety is a protease recognition sequence. In a further aspect, the protease recognition sequence is a TEV protease recognition sequence. In a still further aspect, the TEV protease recognition sequence is EXLYZQ (SEQ ID NO: 1), in which X is any amino acid and Z is any amino acid that contains a hydrophobic residue. In yet a further aspect, the TEV protease recognition sequence is ENLYFQ (SEQ ID NO: 1).

In various aspects, the leader sequence is LKQINVIAGVKEPIRAYG (SEQ ID NO: 2) or LKQINVIAGVKPIRAYG (SEQ ID NO: 3). In a further aspect, the leader sequence is LKQINVIAGVKEPIRAYG (SEQ ID NO: 2).

In various aspects, R¹ is selected from —CO₂H and a structure:

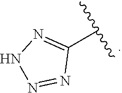 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

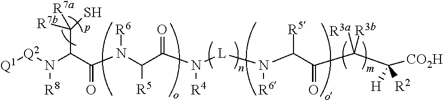

In various aspects, the compound has a structure represented by a formula:

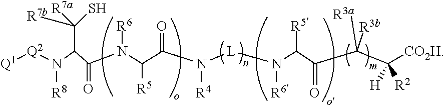

In various aspects, the compound has a structure represented by a formula:

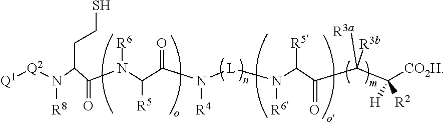

In various aspects, the compound has a structure represented by a formula:

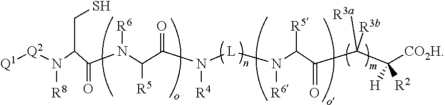

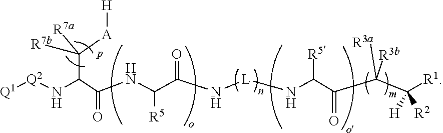

In various aspects, the compound has a structure represented by a formula:

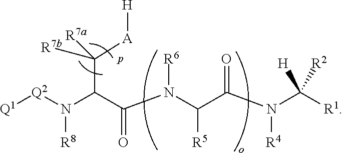

In various aspects, the compound has a structure represented by a formula:

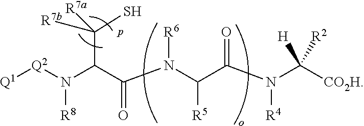

In various aspects, the compound has a structure represented by a formula:

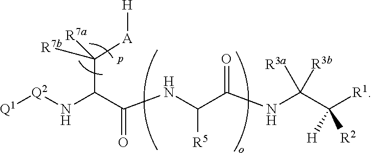

In a further aspect, o is 1, 2, 3, 4, 5, 6, 7, 8, or 9.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

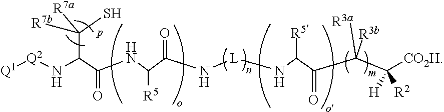

In various aspects, the compound has a structure represented by a formula:

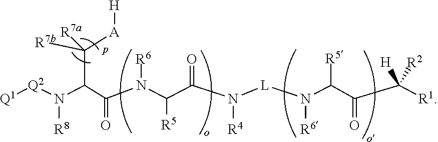

In various aspects, the compound has a structure represented by a formula:

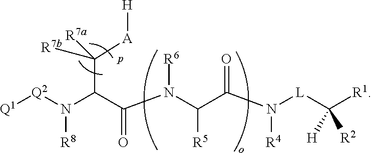

In various aspects, the compound has a structure represented by a formula:

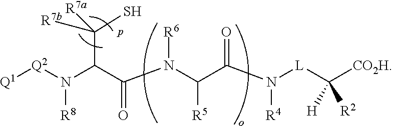

In a further aspect, o is 1, 2, 3, 4, 5, 6, 7, 8, or 9.

In various aspects, the compound has a structure represented by a formula:

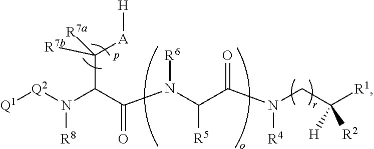

wherein r is 2, 3, or 4.

In various aspects, the compound has a structure represented by a formula:

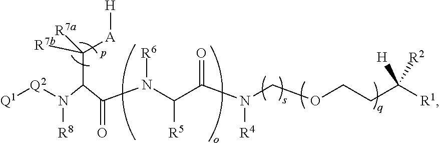

wherein s is 1 or 2.

In various aspects, the compound has a structure represented by a formula:

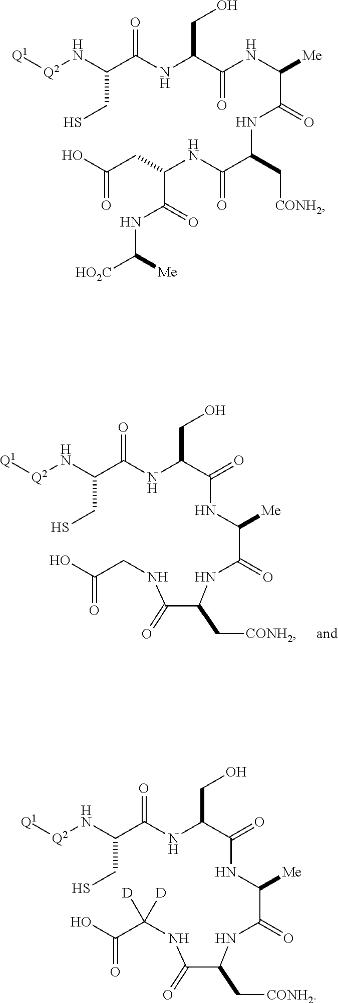

### C. THIOETHER COMPOUNDS

In one aspect, disclosed are thioether compounds produced by a disclosed method. Thus, in various aspects, the method produces a thioether compound having a structure represented by a formula:

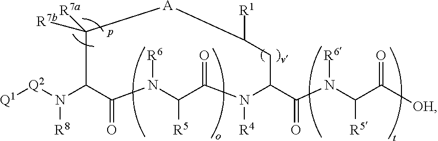

wherein v′ is 0, 1, 2, or 3.

In various aspects, the method further comprises addition of a reducing agent. In a further aspect, the method further comprises addition of a protease.

In various aspects, the method produces a thioether compound having a structure represented by a formula:

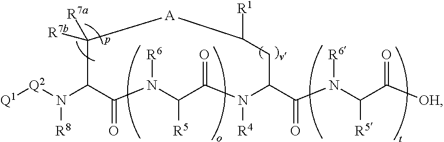

wherein v′ is 0, 1, 2, or 3.

In various aspects, the thioether compound is selected from:

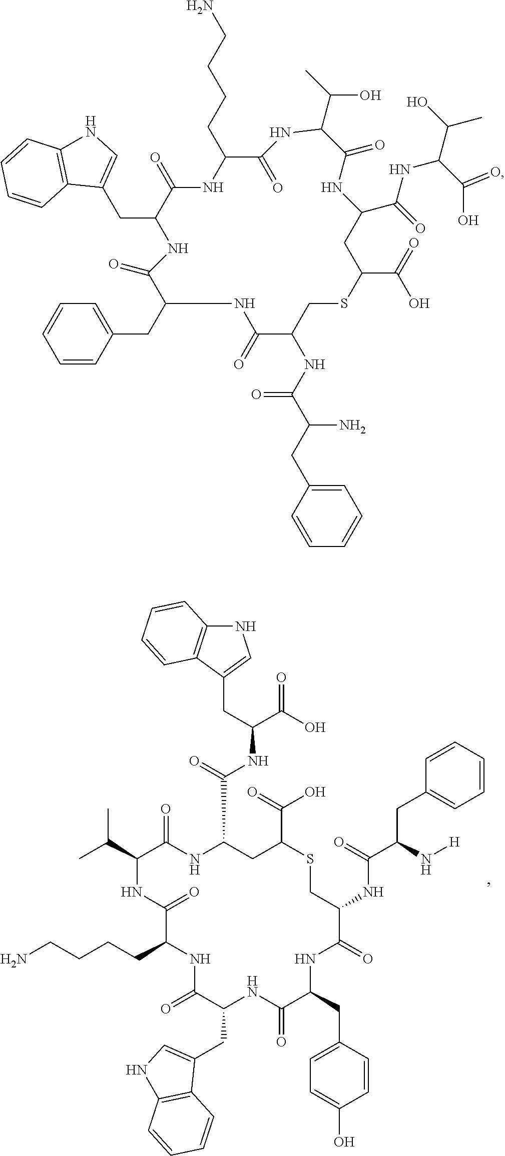 [SMILES: CC(O)C(NC(=O)C1CC(C(=O)O)SCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O ; CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSC(C(=O)O)C[C@@H](C(=O)N[C@@H](Cc2c[nH]c3ccccc23)C(=O)O)NC1=O]

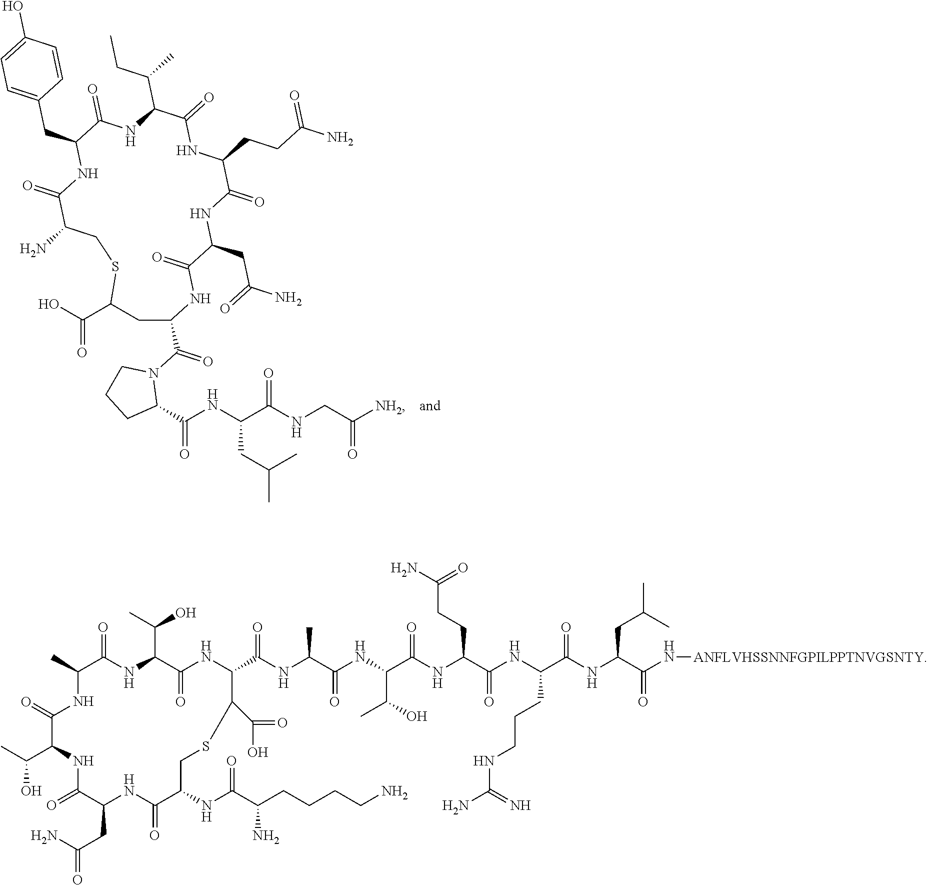

In various aspects, the method produces a thioether compound having a structure represented by a formula:

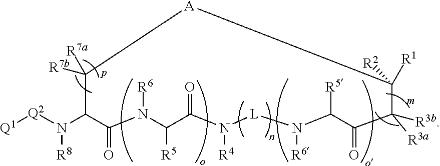

In various aspects, the thioether compound has a structure represented by a formula:

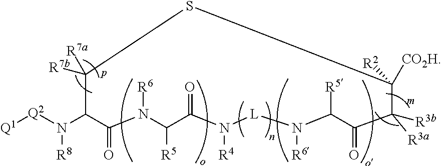

In various aspects, the thioether compound has a structure represented by a formula:

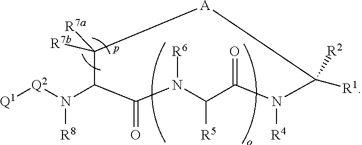

In various aspects, the thioether compound has a structure represented by a formula:

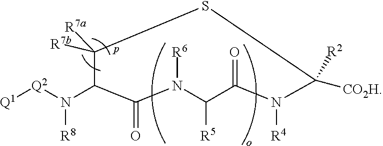

In various aspects, the thioether compound has a structure represented by a formula:

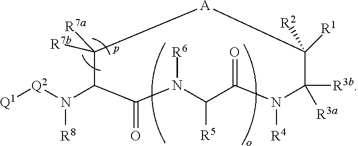

In various aspects, the thioether compound has a structure represented by a formula:

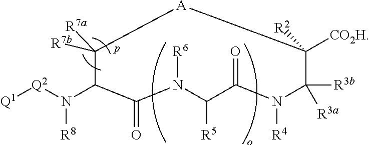

In various aspects, the thioether compound has a structure represented by a formula:

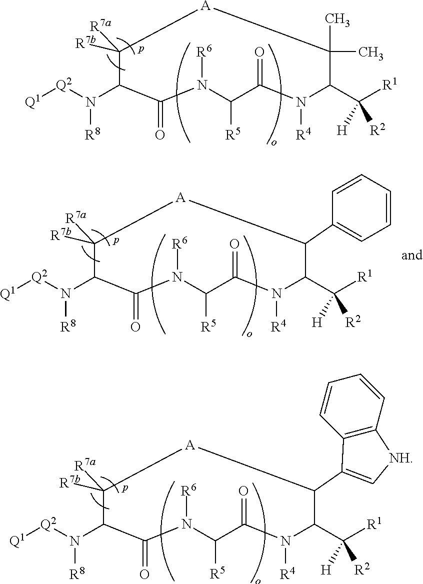

In various aspects, the thioether compound has a structure represented by a formula:

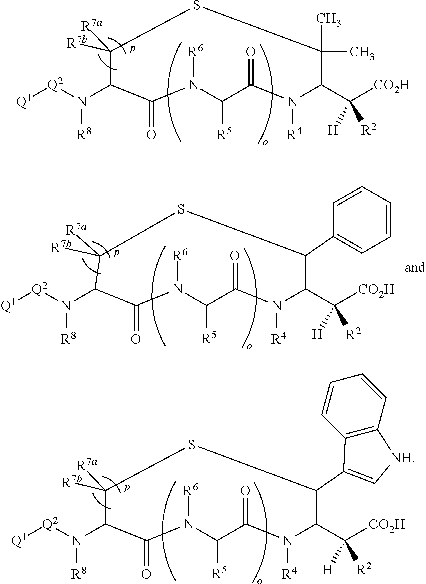

In various aspects, the thioether compound has a structure represented by a

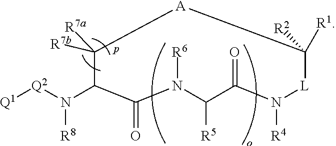

In various aspects, the thioether compound has a structure represented by a formula:

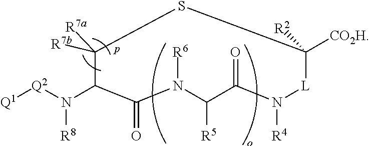

In various aspects, the thioether compound has a structure represented by a formula selected from:

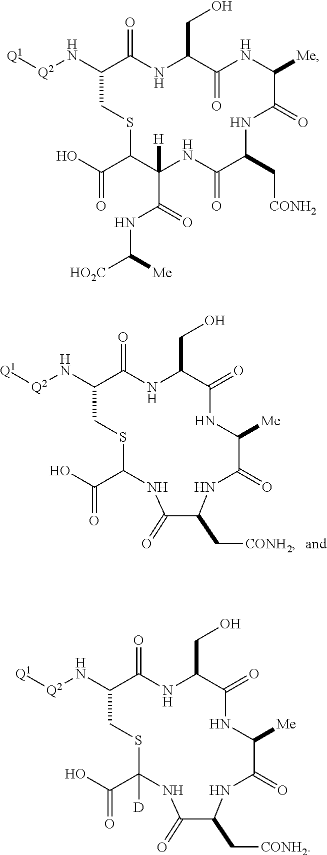

In various aspects, the thioether compound has a structure represented by a

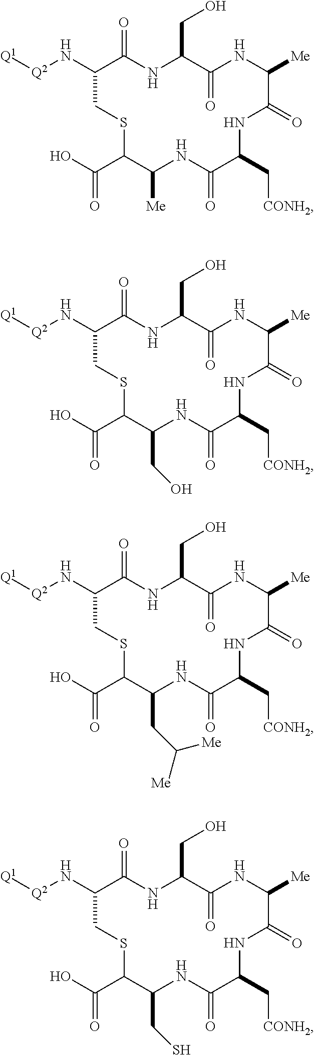

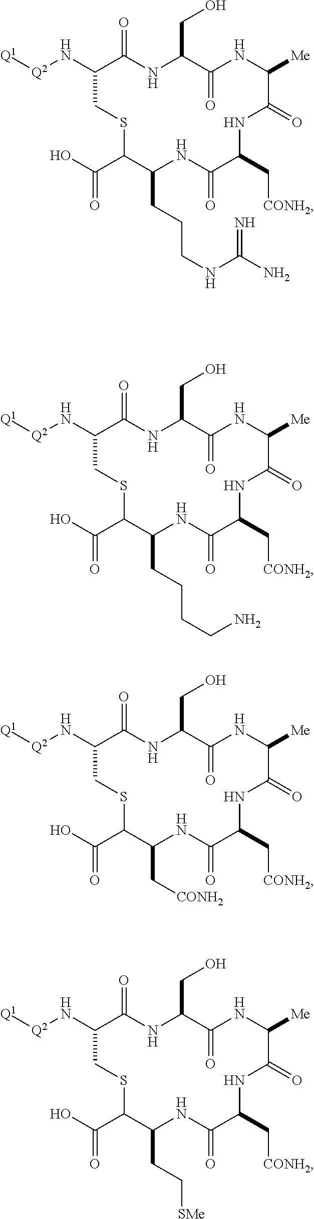

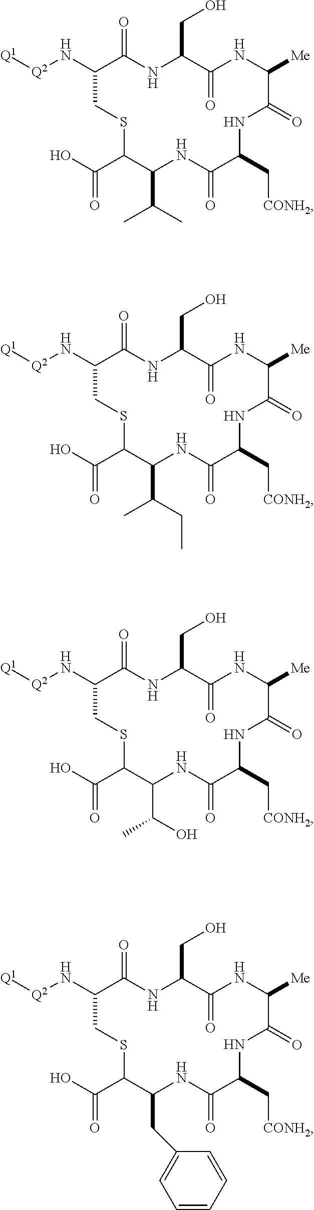

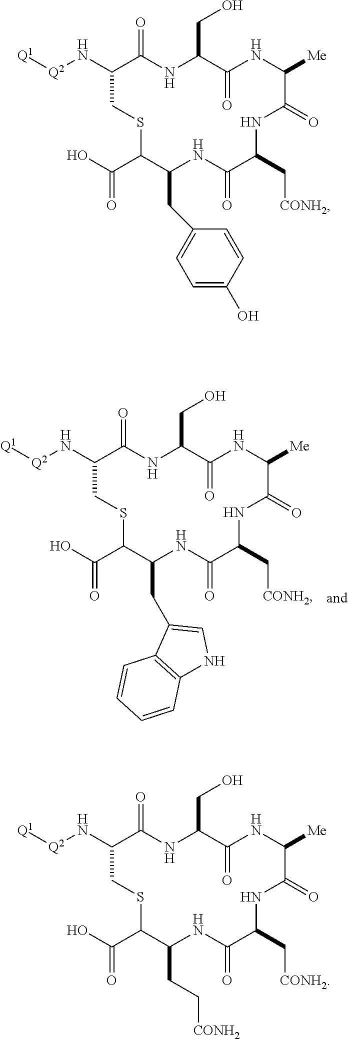

In various aspects, the thioether compound has a structure represented by a

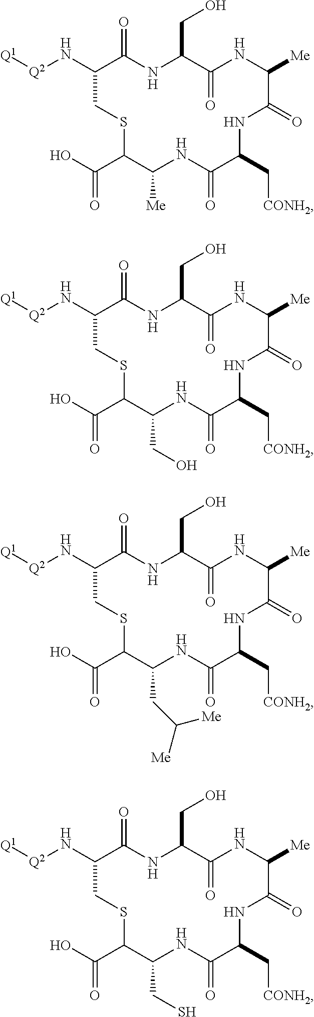

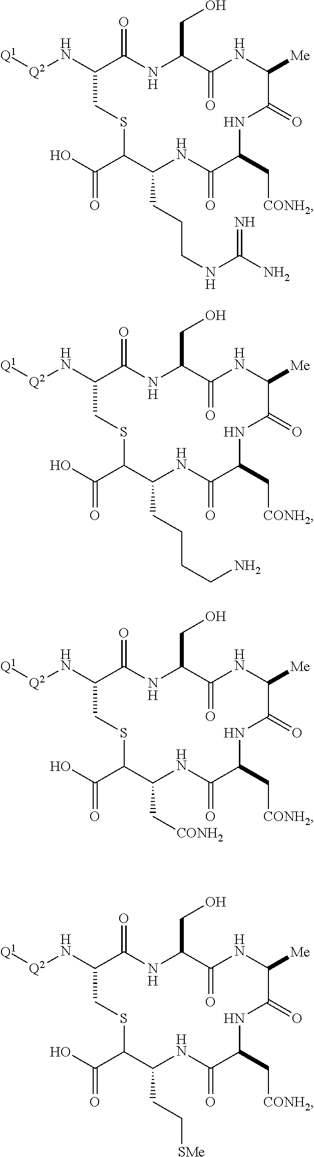

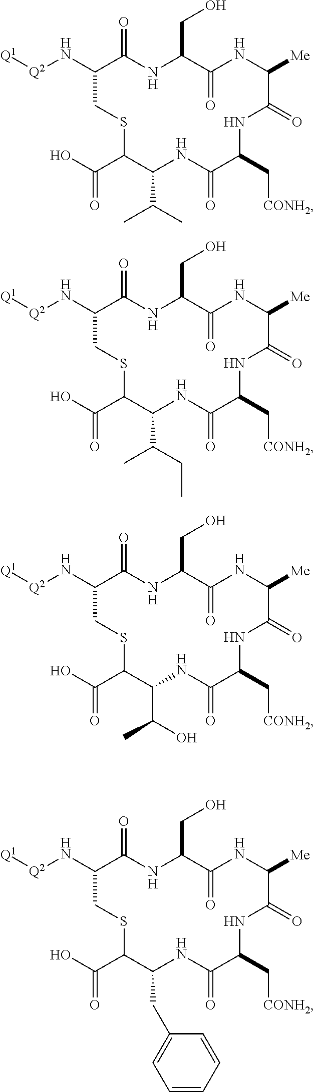

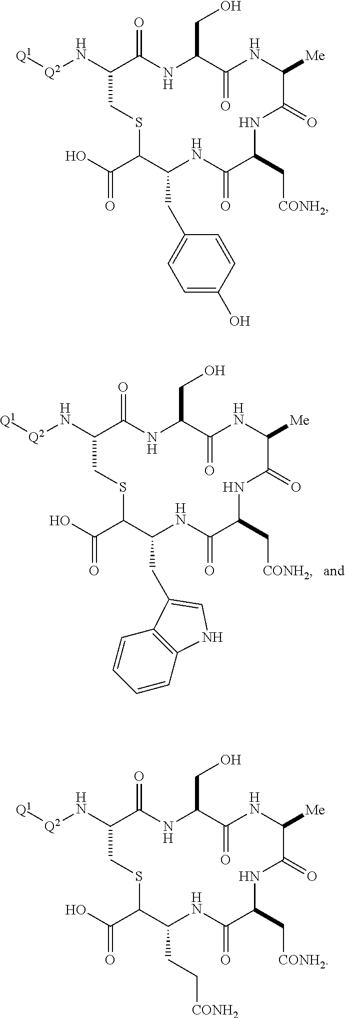

In various aspects, the thioether compound has a structure represented by a

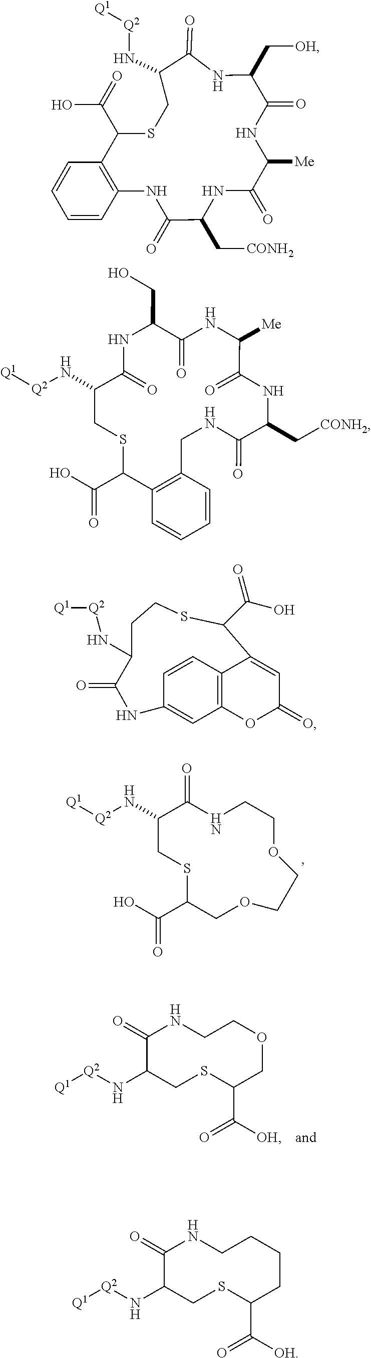

In various aspects, the thioether compound is a sactipeptide. In a further aspect, the sactipeptide has a structure represented by a formula selected from:

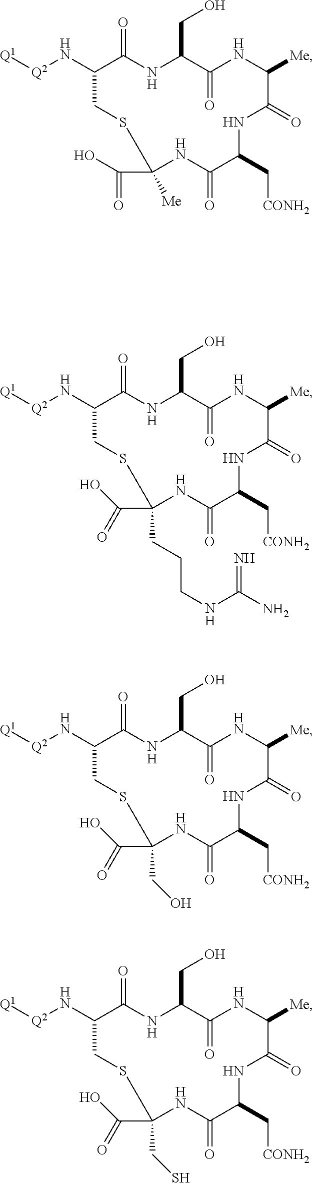

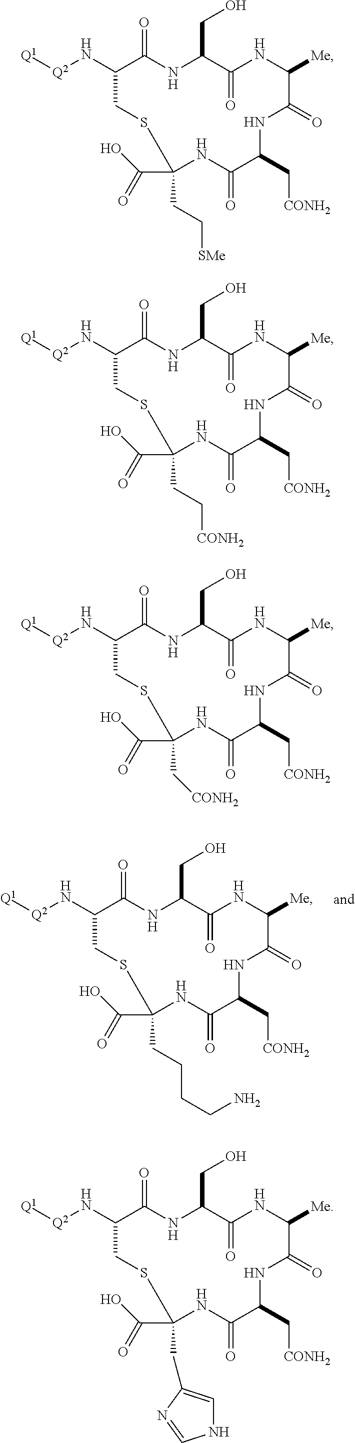

In various aspects, the thioether compound is a ranthipeptide. In a further aspect, the ranthipeptide has a structure represented by a formula selected from:

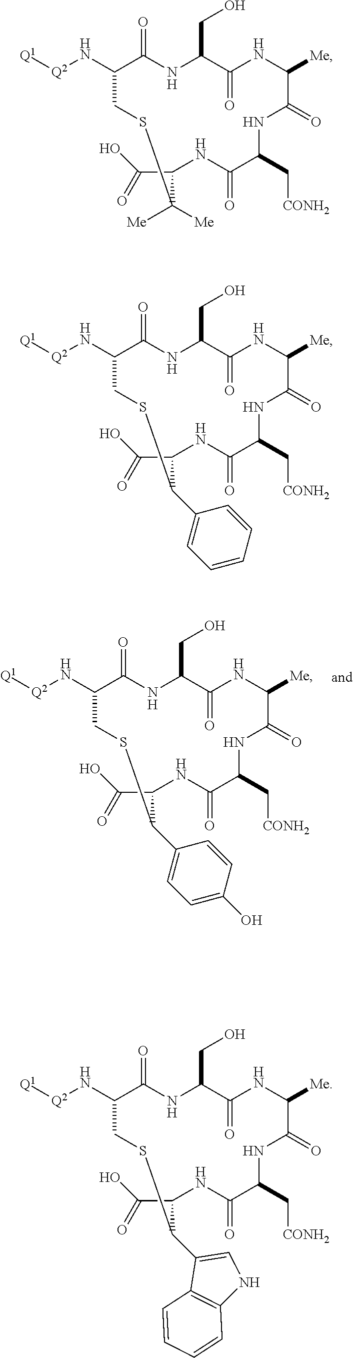

In various aspects, the method produces a thioether compound having a structure represented by a formula:

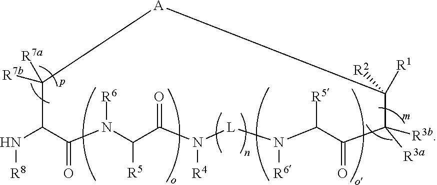

In various aspects, the thioether compound has a structure represented by a formula:

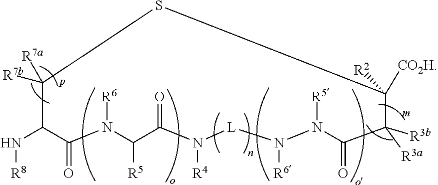

In various aspects, the thioether compound has a structure represented by a formula:

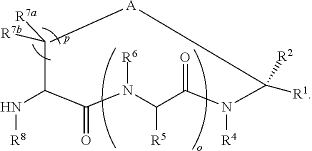

In various aspects, the thioether compound has a structure represented by a

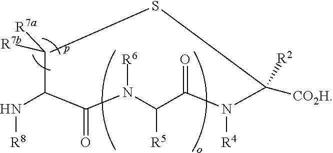

In various aspects, the thioether compound has a structure represented by a formula:

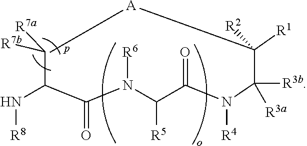

In various aspects, the thioether compound has a structure represented by a formula:

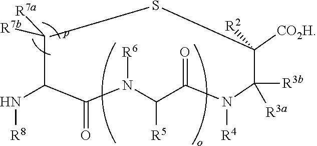

In various aspects, the thioether compound has a structure represented by a formula:

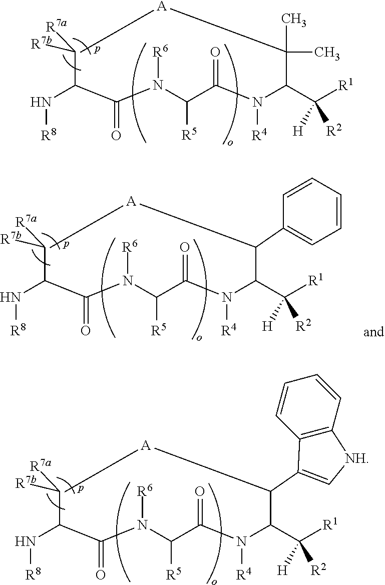

In various aspects, the thioether compound has a structure represented by a

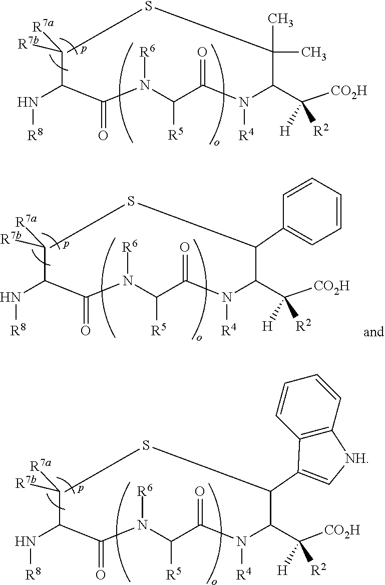

In various aspects, the thioether compound has a structure represented by a formula:

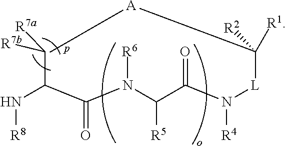

In various aspects, the thioether compound has a structure represented by a formula:

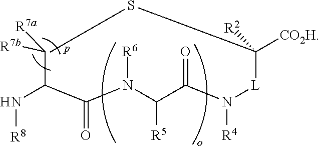

In various aspects, the thioether compound has a structure represented by a formula:

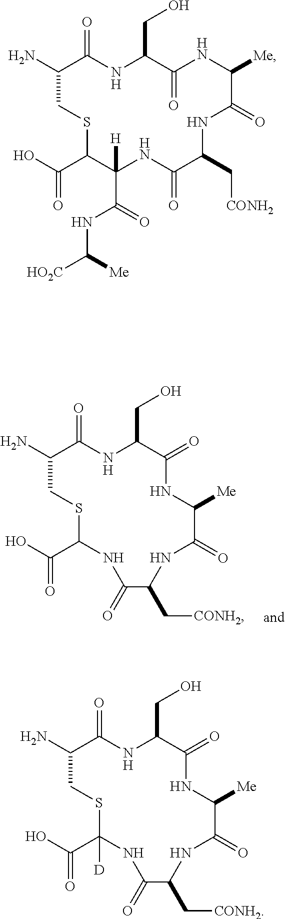 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula:

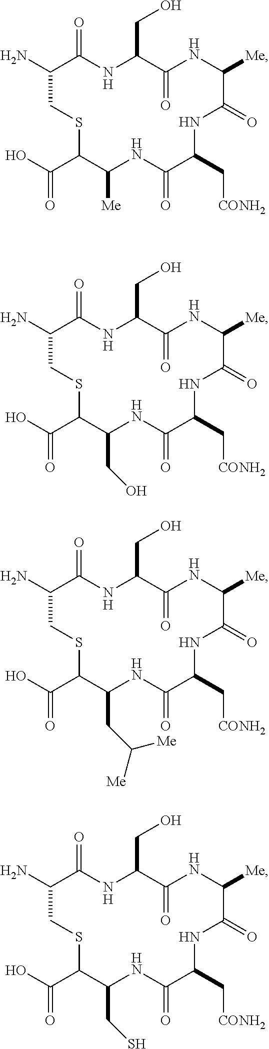 [SMILES: C[C@@H]1NC(=O)[C@H](CO)NC(=O)[C@@H](N)CSC(C(=O)O)[C@H](C)NC(=O)[C@H](CC(N)=O)NC1=O ; C[C@@H]1NC(=O)[C@H](CO)NC(=O)[C@@H](N)CSC(C(=O)O)[C@H](CO)NC(=O)[C@H](CC(N)=O)NC1=O ; CC(C)C[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H](CS)NC(=O)[C@H](CC(N)=O)NC1=O]

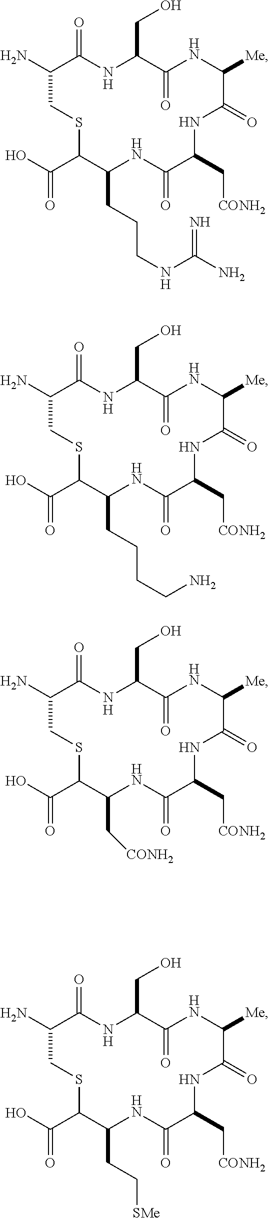

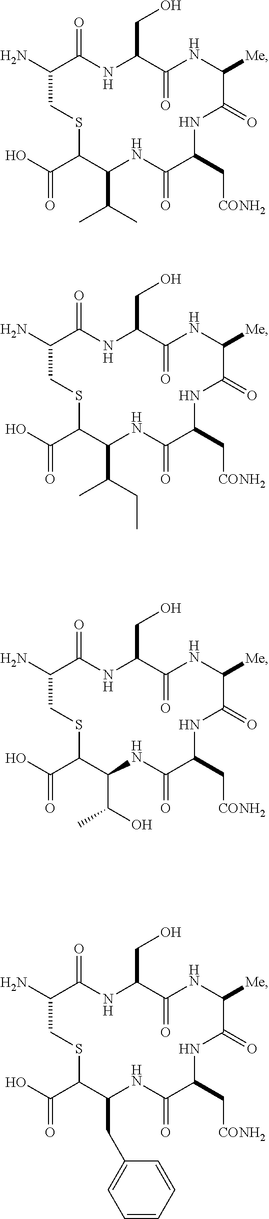 [SMILES: CC(C)[C@@H]1NC(=O)[C@H](CC(N)=O)NC(=O)[C@H](C)NC(=O)[C@H](CO)NC(=O)[C@@H](N)CSC1C(=O)O ; CCC(C)[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H]([C@@H](C)O)NC(=O)[C@H](CC(N)=O)NC1=O ; C[C@@H]1NC(=O)[C@H](CO)NC(=O)[C@@H](N)CSC(C(=O)O)[C@H](Cc2ccccc2)NC(=O)[C@H](CC(N)=O)NC1=O]

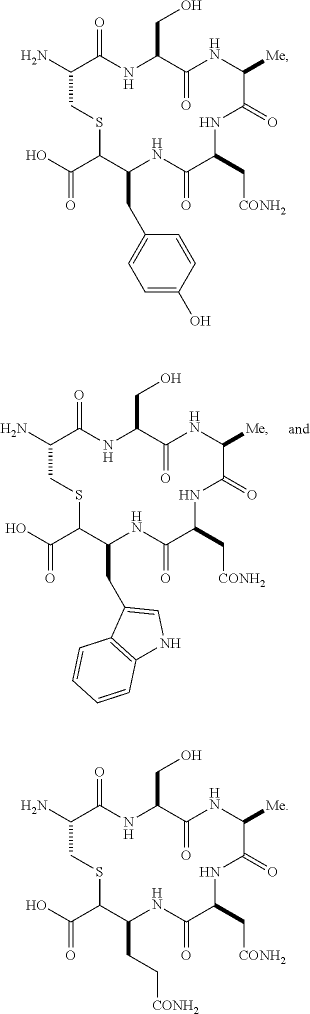 [SMILES: C[C@@H]1NC(=O)[C@H](CO)NC(=O)[C@@H](N)CSC(C(=O)O)[C@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@H](Cc2c[nH]c3ccccc23)NC(=O)[C@H](CC(N)=O)NC1=O ; C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula:

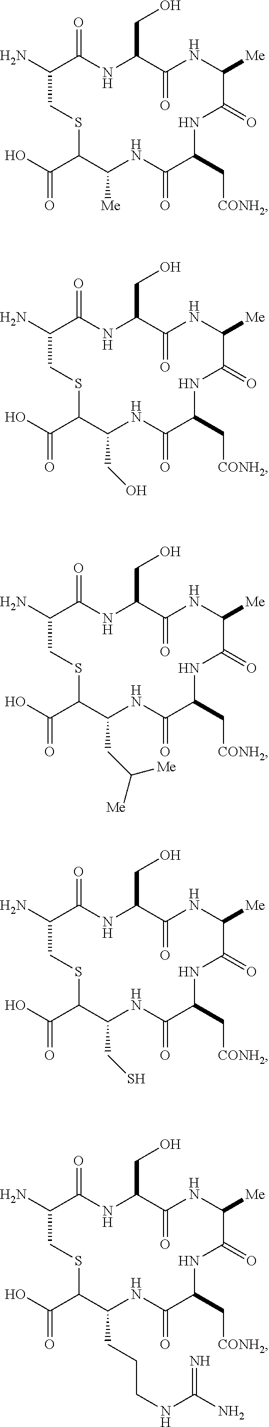 [SMILES: C[C@@H]1NC(=O)[C@H](CO)NC(=O)[C@@H](N)CSC(C(=O)O)[C@@H](C)NC(=O)[C@H](CC(N)=O)NC1=O ; C[C@@H]1NC(=O)[C@H](CO)NC(=O)[C@@H](N)CSC(C(=O)O)[C@@H](CO)NC(=O)[C@H](CC(N)=O)NC1=O ; CC(C)C[C@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@@H](CS)NC(=O)[C@H](CC(N)=O)NC1=O ; C[C@@H]1NC(=O)[C@H](CO)NC(=O)[C@@H](N)CSC(C(=O)O)[C@@H](CCCNC(=N)N)NC(=O)[C@H](CC(N)=O)NC1=O]

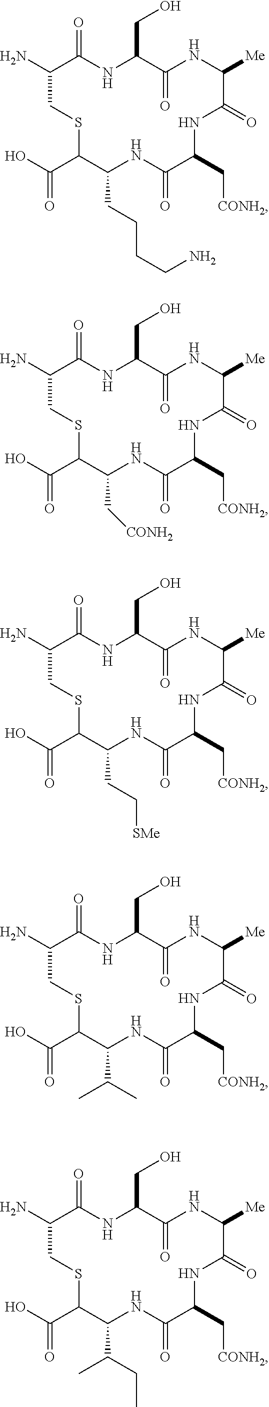

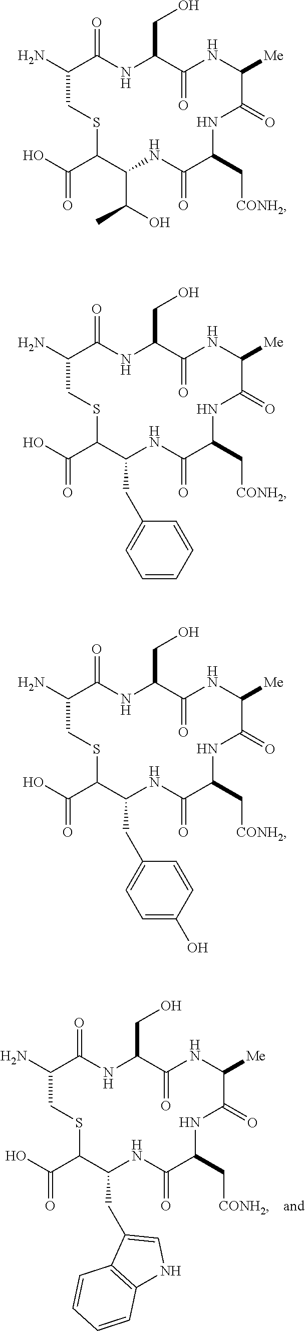 [SMILES: C[C@@H]1NC(=O)[C@H](CO)NC(=O)[C@@H](N)CSC(C(=O)O)[C@@H]([C@H](C)O)NC(=O)[C@H](CC(N)=O)NC1=O ; C[C@@H]1NC(=O)[C@H](CO)NC(=O)[C@@H](N)CSC(C(=O)O)[C@@H](Cc2ccccc2)NC(=O)[C@H](CC(N)=O)NC1=O ; C[C@@H]1NC(=O)[C@H](CO)NC(=O)[C@@H](N)CSC(C(=O)O)[C@@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@@H](Cc2c[nH]c3ccccc23)NC(=O)[C@H](CC(N)=O)NC1=O]

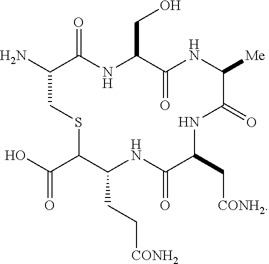 [SMILES: C[C@@H]1NC(=O)[C@H](CO)NC(=O)[C@@H](N)CSC(C(=O)O)[C@@H](CCC(N)=O)NC(=O)[C@H](CC(N)=O)NC1=O]

In various aspects, the thioether compound has a structure represented by a formula:

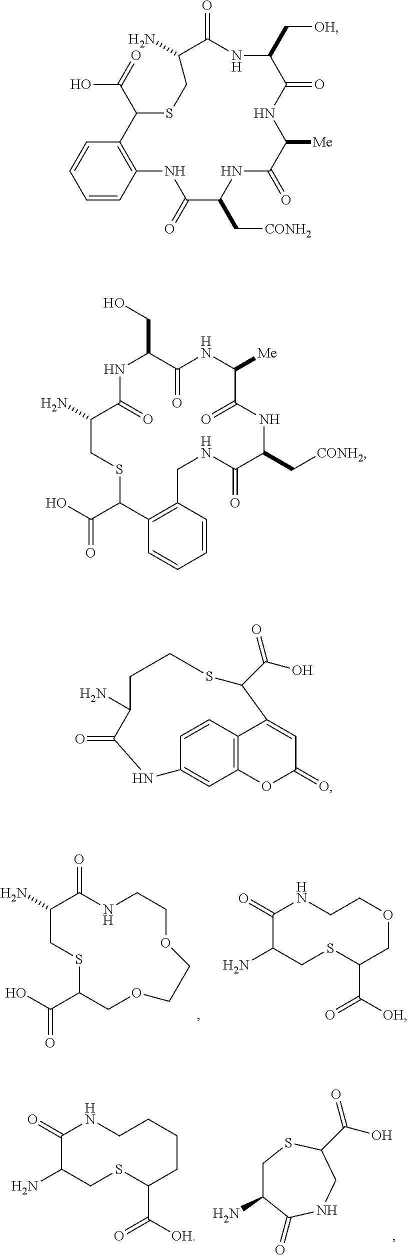

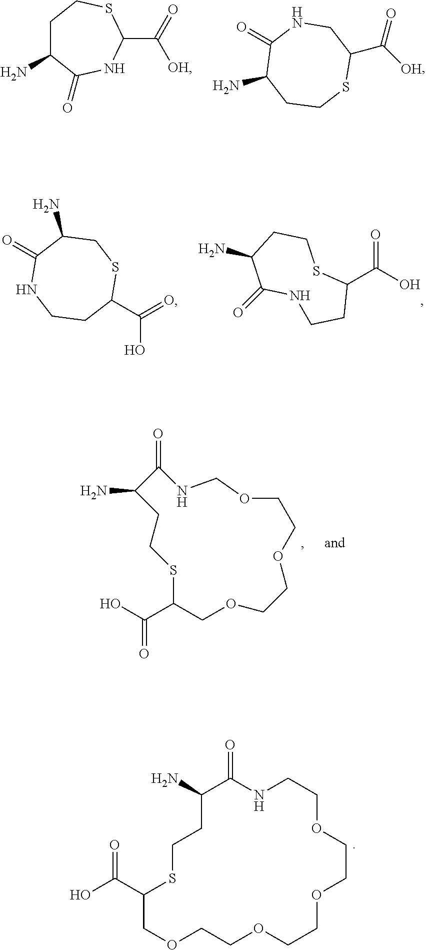 [SMILES: N[C@H]1CCSC(C(=O)O)NC1=O ; N[C@@H]1CCSC(C(=O)O)CNC1=O ; N[C@H]1CSC(C(=O)O)CCNC1=O ; N[C@H]1CCSC(C(=O)O)CCNC1=O ; N[C@@H]1CCSC(C(=O)O)COCCOCCOCNC1=O ; N[C@@H]1CCSC(C(=O)O)COCCOCCOCCOCCNC1=O]

In various aspects, the thioether compound is a sactipeptide. In a further aspect, the sactipeptide has a structure represented by a formula selected from:

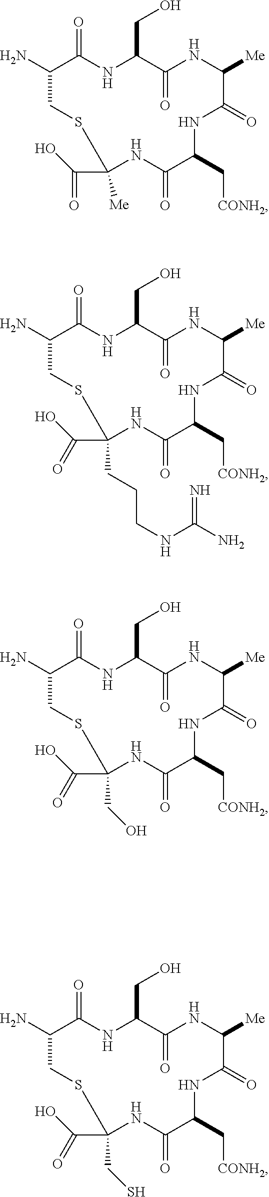 [SMILES: C[C@@H]1NC(=O)[C@H](CO)NC(=O)[C@@H](N)CS[C@](C)(C(=O)O)NC(=O)[C@H](CC(N)=O)NC1=O ; C[C@@H]1NC(=O)[C@H](CO)NC(=O)[C@@H](N)CS[C@](CCCNC(=N)N)(C(=O)O)NC(=O)[C@H](CC(N)=O)NC1=O ; C[C@@H]1NC(=O)[C@H](CO)NC(=O)[C@@H](N)CS[C@](CO)(C(=O)O)NC(=O)[C@H](CC(N)=O)NC1=O ; C[C@@H]1NC(=O)[C@H](CO)NC(=O)[C@@H](N)CS[C@](CS)(C(=O)O)NC(=O)[C@H](CC(N)=O)NC1=O]

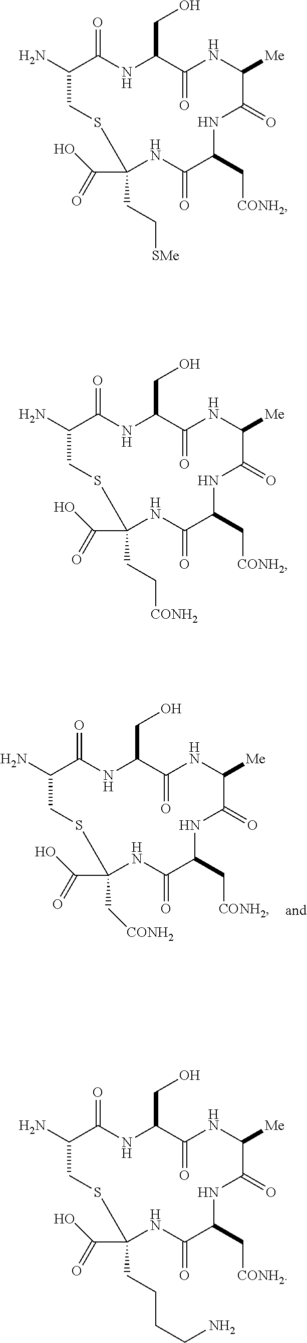 [SMILES: CSCC[C@@]1(C(=O)O)NC(=O)[C@H](CC(N)=O)NC(=O)[C@H](C)NC(=O)[C@H](CO)NC(=O)[C@@H](N)CS1 ; C[C@@H]1NC(=O)[C@H](CO)NC(=O)[C@@H](N)CS[C@](CCC(N)=O)(C(=O)O)NC(=O)[C@H](CC(N)=O)NC1=O ; C[C@@H]1NC(=O)[C@H](CO)NC(=O)[C@@H](N)CS[C@](CC(N)=O)(C(=O)O)NC(=O)[C@H](CC(N)=O)NC1=O ; C[C@@H]1NC(=O)[C@H](CO)NC(=O)[C@@H](N)CS[C@](CCCCN)(C(=O)O)NC(=O)[C@H](CC(N)=O)NC1=O]

In various aspects, the thioether compound is a ranthipeptide. In a further aspect, the ranthipeptide has a structure represented by a formula selected from:

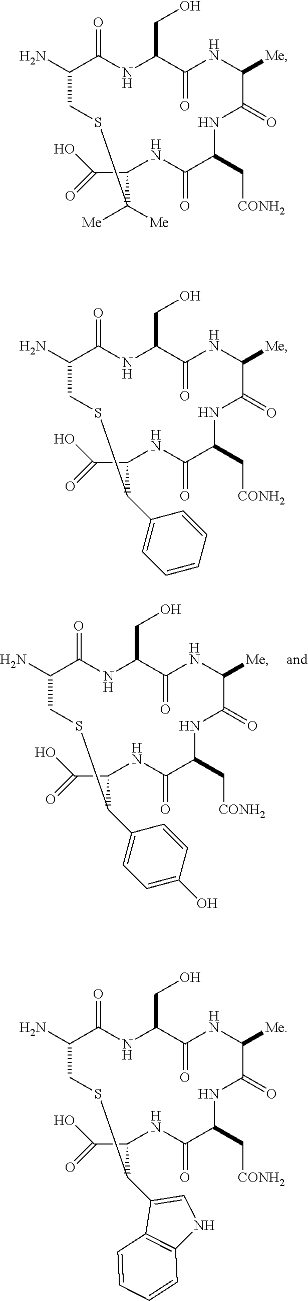

In various aspects, the thioether compound is selected from:

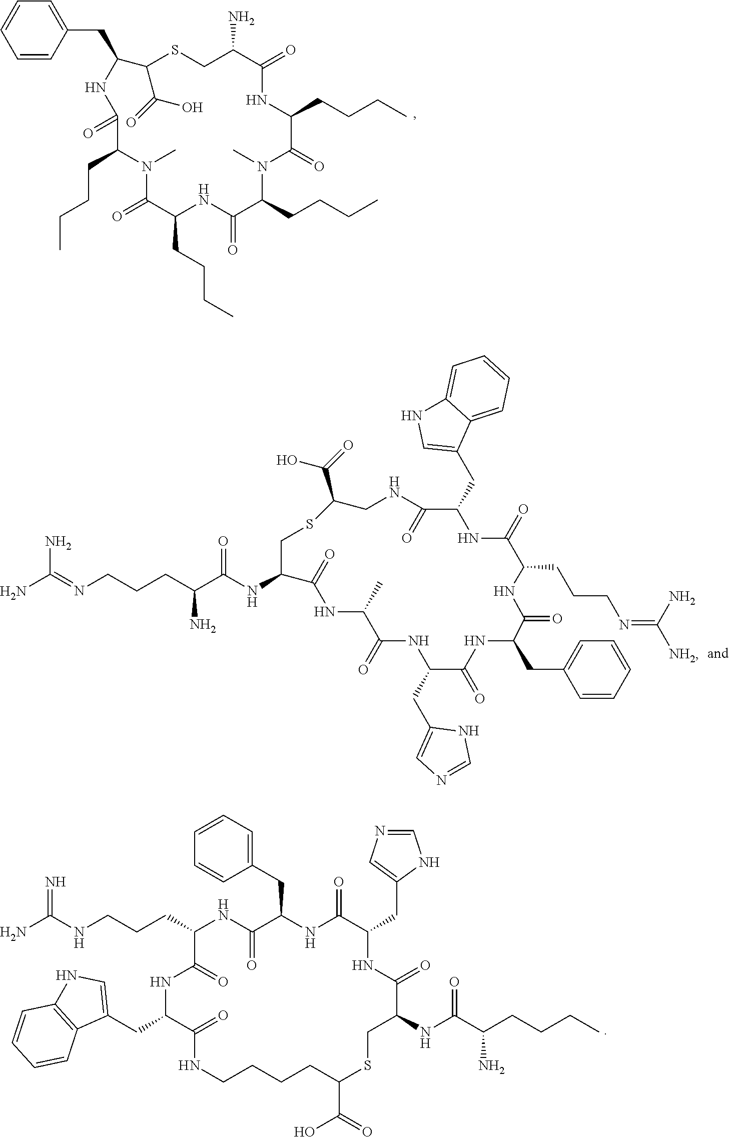 [SMILES: CCCC[C@@H]1NC(=O)[C@@H](N)CSC(C(=O)O)[C@H](Cc2ccccc2)NC(=O)[C@H](CCCC)N(C)C(=O)[C@H](CCCC)NC(=O)[C@H](CCCC)N(C)C1=O ; C[C@H]1NC(=O)[C@@H](NC(=O)[C@@H](N)CCCN=C(N)N)CS[C@@H](C(=O)O)CNC(=O)[C@H](Cc2c[nH]c3ccccc23)NC(=O)[C@H](CCCN=C(N)N)NC(=O)[C@@H](Cc2ccccc2)NC(=O)[C@H](Cc2cnc[nH]2)NC1=O ; CCCC[C@H](N)C(=O)N[C@H]1CSC(C(=O)O)CCCCNC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O]

In various aspects, the thioether compound is selected from:

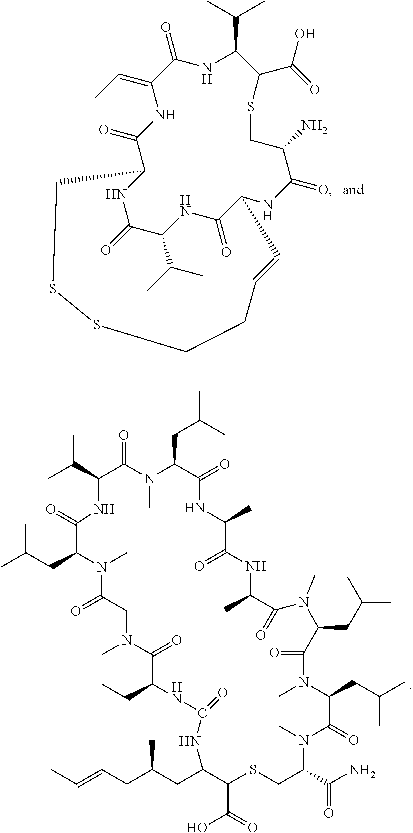 [SMILES: C/C=C1\NC(=O)[C@@H]2CSSCC/C=C/[C@H](NC(=O)[C@@H](N)CSC(C(=O)O)[C@H](C(C)C)NC1=O)C(=O)N[C@H](C(C)C)C(=O)N2 ; "and" ; C/C=C/C[C@@H](C)CC1NC(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@H](C(N)=O)CSC1C(=O)O]

### D. ANALOGS OF PEPTIDE THERAPEUTICS

In one aspect, disclosed are thioether compounds prepared by a disclosed method, wherein the thioether compound is an analog of a peptide therapeutic. Exemplary peptide therapeutics include, but are not limited to, octreotide, setmalanotide, romidepsin, bremelanotide, pramlintide, oxytocin, setmelanotide, or cyclosporin.

Thus, in one aspect, disclosed are compounds having a structure selected from:

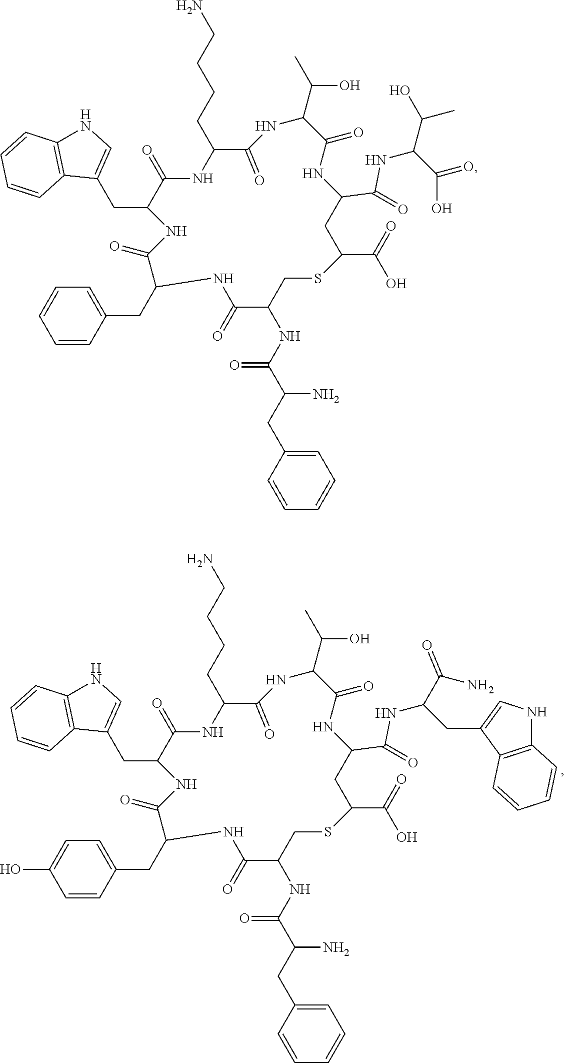 [SMILES: CC(O)C(NC(=O)C1CC(C(=O)O)SCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O ; CC(O)C1NC(=O)C(CCCCN)NC(=O)C(Cc2c[nH]c3ccccc23)NC(=O)C(Cc2ccc(O)cc2)NC(=O)C(NC(=O)C(N)Cc2ccccc2)CSC(C(=O)O)CC(C(=O)NC(Cc2c[nH]c3ccccc23)C(N)=O)NC1=O]

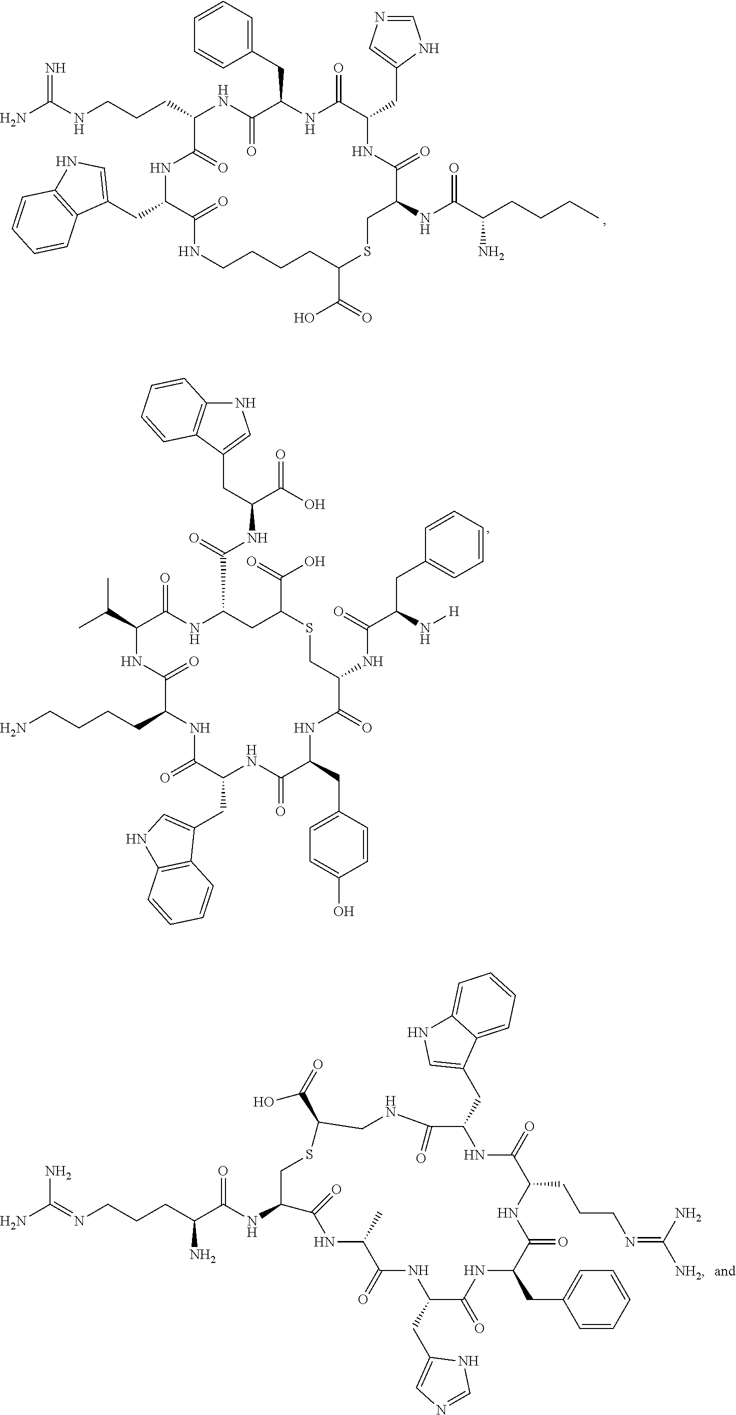

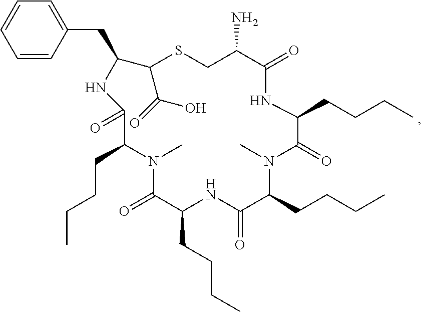 [SMILES: CCCC[C@@H]1NC(=O)[C@@H](N)CSC(C(=O)O)[C@H](Cc2ccccc2)NC(=O)[C@H](CCCC)N(C)C(=O)[C@H](CCCC)NC(=O)[C@H](CCCC)N(C)C1=O]

or a pharmaceutically acceptable salt thereof.

Also disclosed are compounds selected from:

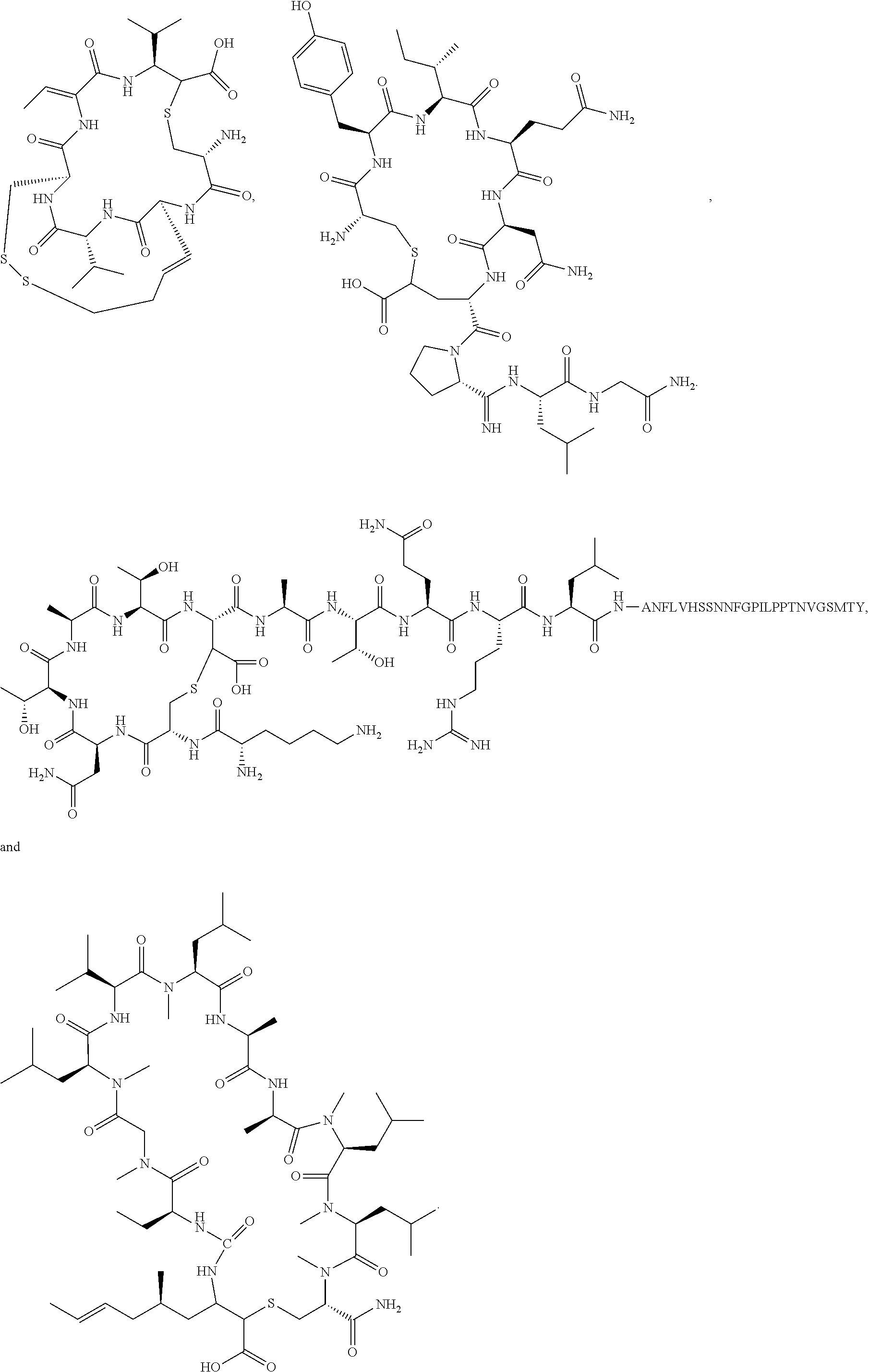

or a pharmaceutically acceptable salt thereof.

### E. METHODS OF CHEMICALLY MODIFYING A COMPOUND

In one aspect, disclosed are methods of chemically modifying a compound to install a thioether linkage, the method comprising reacting the compound with PapB, wherein the compound has a structure represented by a formula:

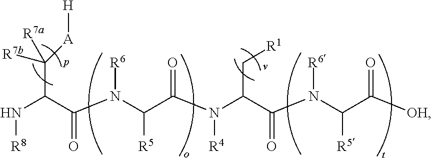

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

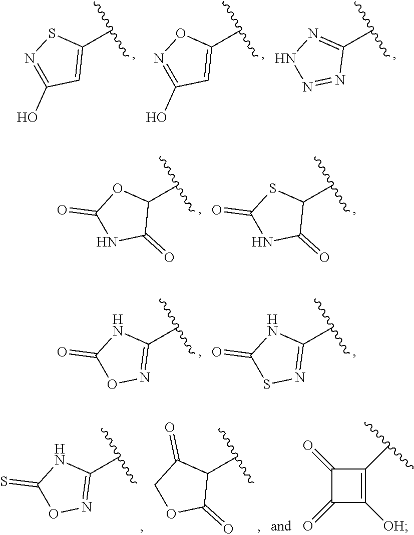

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are methods of chemically modifying a compound to install a thioether linkage, the method comprising reacting the compound with PapB, wherein the compound has a structure represented by a formula:

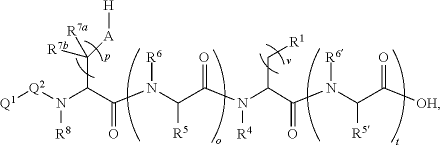

wherein o is 0, 1, 2, 3, 4, 5, 6, 7, 8, or 9; wherein p is 1 or 2; wherein t is an integer from 0 to 500; wherein v is 1, 2, 3, 4, or 5; wherein A is S or Se; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

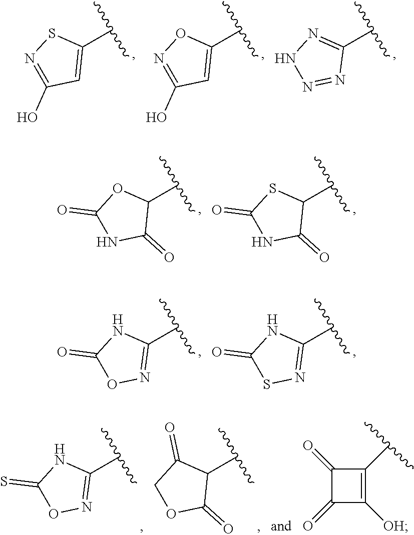

wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl; and wherein R⁸ is selected from hydrogen and methyl, provided that the compound is not PapA.

Also disclosed are methods of chemically modifying a compound to install a thioether linkage, the method comprising reacting the compound with PapB, wherein the compound has a structure represented by a formula:

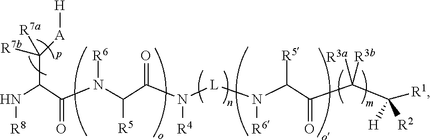

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl) (OCH₂CH₂)_{q}, and a structure selected from:

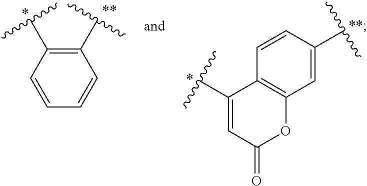

wherein q is 1, 2, 3, or 4; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

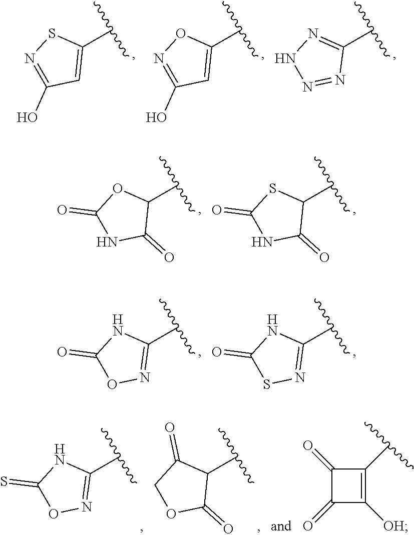

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

Also disclosed are methods of chemically modifying a compound to install a thioether linkage, the method comprising reacting the compound with PapB, wherein the compound has a structure represented by a formula:

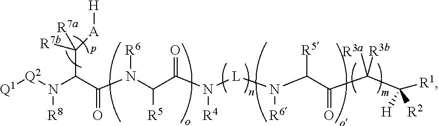

wherein m is 0, 1, 2, 3, or 4; wherein n is 0 or 1; wherein each of o and o′ is independently 0, 1, 2, 3, 4, 5, 6, 7, 8, or 9; wherein p is 1 or 2; wherein A is S or Se; wherein L, when present, is selected from C2-C4 alkyl, —(C1-C4 alkyl) (OCH₂CH₂)_{q}, and a structure selected from:

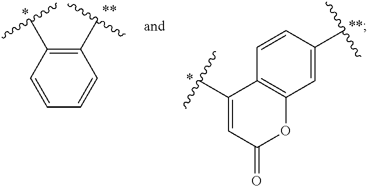

wherein q is 1, 2, 3, or 4; wherein Q¹ is a leader sequence; wherein Q² is a cleavable moiety; wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from:

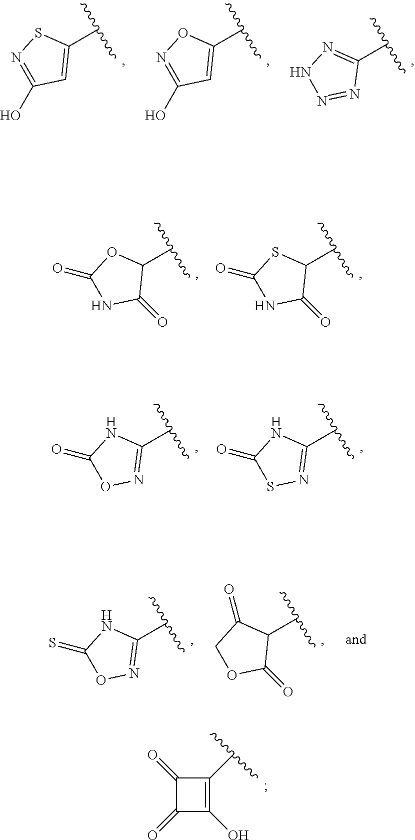

wherein R² is a residue of a side chain of amino acid, provided that the amino acid is not isoleucine or threonine; wherein each of R^{3a} and R^{3b}, when present, is independently selected from C2-C5 alkynyl, C1-C5 azido, and a residue of a side chain of an amino acid; wherein R⁴ is selected from hydrogen and methyl; wherein each occurrence of R⁵ and R^{5′}, when present, is independently a residue of a side chain of amino acid; wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle; wherein each of R^{7a} and R^{7b}, when present, is independently selected from hydrogen and C1-C4 alkyl, provided that the compound is not PapA.

In various aspects, o is independently 0, 1, 2, 3, 4, 5, 6, or 7.

In various aspects, t is 0.

In various aspects, v is 1 or 2.

In various aspects, R¹ is —CO₂H or a structure:

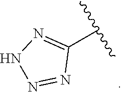 [SMILES: *c1nn[nH]n1]

In various aspects, R¹ is —CO₂H.

In various aspects, the cleavable moiety is a chemically cleavable moiety. Exemplary chemical cleavable moieties include, but are not limited to, CO₂ (C4-C8 alkylene)-OC(O)—. In a further aspect, the chemically cleavable moiety is —CO₂CH₂CH═CHCH₂OC(O)—.

In various aspects, the cleavable moiety is an enzymatically cleavable moiety such as, for example, a protease recognition sequence. In a further aspect, the protease recognition sequence is TEV recognition sequence.

In various aspects, the compound comprises one or more D-amino acid residues. In a further aspect, the compound comprises one or more β-amino acid residues. In a still further aspect, the compound comprises one or more N-methylated amino acid residues.

In various aspects, PapB installs a single thioether linkage in the compound.

In a further aspect, PapB installs two or more thioether linkages in the compound.

In various aspects, the compound has a structure represented by a formula:

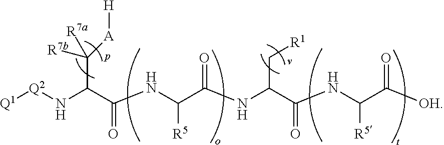

In various aspects, the compound has a structure represented by a formula:

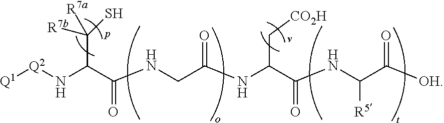

In various aspects, the method produces a thioether compound having a structure represented by a formula:

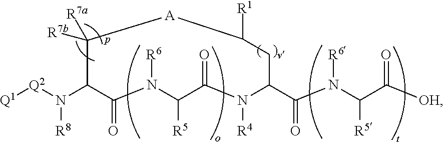

wherein v′ is 0, 1, 2, or 3.

In various aspects, the method further comprises addition of a reducing agent. In a further aspect, the method further comprises addition of a protease.

In various aspects, the method produces a thioether compound having a structure represented by a formula:

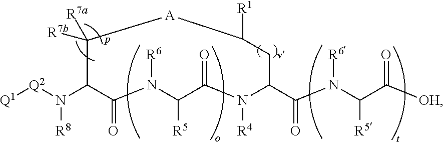

wherein v′ is 0, 1, 2, or 3.

In various aspects, the thioether compound is selected from:

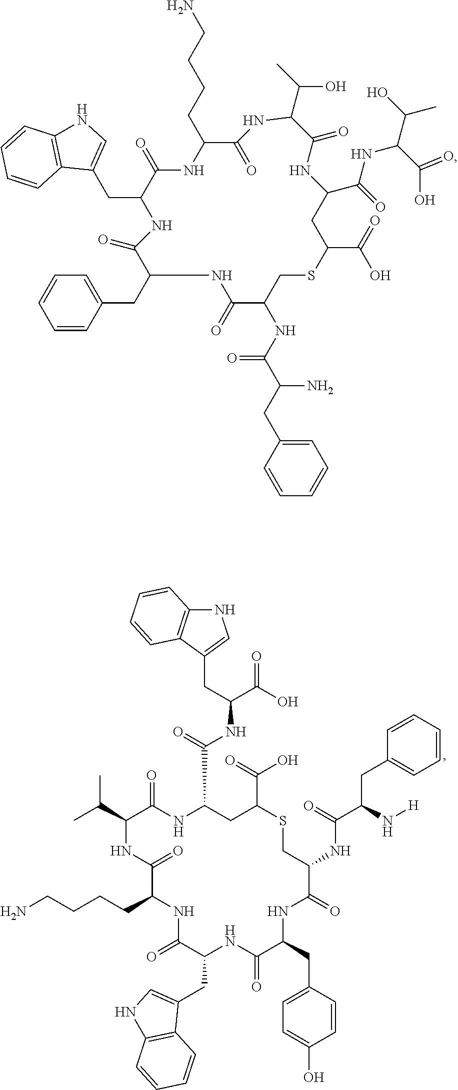 [SMILES: CC(O)C(NC(=O)C1CC(C(=O)O)SCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O ; CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSC(C(=O)O)C[C@@H](C(=O)N[C@@H](Cc2c[nH]c3ccccc23)C(=O)O)NC1=O]

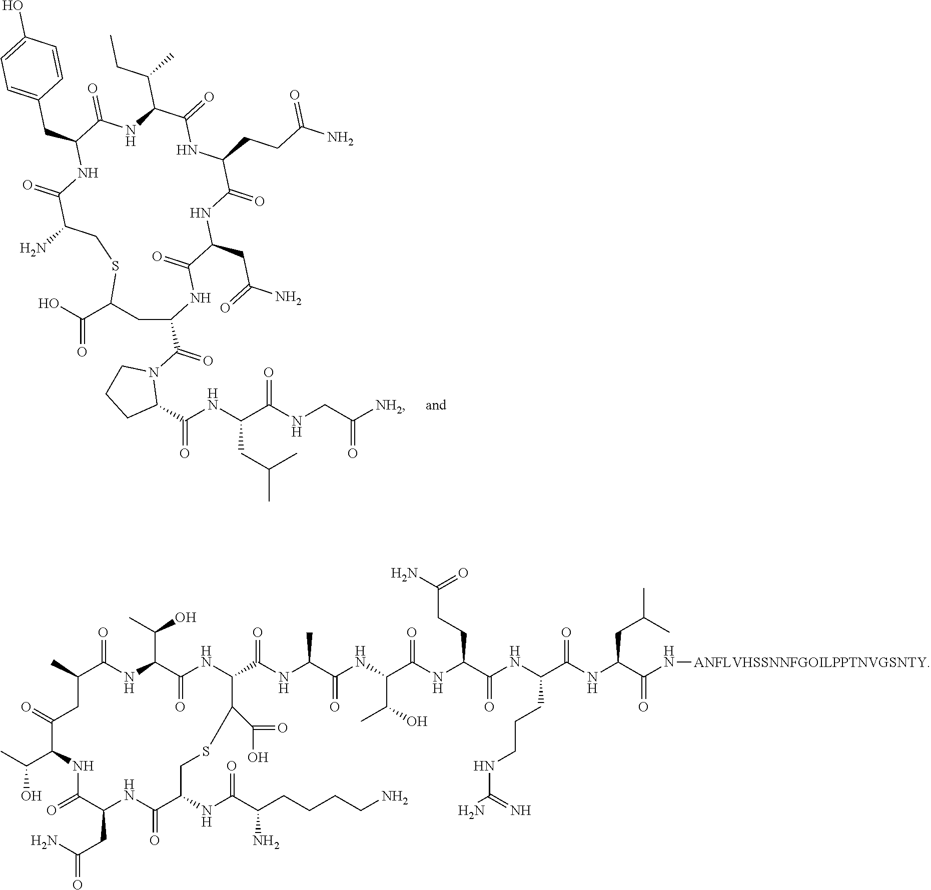

In various aspects, m is 0. In a further aspect, m is 1.

In various aspects, n is 0. In a further aspect, n is 1.

In various aspects, o is 0, 1, 2, 3, 4, 5, 6, or 7. In a further aspect, o is 1, 2, 3, 4, 5, 6, 7, 8, or 9. In a still further aspect, o is 1, 2, 3, or 4.

In various aspects, p is 1. In a further aspect, p is 2.

In various aspects, A is S. In a further aspect, A is Se.

In various aspects, L is C2-C4 alkyl. In a further aspect, L is —(C1-C4 alkyl) (OCH₂CH₂) q. In a still further aspect, L is a structure selected from:

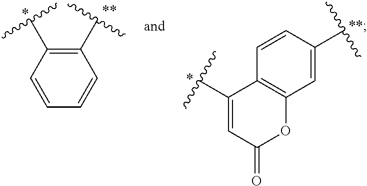 [SMILES: *c1ccccc1* ; *c1ccc2c(*)cc(=O)oc2c1]

In various aspects, the cleavable moiety is a protease recognition sequence. In a further aspect, the protease recognition sequence is a TEV protease recognition sequence. In a still further aspect, the TEV protease recognition sequence is EXLYZQ (SEQ ID NO: 1), in which X is any amino acid and Z is any amino acid that contains a hydrophobic residue. In yet a further aspect, the TEV protease recognition sequence is ENLYFQ (SEQ ID NO: 1).

In various aspects, the leader sequence is LKQINVIAGVKEPIRAYG (SEQ ID NO: 2) or LKQINVIAGVKPIRAYG (SEQ ID NO: 3). In a further aspect, the leader sequence is LKQINVIAGVKEPIRAYG (SEQ ID NO: 2).

In various aspects, R¹ is selected from —CO₂H and a structure:

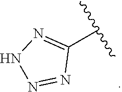 [SMILES: *c1nn[nH]n1]

In various aspects, R¹ is —CO₂H.

In various aspects, R² is a residue of a side chain of an amino acid selected from alanine, valine, leucine, serine, cysteine, methionine, arginine, lysine, asparagine, glycine, phenylalanine, tyrosine, and tryptophan. In a further aspect, R² is a residue of a side chain of an amino acid selected from alanine, leucine, serine, cysteine, methionine, arginine, lysine, asparagine, and glycine.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, R⁴ is hydrogen. In a further aspect, R⁴ is methyl.

In various aspects, each occurrence of R⁵, when present, is independently a residue of a side chain of an amino acid selected from alanine, valine, leucine, serine, cysteine, methionine, arginine, lysine, asparagine, glycine, phenylalanine, tyrosine, and tryptophan.

In various aspects, each occurrence of R⁶, when present, is hydrogen. In a further aspect, each occurrence of R⁶, when present, is methyl.

In various aspects, each of R^{7a} and R^{7b}, when present, is hydrogen. In a further aspect, each of R^{7a} and R^{7b}, when present, is methyl.

In various aspects, the compound has a structure represented by a formula:

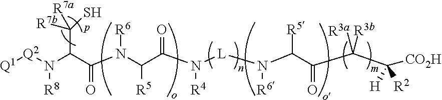

In various aspects, the compound has a structure represented by a formula:

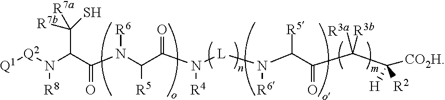

In various aspects, the compound has a structure represented by a formula:

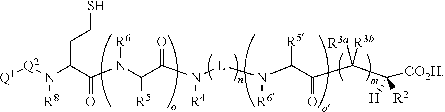

In various aspects, the compound has a structure represented by a formula:

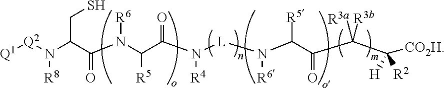

In various aspects, the compound has a structure represented by a formula:

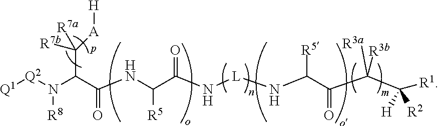

In various aspects, the compound has a structure represented by a formula:

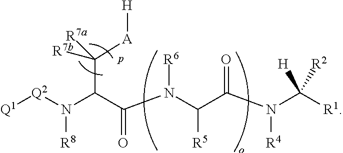

In various aspects, the compound has a structure represented by a formula:

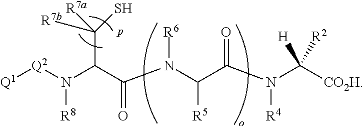

In various aspects, the compound has a structure represented by a formula:

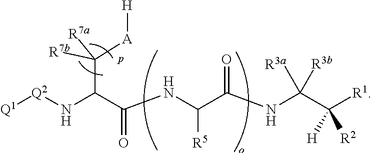

In a further aspect, o is 1, 2, 3, 4, 5, 6, 7, 8, or 9.

In various aspects, one of R^{3a} and R^{3b}, when present, is hydrogen, and one of R^{3a} and R^{3b}, when present, is a residue of a side chain of an amino acid selected from alanine, valine, leucine, serine, cysteine, methionine, arginine, lysine, asparagine, glycine, phenylalanine, tyrosine, and tryptophan.

In various aspects, the compound has a structure represented by a formula:

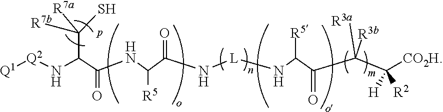

In various aspects, the compound has a structure represented by a formula:

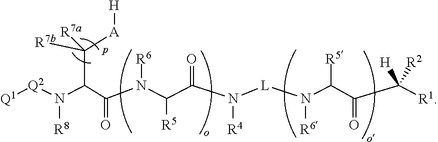

In various aspects, the compound has a structure represented by a formula:

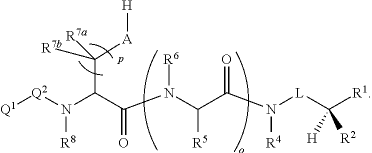

In various aspects, the compound has a structure represented by a formula:

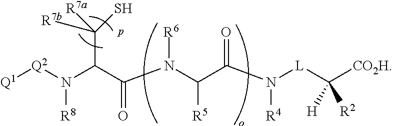

In a further aspect, o is 1, 2, 3, 4, 5, 6, 7, 8, or 9.

In various aspects, the compound has a structure represented by a formula:

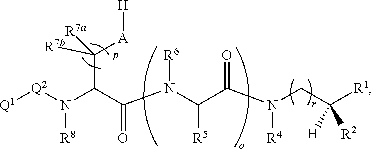

wherein r is 2, 3, or 4.

In various aspects, the compound has a structure represented by a formula:

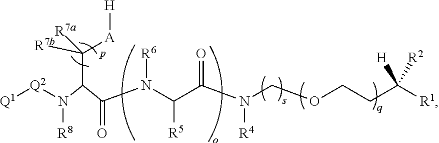

wherein s is 1 or 2.

In various aspects, the compound has a structure represented by a formula:

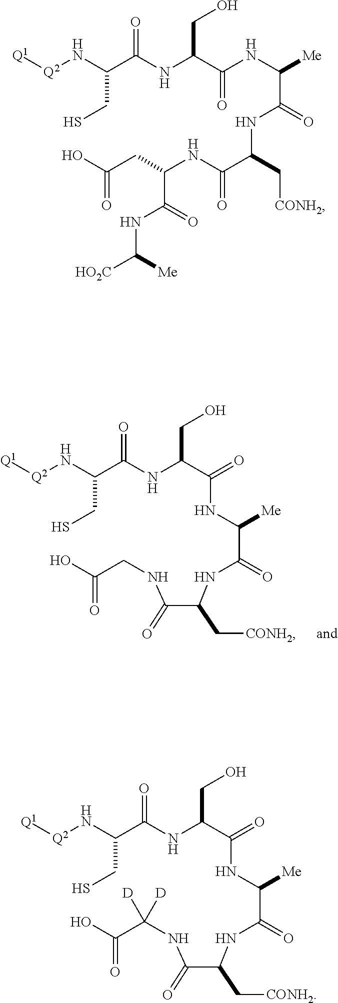

In various aspects, PapB installs a single thioether linkage in the compound.

In a further aspect, PapB installs two or more thioether linkages in the compound.

In various aspects, the method produces a thioether compound having a structure represented by a formula:

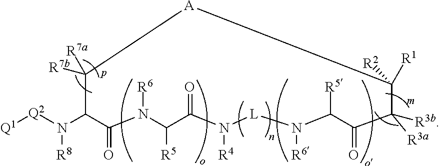

In various aspects, the thioether compound has a structure represented by a formula:

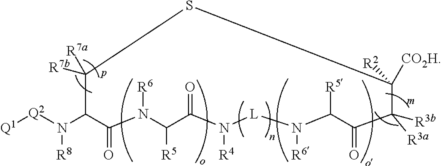

In various aspects, the thioether compound has a structure represented by a formula:

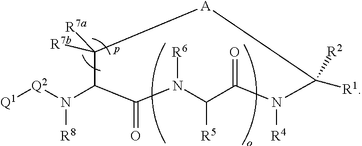

In various aspects, the thioether compound has a structure represented by a formula:

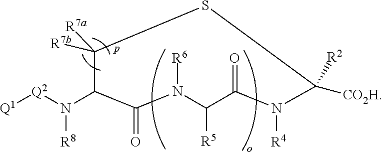

In various aspects, the thioether compound has a structure represented by a formula:

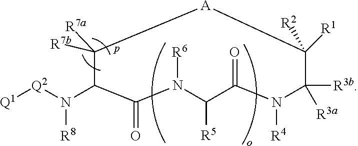

In various aspects, the thioether compound has a structure represented by a formula:

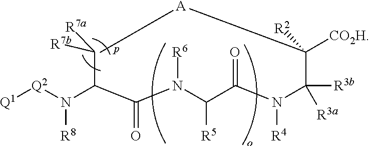

In various aspects, the thioether compound has a structure represented by a formula:

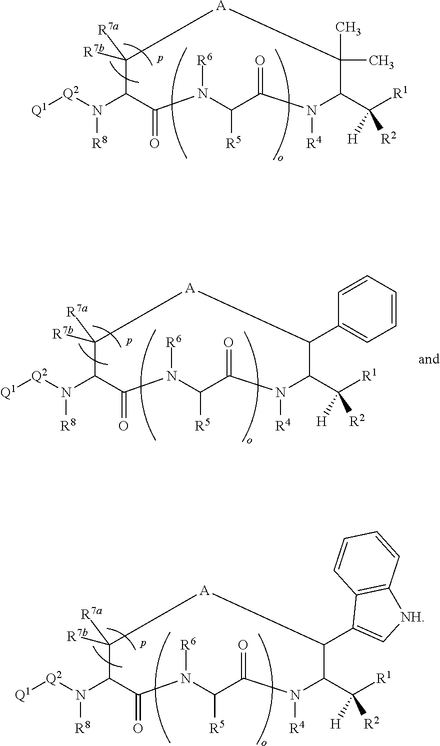

In various aspects, the thioether compound has a structure represented by a formula:

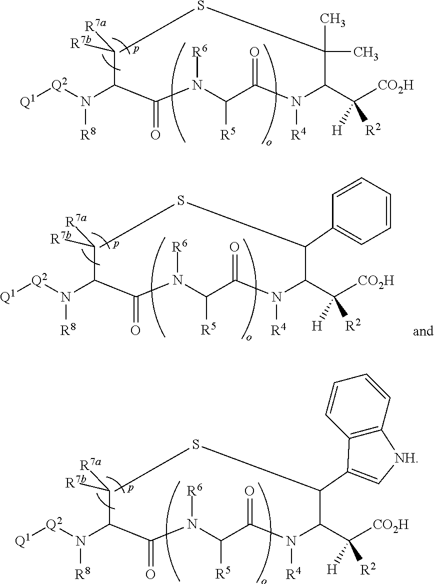

In various aspects, the thioether compound has a structure represented by a formula:

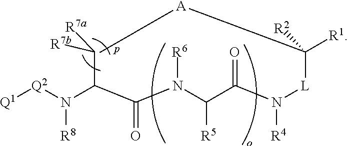

In various aspects, the thioether compound has a structure represented by a formula:

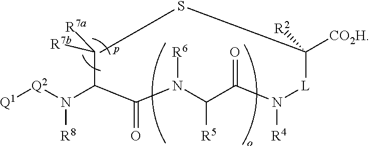

In various aspects, the thioether compound has a structure represented by a

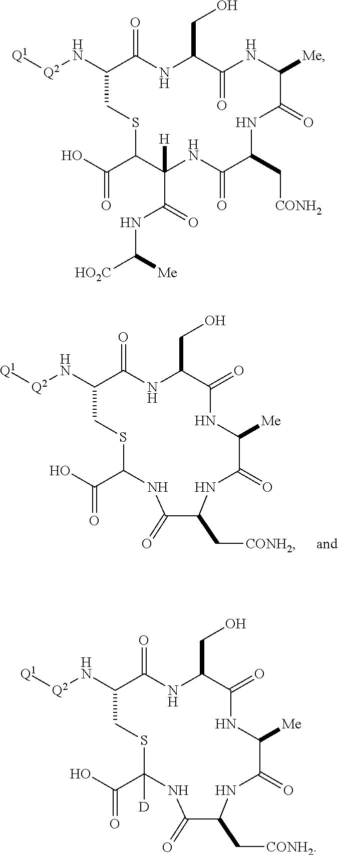

In various aspects, the thioether compound has a structure represented by a formula:

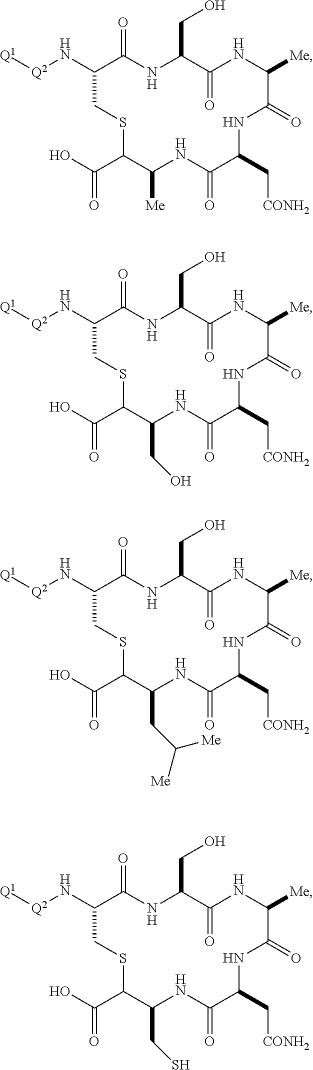

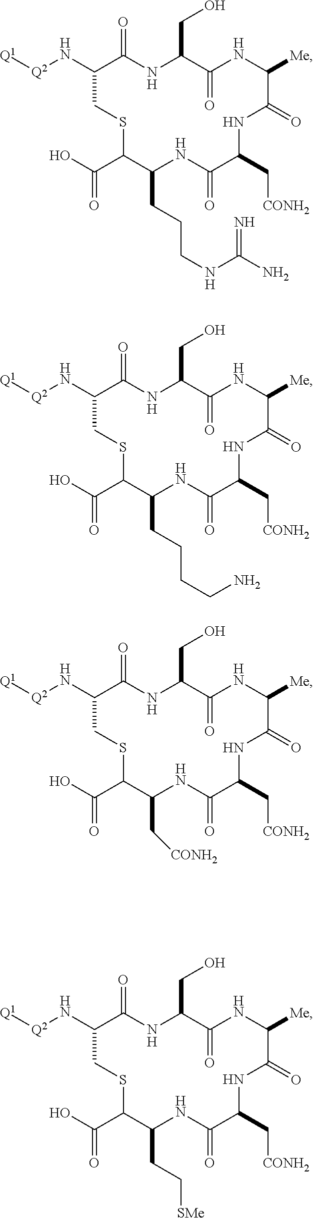

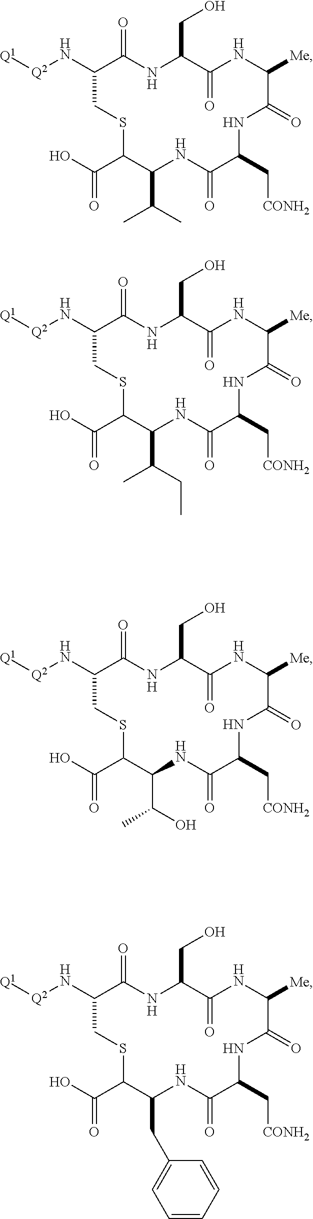

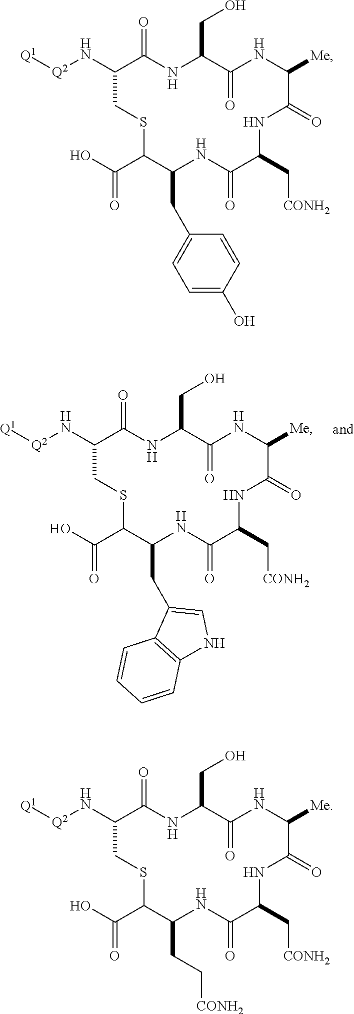

In various aspects, the thioether compound has a structure represented by a formula:

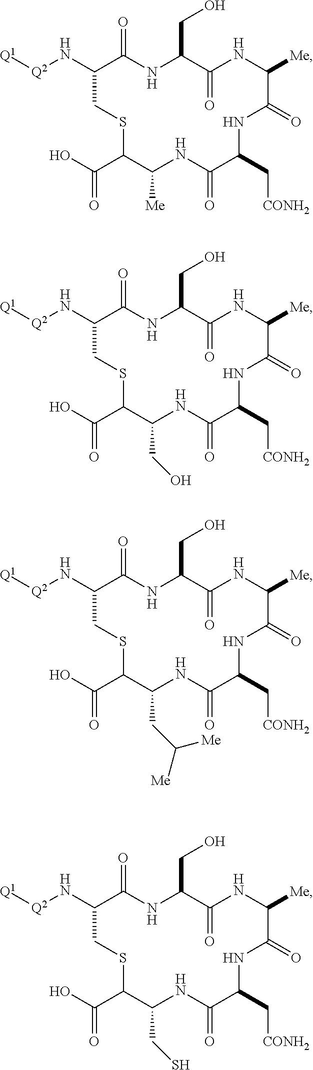

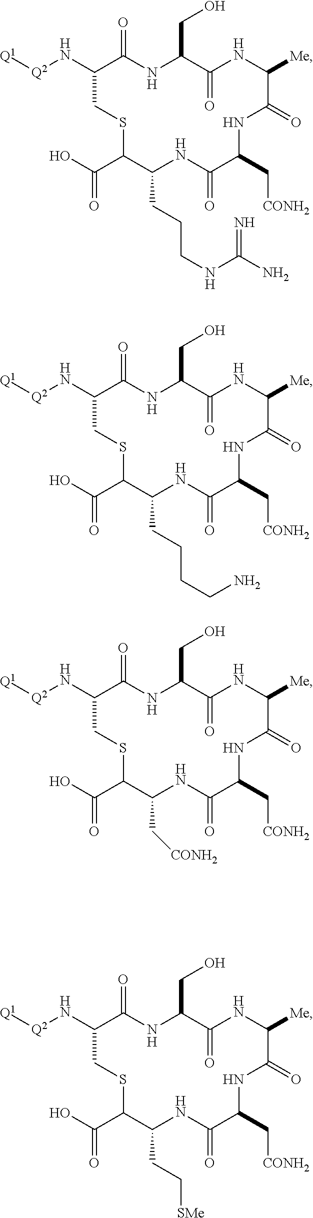

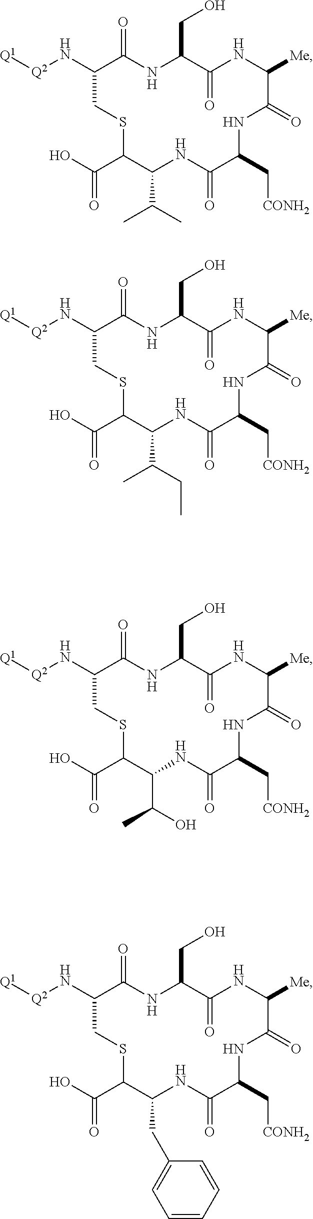

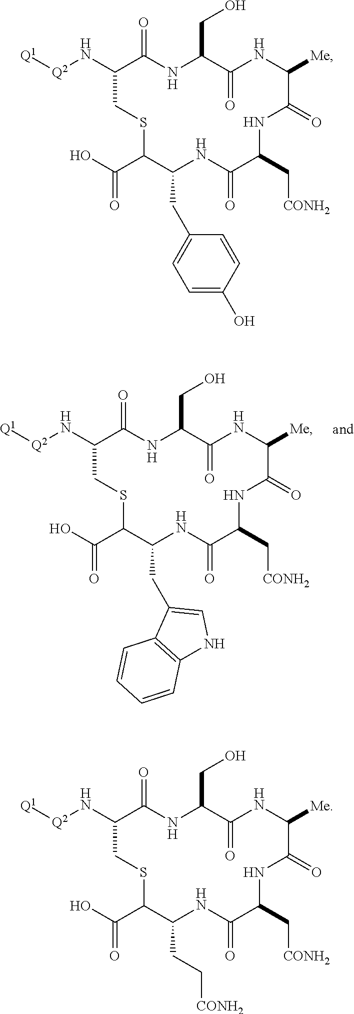

In various aspects, the thioether compound has a structure represented by a formula:

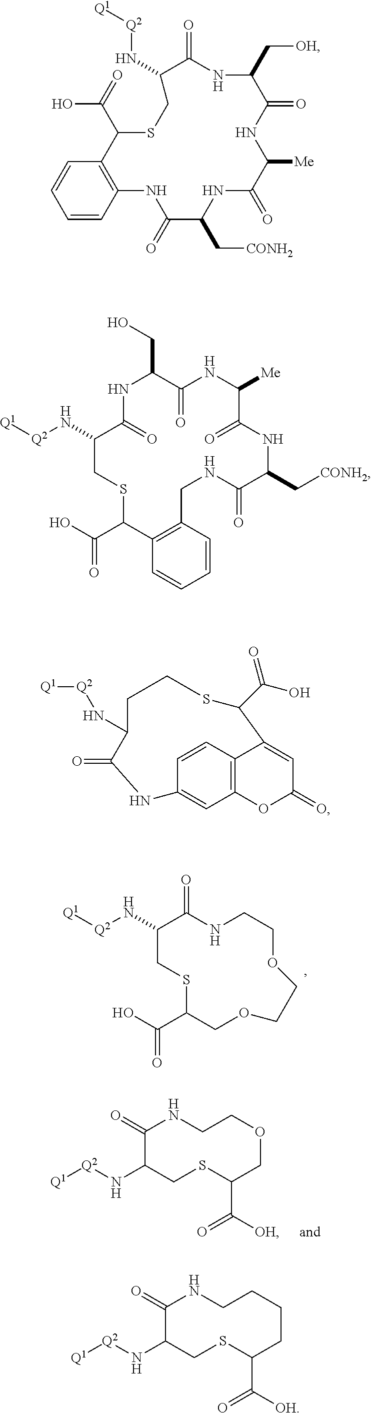

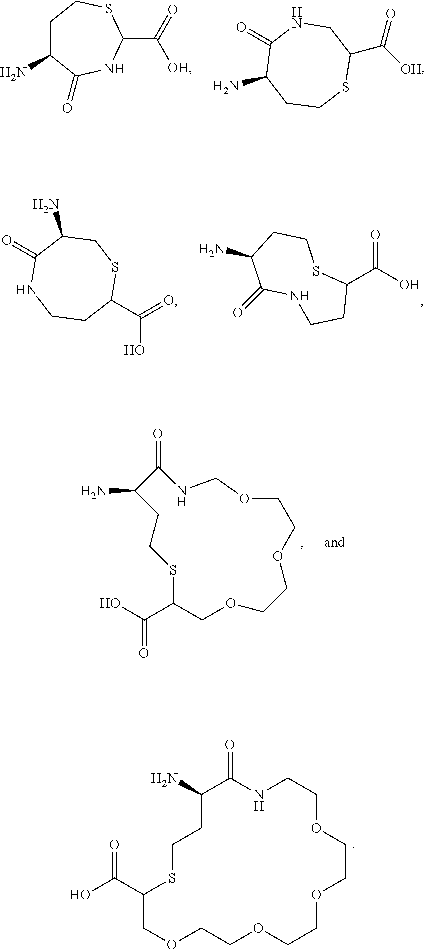 [SMILES: N[C@H]1CCSC(C(=O)O)NC1=O ; N[C@@H]1CCSC(C(=O)O)CNC1=O ; N[C@H]1CSC(C(=O)O)CCNC1=O ; N[C@H]1CCSC(C(=O)O)CCNC1=O ; N[C@@H]1CCSC(C(=O)O)COCCOCCOCNC1=O ; N[C@@H]1CCSC(C(=O)O)COCCOCCOCCOCCNC1=O]

In various aspects, the thioether compound is a sactipeptide. In a further aspect, the sactipeptide has a structure represented by a formula selected from:

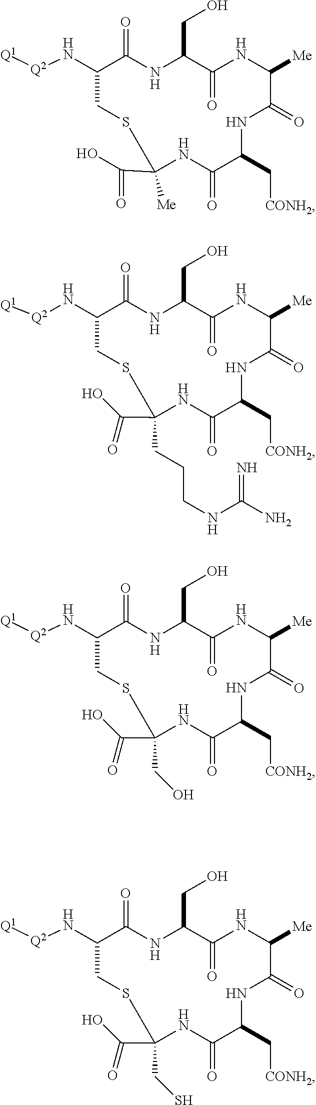

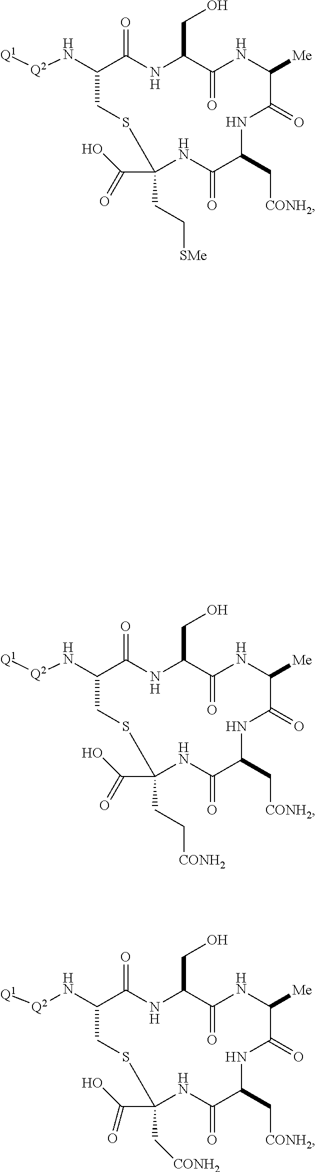

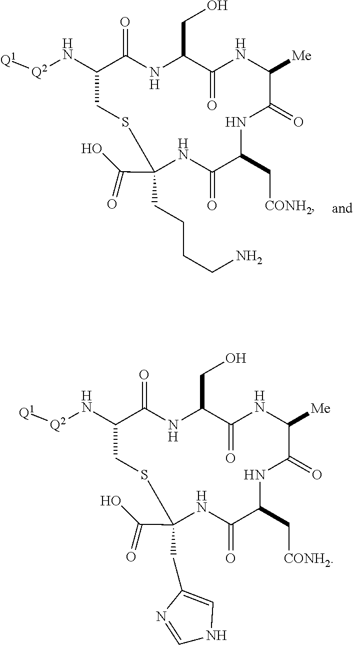

In various aspects, the thioether compound is a ranthipeptide. In a further aspect, the ranthipeptide has a structure represented by a formula selected from:

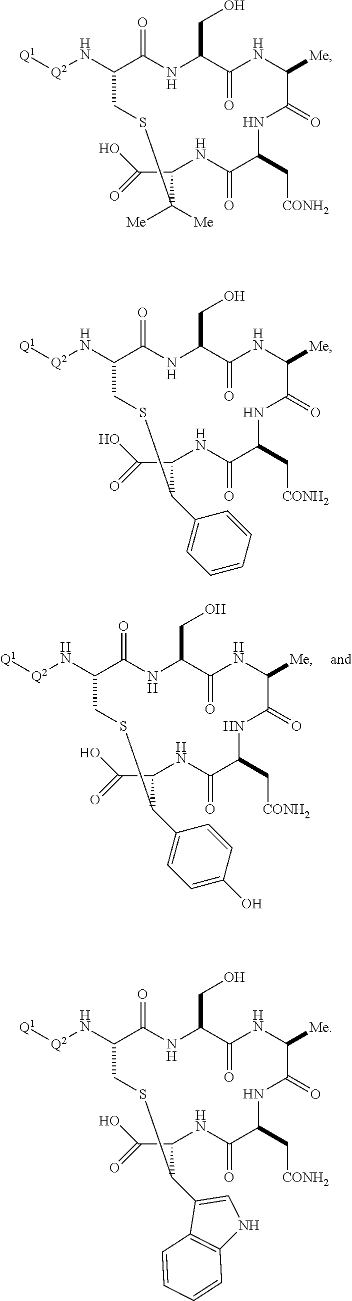

In various aspects, the method further comprises addition of a reducing agent. In a further aspect, the reducing agent comprises dithionite, flavodoxin, flavodoxin reductase, titanium citrate, reduced nicotinamide adenine dinucleotide phosphate, or any combination thereof.

In various aspects, the method further comprises addition of a protease. In a further aspect, the protease is TEV protease.

In various aspects, the method produces a thioether compound having a structure represented by a formula:

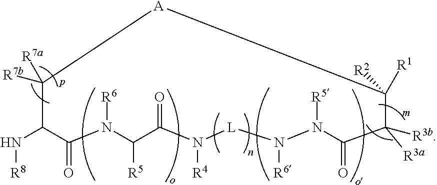

In various aspects, the thioether compound has a structure represented by a formula:

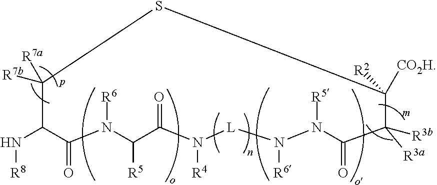

In various aspects, the thioether compound has a structure represented by a formula:

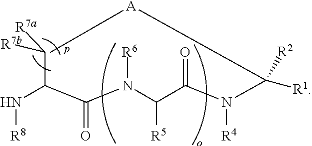

In various aspects, the thioether compound has a structure represented by a

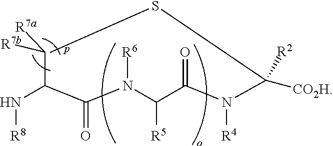

In various aspects, the thioether compound has a structure represented by a formula:

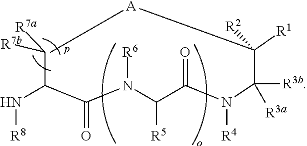

In various aspects, the thioether compound has a structure represented by a formula:

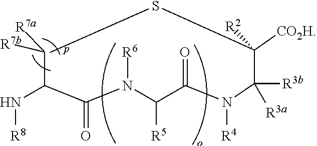

In various aspects, the thioether compound has a structure represented by a formula:

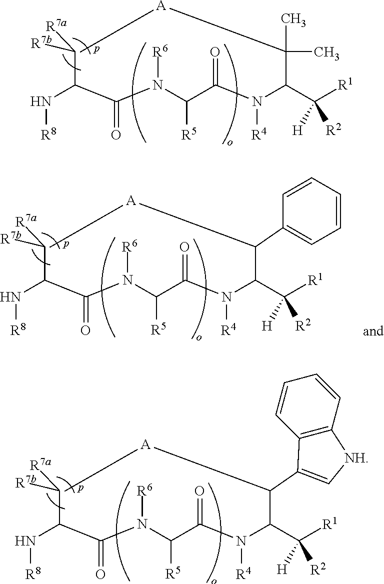

In various aspects, the thioether compound has a structure represented by a

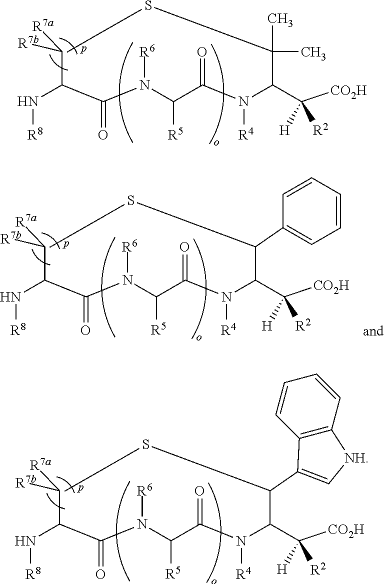

In various aspects, the thioether compound has a structure represented by a formula:

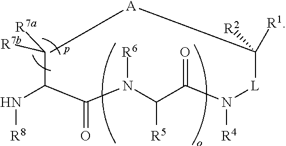

In various aspects, the thioether compound has a structure represented by a formula:

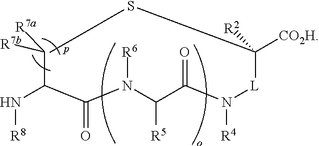

In various aspects, the thioether compound has a structure represented by a formula:

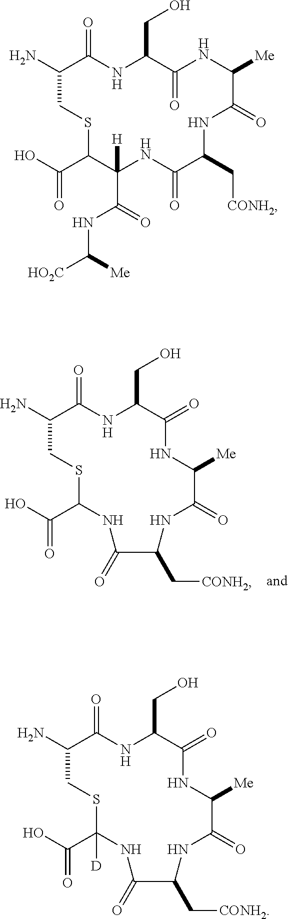 [SMILES: C[C@H](NC(=O)[C@H]1NC(=O)[C@H](CC(N)=O)NC(=O)[C@H](C)NC(=O)[C@H](CO)NC(=O)[C@@H](N)CSC1C(=O)O)C(=O)O ; C[C@@H]1NC(=O)[C@H](CO)NC(=O)[C@@H](N)CSC(C(=O)O)NC(=O)[C@H](CC(N)=O)NC1=O ; [2H]C1(C(=O)O)NC(=O)[C@H](CC(N)=O)NC(=O)[C@H](C)NC(=O)[C@H](CO)NC(=O)[C@@H](N)CS1]

In various aspects, the thioether compound has a structure represented by a formula:

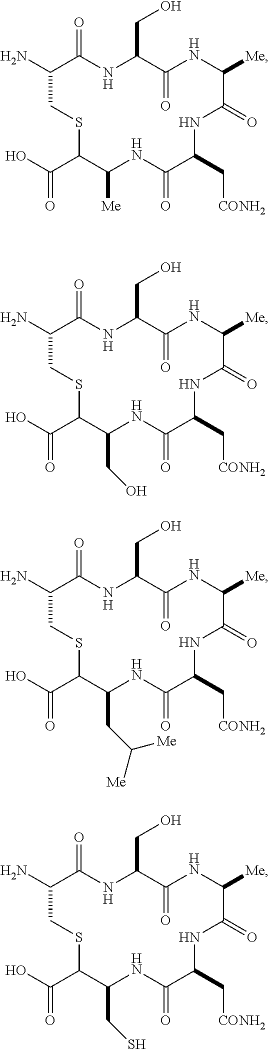 [SMILES: C[C@@H]1NC(=O)[C@H](CO)NC(=O)[C@@H](N)CSC(C(=O)O)[C@H](C)NC(=O)[C@H](CC(N)=O)NC1=O ; C[C@@H]1NC(=O)[C@H](CO)NC(=O)[C@@H](N)CSC(C(=O)O)[C@H](CO)NC(=O)[C@H](CC(N)=O)NC1=O ; CC(C)C[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H](CS)NC(=O)[C@H](CC(N)=O)NC1=O]

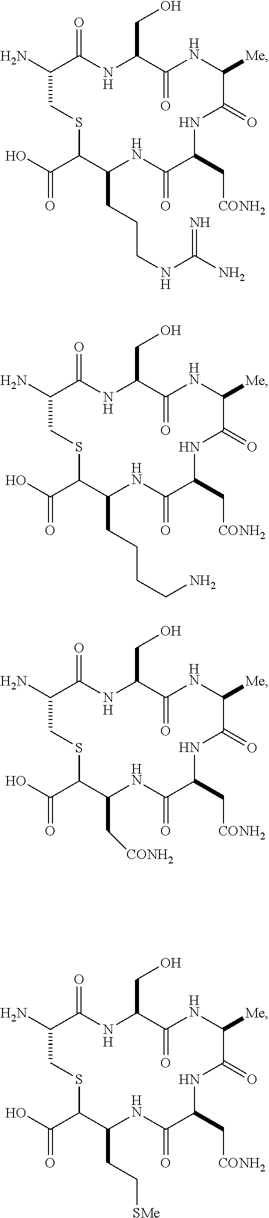

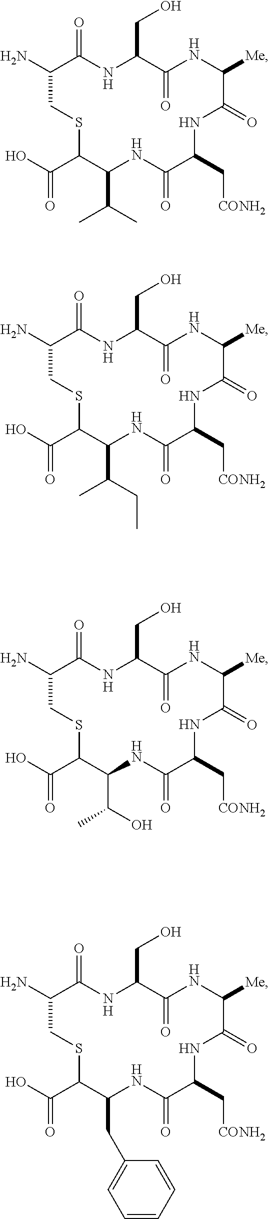 [SMILES: CC(C)[C@@H]1NC(=O)[C@H](CC(N)=O)NC(=O)[C@H](C)NC(=O)[C@H](CO)NC(=O)[C@@H](N)CSC1C(=O)O ; CCC(C)[C@@H]1NC(=O)[C@H](CC(N)=O)NC(=O)[C@H](C)NC(=O)[C@H](CO)NC(=O)[C@@H](N)CSC1C(=O)O ; C[C@@H]1NC(=O)[C@H](CO)NC(=O)[C@@H](N)CSC(C(=O)O)[C@H]([C@@H](C)O)NC(=O)[C@H](CC(N)=O)NC1=O ; C[C@@H]1NC(=O)[C@H](CO)NC(=O)[C@@H](N)CSC(C(=O)O)[C@H](Cc2ccccc2)NC(=O)[C@H](CC(N)=O)NC1=O]

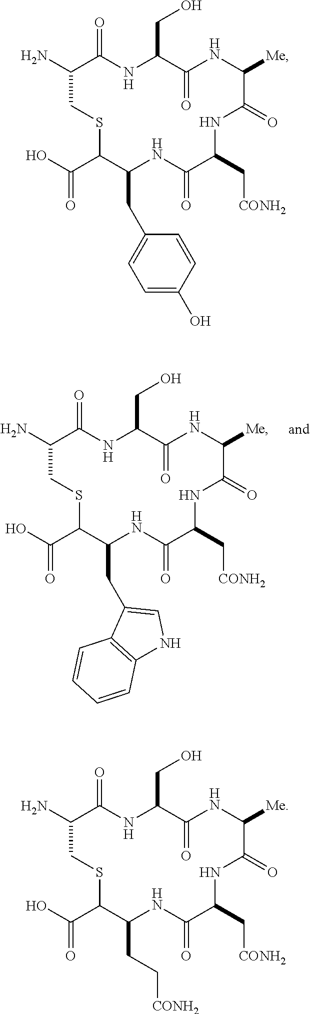 [SMILES: C[C@@H]1NC(=O)[C@H](CO)NC(=O)[C@@H](N)CSC(C(=O)O)[C@H](Cc2ccc(O)cc2)NC(=O)[C@H](CC(N)=O)NC1=O ; C[C@@H]1NC(=O)[C@H](CO)NC(=O)[C@@H](N)CSC(C(=O)O)[C@H](Cc2c[nH]c3ccccc23)NC(=O)[C@H](CC(N)=O)NC1=O ; C[C@@H]1NC(=O)[C@H](CO)NC(=O)[C@@H](N)CSC(C(=O)O)[C@H](CCC(N)=O)NC(=O)[C@H](CC(N)=O)NC1=O]

In various aspects, the thioether compound has a structure represented by a formula:

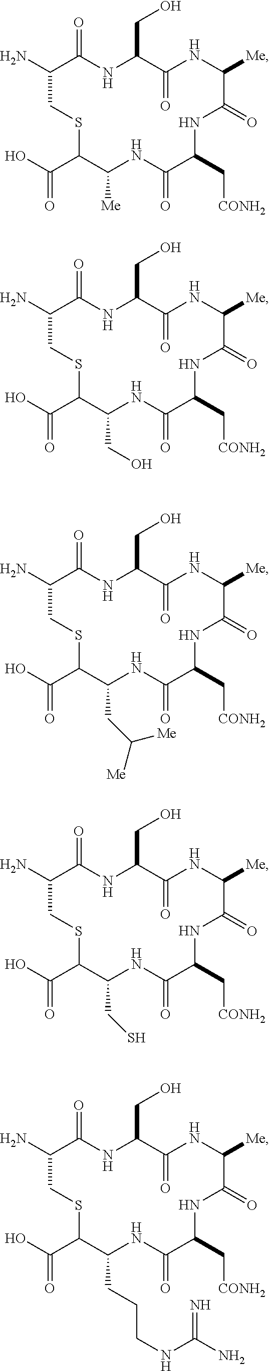

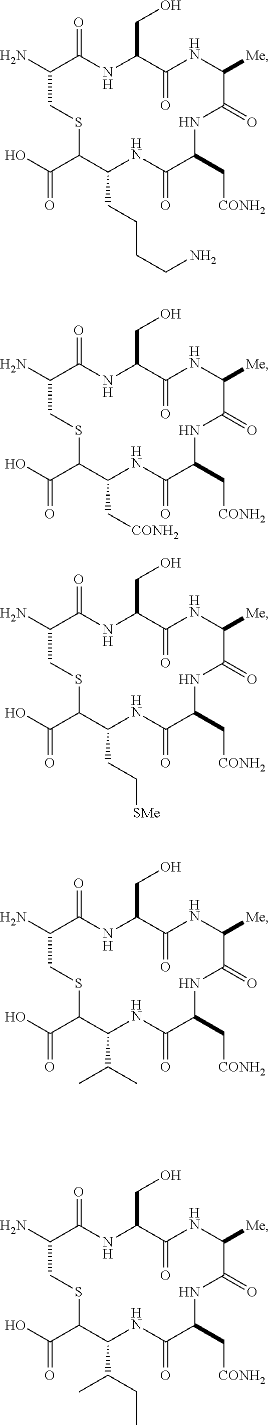

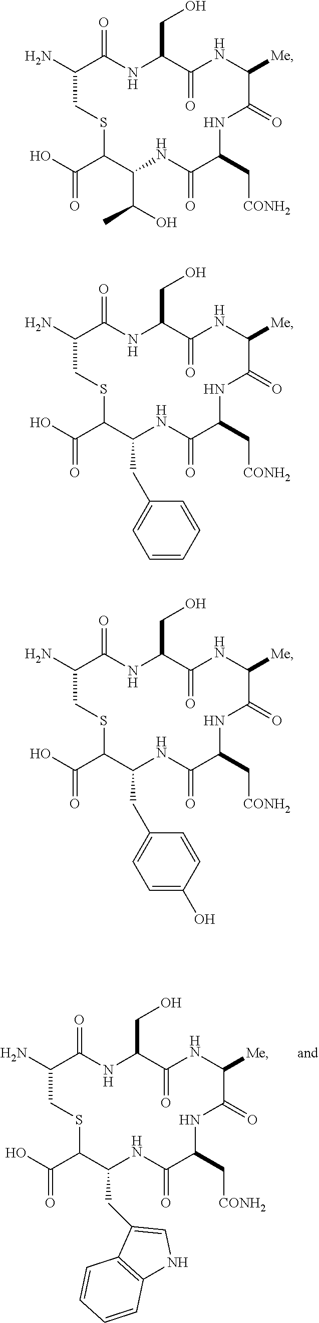

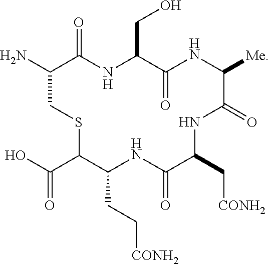 [SMILES: C[C@@H]1NC(=O)[C@H](CO)NC(=O)[C@@H](N)CSC(C(=O)O)[C@@H](CCC(N)=O)NC(=O)[C@H](CC(N)=O)NC1=O]

In various aspects, the thioether compound has a structure represented by a formula:

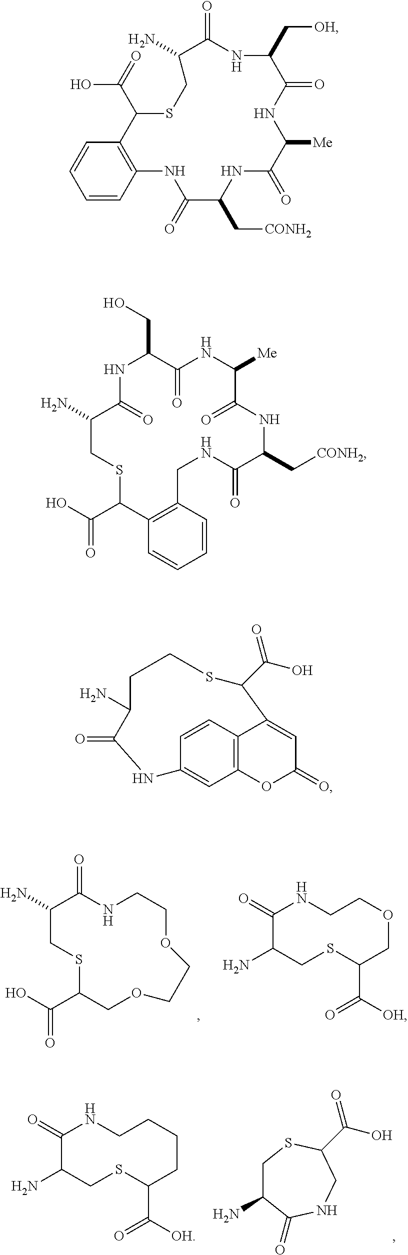

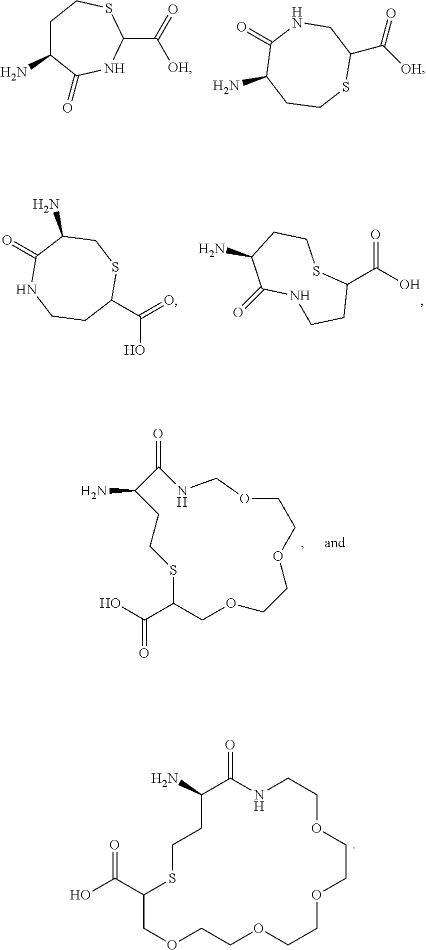 [SMILES: N[C@H]1CCSC(C(=O)O)NC1=O ; N[C@@H]1CCSC(C(=O)O)CNC1=O ; N[C@H]1CSC(C(=O)O)CCNC1=O ; N[C@H]1CCSC(C(=O)O)CCNC1=O ; N[C@@H]1CCSC(C(=O)O)COCCOCCOCNC1=O ; N[C@@H]1CCSC(C(=O)O)COCCOCCOCCOCCNC1=O]

In various aspects, the thioether compound is a sactipeptide. In a further aspect, the sactipeptide has a structure represented by a formula selected from:

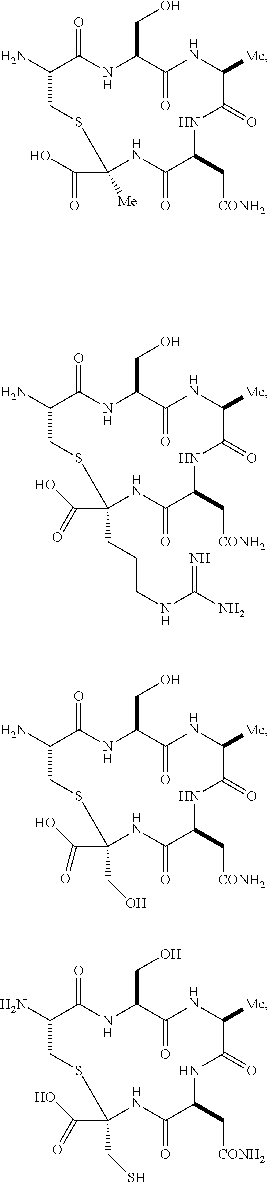 [SMILES: C[C@@H]1NC(=O)[C@H](CO)NC(=O)[C@@H](N)CS[C@](C)(C(=O)O)NC(=O)[C@H](CC(N)=O)NC1=O ; C[C@@H]1NC(=O)[C@H](CO)NC(=O)[C@@H](N)CS[C@](CCCNC(=N)N)(C(=O)O)NC(=O)[C@H](CC(N)=O)NC1=O ; C[C@@H]1NC(=O)[C@H](CO)NC(=O)[C@@H](N)CS[C@](CO)(C(=O)O)NC(=O)[C@H](CC(N)=O)NC1=O ; C[C@@H]1NC(=O)[C@H](CO)NC(=O)[C@@H](N)CS[C@](CS)(C(=O)O)NC(=O)[C@H](CC(N)=O)NC1=O]

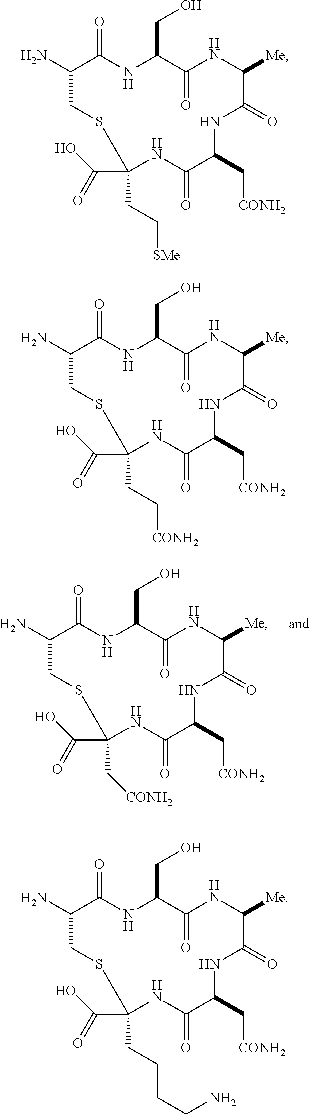

In various aspects, the thioether compound is a ranthipeptide. In a further aspect, the ranthipeptide has a structure represented by a formula selected from:

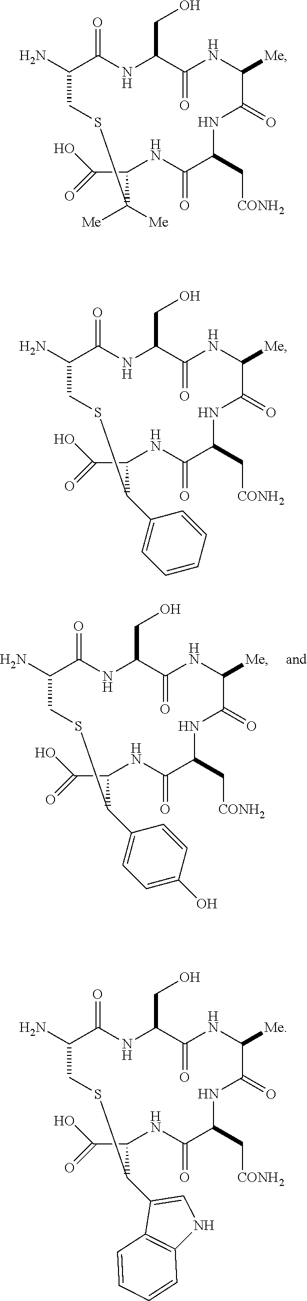

In various aspects, the thioether compound is selected from:

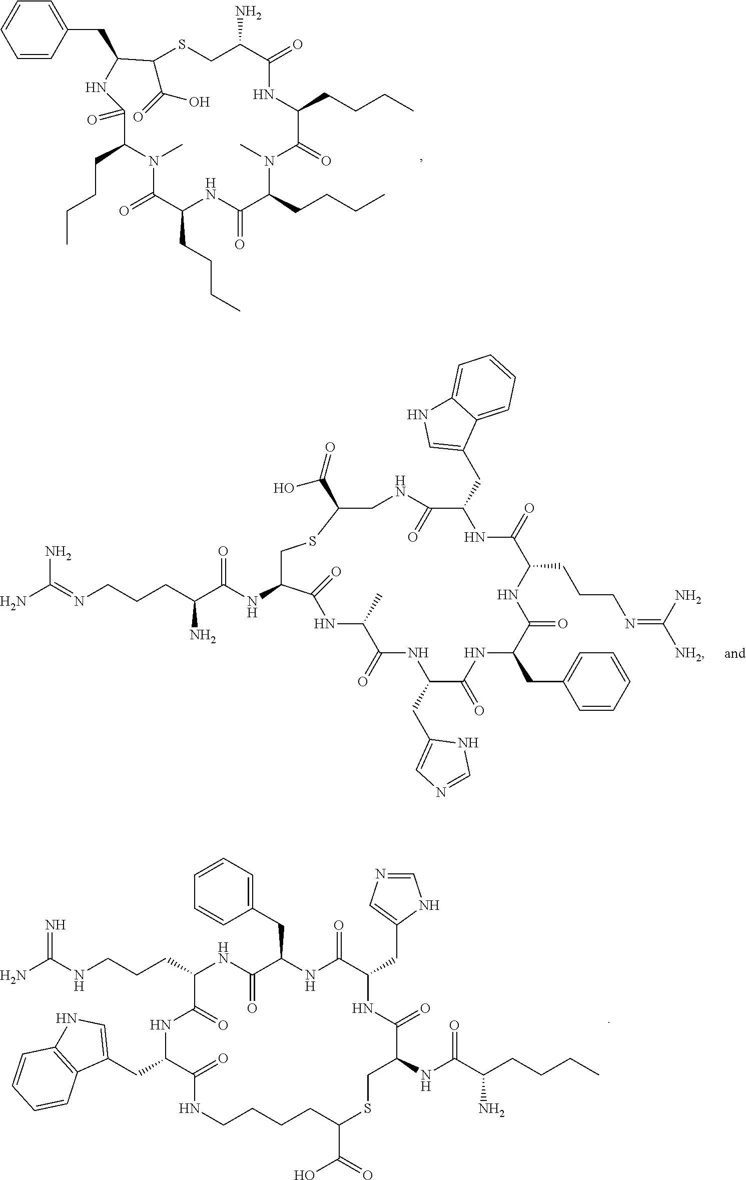 [SMILES: CCCC[C@@H]1NC(=O)[C@@H](N)CSC(C(=O)O)[C@H](Cc2ccccc2)NC(=O)[C@H](CCCC)N(C)C(=O)[C@H](CCCC)NC(=O)[C@H](CCCC)N(C)C1=O ; C[C@H]1NC(=O)[C@@H](NC(=O)[C@@H](N)CCCN=C(N)N)CS[C@@H](C(=O)O)CNC(=O)[C@H](Cc2c[nH]c3ccccc23)NC(=O)[C@H](CCCN=C(N)N)NC(=O)[C@@H](Cc2ccccc2)NC(=O)[C@H](Cc2cnc[nH]2)NC1=O ; CCCC[C@H](N)C(=O)N[C@H]1CSC(C(=O)O)CCCCNC(=O)[C@H](Cc2c[nH]c3ccccc23)NC(=O)[C@H](CCCNC(=N)N)NC(=O)[C@@H](Cc2ccccc2)NC(=O)[C@H](Cc2cnc[nH]2)NC1=O]

In various aspects, the thioether compound is selected from:

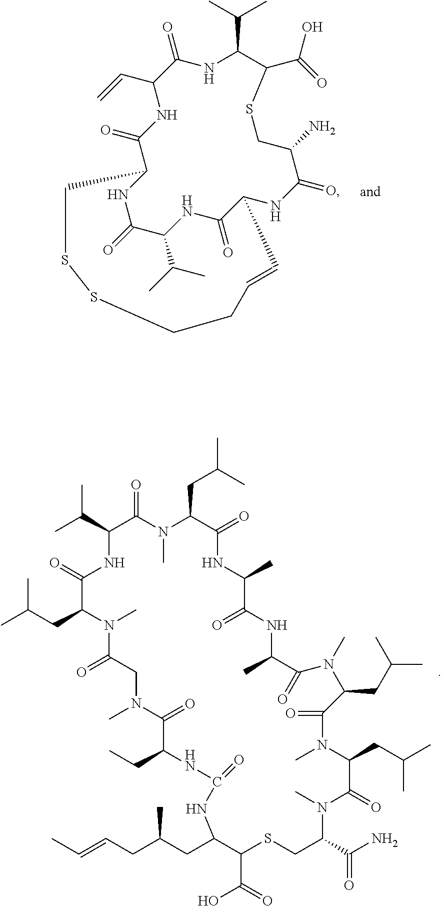 [SMILES: C=CC1NC(=O)[C@@H]2CSSCC/C=C/[C@H](NC(=O)[C@@H](N)CSC(C(=O)O)[C@H](C(C)C)NC1=O)C(=O)N[C@H](C(C)C)C(=O)N2 ; C/C=C/C[C@@H](C)CC1NC(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@H](C(N)=O)CSC1C(=O)O]

### F. PEPTIDES

### 1. Peptide Substrates

In one aspect, the invention relates to chemically modifying a peptide sequence, wherein the peptide sequence comprises X—Yₙ—Z; wherein X is a penicillamine or an amino acid residue comprising a —SH group or an amino acid residue comprising a —SeH group; wherein Y is a series of amino acid residues where n=0, 1, 2, 3, 4, 5, 6, 7, 8, or 9; wherein Z is an aspartic acid residue, a glutamic acid residue, a hydroxy-glutamic acid residue, 2-amino-3-(2H-tetrazol-5-yl) propanoic acid, or a carboxyl-functionalized amino acid residue; and wherein the peptide sequence is not PapA.

In a further aspect, the peptide sequence comprises the sequence C—Yₐ—C-D-Y_{b}-D; wherein C is a cysteine residue, D is an aspartic acid residue, Y is a series of amino acid residues, a=1, 2, 3, 4, 5, 6, or 7, and b=0, 1, 2, 3, 4, 5, 6, or 7.

In a further aspect, the peptide sequence comprises the sequence C—Yₓ-D-Y_{y}—C—Y_{z}-D; wherein C is a cysteine residue, D is an aspartic acid residue, Y is a series of amino acid residues, x=0, 1, 2, 3, 4, 5, 6, or 7, y=1, 2, 3, 4, 5, 6, 7, or 8, and z=0, 1, 2, 3, 4, 5, 6, or 7.

In a further aspect, the peptide sequence comprises octreotide or vapreotide. In a yet further aspect, the peptide sequence comprises octreotide. In a yet further aspect, the peptide sequence comprises vapreotide.

In a further aspect, the peptide sequence comprises DFCFDWKTET (SEQ ID NO: 3), wherein the first and fourth positions are D-amino acids.

In a further aspect, the peptide sequence comprises FCFAKTETA.

In various aspects, the peptide sequence further comprises a leader sequence of LKQINVIAGVKEPIRAYG (SEQ ID NO: 2) or LKQINVIAGVKPIRAYG (SEQ ID NO: 3). In a further aspect, the peptide sequence further comprises a leader sequence of LKQINVIAGVKEPIRAYG (SEQ ID NO: 3).

In various aspects, the peptide sequence further comprises a TEV protease recognition sequence. In a further aspect, the TEV protease recognition sequence is EXLYZQ (SEQ ID NO: 1), in which X is any amino acid and Z is any amino acid that contains a hydrophobic residue. In yet a further aspect, the TEV protease recognition sequence is ENLYFQ (SEQ ID NO: 1).

In a further aspect, the peptide sequence comprises one or more D-amino acid residues.

In a further aspect, the peptide sequence comprises one or more β-amino acid residues.

In a further aspect, the peptide sequence comprises one or more N-methylated amino acids.

In one aspect, the peptide sequence is a modified PapA sequence, wherein wherein the modified PapA sequence comprises Cys-Yₙ-Asp, wherein Y is a series of amino acid residues and n=0, 1, 2, 4, 5, 6, or 7.

In a further aspect, the modified PapA sequence comprises minimal substrate PapA.

In a further aspect, the modified PapA sequence is LKQINVIAGVKEPIRAYGCDSNNAANA (SEQ ID NO: 6), LKQINVIAGVKEPIRAYGCSDNNAAA (SEQ ID NO: 7), LKQINVIAGVKEPIRAYGCSNDAAA (SEQ ID NO: 8), LKQINVIAGVKEPIRAYGCSAANDA (SEQ ID NO: 9), LKQINVIAGVKEPIRAYGCSAAANDA (SEQ ID NO: 10), or LKQINVIAGVKEPIRAYGCSAAAANDA (SEQ ID NO: 11). In a still further aspect, the modified PapA sequence is LKQINVIAGVKEPIRAYGCDSNNAANA (SEQ ID NO: 6). In a still further aspect, the modified PapA sequence is LKQINVIAGVKEPIRAYGCSDNNAAA (SEQ ID NO: 7). In a still further aspect, the modified PapA sequence is LKQINVIAGVKEPIRAYGCSNDAA A (SEQ ID NO: 8). In a still further aspect, the modified PapA sequence is LKQINVIAGVKEPIRAYGCSAANDA (SEQ ID NO: 9). In a still further aspect, the modified PapA sequence is LKQINVIAGVKEPIRAYGCSAAANDA (SEQ ID NO: 10). In a still further aspect, the modified PapA sequence is LKQINVIAGVKEPIRAYGCSAAAANDA (SEQ ID NO: 11).

In a further aspect, the modified PapA sequence is LKQINVIAGVKEPIRAYGAAACSANDA (SEQ ID NO: 12), LKQINVIAGVKEPIRAYGAAACSANDACSANDA (SEQ ID NO: 13), LKQINVIAGVKEPIRAYGAAACSACDAADA (SEQ ID NO: 14), LKQINVIAGVKEPIRAYGAAAASACDAADA (SEQ ID NO: 15), or LKQINVIAGVKEPIRAYGAAACSAADAAADA (SEQ ID NO: 16). In a still further aspect, the modified PapA sequence is LKQINVIAGVKEPIRAYGAAACSANDA (SEQ ID NO: 12). In a still further aspect, the modified PapA sequence is LKQINVIAGVKEPIRAYGAAACSANDACSANDA (SEQ ID NO: 13). In a still further aspect, the modified PapA sequence is LKQINVIAGVKEPIRAYGAAACSACDAADA (SEQ ID NO: 14). In a still further aspect, the modified PapA sequence is LKQINVIAGVKEPIRAYGAAAASACDAADA (SEQ ID NO: 15). In a still further aspect, the modified PapA sequence is LKQINVIAGVKEPIRAYGAAACSAADAAADA (SEQ ID NO: 16).

In a further aspect, the modified PapA sequence comprises one or more D-amino acid residues.

In a further aspect, the modified PapA sequence comprises one or more β-amino acid residues.

In a further aspect, the modified PapA sequence comprises one or more N-methylated amino acid residues.

a. X Groups

In various aspects, X is a penicillamine or an amino acid residue comprising a —SH group or an amino acid residue comprising a —SeH group.

In a further aspect, X is a penicillamine.

In a further aspect, X is an amino acid residue comprising a —SH group. In a still further aspect, X is cysteine, homocysteine, D-cysteine, or D-homocysteine. In a still further aspect, X is homocysteine. In a still further aspect, X is D-cysteine. In a still further aspect, X is D-homocysteine. In a yet further aspect, X is cysteine.

In a further aspect, X is an amino acid residue comprising a —SeH group. In a still further aspect, X is selenocysteine or homoselenocysteine. In a still further aspect, X is selenocysteine. In a yet further aspect, X is homoselenocysteine.

b. Yₙ Groups

In various aspects, Yₙ is a series of amino acid residues where n=0, 1, 2, 3, 4, 5, 6, 7, 8, or 9.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Yₙ comprises one or more D-amino acids.

In a further aspect, Yₙ comprises one or more β-amino acids.

In a further aspect, Yₙ comprises one or more N-methylated amino acids.

In various aspects, n is 0, 1, 2, 3, 4, 5, 6, 7, 8, or 9. In a further aspect, n is 0, 1, 2, 3, 4, 5, 6, 7, or 8. In a still further aspect, n is 0, 1, 2, 3, 4, 5, 6, or 7. In a still further aspect, n is 0, 1, 2, 3, 4, 5, or 6. In a still further aspect, n is 0, 1, 2, 3, 4, or 5. In a still further aspect, n is 0, 1, 2, 3, or 4. In a still further aspect, n is 0, 1, 2, or 3. In a still further aspect, n is 0, 1, or 2. In a yet further aspect, n is 0 or 1. In a yet further aspect, n is 0. In a yet further aspect, n is 1. In a yet further aspect, n is 2. In a yet further aspect, n is 3. In a yet further aspect, n is 4. In a yet further aspect, n is 5. In a yet further aspect, n is 6. In a yet further aspect, n is 7. In a yet further aspect, n is 8. In a yet further aspect, n is 9.

c. Z Groups

In various aspects, Z is an aspartic acid residue, a glutamic acid residue, a hydroxy-glutamic acid residue, 2-amino-3-(2H-tetrazol-5-yl) propanoic acid, or a carboxyl-functionalized amino acid residue.

In a further aspect, Z is aspartic acid or glutamic acid. In a yet further aspect, Z is aspartic acid. In a yet further aspect, Z is glutamic acid.

In a further aspect, Z is a hydroxy-glutamic acid residue.

In a further aspect, Z is 2-amino-3-(2H-tetrazol-5-yl) propanoic acid.

In a further aspect, Z is a a carboxyl-functionalized amino acid residue.

Examples of carboxyl-functionalized amino acid residues include, but are not limited to, (2S,3S)-2-amino-3-methylsuccinic acid, (2S,3R)-2-amino-3-methylsuccinic acid, (2S,3S)-2-amino-3-methylpentanedioic acid, (2S,3R)-2-amino-3-methylpentanedioic acid, (2S,4S)-2-amino-4-methylpentanedioic acid, (2S,4R)-2-amino-4-methylpentanedioic acid, and homoglutamic acid.

d. Yₐ Groups

In a further aspect, Yₐ is a series of amino acid residues where a=0, 1, 2, 3, 4, 5, 6, or 7.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Yₐ comprises one or more D-amino acids.

In a further aspect, Yₐ comprises one or more β-amino acids.

In a further aspect, Yₐ comprises one or more N-methylated amino acids.

In a further aspect, a is 0, 1, 2, 3, 4, 5, 6, or 7. In a further aspect, a is 0, 1, 2, 3, 4, 5, or 6. In a still further aspect, a is 0, 1, 2, 3, 4, or 5. In a still further aspect, a is 0, 1, 2, 3, or 4. In a still further aspect, a is 0, 1, 2, or 3. In a still further aspect, a is 0, 1, or 2. In a yet further aspect, a is 0 or 1. In a yet further aspect, a is 0. In a yet further aspect, a is 1. In a yet further aspect, a is 2. In a yet further aspect, a is 3. In a yet further aspect, a is 4. In a yet further aspect, a is 5. In a yet further aspect, a is 6. In a yet further aspect, a is 7.

e. Y_{b} Groups

In a further aspect, Y_{b} is a series of amino acid residues where b=0, 1, 2, 3, 4, 5, 6, or 7.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Y_{b} comprises one or more D-amino acids.

In a further aspect, Y_{b} comprises one or more β-amino acids.

In a further aspect, Y_{b} comprises one or more N-methylated amino acids.

In a further aspect, b is 0, 1, 2, 3, 4, 5, 6, or 7. In a further aspect, b is 0, 1, 2, 3, 4, 5, or 6. In a still further aspect, b is 0, 1, 2, 3, 4, or 5. In a still further aspect, b is 0, 1, 2, 3, or 4. In a still further aspect, b is 0, 1, 2, or 3. In a still further aspect, b is 0, 1, or 2. In a yet further aspect, b is 0 or 1. In a yet further aspect, b is 0. In a yet further aspect, b is 1. In a yet further aspect, b is 2. In a yet further aspect, b is 3. In a yet further aspect, b is 4. In a yet further aspect, b is 5. In a yet further aspect, b is 6. In a yet further aspect, b is 7.

f. Yₓ Groups

In a further aspect, Yₓ is a series of amino acid residues where x=0, 1, 2, 3, 4, 5, or 6.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Yₓ comprises one or more D-amino acids.

In a further aspect, Yₓ comprises one or more β-amino acids.

In a further aspect, Yₓ comprises one or more N-methylated amino acids.

In a further aspect, x is 0, 1, 2, 3, 4, 5, or 6. In a still further aspect, x is 0, 1, 2, 3, 4, or 5. In a still further aspect, x is 0, 1, 2, 3, or 4. In a still further aspect, x is 0, 1, 2, or 3. In a still further aspect, x is 0, 1, or 2. In a yet further aspect, x is 0 or 1. In a yet further aspect, x is 0. In a yet further aspect, x is 1. In a yet further aspect, x is 2. In a yet further aspect, x is 3. In a yet further aspect, x is 4. In a yet further aspect, x is 5. In a yet further aspect, x is 6.

g. Y_{y} Groups

In a further aspect, Y_{y} is a series of amino acid residues where y=0, 1, 2, 3, 4, 5, 6, 7, or 8.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Y_{y} comprises one or more D-amino acids.

In a further aspect, Y_{y} comprises one or more β-amino acids.

In a further aspect, Y_{y} comprises one or more N-methylated amino acids.

In a further aspect, y is 0, 1, 2, 3, 4, 5, 6, 7, or 8. In a still further aspect, y is 0, 1, 2, 3, 4, 5, 6, or 7. In a still further aspect, y is 0, 1, 2, 3, 4, 5, or 6. In a still further aspect, y is 0, 1, 2, 3, 4, or 5. In a still further aspect, y is 0, 1, 2, 3, or 4. In a still further aspect, y is 0, 1, 2, or 3. In a still further aspect, y is 0, 1, or 2. In a yet further aspect, y is 0 or 1. In a yet further aspect, y is 0. In a yet further aspect, y is 1. In a yet further aspect, y is 2. In a yet further aspect, y is 3. In a yet further aspect, y is 4. In a yet further aspect, y is 5. In a yet further aspect, y is 6. In a yet further aspect, y is 7. In a yet further aspect, y is 8.

h. Y_{z} Groups

In a further aspect, Y_{z} is a series of amino acid residues where z=0, 1, 2, 3, 4, 5, 6, or 7.

Examples of amino acid residues include, but are not limited to, natural amino acid residues, unnatural amino acid residues, D-amino acid residues, β-amino acid residues, and N-methylated amino acid residues.

Natural amino acid residues may include glycine, alanine, valine, leucine, isoleucine, proline, phenylalanine, methionine, tryptophan, serine, cysteine, threonine, tyrosine, asparagine, glutamine, aspartic acid, glutamic acid, arginine, histidine, and lysine.

Unnatural amino acid residues may include, but are not limited to, p-ethylthiocarbonyl-L-phenylalanine, p-(3-oxobutanoyl)-L-phenylalanine, 1,5-dansyl-alanine, 7-amino-coumarin amino acid, 7-hydroxy-coumarin amino acid, nitrobenzyl-serine, O-(2-nitrobenzyl)-L-tyrosine, p-carboxymethyl-L-phenylalanine, p-cyano-L-phenylalanine, m-cyano-L-phenylalanine, biphenylalanine, 3-amino-L-tyrosine, bipyridyl alanine, p-(2-amino-1-hydroxyethyl)-L-phenylalanine, p-isopropylthiocarbonyl-L-phenylalanine, 3-nitro-L-tyrosine and p-nitro-L-phenylalanine. Also, a p-propargyloxyphenylalanine, a 3,4-dihydroxy-L-phenyalanine (DHP), a 3,4,6-trihydroxy-L-phenylalanine, a 3,4,5-trihydroxy-L-phenylalanine, 4-nitro-phenylalanine, a p-acetyl-L-phenylalanine, O-methyl-L-tyrosine, an L-3-(2-naphthyl) alanine, a 3-methyl-phenylalanine, an O-4-allyl-L-tyrosine, a 4-propyl-L-tyrosine, a 3-nitro-tyrosine, a 3-thiol-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

In a further aspect, Y_{z} comprises one or more D-amino acids.

In a further aspect, Y_{z} comprises one or more β-amino acids.

In a further aspect, Y_{z} comprises one or more N-methylated amino acids.

In a further aspect, z is 0, 1, 2, 3, 4, 5, 6, or 7. In a further aspect, z is 0, 1, 2, 3, 4, 5, or 6. In a still further aspect, z is 0, 1, 2, 3, 4, or 5. In a still further aspect, z is 0, 1, 2, 3, or 4. In a still further aspect, z is 0, 1, 2, or 3. In a still further aspect, z is 0, 1, or 2. In a yet further aspect, z is 0 or 1. In a yet further aspect, z is 0. In a yet further aspect, z is 1. In a yet further aspect, z is 2. In a yet further aspect, z is 3. In a yet further aspect, z is 4. In a yet further aspect, z is 5. In a yet further aspect, z is 6. In a yet further aspect, z is 7.

It is contemplated that each disclosed derivative can be optionally further substituted. It is also contemplated that any one or more derivative can be optionally omitted from the invention. It is understood that a disclosed compound can be provided by the disclosed methods.

### 2. Example Peptide Products

In one aspect, the invention relates to product compounds having a structure selected from:

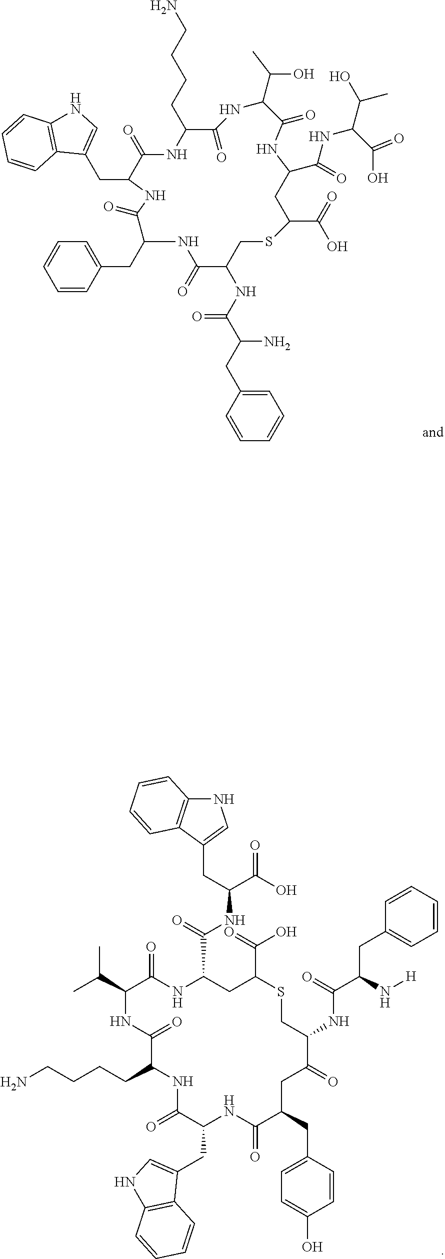

In a further aspect, the compound is:

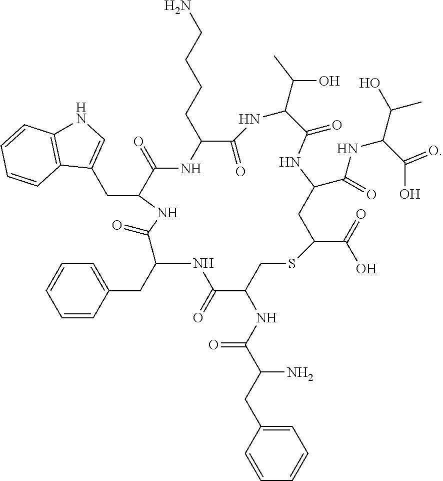 [SMILES: CC(O)C(NC(=O)C1CC(C(=O)O)SCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O]

In a further aspect, the compound is:

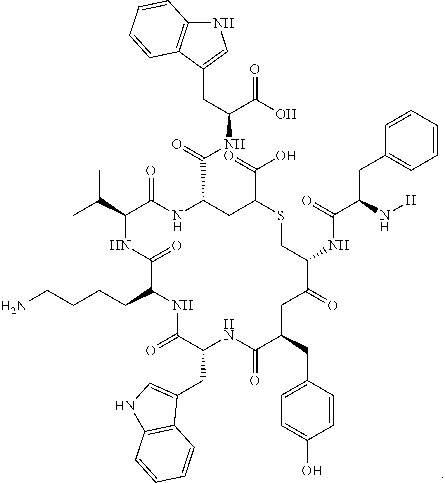

### G. METHODS OF CHEMICALLY MODIFYING A PEPTIDE SEQUENCE

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Preferred methods include, but are not limited to, those described below. During any of the following synthetic sequences, it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Reactions used to generate the compounds of this invention are prepared by employing reactions as shown in the following Reaction Schemes, as described and exemplified below. The following examples are provided so that the invention might be more fully understood, are illustrative only, and should not be construed as limiting.

In one aspect, the invention relates to methods of chemically modifying a peptide sequence to install a thioether linkage, the method comprising reacting the peptide substrate with PapB.

In a further aspect, the peptide sequence further comprises a leader sequence of LKQINVIAGVKEPIRAYG (SEQ ID NO: 2) or LKQINVIAGVKPIRAYG (SEQ ID NO: 3). The leader sequence facilitates recognition of the full peptide sequence by PapB. However, the leader sequence is not required.

In a further aspect, the method further comprises addition of a protease. In a further aspect, the peptide sequence comprises a protease recognition sequence. A protease in conjunction with a peptide sequence comprising a protease recognition sequence allows for cleavage of a desired product from the leader sequence. In a further aspect, the protease is a TEV protease. TEV protease in conjunction with a peptide sequence comprising a TEV protease recognition sequence allows for cleavage of a desired product from the leader sequence. In a further aspect, the peptide sequence comprises a TEV protease recognition sequence. In a still further aspect, the TEV protease recognition sequence is EXLYZQ (SEQ ID NO: 1), in which X is any amino acid and Z is any amino acid that contains a hydrophobic residue. In yet a further aspect, the TEV protease recognition sequence is ENLYFQ (SEQ ID NO: 1).

In a further aspect, the method further comprises addition of a reducing agent. Examples of reducing agents include, but are not limited to, dithionite, flavodoxin, flavodoxin reductase, titanium citrate, reduced nicotinamide adenine dinucleotide phosphate, and Hantzsch esters. In a still further aspect, the reducing agent comprises comprises dithionite, flavodoxin, flavodoxin reductase, titanium citrate, reduced nicotinamide adenine dinucleotide phosphate, or any combination thereof. In a still further aspect, the reducing agent comprises dithionite, flavodoxin, flavodoxin reductase, and titanium citrate. In a still further aspect, the reducing agent comprises dithionite, flavodoxin, and flavodoxin reductase. In a still further aspect, the reducing agent comprises dithionite and flavodoxin. In a still further aspect, the reducing agent comprises reduced nicotinamide adenine dinucleotide phosphate, titanium citrate, flavodoxin reductase, and flavodoxin. In a still further aspect, the reducing agent comprises reduced nicotinamide adenine dinucleotide phosphate, titanium citrate, and flavodoxin reductase. In a still further aspect, the reducing agent comprises reduced nicotinamide adenine dinucleotide phosphate and titanium citrate. In a still further aspect, the reducing agent comprises dithionite. In a still further aspect, the reducing agent comprises flavodoxin. In a still further aspect, the reducing agent comprises flavodoxin reductase. In a still further aspect, the reducing agent comprises titanium citrate. In a still further aspect, the reducing agent comprises reduced nicotinamide adenine dinucleotide phosphate.

In a further aspect, PapB installs two or more thioether linkages in the peptide sequence. For example, the peptide sequence may comprise the the sequence C—Yₐ—C-D-Y_{b}-D; wherein C is a cysteine residue, D is an aspartic acid residue, Y is a series of amino acid residues, a=1, 2, 3, 4, 5, 6, or 7, and b=0, 1, 2, 3, 4, 5, 6, or 7. In this example, thioether linkages are installed between the first cysteine residue and the first aspartic acid residue and between the second cysteine residue and the second aspartic acid residue, yielding nested crosslinks. By way of example, the peptide sequence may also comprise the sequence C—Yₓ-D-Y_{y}—C—Y_{z}-D; wherein C is a cysteine residue, D is an aspartic acid residue, Y is a series of amino acid residues, x=0, 1, 2, 3, 4, 5, 6, or 7, y=1, 2, 3, 4, 5, 6, 7, or 8, and z=0, 1, 2, 3, 4, 5, 6, or 7. In this example, thioether linkages are installed between the first cysteine residue and the first aspartic acid residue and between the second cysteine residue and the second aspartic acid residue, yielding in-line crosslinks.

### H. METHODS OF CHEMICALLY MODIFYING A MODIFIED PAPA SEQUENCE

In one aspect, the invention relates to methods of chemically modifying a modified PapA sequence to install a thioether linkage, the method comprising reacting the modified PapA sequence with PapB.

In a further aspect, the modified PapA sequence comprises minimal substrate PapA.

In a further aspect, the modified PapA sequence further comprises a leader sequence of LKQINVIAGVKEPIRAYG (SEQ ID NO: 2) or LKQINVIAGVKPIRAYG (SEQ ID NO: 3). The leader sequence facilitates recognition of the full peptide sequence by PapB. However, the leader sequence is not required.

In a further aspect, the method further comprises addition of a protease. In a further aspect, the modified PapA sequence comprises a protease recognition sequence. A protease in conjunction with a modified PapA sequence comprising a protease recognition sequence allows for cleavage of a desired product from the leader sequence. In a further aspect, the protease is a TEV protease. TEV protease in conjunction with a modified PapA sequence comprising a TEV protease recognition sequence allows for cleavage of a desired product from the leader sequence. In a further aspect, the modified PapA sequence comprises a TEV protease recognition sequence. In a still further aspect, the TEV protease recognition sequence is EXLYZQ (SEQ ID NO: 1), in which X is any amino acid and Z is any amino acid that contains a hydrophobic residue. In yet a further aspect, the TEV protease recognition sequence is ENLYFQ (SEQ ID NO: 1).

In a further aspect, the method further comprises addition of a reducing agent. Examples of reducing agents include, but are not limited to, dithionite, flavodoxin, flavodoxin reductase, titanium citrate, reduced nicotinamide adenine dinucleotide phosphate, and Hantzsch esters. In a still further aspect, the reducing agent comprises comprises dithionite, flavodoxin, flavodoxin reductase, titanium citrate, reduced nicotinamide adenine dinucleotide phosphate, or any combination thereof. In a still further aspect, the reducing agent comprises dithionite, flavodoxin, flavodoxin reductase, and titanium citrate. In a still further aspect, the reducing agent comprises dithionite, flavodoxin, and flavodoxin reductase. In a still further aspect, the reducing agent comprises dithionite and flavodoxin. In a still further aspect, the reducing agent comprises reduced nicotinamide adenine dinucleotide phosphate, titanium citrate, flavodoxin reductase, and flavodoxin. In a still further aspect, the reducing agent comprises reduced nicotinamide adenine dinucleotide phosphate, titanium citrate, and flavodoxin reductase. In a still further aspect, the reducing agent comprises reduced nicotinamide adenine dinucleotide phosphate and titanium citrate. In a still further aspect, the reducing agent comprises dithionite. In a still further aspect, the reducing agent comprises flavodoxin. In a still further aspect, the reducing agent comprises flavodoxin reductase. In a still further aspect, the reducing agent comprises titanium citrate. In a still further aspect, the reducing agent comprises reduced nicotinamide adenine dinucleotide phosphate.

In a further aspect, PapB installs two or more thioether linkages in the peptide sequence. For example, the peptide sequence may comprise the the sequence C—Yₐ—C-D-Y_{b}-D; wherein C is a cysteine residue, D is an aspartic acid residue, Y is a series of amino acid residues, a=1, 2, 3, 4, 5, 6, or 7, and b=0, 1, 2, 3, 4, 5, 6, or 7. In this example, thioether linkages are installed between the first cysteine residue and the first aspartic acid residue and between the second cysteine residue and the second aspartic acid residue, yielding nested crosslinks. By way of example, the peptide sequence may also comprise the sequence C—Yₓ-D-Y_{y}—C—Y_{z}-D; wherein C is a cysteine residue, D is an aspartic acid residue, Y is a series of amino acid residues, x=0, 1, 2, 3, 4, 5, 6, or 7, y=1, 2, 3, 4, 5, 6, 7, or 8, and z=0, 1, 2, 3, 4, 5, 6, or 7. In this example, thioether linkages are installed between the first cysteine residue and the first aspartic acid residue and between the second cysteine residue and the second aspartic acid residue, yielding in-line crosslinks.

### I. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Methods

a. Cloning and Expression of PapB

The plasmids PapB and pPH151 were co-transformed into Escherichia coli BL21 (DE3) T1 resistant cells (NEB C2527). The plasmid pPH151 contains the suf operon that encodes for sufABCDE proteins that aid in sulfur liberation, act as a Fe—S scaffold, and donate Fe—S clusters to apo proteins. The suf operon is frequently included with radical SAM enzymes as it assists in assembling iron-sulfur clusters in heterologously expressed proteins. The transformation mixture was suspended in SOC recovery media and shaken at 200 rpm for 1 h at 37° C. The mixture was plated on agar Lennox broth (LB) plates containing 34 μg/mL chloramphenicol and 34 μg/mL kanamycin and placed in an oven set to 37° C. for 16 h. An overnight culture (0.15 L) of LB containing 34 μg/mL chloramphenicol and 34 μg/mL kanamycin was inoculated with a single colony from the plate. Twelve aliquots (12 mL each) of overnight culture were used to inoculate twelve 2.8 L Fernbach flasks containing 1 L each of LB supplemented with 34 μg/mL chloramphenicol and 34 μg/mL kanamycin. The cultures were grown at 37° C. and 180 rpm to an OD600 nm of ˜0.35, at which point 0.1 mM iron (III) chloride (0.1 mM) and L-cysteine hydrochloride monohydrate (0.1 mM) were added. At OD600 nm of ˜0.5, the flasks were immersed in an ice bath and cooled for 20 min before inducing with 1 mM isopropyl β-D-1-thiogalactopyranoside (IPTG). The cultures were grown overnight (˜16 h), and the cells were harvested by centrifugation at 6500×g. Typical yield is ˜45 g of wet cell paste per 12 L of growth. The cell pellets were flash-frozen in liquid N2 and stored at −80° C. until use.

b. Purification OF PapB

PapB was purified inside of a Coy Laboratories anaerobic chamber maintained with a 98% N₂/2% H₂ atmosphere. Cell paste (15 g) was resuspended in a metal beaker with 0.1 L of 0.05 M KPi (pH 7.4) buffer containing 0.5 M KCl, 0.05 M imidazole, 20% glycerol (v/v) 0.1 mg/mL lysozyme, 10 μg/mL DNAse and 2 complete EDTA-free Protease Inhibitor Cocktail tablets (Fisher Scientific NC0939481). The suspension was stirred for 30 min on ice after which the cells were lysed with a Branson digital sonifier operated at 50% amplitude for a total of 17 min (25 s on/35 s off) while stirring on ice. The resulting liquid was centrifuged at 18,442×g for 45 min at 4° C. Three 5 mL HisTrap HP columns (GE healthcare) charged with nickel sulfate were serially connected and equilibrated with loading buffer containing 0.05 M KPi (pH 7.4), 0.5 M KCl, 20% glycerol (v/v) and 0.05 M imidazole. The clarified lysate was loaded onto the columns at 3 mL/min. The columns were washed with eight column volumes (CV) of loading buffer and PapB was eluted with a linear gradient over 8 CV to 0.5 M imidazole in the loading buffer. Fractions containing PapB were identified by brown color and sodium dodecyl sulfate polyacrylamide gel electrophoresis (SDS-PAGE) gel.

The pooled fractions were further purified by an amylose resin (NEB E8022S) column equilibrated in loading buffer containing 0.05 M KPi (pH 7.4), 0.5 M KCl, and 0.05 M imidazole. The column was washed with 0.15 L of loading buffer and eluted with loading buffer containing 10 mM maltose. The resulting dark brown fractions were pooled and solid dithiothreitol (DTT) powder was added to combined fractions to a final concentration of 2 mM. An aliquot (1 mL) of 90 μM TEV protease was added, and the mixture was stirred for 14 h at room temperature. The cleaved MBP was removed from PapB through three serially connected 5 mL HisTraps equilibrated in loading buffer. The flowthrough from this column contained cleaved PapB. The resulting PapB protein was desalted into buffer containing 0.05 M PIPES·NaOH (pH 7.4), 0.3 M NaCl, 2 mM DTT and 20% glycerol (v/v). The concentration of PapB was determined by the Bradford method using bovine serum albumin (BSA) as a standard. PapB was reconstituted by mixing 12 molar equivalents of 0.1 M FeCl₃ hexahydrate and Na₂S nonahydrate as follows. Aliquots (5 L) of the FeCl₃ hexahydrate and Na₂S were added individually, allowing 15 sec between additions to ensure thorough mixing. The FeCl₃ was added to completion first, following by the addition of the Na₂S. The reconstitution mixture was stirred for 4 h at room temperature. The resulting solution was centrifuged for 10 min at 16,000×g for 10 min to remove any debris and desalted on a BioGel P6 DG desalting gel 100-200 mesh (wet) (Bio-Rad) into buffer containing 0.05 M PIPES·NaOH (pH 7.4), 0.3 M NaCl, 2 mM DTT, and 20% glycerol (v/v). The protein was concentrated to ˜3 mL with an Amicon concentrator under N₂ with a YM-10 membrane (Millipore).

The reconstituted PapB was further purified by a Cytiva XK26 (1000 mm) S-300 column equilibrated with buffer containing 0.05 M PIPES·NaOH (pH 7.4), 0.3 M KCl, 2 mM DTT and 10% glycerol (v/v). The protein was eluted isocratically at 2.7 mL/min, and fractions containing PapB were identified by dark brown color and visual inspection of a Coomassie-stained SDS-PAGE gel. The pooled fractions were concentrated to ˜0.5 mL. Aliquots were flash-frozen in liquid N2 and stored at −80° C. PapB was quantified by Bradford assay with BSA as a standard. A typical yield from the purification outlined above is 16.5 mg pure protein for 15 g wet cell paste.

c. Amino Acid Analysis and Iron Concentration Determination

The correction factor for the Bradford assays was determined by direct amino acid analysis on three independent preparations of the protein. Amino acid analysis was carried out by the Molecular Structure Facility at the University of California-Davis as follows. A 0.1 mL aliquot of concentrated PapB was desalted into solution containing 10 mM NaOH using an Illustra NICK column (GE Healthcare). The protein samples were hydrolyzed in a solution containing 6 M HCl and 1% phenol at 110° C. in a vacuum and resuspended in a norleucine solution as an internal standard. The PapB samples were analyzed by Hitachi 8800 amino acid analyzer that was calibrated with amino acid standards for protein hydrolysate on the Na-based Hitachi 8800 (Sigma, A-9906). These standards were verified by the National Institute of Standards and Technology (NIST) standard reference material 2389a. PapB samples were sent through a Concise ion-exchange column (AminoSep Beckman Style Na+, part #AAA-99-6312) with a secondary ninhydrin reaction for detection using Pickering Na buffers. The correction factor for the Bradford assays was determined to be 0.60 based on results from the three independent purifications and this factor was used in all subsequent protein concentration determinations to correct the values obtained from the less cumbersome Bradford determinations

The iron content of reconstituted PapB was determined through inductively coupled plasma-mass spectrometer (ICP-MS) on the same three separate enzyme preparations. This was done at the Center for Water, Ecosystems and Climate Science in the Department of Geology and Geophysics at the University of Utah as follows. PapB preparations were diluted to a concentration of 2-5 μM with 10% trace metal grade nitric acid before submission. The iron concentration was performed with a triple quadrupole inductively coupled plasma-mass spectrometer (ICP-MS, Agilent 8900, Santa Clara, CA). A 10 nm In/mL was added as an internal standard. An external calibration curve was prepared from 1000 mg/L single elemental standard (Inorganic Ventures, Christiansburg, VA). Fe Concentrations in six calibration solutions were 0, 8.3, 20.7, 66.2, 165.5 and 331.1 ng Fe/mL; all solutions contained 10 ng In/mL. The blanks, calibration solutions and diluted samples were run by ICP-MS using a double-pass quartz spray chamber, PTFE nebulizer and dual-syringe introduction system (Teledyne, AVX72000), platinum cones, and sapphire injector in a quartz platinum-shielded torch. In and Fe were detected at masses of 115 and 56, with a flow of 8 mL He/min in a collision cell. The Certified Reference Manual CRM 1643f (National Institute of Standards and Technology, Gaithersburg, MD) was diluted 1:20 and run with the samples as well as the calibration curve as a quality control for the calibration. The Fe in the CRM 1643f was measured to be 10% within the certified value.

d. TEV Protease Purification

SG1200008 pRARE chemically competent cells were transformed with pNB512. The transformation was suspended in SOC recovery media and shaken at 200 rpm for 45 min at 37° C. The mixture was plated on agar Lennox broth (LB) plates containing 34 μg/mL chloramphenicol and 100 mg/mL ampicillin and placed in an oven set to 37° C. for 16 h. An overnight culture (0.15 L) of LB containing 34 mg/mL chloramphenicol and 34 mg/mL ampicillin was inoculated with a single colony from the plate. Twelve aliquots (0.010 L each) of overnight culture were used to inoculate twelve 2.8 L Fernbach flasks containing 1 L each of LB supplemented with 34 μg/mL chloramphenicol and 100 μg/mL ampicillin. The cultures were grown at 37° C. and 175 rpm to an OD₆₀₀ₙₘ of ˜0.49, at which point 1 mM IPTG was added to each flask and the temperature was turned down to 16° C. The cultures were grown overnight (˜16 h), and the cells were harvested by centrifugation at 6500×g. The cell pellets were flash-frozen in liquid N₂ and stored at −80° C. until use.

Cell paste (15 g) was resuspended in a metal beaker with 0.1 L of 0.05 M KPi (pH 7.4) buffer containing 0.5M KCl, 0.05 M imidazole, 100 mg/mL lysozyme, 10 mg/mL PMSF, and 20% (v/v) glycerol. The suspension was stirred for 2 h at 4° C. The cells were lysed with a Branson digital sonifier operated at 50% amplitude for a total of 15 min (10 s on/20 s off) while stirring on ice. The resulting liquid was centrifuged at 18,442×g for 50 min at 4° C. Two 5 mL HisTrap HP columns (GE healthcare) charged with nickel sulfate were serially connected and equilibrated with loading buffer containing 0.05 M KPi (pH 7.4), 0.5 M KCl, and 0.05 M imidazole. The clarified lysate was loaded onto the columns at 3 mL/min. The columns were washed with eight column volumes (CV) of loading buffer and TEV protease was eluted with a linear gradient over 8 CV to 0.5 M imidazole in the loading buffer. Fractions containing TEV protease were identified by SDS-PAGE, pooled and dialyzed three times against 4 L of 0.05 M KPi (pH 7.4), 0.5 M KCl, 0.05 M imidazole and 20% glycerol. The dialyzed protein was concentrated to a minimal volume followed by the addition of glycerol to a final concentration of 50% glycerol (v/v). The aliquots were flash frozen in liquid nitrogen and stored at −80° C. until use.

e. Synthesis of Minimal Substrate PapA (msPapA) and Variants

PapA peptides were synthesized on either a PS3 peptide synthesizer (Protein Technologies Inc.) or a Prelude peptide synthesizer (Protein Technologies Inc.). Compared to the previously reported msPapA peptide (Van der Donk, W. A.; Bindman, N. A. Nat. Prod.: Discourse, Delivery, and Design, John Wiley & Sons: Oxford, 2014; pp 197-218), the N-terminal methionine was removed in all peptide syntheses. The syntheses used standard Fmoc procedures from the manufacturer and were carried out on a 0.025 mmol scale. All natural Fmoc-amino acids were purchased from Protein Technologies Inc. N-Alpha-Fmoc-S-trityl-D-cysteine and Fmoc-D-aspartic acid α-tert-butyl ester were purchased from Chem Impex (04314). For the synthesis, 150 mg of 2-chlorotrityl chloride resin 100-200 mesh (ChemPep) was loaded with 9.3 mg of Fmoc-Ala-OH (˜0.2 mmol/g resin). The resin was washed three times with 5 mL DMF and three times with 5 mL dichloromethane (DCM). The 9.3 mg of Fmoc-Ala-OH was dissolved in 1 mL of 1:1 dichloromethane (DCM):N,N-dimethylformaide (DMF) with 0.15 mmol diisopropylethylamine (DIPEA). This solution was added to the resin and gently shaken for 1 h. The Fmoc-Ala/DIPEA solution was then removed, and the resin was washed three times with 5 mL of DCM. The uncapped sites on the resin were capped by washing the resin with 20 mL of 17:2:1 DCM:methanol:DIPEA. The resin was then washed three times with 5 mL of DCM and three times with 5 mL of DMF. The resin was then transferred to the reaction vessel.

All Fmoc-amino acids (0.15 mmol, 6 equivalents) were coupled by in situ activation with N-[(dimethylamino)-1H-1,2,3-triazo[4,5-b]pyridin-1-ylmethylene]-N-methylmethanaminium hexafluorophosphate-N-oxide (HATU) (0.15 mmol, 6 equivalents; ChemPep) in 0.6 M N-methylmorpholine. The peptides were deprotected and cleaved from the resin by adding 5 mL of cleavage solution (87.5% (v/v) TFA, 5% (v/v) thioanisole, 3% (v/v) ethane dithiol, 2.5% (v/v) triisopropylsilane, and 2% (v/v) anisole) followed by stirring for 2 h at room temperature. The cleavage reaction was filtered into 30 mL of ice-cold diethyl ether to precipitate the peptide. The solution was poured over a Büchner funnel filter and vacuumed to collect the peptide precipitate. The peptide dried on the vacuum for 15 min before being washed with 80 mL of ice-cold diethyl ether. After drying for an additional hour, the peptide was resuspended in 20 mL of water and sonicated for 15 min to aid in the dissolution of the peptide. The solution was then flash frozen in liquid nitrogen and lyophilized.

The peptides were purified using high-performance liquid chromatography (HPLC) with a Phenomenex Jupiter C18 preparative column (21.2 mm×250 mm, 5 μm particle size, 300 Å pore size) with buffer A as 0.1% trifluoroacetic acid (TFA, HPLC grade) in nanopure water and buffer B as 0.1% TFA (HPLC Grade) in acetonitrile (ACN, HPLC grade). The separation was carried out at a flow rate of 5 mL/min with a linear gradient of buffer A from 88 to 60% over 65 min. Fractions were analyzed by LC-MS using one of two HPLC-MS/MS setups (Vanquish UHPLC with a diode array detector connected to a Q-Exactive or an Ultimate 3000 HPLC with a diode array detector interfaced to a LTQ OrbiTrap XL mass spectrometer) fitted with a Hypersil GOLD C18 column (2.1 mm×150 mm, 1.9 μm particle size) for separations at 0.2 mL/min. The LC-MS program for peptide fraction identification was set up as follows: buffer A was LC-MS Optima water (Fisher)/0.1% (v/v) LC-MS Optima TFA (Fisher) and buffer B was LC-MS Optima acetonitrile (Fisher)/0.1% (v/v) LC-MS Optima TFA (Fisher). The 12 min separation consisted of washing the column with 100% A for 3 min, followed by a linear gradient to 100% B from 3 to 6 min, followed by washing the column with 100% B from 6 to 9 min, and finally reequilibration in 100% A from 9 to 12 min. The MS detectors operated in positive ion mode and the FT analyzer settings are as follows: 70,000 resolution for the Q-Exactive and 100,000 resolution for the LTQ OrbiTrap, 1 microscan, and 200 ms maximum injection time. MS data analysis used Xcalibur software (Thermo Fisher).

f. Enzymatic Reactions of msPapA Peptides with PapB

Assays were conducted in a Coy Laboratories anaerobic chamber with 98% N₂/2% H₂ atmosphere at room temperature. All reactions contained 0.05 M PIPES·NaOH (pH 7.4), 2 mM DTT, 2.4 mM SAM (enzymatically synthesized and purified as previously described (deGruyter, J. N., et al. Biochem. 2017, 56 (30), 3863-3873)), ˜100-400 μM msPapA variants (concentration determined by peptide dry weight or by spectroscopic analysis in the case of Y19W), and 430 nm-10 μM PapB. Either dithionite (dT) or flavodoxin (FldA), flavodoxin reductase (FPR) and NADPH were used to reduce PapB. For the assays that used chemical reductant, the total concentration was 2 mM dT. For the assays that used the biological reducing system, the mixtures contained 25 μM FldA, 2 μM FPR and 2 mM NADPH. The total volume of the reactions ranged from 0.1 mL for initial screenings to 0.5 mL for MS/MS Collision Induced Dissociation (CID) fragmentation experiments described below. Control reactions in the absence of dT, SAM and PapB were also conducted. Reactions were initiated with the addition of PapB and quenched at times ranging from 15 s to 2 h by the addition of 10% of the reaction volume of 30% (w/v) trichloroacetic acid (TCA, ACS grade). The samples were centrifuged at 16,000×g for 10 min in a microcentrifuge to pellet the precipitated PapB.

g. Alkylation of msPapA Peptides and Variants

After initial incubation, half of the enzymatic reaction and half of the control reaction was aliquoted for alkylation by iodoacetic acid. A 500 mM stock of iodoacetic acid (IAC) was prepared in the dark and added to the enzymatic reaction to a final concentration of 10 mM (5× excess of the DTT concentration). These reactions were allowed to incubate in the dark for six additional hours before quenching with the addition of 10% of the reaction volume of 30% (w/v) TCA. The samples were then centrifuged at 16,000×g for 10 min in a microcentrifuge to pellet the precipitated PapB.

h. TEV Protease Cleavage of Peptides

Where TEV cleavage was necessary, 90 μM TEV protease was added directly to the full PapB assay after initial incubation in a 1:1 volume ratio. The TEV-assay combination incubated for 4 h before quenching by the addition of 10% of the reaction volume of 30% (w/v) TCA. The samples were then centrifuged at 16,000×g for 10 min in a microcentrifuge to pellet the precipitated PapB and TEV protease.

i. U/HPLC-MS Analysis of Enzymatic Reactions and Controls

The assays were analyzed using either a Vanquish UHPLC with a diode-array detector connected to a Q-Exactive mass spectrometer or an Ultimate 3000 HPLC with a diode-array detector connected to a LTQ OrbiTrap XL mass spectrometer. Each was operated in positive ion mode, the FT analyzer was set to 100,000 resolution, 1 microscan, and 200 ms maximum injection time. Xcalibur software was used to analyze data. A 20 μL aliquot was injected onto a Hypersil GOLD C18 column (2.1 mm×150 mm, 1.9 μm particle size) (Thermo Fisher) pre-equilibrated in 0.1% (v/v) LC-MS Optima TFA (Fisher in LC-MS Optima water (Fisher). Chromatographic steps were carried out at 0.2 mL/min with buffer A containing 0.1% (v/v) TFA in Optima water and buffer B containing Optima grade acetonitrile with 0.1% (v/v) TFA. The separation consisted of washing with 100% A from 0 to 3 min, followed by a linear gradient from 100% to 0% A from 3 to 6 min, washing with 0% A from 6 to 10 min, and reequilibration with 100% A from 10 to 14 min.

j. Collision-Induced Dissociation (CID) Fragmentation of Unmodified and Modified PapB

Enzymatic reactions were conducted on a 0.5 mL scale as described above to obtain sufficient material. After quenching the reaction with TCA and centrifugation to remove precipitated protein, the reaction mixtures were desalted using C18 ZipTips (Millipore) following the manufacturer's protocols. The analyzer was first tuned to the mass of each msPapA peptide. The 3⁺ charge state corresponding to each msPapA peptide was isolated in the CID cell using an isolation width of 1.7-2.4 m/z (depending on complete or incomplete peptide turnover), 0.1 ms activation time, a resolution of 70,000, and fragmented using a Normalized Collision Energy (NCE) of 25. The fragmentation analysis used mMass software.

### 2. Characterization of Purified PapB

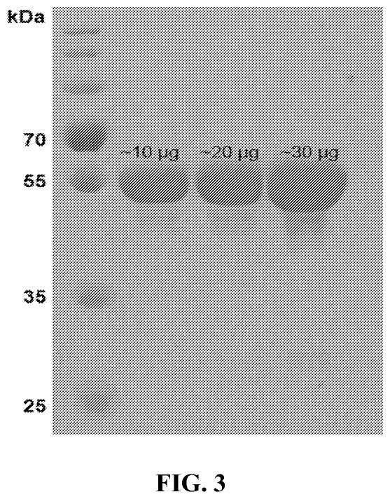

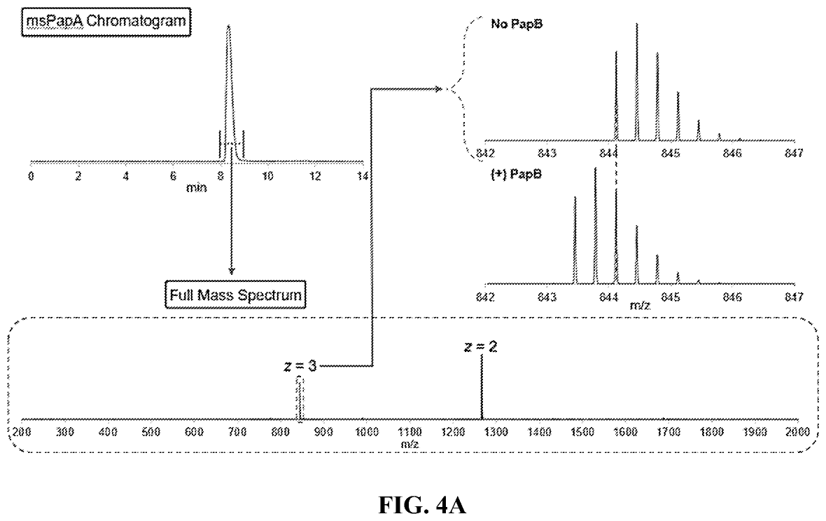

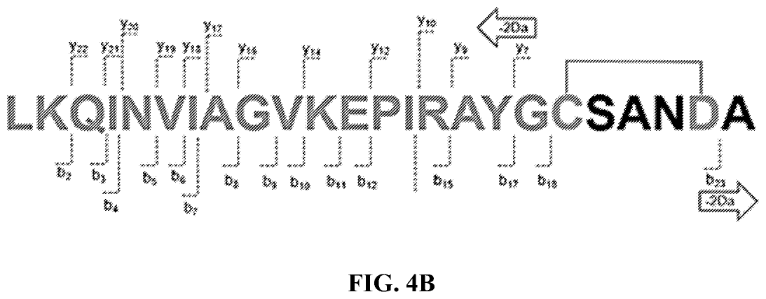

PapB was obtained to homogeneity using His affinity chromatography for the initial separation, followed by TEV cleavage and amylose chromatography to remove the MBP, reconstitution with Fe/S. Gel filtration was used to remove higher molecular weight complexes (FIG. 3). Since previous sequence analysis and ferrozine assays indicate that PapB likely has three [4Fe-4S] clusters—a 12-fold molar excess of iron and sulfide were added to the maturase for reconstitution. Amino acid and ICP-MS analysis of protein from multiple independent purifications show that the purified protein obtained by this procedure contains 13.5±0.3 mol of iron per mol of PapB. This is consistent with three [4Fe-4S] clusters per polypeptide chain. The enzymatic activity of PapB was established with HPLC-purified msPapA (FIG. 4A and FIG. 4B). The peptide elutes at 8 min under the conditions used in the separation (FIG. 4A, top left) and HR-MS/MS reveals two clearly visible charge states (FIG. 4A, bottom). Expansion of the +3-charge state (FIG. 4A, top right) reveals an isotopic envelope with the monoisotopic peak at m/z of 844.1201, which is within 0.5 ppm of the calculated unmodified peptide (calc: m/z 844.1197). In the presence of PapB, dithionite (dT), and SAM the monoisotopic peak of the +3 charge state shifts by 0.6716, which corresponds to a loss of 2 Da from the peptide. This is within 0.8 ppm of the expected mass for a singly crosslinked peptide. To validate that a thioether cross link had formed the modification reaction was carried out in bulk and the resulting sample was desalted and subjected to HR-MS/MS analysis. As expected for a crosslinked ranthipeptide (Precord, T. W., et al. ACS Chem. Biol. 2019, 14 (9), 1981-89), no fragments are seen between the C19 and D23 in the reacted peptide. Additionally, a 2 Da loss was observed in the b₂₃ ion and in every y series ion above y₇ (FIG. 4B). The fragmentation data is shown in Table 1 below. It is noted that under these conditions, complete conversion of msPapA to a singly crosslinked peptide is routinely observed using 0.1 nmol PapB and 20 mmol msPapA in 5 min.

TABLE 1
LKQINVIAGVKEPIRAYGCSANDA

| | | Expected | Observed | |
|---|---|---|---|---|
| | | Monoisotopic | Monoisotopic | Ppm |
| Ion | Sequence | Mass | Mass | Error |
| b2* | LK* | 242.1863 | 242.1856 | -2.89 |
| b3* | LKQ* | 370.2449 | 370.2442 | -1.89 |
| b4* | LKQI* | 483.3289 | 483.3282 | -1.44 |
| b5* | LKQIN* | 597.3719 | 597.3712 | -1.17 |
| b6* | LKQINV* | 696.4403 | 696.4391 | -1.72 |
| b7* | LKQINVI* | 809.5244 | 809.5232 | -1.48 |
| b8* | LKQINVIA* | 880.5615 | 880.5606 | -1.02 |
| b9* | LKQINVIAG* | 937.5829 | 937.5822 | -0.74 |
| b10* | LKQINVIAGV* | 1036.6513 | 1036.6502 | -1.06 |
| b11* | LKQINVIAGVK* | 1164.7463 | 1164.7461 | -0.17 |
| b12* | LKQINVIAGVKE* | 1293.7889 | 1293.7885 | -0.31 |
| b13* | LKQINVIAGVKEP* | 695.9245 | 695.9245 | 0 |
| b14* | LKQINVIAGVKEPI* | 752.4665 | 752.4646 | -2.52 |
| b15* | LKQINVIAGVKEPIR* | 830.5171 | 830.5162 | -1.08 |
| b16* | LKQINVIAGVKEPIRA* | 866.0356 | 866.0344 | -1.39 |
| b17* | LKQINVIAGVKEPIRAY* | 947.5673 | 947.5666 | -0.74 |
| b18* | LKQINVIAGVKEPIRAYG* | 976.0780 | 976.0778 | -0.20 |
| b19* | (-1 Da) LKQINVIAGVKEPIRAYGC* | 1027.0787 | Not Found | N/A |
| b20* | (-1 Da) LKQINVIAGVKEPIRAYGCS* | 1106.1133 | Not Found | N/A |
| b21* | (-1 Da) | 1127.6043 | Not Found | N/A |
| | LKQINVIAGVKEPIRAYGCSA* | | | |
| b22* | (-1 Da) | 1163.1347 | Not Found | N/A |
| | LKQINVIAGVKEPIRAYGCSAN* | | | |
| b23* | (-2 Da) | 813.7653 | 813.7641 | -1.47 |
| | LKQINVIAGVKEPIRAYGCSAND* | | | |
| [M] | (-2 Da) | 843.4478 | 843.4468 | -1.18 |
| | LKQINVIAGVKEPIRAYGCSANDA* | | | |
| y23* | (-2 Da) | 805.7532 | 805.7520 | -1.49 |
| | KQINVIAGVKEPIRAYGCSANDA* | | | |
| y22* | (-2 Da) | 1144.0786 | 1144.0780 | -0.52 |
| | QINVIAGVKEPIRAYGCSANDA* | | | |
| y21* | (-2 Da) | 1080.0493 | 1080.0490 | -0.27 |
| | INVIAGVKEPIRAYGCSANDA* | | | |
| y20* | (-2 Da) NVIAGVKEPIRAYGCSANDA* | 1023.5073 | 1023.5069 | -0.39 |
| y19* | (-2 Da) VIAGVKEPIRAYGCSANDA* | 966.4858 | 966.4852 | -0.62 |
| y18* | (-2 Da) IAGVKEPIRAYGCSANDA* | 916.9516 | 916.9512 | -0.43 |
| y17* | (-2 Da) AGVKEPIRAYGCSANDA* | 860.4096 | 860.4087 | -1.04 |
| y16* | (-2 Da) GVKEPIRAYGCSANDA* | 824.8910 | 824.8902 | -0.97 |
| y15* | (-2 Da) VKEPIRAYGCSANDA* | 796.3803 | 796.3782 | -2.63 |
| y14* | (-2 Da) KEPIRAYGCSANDA* | 746.8461 | 746.8451 | -1.34 |
| y13* | (-2 Da) EPIRAYGCSANDA* | 682.7986 | 682.7983 | -0.44 |
| y12* | (-2 Da) PIRAYGCSANDA* | 1235.5473 | 1235.5469 | -0.32 |
| y11* | (-2 Da) IRAYGCSANDA* | 1138.4946 | 1138.4941 | -0.44 |
| y10* | (-2 Da) RAYGCSANDA* | 1025.4105 | 1025.4103 | -0.19 |
| y9* | (-2 Da) AYGCSANDA* | 869.3094 | 869.3081 | -1.49 |
| 18* | (-2 Da) YGCSANDA* | 798.2723 | 798.2708 | -1.88 |
| y7* | (-2 Da) GCSANDA* | 635.2090 | 635.2080 | -1.57 |
| y6* | (-2 Da) CSANDA* | 578.1875 | Not Found | N/A |
| y5* | (-1 Da) SANDA* | 476.1861 | Not Found | N/A |
| y4* | (-1 Da) ANDA* | 389.1541 | Not Found | N/A |
| y3* | (-1 Da) NDA* | 318.1170 | Not Found | N/A |
| y2* | (-1 Da) DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

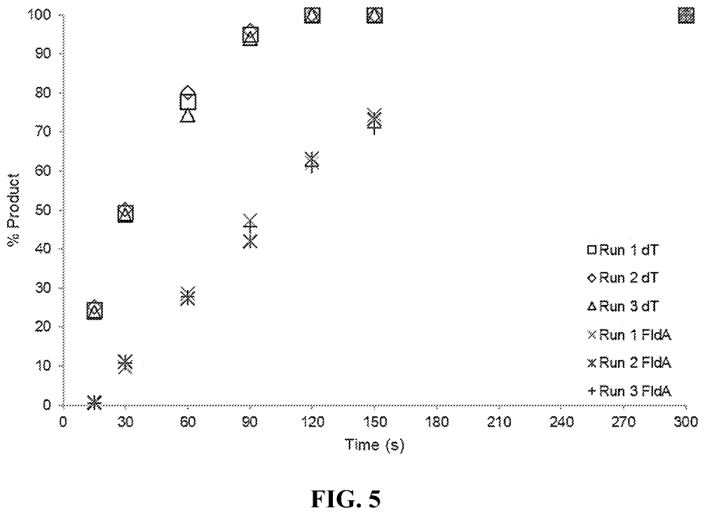

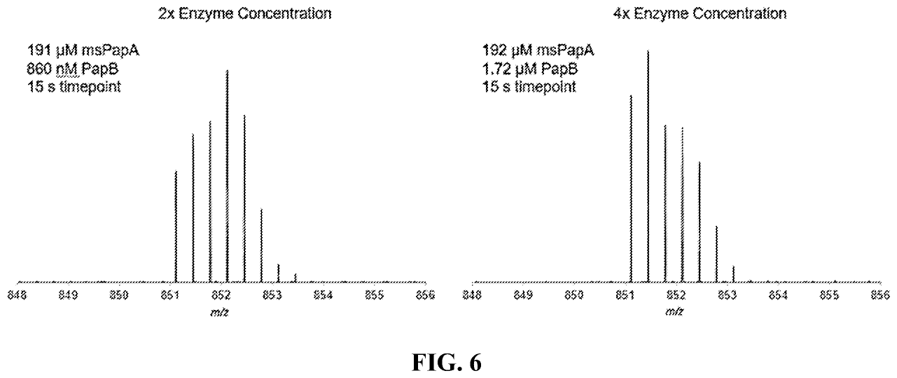

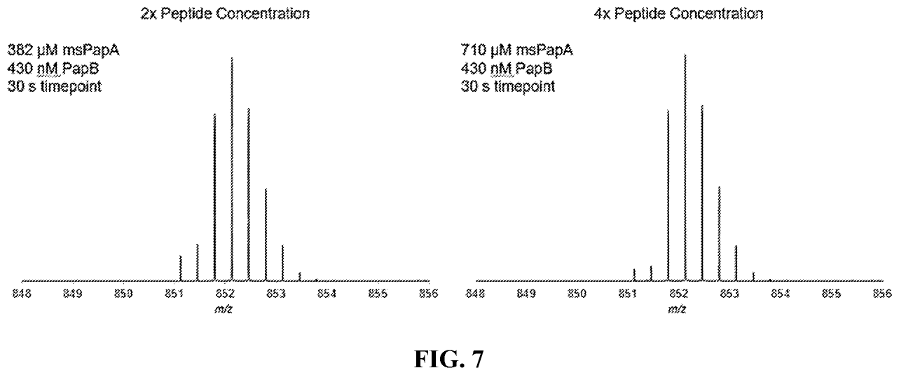

Next, the kinetics of the modification reaction catalyzed by PapB in the presence of dT or a biological reducing system (FldA/FPR/NADPH) were assessed (FIG. 5). In these experiments, the enzyme concentration was kept low (430 nM) relative to the peptide (191 μM; established by tryptophan absorbance). Under these conditions, both show robust turnover, with dT showing roughly 3-fold faster kinetics than that observed with the biological reducing system. Three replicate runs were conducted using either dT or the biological reducing system of FldA/FPR/NADPH. At each timepoint, an aliquot was taken from the initial assay batch quenched with TCA. Both the biological reducing system and the chemical reducing system had 100% substrate conversion after 300 s. At 15 s, 2- and 4-fold increases in the concentration of PapB results in conversion of unmodified msPapA to modified msPapA that is roughly 2- and 4-fold greater than initial conditions, suggesting that activity is proportional to PapB concentration. However, increasing the msPapA concentration by 2- and 4-fold did not alter the distribution of the reaction, suggesting that peptide concentration was saturating. Therefore, the rate that is measured in these experiments is a good approximation of k_{cat} for PapB (FIG. 5-FIG. 7). Using the linear portions of the curves turnover numbers of 7.4±0.1 s⁻¹ with dT and 2.6=0.2 s⁻¹ are estimated with the biological reducing system.

### 3. Leveraging Substrate Promiscuity of a Radical Sam RiPP Maturase Towards Intramolecular Peptide Crosslinking Applications

a. PapB Modifies Expanded and Contracted C(X₃)D Motifs

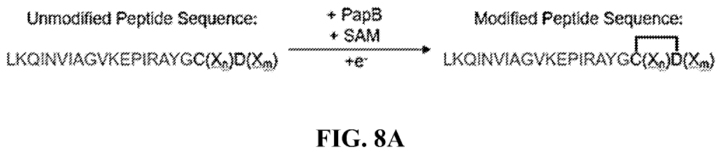

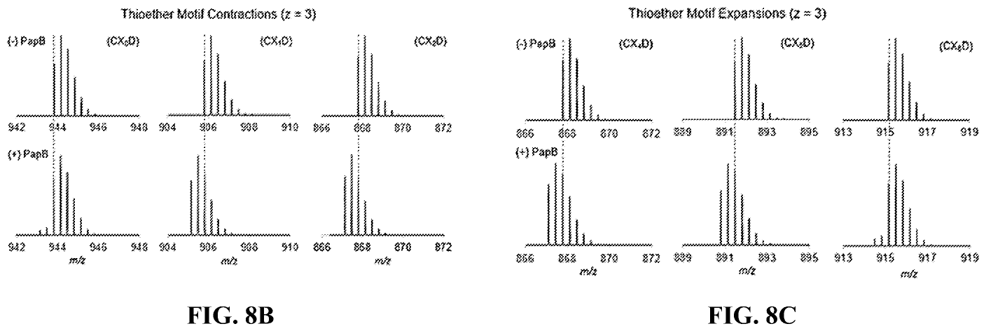

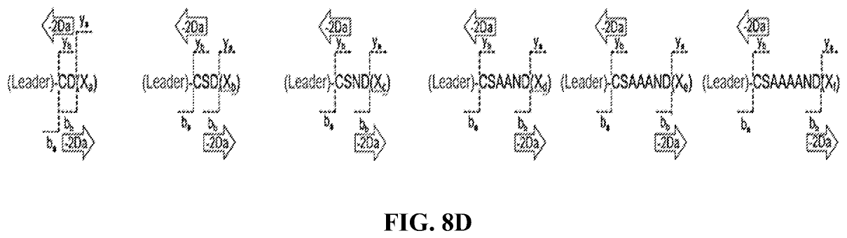

To assess the sequence dependence of the modification, minimal substrates containing 0-6 amino acids between the crosslinked Cys and Asp were synthesized and incubated with PapB (FIG. 8A). In each case, a loss of 2 Da is observed upon the addition of PapB (compare FIG. 8B and FIG. 8C). While the reactions with 1-5 intervening residues appear to go to completion, CX₀D (FIG. 8B) and CX₆D (FIG. 8C) did not fully react--suggesting that PapB does not processes these motifs efficiently. The observed monoisotopic masses for each processed and unprocessed species of peptide agree (to within <4 ppm error) with the expected monoisotopic masses (Table 2).

TABLE 2

| | Expected | Observed | |
|---|---|---|---|
| | Monoisotopic | Monoisotopic | Ppm |
| Sequence | Mass | Mass | Error |
| msPapA | (-) PapB: 844.1197 | 844.1201 | 0.474 |
| (CX3D) | (+) PapB (-2 Da): | 843.4485 | 0.823 |
| | 843.4478 | | |
| Leader- | (-) PapB: 943.8247 | 943.8253 | 0.645 |
| CDSNNAANA | (+) PapB (-2 Da): | | |
| (CXD) | 943.1555 | 943.1573 | 1.908 |
| Leader- | (-) PapB: 905.8131 | 905.8166 | 3.863 |
| CSDNNAAA | (+) PapB (-2 Da): | 905.1418 | 0.662 |
| (CX1D) | 905.1412 | | |
| Leader- | (-) PapB: 867.7988 | 867.7999 | 1.267 |
| CSNDAAA | (+) PapB (-2 Da): | 867.1278 | 1.038 |
| (CX2D) | 867.1269 | | |
| Leader- | (-) PapB: 867.7988 | 867.7972 | -1.843 |
| CSAANDA | (+) PapB (-2 Da): | 867.1287 | 2.076 |
| (CX4D) | 867.1269 | | |
| Leader- | (-) PapB: 891.4778 | 891.4777 | -0.112 |
| CSAAANDA | (+) PapB (-2 Da): | 890.8065 | 0.674 |
| (CX5D) | 890.8059 | | |
| Leader- | (-) PapB: 915.1568 | 915.1564 | -0.437 |
| CSAAAANDA | (+) PapB (-2 Da): | 914.4846 | -0.437 |
| (CX6D) | 914.4850 | | |

z = 3 in all cases

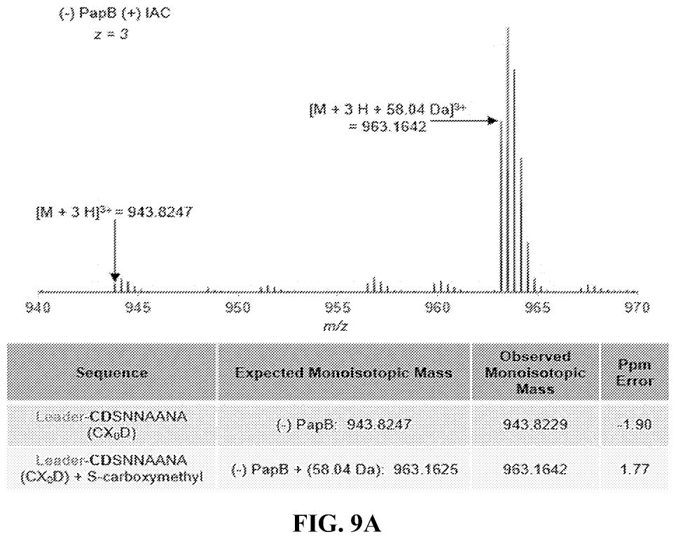

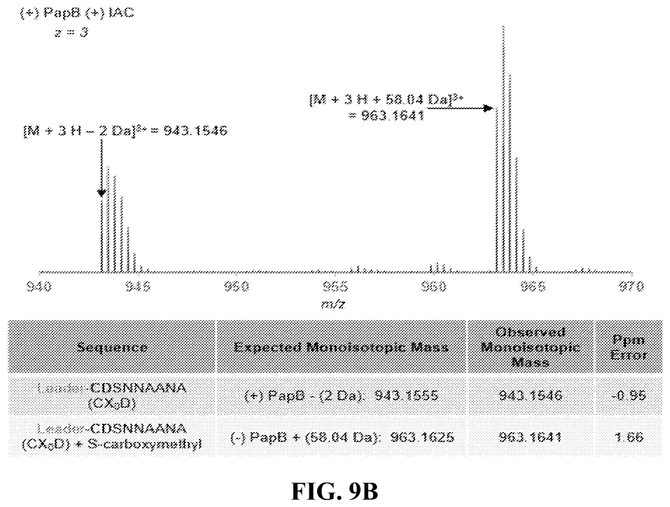

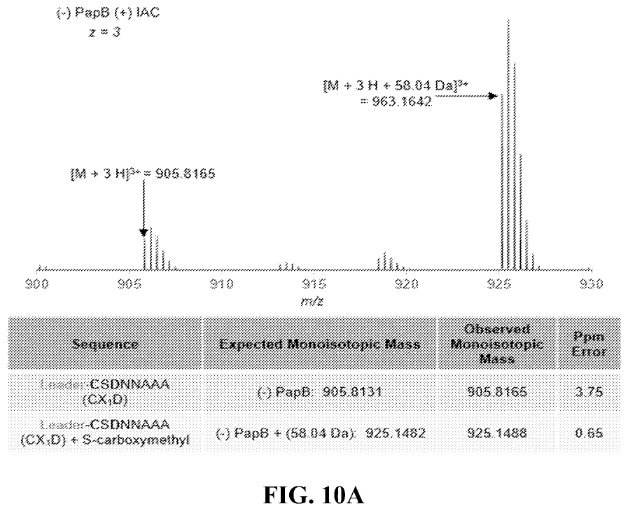

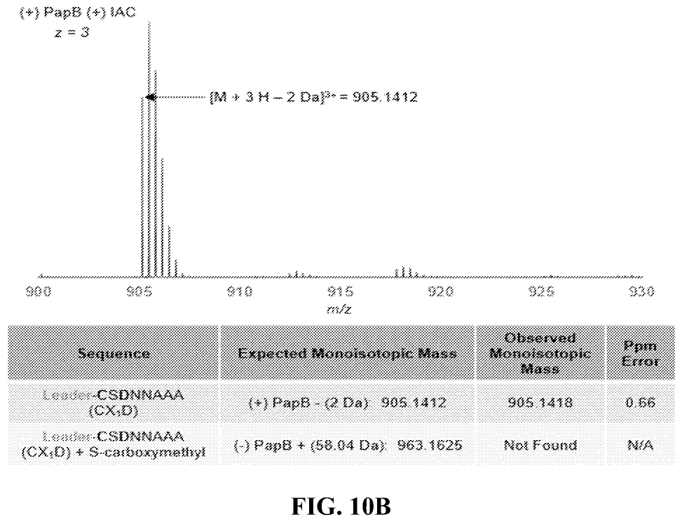

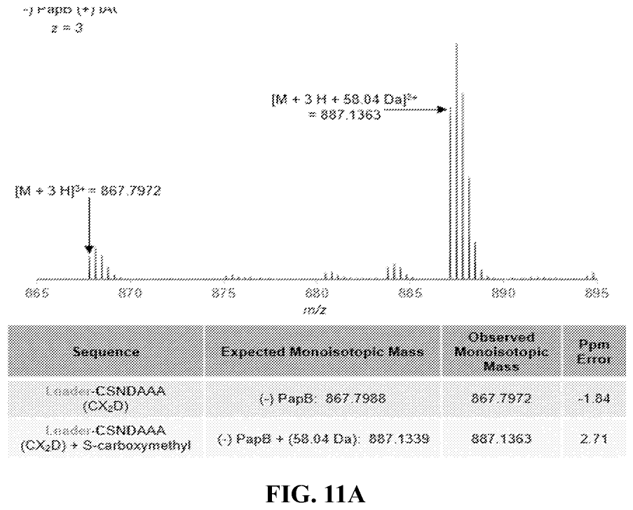

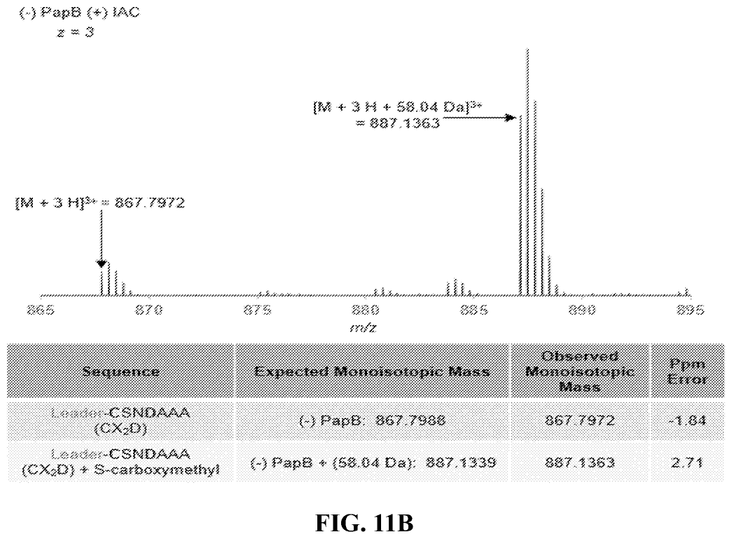

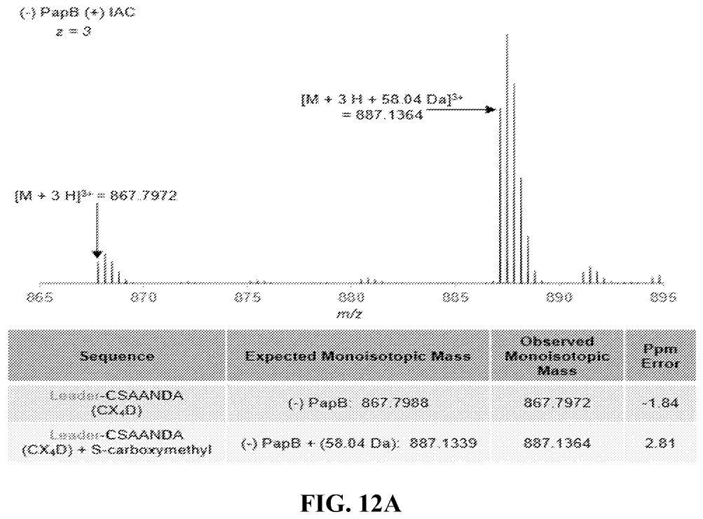

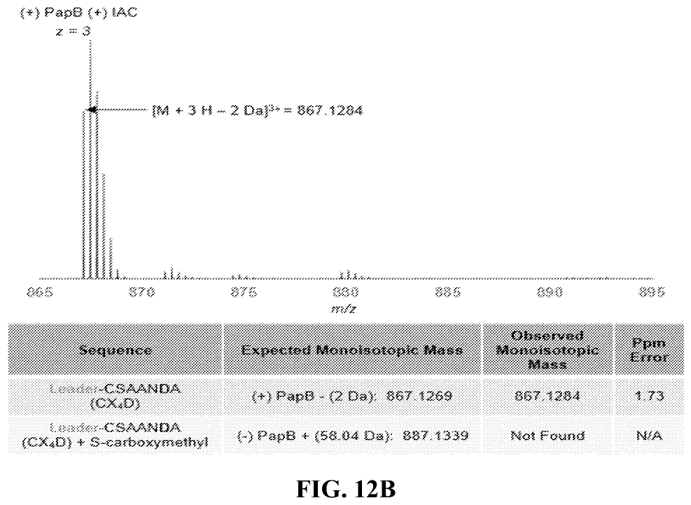

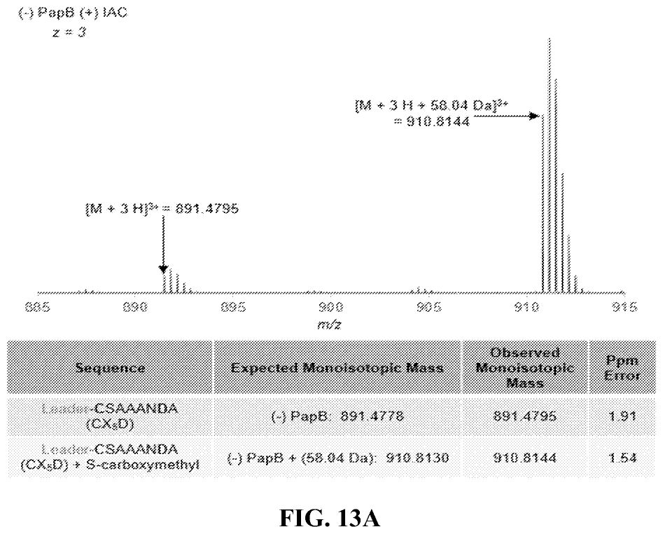

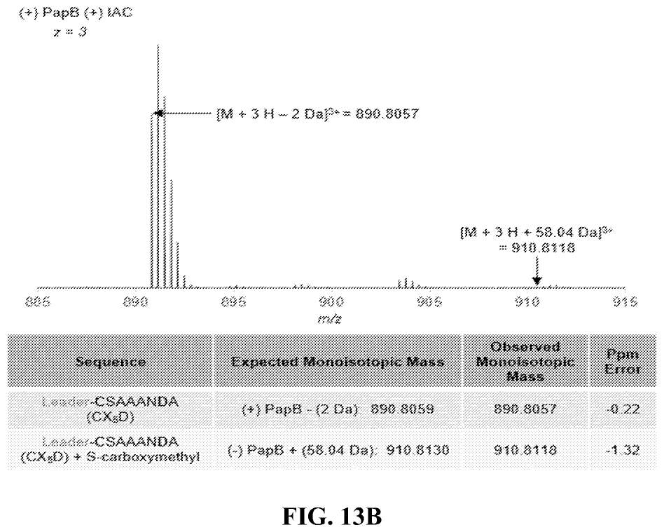

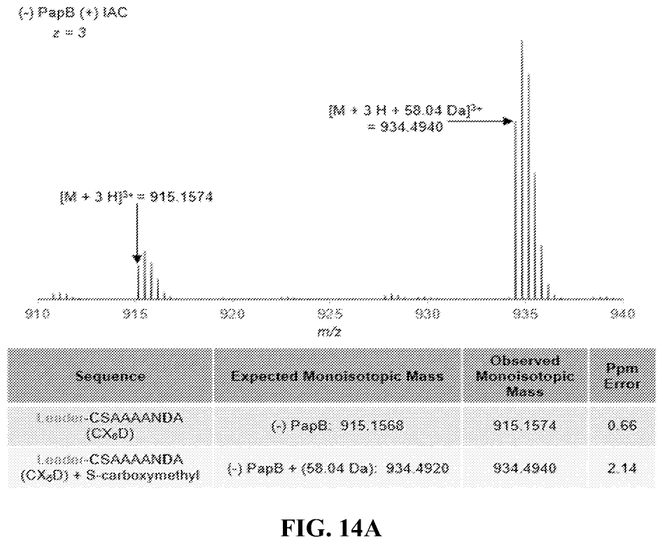

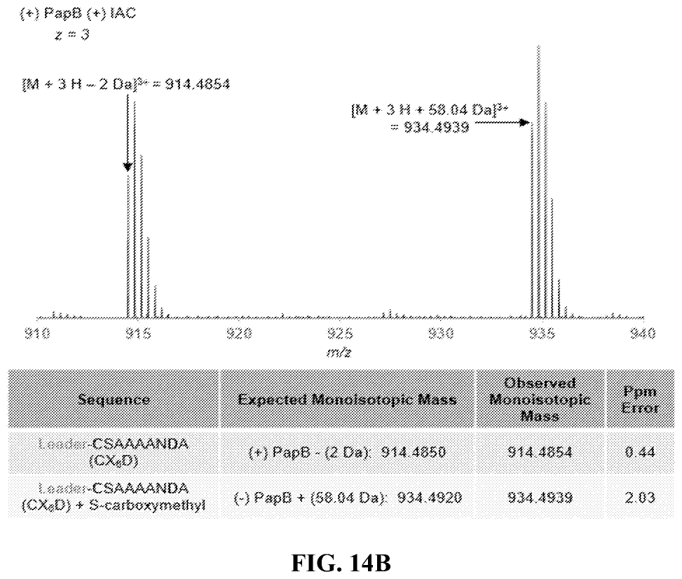

Treatment with iodoacetic acid (IAC) suggests that no free thiols are present in the treated samples, other than the C in the unmodified portion of CX₀D and CX₆D (FIG. 9-FIG. 14). This shows that PapB has introduced a thioether crosslink in each peptide.

The location of modification in each msPapA peptide variant was investigated by collision-induced dissociation (CID) MS/MS. The modified msPapA peptides were analyzed and compared to the unmodified control peptides. In each case, the samples were introduced to the mass spectrometer by direct infusion after quenching with TCA and removal of excess salts. The +3 charge state envelope was isolated and fragmented in the CID cell of the instrument. The fragmentation data showing all b and y ions that could be identified are shown in Tables 3-8 below. In general, the unmodified peptides all displayed fragmentation between Cys and Asp residues. Upon modification, by the addition of PapB, no fragmentation peaks are observable between those two residues. In the case of the b fragments, no change of mass is observed until after the Asp residue, after which a −2 Da loss is seen in each fragment. By contrast, a −2 Da loss is observed after the Cys residue in each y fragment.

TABLE 3
LKQINVIAGVKEPIRAYGCDSNNAANA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1850 | -5.36 |
| b2* | LK* | 242.1863 | 242.1862 | -0.43 |
| b3 | LKQ | 370.2449 | 370.2432 | -4.59 |
| b3* | LKQ* | 370.2449 | 370.2450 | 0.27 |
| b4 | LKQI | 483.3289 | 483.3269 | -4.14 |
| b4* | LKQI* | 483.3289 | 483.3290 | 0.21 |
| b5 | LKQIN | 597.3719 | 597.3692 | -4.52 |
| b5* | LKQIN* | 597.3719 | 597.3708 | -1.84 |
| b6 | LKQINV | 696.4403 | 696.4371 | -4.60 |
| b6* | LKQINV* | 696.4403 | 696.4408 | 0.72 |
| b7 | LKQINVI | 809.5244 | 809.5222 | -2.72 |
| b7* | LKQINVI* | 809.5244 | 809.5245 | 0.12 |
| b8 | LKQINVIA | 880.5615 | 880.5570 | -5.11 |
| b8* | LKQINVIA* | 880.5615 | 880.5612 | -0.34 |
| b9 | LKQINVIAG | 937.5829 | 937.5833 | 0.43 |
| b9* | LKQINVIAG* | 937.5829 | 937.5838 | 0.96 |
| b10 | LKQINVIAGV | 1036.6513 | 1036.6462 | -4.92 |
| b10* | LKQINVIAGV* | 1036.6513 | 1036.6520 | 0.67 |
| b11 | LKQINVIAGVK | 1164.7463 | 1164.7429 | -2.92 |
| b11* | LKQINVIAGVK* | 1164.7463 | 1164.7484 | 1.80 |
| b12 | LKQINVIAGVKE | 1293.7889 | 1293.7865 | -1.86 |
| b12* | LKQINVIAGVKE* | 1293.7889 | 1293.7904 | 1.16 |
| b13 | LKQINVIAGVKEP | 1390.8417 | Not Found | N/A |
| b13* | LKQINVIAGVKEP* | 1390.8417 | 1390.8349 | -4.89 |
| b14 | LKQINVIAGVKEPI | 1503.9257 | 1503.9224 | -2.19 |
| b14* | LKQINVIAGVKEPI* | 1503.9257 | 1503.9261 | 0.27 |
| b15 | LKQINVIAGVKEPIR | 1660.0268 | 1660.0205 | -3.79 |
| b15* | LKQINVIAGVKEPIR* | 1660.0268 | 1660.0196 | -4.33 |
| b16 | LKQINVIAGVKEPIRA | 1731.0640 | 1731.0599 | -2.36 |
| b16* | LKQINVIAGVKEPIRA* | 1731.0640 | 1731.0645 | 0.29 |
| b17 | LKQINVIAGVKEPIRAY | 1894.1273 | Not Found | N/A |
| b17* | LKQINVIAGVKEPIRAY* | 1894.1273 | Not Found | N/A |
| b18 | LKQINVIAGVKEPIRAYG | 976.0780 | 976.0725 | -5.63 |
| b18* | LKQINVIAGVKEPIRAYG* | 976.0780 | 976.0777 | -0.31 |
| b19 | LKQINVIAGVKEPIRAYGC | 1027.5826 | 1027.5796 | -2.92 |
| b19* | (-1 Da) LKQINVIAGVKEPIRAYGC* | 1027.0787 | Not Found | N/A |
| b20 | LKQINVIAGVKEPIRAYGCD | 1085.0961 | 1085.0921 | -3.69 |
| b20* | (-2 Da) LKQINVIAGVKEPIRAYGCD* | 1084.0882 | 1084.0874 | -0.74 |
| b21 | LKQINVIAGVKEPIRAYGCDS | 1128.6121 | 1128.6078 | -3.81 |
| b21* | (-2 Da) LKQINVIAGVKEPIRAYGCDS* | 1127.6043 | 1127.6063 | 1.77 |
| b22 | LKQINVIAGVKEPIRAYGCDSN | 1185.6336 | 1185.6302 | -2.87 |
| b22* | (-2 Da) LKQINVIAGVKEPIRAYGCDSN* | 1184.6257 | 1184.6283 | 2.19 |
| b23 | LKQINVIAGVKEPIRAYGCDSNN | 828.7724 | 828.7695 | -3.50 |
| b23* | (-2 Da) LKQINVIAGVKEPIRAYGCDSNN* | 828.1006 | 828.1005 | -0.12 |
| b24 | LKQINVIAGVKEPIRAYGCDSNNA | 852.4515 | 852.4478 | -4.34 |
| b24* | (-2 Da) | 851.7796 | 851.7794 | -0.23 |
| | LKQINVIAGVKEPIRAYGCDSNNA* | | | |
| b25 | LKQINVIAGVKEPIRAYGCDSNNAA | 876.1305 | 876.1286 | -2.17 |
| b25* | (-2 Da) | 875.4586 | Not Found | N/A |
| | LKQINVIAGVKEPIRAYGCDSNNAA* | | | |
| b26 | LKQINVIAGVKEPIRAYGCDSNNAAN | 914.1448 | 914.1410 | -4.16 |
| b26* | (-2 Da) | 913.4729 | 913.4714 | -1.64 |
| | LKQINVIAGVKEPIRAYGCDSNNAAN* | | | |
| [M] | LKQINVIAGVKEPIRAYGCDSNNAANA | 943.8274 | 943.8234 | -4.24 |
| [M]* | (-2 Da) | 943.1555 | 943.1543 | -1.27 |
| | LKQINVIAGVKEPIRAYGCDSNNAANA* | | | |
| y26 | KQINVIAGVKEPIRAYGCDSNNAANA | 906.1327 | 906.1325 | -0.22 |
| y26* | (-2 Da) | 905.4608 | 905.4659 | 5.63 |
| | KQINVIAGVKEPIRAYGCDSNNAANA* | | | |
| y25 | QINVIAGVKEPIRAYGCDSNNAANA | 863.4344 | 863.4344 | 0.00 |
| y25* | (-2 Da) | 862.7625 | 862.7652 | 3.13 |
| | QINVIAGVKEPIRAYGCDSNNAANA* | | | |
| y24 | INVIAGVKEPIRAYGCDSNNAANA | 820.7482 | Not Found | N/A |
| y24* | (-2 Da) | 820.0763 | Not Found | N/A |
| | INVIAGVKEPIRAYGCDSNNAANA* | | | |
| y23 | NVIAGVKEPIRAYGCDSNNAANA | 783.0535 | 783.0503 | -4.08 |
| y23* | (-2 Da) NVIAGVKEPIRAYGCDSNNAANA* | 782.3816 | 782.3773 | -5.50 |
| y22 | VIAGVKEPIRAYGCDSNNAANA | 1117.0551 | 1117.0495 | -5.01 |
| y22* | (-2 Da) VIAGVKEPIRAYGCDSNNAANA* | 1116.0473 | 1116.0499 | 2.33 |
| y21 | IAGVKEPIRAYGCDSNNAANA | 1067.5209 | 1067.5218 | 0.84 |
| y21* | (-2 Da) IAGVKEPIRAYGCDSNNAANA* | 1066.5131 | Not Found | N/A |
| y20 | AGVKEPIRAYGCDSNNAANA | 1010.9789 | 1010.9735 | -5.34 |
| y20* | (-2 Da) AGVKEPIRAYGCDSNNAANA* | 1009.9711 | 1009.9712 | 0.10 |
| y19 | GVKEPIRAYGCDSNNAANA | 975.4603 | 975.4565 | -3.90 |
| y19* | (-2 Da) GVKEPIRAYGCDSNNAANA* | 974.4525 | 974.4523 | -0.20 |
| y18 | VKEPIRAYGCDSNNAANA | 946.9496 | Not Found | N/A |
| y18* | (-2 Da) VKEPIRAYGCDSNNAANA* | 945.9418 | Not Found | N/A |
| y17 | KEPIRAYGCDSNNAANA | 1793.8235 | 1793.8133 | -5.69 |
| y17* | (-2 Da) KEPIRAYGCDSNNAANA* | 1791.8079 | Not Found | N/A |
| y16 | EPIRAYGCDSNNAANA | 1665.7286 | 1665.7236 | -3.00 |
| y16* | (-2 Da) EPIRAYGCDSNNAANA* | 1663.7129 | 1663.7163 | 2.04 |
| y15 | PIRAYGCDSNNAANA | 1536.6860 | 1536.6802 | -3.77 |
| y15* | (-2 Da) PIRAYGCDSNNAANA* | 1534.6703 | 1534.6654 | -3.19 |
| y14 | IRAYGCDSNNAANA | 1439.6332 | Not Found | N/A |
| y14* | (-2 Da) IRAYGCDSNNAANA* | 1437.6176 | Not Found | N/A |
| y13 | RAYGCDSNNAANA | 1326.5491 | 1326.5424 | -5.05 |
| y13* | (-2 Da) RAYGCDSNNAANA* | 1324.5335 | Not Found | N/A |
| y12 | AYGCDSNNAANA | 1170.4480 | 1170.4422 | -4.09 |
| y12* | (-2 Da) AYGCDSNNAANA* | 1168.4324 | Not Found | N/A |
| y11 | YGCDSNNAANA | 1099.4109 | 1099.4099 | -0.91 |
| y11* | (-2 Da) YGCDSNNAANA* | 1097.3953 | 1097.3949 | -0.36 |
| y10 | GCDSNNAANA | 936.3476 | 936.3507 | 3.31 |
| y10* | (-2 Da) GCDSNNAANA* | 934.3319 | 934.3333 | 1.50 |
| y9 | CDSNNAANA | 879.3261 | Not Found | N/A |
| y9* | (-2 Da) CDSNNAANA* | 877.3105 | Not Found | N/A |
| y8 | DSNNAANA | 776.3169 | 776.3141 | -3.61 |
| y8* | (-1 Da) DSNNAANA* | 775.3091 | Not Found | N/A |
| y7 | SNNAANA | 661.2900 | 661.2875 | -3.78 |
| y7* | SNNAANA* | 661.2900 | 661.2896 | -0.60 |
| y6 | NNAANA | 574.2580 | 574.2559 | -3.65 |
| y6* | NNAANA* | 574.2580 | 574.2578 | -0.35 |
| y5 | NAANA | 460.2150 | 460.2127 | -5.00 |
| y5* | NAANA* | 460.2150 | 460.2151 | 0.22 |
| y4 | AANA | 346.1721 | 346.1704 | -4.91 |
| y4* | AANA* | 346.1721 | 346.1722 | 0.29 |
| y3 | ANA | 275.1350 | 275.1340 | -3.63 |
| y3* | ANA* | 275.1350 | 275.1349 | -0.36 |
| y2 | NA | 204.0979 | 204.0967 | -5.87 |
| y2* | NA* | 204.0979 | 204.0980 | 0.49 |

Unmodified indicates z = 1 charge state.
Underline indicates z = 2 charge state.
Italics indicates z = 3 charge state.
*Indicates the fragment originated from the modified peptide

TABLE 4
LKQINVIAGVKEPIRAYGCSDNNAAA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1859 | -1.65 |
| b2* | LK* | 242.1863 | 242.1864 | 0.41 |
| b3 | LKQ | 370.2449 | 370.2442 | -1.89 |
| b3* | LKQ* | 370.2449 | 370.2454 | 1.35 |
| b4 | LKQI | 483.3289 | 483.3268 | -4.34 |
| b4* | LKQI* | 483.3289 | 483.3294 | 1.03 |
| b5 | LKQIN | 597.3719 | 597.3690 | -4.85 |
| b5* | LKQIN* | 597.3719 | 597.3723 | 0.670 |
| b6 | LKQINV | 696.4403 | 696.4369 | -4.88 |
| b6* | LKQINV* | 696.4403 | 696.4411 | 1.15 |
| b7 | LKQINVI | 809.5244 | 809.5213 | -3.83 |
| b7* | LKQINVI* | 809.5244 | 809.5250 | 0.74 |
| b8 | LKQINVIA | 880.5615 | 880.5576 | -4.43 |
| b8* | LKQINVIA* | 880.5615 | 880.5621 | 0.68 |
| b9 | LKQINVIAG | 937.5829 | 937.5807 | -2.35 |
| b9* | LKQINVIAG* | 937.5829 | 937.5834 | 0.53 |
| b10 | LKQINVIAGV | 1036.6513 | 1036.6462 | -4.92 |
| b10* | LKQINVIAGV* | 1036.6513 | 1036.6518 | 0.48 |
| b11 | LKQINVIAGVK | 1164.7463 | 1164.7416 | -4.04 |
| b11* | LKQINVIAGVK* | 1164.7463 | 1164.7469 | 0.52 |
| b12 | LKQINVIAGVKE | 1293.7889 | 1293.7892 | 0.23 |
| b12* | LKQINVIAGVKE* | 1293.7889 | 1293.7904 | 1.15 |
| b13 | LKQINVIAGVKEP | 1390.8417 | Not Found | N/A |
| b13* | LKQINVIAGVKEP* | 1390.8417 | 1390.8405 | -0.86 |
| b14 | LKQINVIAGVKEPI | 1503.9257 | 1503.9269 | 0.80 |
| b14* | LKQINVIAGVKEPI* | 1503.9257 | 1503.9231 | -1.72 |
| b15 | LKQINVIAGVKEPIR | 1660.0268 | 1660.0259 | -0.54 |
| b15* | LKQINVIAGVKEPIR* | 1660.0268 | 1660.0241 | -1.63 |
| b16 | LKQINVIAGVKEPIRA | 1731.0640 | 1731.0672 | 1.85 |
| b16* | LKQINVIAGVKEPIRA* | 1731.0640 | 1731.0588 | -3.00 |
| b17 | LKQINVIAGVKEPIRAY | 1894.1273 | 1894.1249 | -1.26 |
| b17* | LKQINVIAGVKEPIRAY* | 1894.1273 | Not Found | N/A |
| b18 | LKQINVIAGVKEPIRAYG | 976.0780 | 976.0740 | -4.09 |
| b18* | LKQINVIAGVKEPIRAYG* | 976.0780 | 976.0784 | 0.41 |
| b19 | LKQINVIAGVKEPIRAYGC | 1027.5826 | 1027.5809 | -1.65 |
| b19* | (-1 Da) LKQINVIAGVKEPIRAYGC* | 1027.0787 | Not Found | N/A |
| b20 | LKQINVIAGVKEPIRAYGCS | 1071.0986 | 1071.0959 | -2.52 |
| b20* | (-2 Da) LKQINVIAGVKEPIRAYGCS* | 1070.5947 | Not Found | N/A |
| b21 | LKQINVIAGVKEPIRAYGCSD | 1128.6121 | 1128.6072 | -4.34 |
| b21* | (-2 Da) LKQINVIAGVKEPIRAYGCSD* | 1127.6043 | 1127.6062 | 1.68 |
| b22 | LKQINVIAGVKEPIRAYGCSDN | 1185.6336 | 1185.6282 | -4.55 |
| b22* | (-2 Da) | 1184.6257 | 1184.6277 | 1.69 |
| b23 | LKQINVIAGVKEPIRAYGCSDNN* | 828.7724 | 828.7675 | -5.91 |
| b23* | (-2 Da) | 828.1006 | 828.1010 | 0.48 |
| | LKQINVIAGVKEPIRAYGCSDNN* | | | |
| b24 | LKQINVIAGVKEPIRAYGCSDNNA | 852.4515 | 852.4475 | -4.69 |
| b24* | (-2 Da) | 851.7796 | 851.7798 | 0.23 |
| | LKQINVIAGVKEPIRAYGCSDNNA* | | | |
| b25 | LKQINVIAGVKEPIRAYGCSDNNAA | 876.1305 | 876.1295 | -1.14 |
| b25* | (-2 Da) | 875.4586 | 875.4587 | 0.11 |
| | LKQINVIAGVKEPIRAYGCSDNNAA* | | | |
| [M] | LKQINVIAGVKEPIRAYGCSDNNAAA | 905.8131 | 905.8138 | 0.77 |
| [M]* | (-2 Da) | 905.1412 | 905.1410 | -0.22 |
| | LKQINVIAGVKEPIRAYGCSDNNAAA* | | | |
| y25 | KQINVIAGVKEPIRAYGCSDNNAAA | 868.1184 | 868.1194 | 1.15 |
| y25* | (-2 Da) | 867.4465 | Not Found | N/A |
| | KQINVIAGVKEPIRAYGCSDNNAAA* | | | |
| y24 | QINVIAGVKEPIRAYGCSDNNAAA | 825.4201 | Not Found | N/A |
| y24* | (-2 Da) | 824.7482 | Not Found | N/A |
| | QINVIAGVKEPIRAYGCSDNNAAA* | | | |
| y23 | INVIAGVKEPIRAYGCSDNNAAA | 782.7339 | Not Found | N/A |
| y23* | (-2 Da) | 782.0620 | Not Found | N/A |
| | INVIAGVKEPIRAYGCSDNNAAA* | | | |
| y22 | NVIAGVKEPIRAYGCSDNNAAA | 745.0392 | 745.0391 | -0.13 |
| y22* | (-2 Da) | 744.3673 | Not Found | N/A |
| | NVIAGVKEPIRAYGCSDNNAAA* | | | |
| y21 | VIAGVKEPIRAYGCSDNNAAA | 1060.0337 | 1060.0328 | -0.85 |
| y21* | (-2 Da) | 1059.0258 | 1059.0323 | 6.13 |
| | VIAGVKEPIRAYGCSDNNAAA* | | | |
| y20 | IAGVKEPIRAYGCSDNNAAA | 1010.4995 | 1010.4992 | -0.30 |
| y20* | (-2 Da) IAGVKEPIRAYGCSDNNAAA* | 1009.4916 | 1009.4903 | -1.28 |
| y19 | AGVKEPIRAYGCSDNNAAA | 953.9574 | 953.9570 | -0.42 |
| y19* | (-2 Da) AGVKEPIRAYGCSDNNAAA* | 952.9496 | 952.9494 | -0.21 |
| y18 | GVKEPIRAYGCSDNNAAA | 918.4389 | 918.4353 | -3.92 |
| y18* | (-2 Da) GVKEPIRAYGCSDNNAAA* | 917.4310 | 917.4311 | 0.11 |
| y17 | VKEPIRAYGCSDNNAAA | 889.9281 | 889.9283 | 0.22 |
| y17* | (-2 Da) VKEPIRAYGCSDNNAAA* | 888.9203 | 888.9226 | 2.59 |
| y16 | KEPIRAYGCSDNNAAA | 1679.7806 | 1679.7807 | 0.06 |
| y16* | (-2 Da) KEPIRAYGCSDNNAAA* | 1677.7649 | 1677.7671 | 1.31 |
| y15 | EPIRAYGCSDNNAAA | 1551.6856 | 1551.6816 | -2.58 |
| y15* | (-2 Da) EPIRAYGCSDNNAAA* | 1549.6700 | 1549.6706 | 0.38 |
| y14 | PIRAYGCSDNNAAA | 1422.6430 | 1422.6373 | -4.01 |
| y14* | (-2 Da) PIRAYGCSDNNAAA* | 1420.6274 | 1420.6277 | 0.21 |
| y13 | IRAYGCSDNNAAA | 1325.5903 | 1325.5886 | -1.28 |
| y13* | (-2 Da) IRAYGCSDNNAAA* | 1323.5746 | 1323.5736 | -0.75 |
| y12 | RAYGCSDNNAAA | 1212.5062 | 1212.5059 | -0.25 |
| y12* | (-2 Da) RAYGCSDNNAAA* | 1210.4906 | 1210.4893 | -1.07 |
| y11 | AYGCSDNNAAA | 1056.4051 | Not Found | N/A |
| y11* | (-2 Da) AYGCSDNNAAA* | 1054.3894 | 1054.3885 | -0.85 |
| y10 | YGCSDNNAAA | 985.3680 | 985.3618 | -6.29 |
| y10* | (-2 Da) YGCSDNNAAA* | 983.3523 | 983.3513 | -1.02 |
| y9 | GCSDNNAAA | 822.3047 | 822.3010 | -4.50 |
| y9* | (-2 Da) GCSDNNAAA* | 820.2890 | 820.2887 | -0.37 |
| y8 | CSDNNAAA | 765.2832 | 765.2798 | -4.44 |
| y8* | (-2 Da) CSDNNAAA* | 763.2675 | 763.2687 | 1.57 |
| y7 | SDNNAAA | 662.2740 | 662.2699 | 6.19 |
| y7* | (-1 Da) SDNNAAA* | 661.2662 | Not Found | N/A |
| y6 | DNNAAA | 575.2420 | 575.2402 | -3.13 |
| y6* | (-1 Da) DNNAAA* | 574.2342 | Not Found | N/A |
| y5 | NNAAA | 460.2150 | 460.2139 | -2.39 |
| y5* | NNAAA* | 460.2150 | 460.2154 | 0.87 |
| y4 | NAAA | 346.1721 | 346.1704 | -4.91 |
| y4* | NAAA* | 346.1721 | 346.1724 | 0.87 |
| y3 | AAA | 232.1292 | 232.1289 | -1.29 |
| y3* | AAA* | 232.1292 | 232.1293 | 0.43 |
| y2 | AA | 161.0921 | 161.0916 | -3.10 |
| y2* | AA* | 161.0921 | 161.0921 | 0 |

Unmodified indicates z = 1 charge state.
Underline indicates z = 2 charge state.
Italics indicates z = 3 charge state.
* Indicates the fragment originated from the modified peptide

TABLE 5
LKQINVIAGVKEPIRAYGCSNDAAA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1850 | −5.36 |
| b2* | LK* | 242.1863 | 242.1862 | −0.41 |
| b3 | LKQ | 370.2449 | 370.2432 | −4.59 |
| b3* | LKQ* | 370.2449 | 370.2450 | 0.27 |
| b4 | LKQI | 483.3289 | 483.3269 | −4.13 |
| b4* | LKQI* | 483.3289 | 483.3290 | 0.20 |
| b5 | LKQIN | 597.3719 | 597.3692 | −4.51 |
| b5* | LKQIN* | 597.3719 | 597.3708 | −1.84 |
| b6 | LKQINV | 696.4403 | 696.4371 | −4.59 |
| b6* | LKQINV* | 696.4403 | 696.4408 | 0.71 |
| b7 | LKQINVI | 809.5244 | 809.5222 | −2.71 |
| b7* | LKQINVI* | 809.5244 | 809.5245 | 0.12 |
| b8 | LKQINVIA | 880.5615 | 880.5580 | −3.97 |
| b8* | LKQINVIA* | 880.5615 | 880.5612 | −0.34 |
| b9 | LKQINVIAG | 937.5829 | 937.5833 | 0.42 |
| b9* | LKQINVIAG* | 937.5829 | 937.5838 | 0.95 |
| b10 | LKQINVIAGV | 1036.6513 | 1036.6472 | −3.95 |
| b10* | LKQINVIAGV* | 1036.6513 | 1036.6520 | 0.67 |
| b11 | LKQINVIAGVK | 1164.7463 | 1164.7429 | −2.91 |
| b11* | LKQINVIAGVK* | 1164.7463 | 1164.7484 | 1.80 |
| b12 | LKQINVIAGVKE | 1293.7889 | 1293.7865 | −1.85 |
| b12* | LKQINVIAGVKE* | 1293.7889 | 1293.7904 | 1.16 |
| b13 | LKQINVIAGVKEP | 1390.8417 | Not Found | N/A |
| b13* | LKQINVIAGVKEP* | 1390.8417 | 1390.8349 | −4.88 |
| b14 | LKQINVIAGVKEPI | 1503.9257 | 1503.9224 | −2.19 |
| b14* | LKQINVIAGVKEPI* | 1503.9257 | 1503.9261 | 0.26 |
| b15 | LKQINVIAGVKEPIR | 1660.0268 | 1660.0205 | −3.79 |
| b15* | LKQINVIAGVKEPIR* | 1660.0268 | 1660.0196 | −4.33 |
| b16 | LKQINVIAGVKEPIRA | 1731.0640 | 1731.0645 | 0.28 |
| b16* | LKQINVIAGVKEPIRA* | 1731.0640 | 1731.0599 | −2.36 |
| b17 | LKQINVIAGVKEPIRAY | 1894.1273 | Not Found | N/A |
| b17* | LKQINVIAGVKEPIRAY* | 1894.1273 | Not Found | N/A |
| b18 | LKQINVIAGVKEPIRAYG | 976.0780 | 976.0755 | −2.56 |
| b18* | LKQINVIAGVKEPIRAYG* | 976.0780 | 976.0777 | −0.30 |
| b19 | LKQINVIAGVKEPIRAYGC | 1027.5826 | 1027.5796 | −2.91 |
| b19* | (−1 Da) LKQINVIAGVKEPIRAYGC* | 1027.0787 | Not Found | N/A |
| b20 | LKQINVIAGVKEPIRAYGCS | 1071.5626 | 1071.5614 | −1.11 |
| b20* | (−1 Da) LKQINVIAGVKEPIRAYGCS* | 1070.5947 | Not Found | N/A |
| b21 | LKQINVIAGVKEPIRAYGCSN | 1128.6121 | 1128.6162 | 3.63 |
| b21* | (−1 Da) | 1127.6043 | Not Found | N/A |
| | LKQINVIAGVKEPIRAYGCSN* | | | |
| b22 | LKQINVIAGVKEPIRAYGCSND | 1185.6336 | 1185.6344 | 0.67 |
| b22* | (−2 Da) | 1184.6257 | 1184.6242 | −1.26 |
| | LKQINVIAGVKEPIRAYGCSND* | | | |
| b23 | LKQINVIAGVKEPIRAYGCSNDA | 814.4372 | 814.4347 | −3.07 |
| b23* | (−2 Da) | 813.7653 | 813.7661 | 0.98 |
| | LKQINVIAGVKEPIRAYGCSNDA* | | | |
| b24 | LKQINVIAGVKEPIRAYGCSNDAA | 838.1162 | 838.1121 | −4.89 |
| b24* | (−2 Da) | 837.4443 | 837.4445 | 0.24 |
| | LKQINVIAGVKEPIRAYGCSNDAA* | | | |
| [M] | LKQINVIAGVKEPIRAYGCSNDAAA | 867.7988 | 867.7947 | −4.72 |
| [M]* | (−2 Da) | 867.1269 | 867.1268 | −0.11 |
| | LKQINVIAGVKEPIRAYGCSNDAAA* | | | |
| y24 | KQINVIAGVKEPIRAYGCSNDAAA | 830.1041 | 830.1020 | −2.53 |
| y24* | (−2 Da) | 829.4322 | 829.4326 | 0.48 |
| | KQINVIAGVKEPIRAYGCSNDAAA* | | | |
| y23 | QINVIAGVKEPIRAYGCSNDAAA | 787.4058 | 787.4063 | 0.63 |
| y23* | (−2 Da) | 786.7339 | 786.7335 | −0.51 |
| | QINVIAGVKEPIRAYGCSNDAAA* | | | |
| y22 | INVIAGVKEPIRAYGCSNDAAA | 1116.5757 | 1116.5719 | −3.40 |
| y22* | (−2 Da) | 1115.5679 | 1115.5670 | −0.81 |
| | INVIAGVKEPIRAYGCSNDAAA* | | | |
| y21 | NVIAGVKEPIRAYGCSNDAAA | 1060.0377 | 1060.0333 | −4.15 |
| y21* | (−2 Da) | 1059.0258 | 1059.0260 | 0.19 |
| | NVIAGVKEPIRAYGCSNDAAA* | | | |
| y20 | VIAGVKEPIRAYGCSNDAAA | 1003.0122 | 1003.0079 | −4.29 |
| y20* | (−2 Da) VIAGVKEPIRAYGCSNDAAA* | 1002.0044 | 1002.0047 | 0.30 |
| y19 | IAGVKEPIRAYGCSNDAAA | 953.4780 | 953.4733 | −4.93 |
| y19* | (−2 Da) IAGVKEPIRAYGCSNDAAA* | 952.4702 | 952.4701 | −0.10 |
| y18 | AGVKEPIRAYGCSNDAAA | 896.9360 | 896.9361 | 0.11 |
| y18* | (−2 Da) AGVKEPIRAYGCSNDAAA* | 895.9281 | 895.9276 | −0.56 |
| y17 | GVKEPIRAYGCSNDAAA | 1721.8275 | 1721.8199 | −4.41 |
| y17* | (−2 Da) GVKEPIRAYGCSNDAAA* | 1719.8119 | 1719.8161 | 2.44 |
| y16 | VKEPIRAYGCSNDAAA | 1664.8061 | 1664.8049 | −0.72 |
| y16* | (−2 Da) VKEPIRAYGCSNDAAA* | 1662.7904 | 1662.7873 | −1.86 |
| y15 | KEPIRAYGCSNDAAA | 1565.7377 | 1565.7379 | 0.13 |
| y15* | (−2 Da) KEPIRAYGCSNDAAA* | 1563.6271 | 1563.6308 | 2.37 |
| y14 | EPIRAYGCSNDAAA | 1437.6427 | 1437.6395 | −2.22 |
| y14* | (−2 Da) EPIRAYGCSNDAAA* | 1435.6271 | 1435.6292 | 1.47 |
| y13 | PIRAYGCSNDAAA | 1308.6001 | 1308.5982 | −1.45 |
| y13* | (−2 Da) PIRAYGCSNDAAA* | 1306.5845 | 1306.5869 | 1.84 |
| y12 | IRAYGCSNDAAA | 1211.5473 | 1211.5478 | 0.41 |
| y12* | (−2 Da) IRAYGCSNDAAA* | 1209.5317 | 1209.5339 | 1.82 |
| y11 | RAYGCSNDAAA | 1098.4633 | 1098.4599 | −3.09 |
| y11* | (−2 Da) RAYGCSNDAAA* | 1096.4476 | 1096.4490 | 1.28 |
| y10 | AYGCSNDAAA | 942.3622 | 942.3587 | −3.71 |
| y10* | (−2 Da) AYGCSNDAAA* | 940.3465 | 940.3490 | 2.66 |
| y9 | YGCSNDAAA | 871.3251 | 871.3208 | −4.94 |
| y9* | (−2 Da) YGCSNDAAA* | 869.3094 | 869.3094 | 0 |
| y8 | GCSNDAAA | 708.2617 | 708.2585 | −4.52 |
| y8* | (−2 Da) GCSNDAAA* | 706.2461 | 706.2460 | 0.14 |
| y7 | CSNDAAA | 651.2403 | 651.2374 | −4.45 |
| y7* | (−2 Da) CSNDAAA* | 649.2246 | 649.2247 | 0.15 |
| y6 | SNDAAA | 548.2311 | 548.2296 | −2.73 |
| y6* | (−1 Da) SNDAAA* | 547.2233 | Not Found | N/A |
| y5 | NDAAA | 461.1991 | 461.1986 | −1.08 |
| y5* | (−1 Da) NDAAA* | 460.1921 | Not Found | N/A |
| y4 | DAAA | 347.1561 | 347.1554 | −2.02 |
| y4* | (−1 Da) DAAA* | 346.1483 | Not Found | N/A |
| y3 | AAA | 232.1292 | 232.1281 | −4.73 |
| y3* | AAA* | 232.1292 | 232.1294 | 0.86 |
| y2 | AA | 161.0921 | 161.0911 | −6.20 |
| y2* | AA* | 161.0921 | 161.0919 | −1.24 |

Unmodified indicates z = 1 charge state.
Underline indicates z = 2 charge state.
Italics indicates z = 3 charge state.
*Indicates the fragment originated from the modified peptide

TABLE 6
LKQINVIAGVKEPIRAYGCSAANDA

| | | Expected | Observed | |
|---|---|---|---|---|
| | | Monoisoptic | Monoisotopic | Ppm |
| Ion | Sequence | Mass | Mass | Error |
| b2 | LK | 242.1863 | 242.1861 | 0.72 |
| b2* | LK* | 242.1863 | 242.1862 | 0.39 |
| b3 | LKQ | 370.2449 | 370.2447 | 0.52 |
| b3* | LKQ* | 370.2449 | 370.2452 | −0.72 |
| b4 | LKQI | 483.3289 | 483.3296 | −1.46 |
| b4* | LKQI* | 483.3289 | 483.3280 | 1.84 |
| b5 | LKQIN | 597.3719 | 597.3721 | −0.31 |
| b5* | LKQIN* | 597.3719 | 597.3736 | −2.82 |
| b6 | LKQINV | 696.4403 | 696.4417 | −2.07 |
| b6* | LKQINV* | 696.4403 | 696.4395 | 1.21 |
| b7 | LKQINVI | 809.5244 | 809.5247 | −0.33 |
| b7* | LKQINVI* | 809.5244 | 809.5220 | 2.95 |
| b8 | LKQINVIA | 880.5615 | 880.5601 | 1.57 |
| b8* | LKQINVIA* | 880.5615 | 880.5634 | −2.18 |
| b9 | LKQINVIAG | 937.5829 | 937.5826 | 0.32 |
| b9* | LKQINVIAG* | 937.5829 | 937.5795 | 3.60 |
| b10 | LKQINVIAGV | 1036.6513 | 1036.6500 | 1.23 |
| b10* | LKQINVIAGV* | 1036.6513 | 1036.6545 | −3.11 |
| b11 | LKQINVIAGVK | 1164.7463 | 1164.7436 | 2.33 |
| b11* | LKQINVIAGVK* | 1164.7463 | 1164.7492 | −2.47 |
| b12 | LKQINVIAGVKE | 1293.7889 | 1293.7860 | 2.23 |
| b12* | LKQINVIAGVKE* | 1293.7889 | 1293.7928 | −2.99 |
| b13 | LKQINVIAGVKEP | 1390.8417 | 1390.8476 | −4.27 |
| b13* | LKQINVIAGVKEP* | 1390.8417 | 1390.8430 | −0.95 |
| b14 | LKQINVIAGVKEPI | 1503.9257 | 1503.9191 | 4.38 |
| b14* | LKQINVIAGVKEPI* | 1503.9257 | 1503.9279 | −1.48 |
| b15 | LKQINVIAGVKEPIR | 1660.0268 | 1660.0259 | 0.57 |
| b15* | LKQINVIAGVKEPIR* | 1660.0268 | 1660.0203 | 3.93 |
| b16 | LKQINVIAGVKEPIRA | 1731.0640 | 1731.0704 | −3.69 |
| b16* | LKQINVIAGVKEPIRA* | 1731.0640 | Not Found | N/A |
| b17 | LKQINVIAGVKEPIRAY | 947.5673 | 947.5654 | 2.05 |
| b17* | LKQINVIAGVKEPIRAY* | 947.5673 | 947.5692 | −1.96 |
| b18 | LKQINVIAGVKEPIRAYG | 976.0780 | 976.0771 | 0.89 |
| b18* | LKQINVIAGVKEPIRAYG* | 976.0780 | 976.0812 | −3.24 |
| b19 | LKQINVIAGVKEPIRAYGC | 1027.5826 | 1027.5856 | −2.92 |
| b19* | (−1 Da) LKQINVIAGVKEPIRAYGC* | 1027.0787 | Not Found | N/A |
| b20 | LKQINVIAGVKEPIRAYGCS | 1071.0986 | 1071.1023 | −3.50 |
| b20* | (−1 Da) LKQINVIAGVKEPIRAYGCS* | 1070.5947 | Not Found | N/A |
| b21 | LKQINVIAGVKEPIRAYGCSA | 1106.6172 | 1106.6200 | −2.57 |
| b21* | (−1 Da) LKQINVIAGVKEPIRAYGCSA* | 1106.1133 | Not Found | N/A |
| b22 | LKQINVIAGVKEPIRAYGCSAA | 761.7596 | 761.7587 | −3.50 |
| b22* | (−1 Da) LKQINVIAGVKEPIRAYGCSAA* | 761.4236 | Not Found | N/A |
| b23 | LKQINVIAGVKEPIRAYGCSAAN | 799.7739 | 799.7731 | 1.04 |
| b23* | (−1 Da) LKQINVIAGVKEPIRAYGCSAAN* | 799.4379 | Not Found | N/A |
| b24 | LKQINVIAGVKEPIRAYGCSAAND | 838.1162 | 838.1166 | −0.53 |
| b24* | (−2 Da) LKQINVIAGVKEPIRAYGCSAAND* | 837.4443 | 837.4470 | −3.19 |
| [M] | LKQINVIAGVKEPIRAYGCSAANDA | 867.7988 | 867.7984 | 0.46 |
| | (−2 Da) | | | |
| [M]* | LKQINVIAGVKEPIRAYGCSAANDA* | 867.1269 | 867.1261 | 0.92 |
| y24 | IKQINVIAGVKEPIRAYGCSAANDA | 830.1041 | Not Found | N/A |
| | (−2 Da) | | | |
| y24* | IKQINVIAGVKEPIRAYGCSAANDA* | 829.4322 | 829.4320 | 0.27 |
| y23 | QINVIAGVKEPIRAYGCSAANDA | 787.4058 | 787.4075 | −2.15 |
| y23* | (−2 Da) QINVIAGVKEPIRAYGCSAANDA* | 786.7339 | Not Found | N/A |
| y22 | INVIAGVKEPIRAYGCSAANDA | 744.7196 | 744.7179 | 2.23 |
| y22* | (−2 Da) INVIAGVKEPIRAYGCSAANDA* | 744.0477 | 744.0462 | 2.04 |
| y21 | NVIAGVKEPIRAYGCSAANDA | 1060.0337 | 1060.0351 | −1.32 |
| y21* | (−2 Da) NVIAGVKEPIRAYGCSAANDA* | 1059.0258 | 1059.0258 | 0 |
| y20 | VIAGVKEPIRAYGCSAANDA | 1003.0122 | 1003.0122 | 0 |
| y20* | (−2 Da) VIAGVKEPIRAYGCSAANDA* | 1002.0044 | 1002.0079 | −3.54 |
| y19 | IAGVKEPIRAYGCSAANDA | 953.4780 | 953.4781 | −0.14 |
| y19* | (−2 Da) IAGVKEPIRAYGCSAANDA* | 952.4702 | 952.4706 | −0.43 |
| y18 | AGVKEPIRAYGCSAANDA | 896.9360 | 896.9361 | −0.12 |
| y18* | (−2 Da) AGVKEPIRAYGCSAANDA* | 895.9281 | 895.9277 | 0.41 |
| y17 | GVKEPIRAYGCSAANDA | 861.4174 | 861.4186 | −1.40 |
| y17* | (−2 Da) GVKEPIRAYGCSAANDA* | 860.4096 | 860.4096 | 0 |
| y16 | VKEPIRAYGCSAANDA | 832.9067 | 832.9060 | 0.81 |
| y16* | (−2 Da) VKEPIRAYGCSAANDA* | 831.8989 | Not Found | N/A |
| y15 | KEPIRAYGCSAANDA | 1565.7377 | 1565.7338 | 2.49 |
| y15* | (−2 Da) KEPIRAYGCSAANDA* | 1563.7220 | 1563.7181 | 2.51 |
| y14 | EPIRAYGCSAANDA | 1437.6427 | 1437.6475 | −3.36 |
| y14* | (−2 Da) EPIRAYGCSAANDA* | 1435.6271 | 1435.6287 | −1.12 |
| y13 | PIRAYGCSAANDA | 1308.6001 | 1308.5977 | 1.84 |
| y13* | (−2 Da) PIRAYGCSAANDA* | 1306.5845 | 1306.5866 | −1.64 |
| y12 | IRAYGCSAANDA | 1211.5473 | 1211.5449 | 1.98 |
| y12* | (−2 Da) IRAYGCSAANDA* | 1209.5317 | 1209.5337 | −1.68 |
| y11 | RAYGCSAANDA | 1098.4633 | 1098.4610 | 2.07 |
| y11* | (−2 Da) RAYGCSAANDA* | 1096.4476 | 1096.4490 | −1.25 |
| y10 | AYGCSAANDA | 942.3622 | 942.3655 | −3.51 |
| y10* | (−2 Da) AYGCSAANDA* | 940.3465 | 940.3492 | −2.82 |
| y9 | YGCSAANDA | 871.3251 | 871.3281 | −0.96 |
| y9* | (−2 Da) YGCSAANDA* | 869.3094 | 869.3102 | −0.96 |
| y8 | GCSAANDA | 708.2617 | 708.2597 | 2.77 |
| y8* | (−2 Da) GCSAANDA* | 706.2461 | 706.2462 | −0.12 |
| y7 | CSAANDA | 651.2403 | 651.2398 | 1.08 |
| y7* | (−2 Da) CSAANDA* | 649.2246 | 649.2239 | 1.08 |
| y6 | SAANDA | 548.2311 | 548.2317 | −1.04 |
| y6* | (−1 Da) SAANDA* | 547.2233 | Not Found | N/A |
| y5 | AANDA | 461.1991 | 461.1989 | 0.42 |
| y5* | (−1 Da) AANDA* | 460.1912 | Not Found | N/A |
| y4 | ANDA | 390.1619 | 390.1614 | 1.26 |
| y4* | (−1 Da) ANDA* | 389.1541 | Not Found | N/A |
| y3 | NDA | 319.1248 | 319.1249 | −0.25 |
| y3* | (−1 Da) NDA* | 318.1170 | Not Found | N/A |
| y2 | DA | 205.0819 | 205.0819 | 0 |
| y2* | (−1 Da) DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 7
LKQINVIAGVKEPIRAYGCSAAANDA

| | | Expected | Observed | |
|---|---|---|---|---|
| | | Monoisotopic | Monoisotopic | Ppm |
| Ion | Sequence | Mass | Mass | error |
| b2 | LK | 242.1863 | 242.1861 | 0.67 |
| b2* | LK* | 242.1863 | 242.1860 | 1.09 |
| b3 | LKQ | 370.2449 | 370.2448 | 0.33 |
| b3* | LKQ* | 370.2449 | 370.2446 | 0.71 |
| b4 | LKQI | 483.3289 | 483.3297 | −1.75 |
| b4* | LKQI* | 483.3289 | 483.3296 | −1.39 |
| b5 | LKQIN | 597.3719 | 597.3723 | −0.70 |
| b5* | LKQIN* | 597.3719 | 597.3721 | −0.35 |
| b6 | LKQINV | 696.4403 | 696.4421 | −2.54 |
| b6* | LKQINV* | 696.4403 | 696.4418 | −2.20 |
| b7 | LKQINVI | 809.5244 | 809.5251 | −0.87 |
| b7* | LKQINVI* | 809.5244 | 809.5248 | −0.53 |
| b8 | LKQINVIA | 880.5615 | 880.5606 | 0.99 |
| b8* | LKQINVIA* | 880.5615 | 880.5603 | 1.33 |
| b9 | LKQINVIAG | 937.5829 | 937.5832 | −0.30 |
| b9* | LKQINVIAG* | 937.5829 | 937.5829 | 0.04 |
| b10 | LKQINVIAGV | 1036.6513 | 1036.6507 | 0.55 |
| b10* | LKQINVIAGV* | 1036.6513 | 1036.6504 | 0.90 |
| b11 | LKQINVIAGVK | 1164.7463 | 1164.7445 | 1.57 |
| b11* | LKQINVIAGVK* | 1164.7463 | 1164.7441 | 1.93 |
| b12 | LKQINVIAGVKE | 1293.7889 | 1293.7871 | 1.41 |
| b12* | LKQINVIAGVKE* | 1293.7889 | 1293.7866 | 1.77 |
| b13 | LKQINVIAGVKEP | 1390.8417 | 1390.8366 | 3.70 |
| b13* | LKQINVIAGVKEP* | 1390.8417 | 1390.8360 | 4.08 |
| b14 | LKQINVIAGVKEPI | 1503.9257 | 1503.9205 | 3.45 |
| b14* | LKQINVIAGVKEPI* | 1503.9257 | 1503.9199 | 3.84 |
| b15 | LKQINVIAGVKEPIR | 1660.0268 | 1660.0275 | −0.43 |
| b15* | LKQINVIAGVKEPIR* | 1660.0268 | 1660.0268 | −0.02 |
| b16 | LKQINVIAGVKEPIRA | 1731.0640 | 1731.0722 | −4.72 |
| b16* | LKQINVIAGVKEPIRA* | 1731.0640 | 1731.0714 | −4.30 |
| b17 | LKQINVIAGVKEPIRAY | 947.5673 | 947.5659 | 1.43 |
| b17* | LKQINVIAGVKEPIRAY* | 947.5673 | 947.5656 | 1.77 |
| b18 | LKQINVIAGVKEPIRAYG | 976.0780 | 976.0778 | 0.24 |
| b18* | LKQINVIAGVKEPIRAYG* | 976.0780 | 976.0774 | 0.58 |
| b19 | LKQINVIAGVKEPIRAYGC | 1027.5826 | 1027.5863 | −3.59 |
| b19* | (−1 Da) LKQINVIAGVKEPIRAYGC* | 1027.0787 | Not Found | N/A |
| b20 | LKQINVIAGVKEPIRAYGCS | 1071.0986 | 1071.0948 | 3.56 |
| b20* | (−1 Da) LKQINVIAGVKEPIRAYGCS* | 1070.5947 | Not Found | N/A |
| b21 | LKQINVIAGVKEPIRAYGCSA | 1106.6172 | 1106.6208 | −3.29 |
| b21* | (−1 Da) LKQINVIAGVKEPIRAYGCSA* | 1106.1133 | Not Found | N/A |
| b22 | LKQINVIAGVKEPIRAYGCSAA | 1142.1357 | 1142.1392 | −3.02 |
| b22* | (−1 Da) LKQINVIAGVKEPIRAYGCSAA* | 1141.6318 | Not Found | N/A |
| b23 | LKQINVIAGVKEPIRAYGCSAAA | 785.4386 | 785.4366 | 2.50 |
| b23* | (−1 Da) LKQINVIAGVKEPIRAYGCSAAA* | 785.1027 | Not Found | N/A |
| b24 | LKQINVIAGVKEPIRAYGCSAAAN | 823.4529 | 823.4529 | −0.03 |
| b24* | (−1 Da) LKQINVIAGVKEPIRAYGCSAAAN* | 823.1170 | Not Found | N/A |
| b25 | LKQINVIAGVKEPIRAYGCSAAAND | 861.7952 | 861.7969 | −1.94 |
| b25* | (−2 Da) | 861.1234 | 861.1252 | −2.05 |
| | LKQINVIAGVKEPIRAYGCSAAAND* | | | |
| [M] | LKQINVIAGVKEPIRAYGCSAAANDA | 891.4778 | 891.4781 | −0.30 |
| [M]* | (−2 Da) | 890.8059 | 890.8029 | 3.38 |
| | LKQINVIAGVKEPIRAYGCSAAANDA* | | | |
| y25 | KQINVIAGVKEPIRAYGCSAAANDA | 853.7831 | Not Found | N/A |
| y25* | (−2 Da) | 853.1112 | 853.1112 | −0.06 |
| | KQINVIAGVKEPIRAYGCSAAANDA* | | | |
| y24 | QINVIAGVKEPIRAYGCSAAANDA | 811.0848 | Not Found | N/A |
| y24* | (−2 Da) QINVIAGVKEPIRAYGCSAAANDA* | 810.4129 | 810.4102 | 3.30 |
| y23 | INVIAGVKEPIRAYGCSAAANDA | 768.3986 | 768.3971 | 1.97 |
| y23* | (−2 Da) INVIAGVKEPIRAYGCSAAANDA* | 767.7267 | 767.7256 | 1.42 |
| y22 | NVIAGVKEPIRAYGCSAAANDA | 730.7039 | Not Found | N/A |
| y22* | (−2 Da) NVIAGVKEPIRAYGCSAAANDA* | 730.0320 | 730.0306 | 1.93 |
| y21 | VIAGVKEPIRAYGCSAAANDA | 1038.5308 | 1038.5288 | 1.94 |
| y21* | (−2 Da) VIAGVKEPIRAYGCSAAANDA* | 1037.5229 | 1037.5214 | 1.44 |
| y20 | JAGVKEPIRAYGCSAAANDA | 988.9966 | 988.9947 | 1.88 |
| y20* | (−2 Da) IAGVKEPIRAYGCSAAANDA* | 987.9887 | 987.9923 | −3.63 |
| y19 | AGVKEPIRAYGCSAAANDA | 932.4545 | 932.4517 | 3.04 |
| y19* | (−2 Da) AGVKEPIRAYGCSAAANDA* | 931.4467 | 931.4463 | 0.45 |
| y18 | GVKEPIRAYGCSAAANDA | 896.9360 | 896.9366 | −0.72 |
| y18* | (−2 Da) GVKEPIRAYGCSAAANDA* | 895.9281 | 895.9309 | −3.13 |
| y17 | VKEPIRAYGCSAAANDA | 868.4252 | 868.4270 | −2.11 |
| y17* | (−2 Da) VKEPIRAYGCSAAANDA* | 867.4174 | 867.4204 | −3.43 |
| y16 | KEPIRAYGCSAAANDA | 818.8910 | 818.8892 | 2.25 |
| y16* | (−2 Da) KEPIRAYGCSAAANDA* | 817.8832 | 817.8842 | −1.20 |
| y15 | EPIRAYGCSAAANDA | 754.8435 | Not Found | N/A |
| y15* | (−2 Da) EPIRAYGCSAAANDA* | 753.8357 | 753.8356 | 0.18 |
| y14 | PIRAYGCSAAANDA | 1379.6372 | 1379.6396 | −1.72 |
| y14* | (−2 Da) PIRAYGCSAAANDA* | 1377.6216 | 1377.6244 | −2.03 |
| y13 | IRAYGCSAAANDA | 1282.5845 | 1282.5840 | 0.37 |
| y13* | (−2 Da) IRAYGCSAAANDA* | 1280.5688 | 1280.5712 | −1.86 |
| y12 | RAYGCSAAANDA | 1169.5004 | 1169.4996 | 0.67 |
| y12* | (−2 Da) RAYGCSAAANDA* | 1167.4847 | 1167.4820 | 2.28 |
| y11 | AYGCSAAANDA | 1013.3993 | 1013.3959 | 3.39 |
| y11* | (−2 Da) AYGCSAAANDA* | 1011.3836 | 1011.3869 | −3.25 |
| y10 | YGCSAAANDA | 942.3622 | 942.3592 | 3.15 |
| y10* | (−2 Da) YGCSAAANDA* | 940.3465 | 940.3457 | 0.89 |
| y9 | GCSAAANDA | 779.2988 | 779.2988 | 0.06 |
| y9* | (−2 Da) GCSAAANDA* | 777.2832 | 777.2856 | −3.14 |
| y8 | CSAAANDA | 722.2774 | Not Found | N/A |
| y8* | (−2 Da) CSAAANDA* | 720.2617 | 720.2620 | −0.47 |
| y7 | SAAANDA | 619.2682 | 619.2665 | 2.69 |
| y7* | (−1 Da) SAAANDA* | 618.2604 | Not Found | N/A |
| y6 | AAANDA | 532.2362 | 532.2351 | 2.02 |
| y6* | (−1 Da) AAANDA* | 531.2283 | Not Found | N/A |
| y5 | AANDA | 461.1991 | 461.1990 | 0.15 |
| y5* | (−1 Da) AANDA* | 460.1912 | Not Found | N/A |
| y4 | ANDA | 390.1619 | 390.1615 | 1.05 |
| y4* | (−1 Da) ANDA* | 389.1541 | Not Found | N/A |
| y3 | NDA | 319.1248 | 319.1249 | −0.39 |
| y3* | (−1 Da) NDA* | 318.1170 | Not Found | N/A |
| y2 | DA | 205.0819 | 205.0819 | −0.20 |
| y2* | (−1 Da) DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 8
LKQINVIAGVKEPIRAYGCSAAAANDA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1863 | −0.12 |
| b2* | LK* | 242.1863 | 242.1861 | 0.64 |
| b3 | LKQ | 370.2449 | 370.2451 | −0.43 |
| b3* | LKQ* | 370.2449 | 370.2451 | −0.48 |
| b4 | LKQI | 483.3289 | 483.3301 | −2.50 |
| b4* | LKQI* | 483.3289 | 483.3279 | 2.07 |
| b5 | LKQIN | 597.3719 | 597.3728 | −1.43 |
| b5* | LKQIN* | 597.3719 | 597.3735 | −2.60 |
| b6 | LKQINV | 696.4403 | 696.4382 | 3.00 |
| b6* | LKQINV* | 696.4403 | 696.4393 | 1.42 |
| b7 | LKQINVI | 809.5244 | 809.5257 | −1.57 |
| b7* | LKQINVI* | 809.5244 | 809.5218 | 3.15 |
| b8 | LKQINVIA | 880.5615 | 880.5612 | 0.29 |
| b8* | LKQINVIA* | 880.5615 | 880.5632 | −1.98 |
| b9 | LKQINVIAG | 937.5829 | 937.5838 | −0.99 |
| b9* | LKQINVIAG* | 937.5829 | 937.5861 | −3.46 |
| b10 | LKQINVIAGV | 1036.6513 | 1036.6514 | −0.13 |
| b10* | LKQINVIAGV* | 1036.6513 | 1036.6543 | −2.92 |
| b11 | LKQINVIAGVK | 1164.7463 | 1164.7452 | 0.91 |
| b11* | LKQINVIAGVK* | 1164.7463 | 1164.7490 | −2.28 |
| b12 | LKQINVIAGVKE | 1293.7889 | 1293.7879 | 0.76 |
| b12* | LKQINVIAGVKE* | 1293.7889 | 1293.7925 | −2.81 |
| b13 | LKQINVIAGVKEP | 1390.8417 | 1390.8374 | 3.06 |
| b13* | LKQINVIAGVKEP* | 1390.8417 | 1390.8428 | −0.78 |
| b14 | LKQINVIAGVKEPI | 1503.9257 | 1503.9215 | 2.81 |
| b14* | LKQINVIAGVKEPI* | 1503.9257 | 1503.9277 | −1.32 |
| b15 | LKQINVIAGVKEPIR | 1660.0268 | 1660.0285 | −1.05 |
| b15* | LKQINVIAGVKEPIR* | 1660.0268 | 1660.0200 | 4.09 |
| b16 | LKQINVIAGVKEPIRA | 1731.0640 | 1731.0562 | 4.53 |
| b16* | LKQINVIAGVKEPIRA* | 1731.0640 | 1731.0643 | −0.16 |
| b17 | LKQINVIAGVKEPIRAY | 947.5673 | 947.5666 | 0.74 |
| b17* | LKQINVIAGVKEPIRAY* | 947.5673 | 947.5690 | −1.77 |
| b18 | LKQINVIAGVKEPIRAYG | 976.0780 | 976.0784 | −0.44 |
| b18* | LKQINVIAGVKEPIRAYG* | 976.0780 | 976.0810 | −3.04 |
| b19 | LKQINVIAGVKEPIRAYGC | 1027.5826 | 1027.5792 | 3.33 |
| b19* | (−1 Da) LKQINVIAGVKEPIRAYGC* | 1027.0787 | Not Found | N/A |
| b20 | LKQINVIAGVKEPIRAYGCS | 1071.0986 | 1071.0955 | 2.89 |
| b20* | (−1 Da) LKQINVIAGVKEPIRAYGCS* | 1070.5947 | Not Found | N/A |
| b21 | LKQINVIAGVKEPIRAYGCSA | 1106.6172 | 1106.6129 | 3.92 |
| b21* | (−1 Da) LKQINVIAGVKEPIRAYGCSA* | 1106.1133 | Not Found | N/A |
| b22 | LKQINVIAGVKEPIRAYGCSAA | 1142.1357 | 1142.1399 | −3.69 |
| b22* | (−1 Da) LKQINVIAGVKEPIRAYGCSAA* | 1141.6318 | Not Found | N/A |
| b23 | LKQINVIAGVKEPIRAYGCSAAA | 785.4386 | 785.4372 | 1.80 |
| b23* | (−1 Da) | 785.1027 | Not Found | N/A |
| | LKQINVIAGVKEPIRAYGCSAAA* | | | |
| b24 | LKQINVIAGVKEPIRAYGCSAAAA | 809.1177 | 809.1163 | 1.78 |
| b24* | (−1 Da) | 808.7817 | Not Found | N/A |
| | LKQINVIAGVKEPIRAYGCSAAAA* | | | |
| b25 | LKQINVIAGVKEPIRAYGCSAAAAN | 847.1320 | 847.1296 | 2.86 |
| b25* | (−1 Da) | 846.7960 | Not Found | N/A |
| | LKQINVIAGVKEPIRAYGCSAAAAN* | | | |
| b26 | LKQINVIAGVKEPIRAYGCSAAAAND | 885.4743 | 885.4760 | −1.91 |
| b26* | LKQINVIAGVKEPIRAYGCSAAAAND* | 884.8024 | 884.8037 | −1.47 |
| | (−2 Da) | | | |
| [M] | LKQINVIAGVKEPIRAYGCSAAAANDA | 915.1568 | 915.1578 | −1.12 |
| [M]* | LKQINVIAGVKEPIRAYGCSAAAANDA* | 914.4850 | 914.4843 | 0.76 |
| | (−2 Da) | | | |
| y26 | KQINVIAGVKEPIRAYGCSAAAANDA | 877.4621 | 877.4594 | 3.04 |
| y26* | KQINVIAGVKEPIRAYGCSAAAANDA* | 876.7903 | 876.7901 | 0.25 |
| | (−2 Da) | | | |
| y25 | QINVIAGVKEPIRAYGCSAAAANDA | 834.7638 | Not Found | N/A |
| y25* | QINVIAGVKEPIRAYGCSAAAANDA* | 834.0919 | 834.0898 | 2.48 |
| | (−2 Da) | | | |
| y24 | INVIAGVKEPIRAYGCSAAAANDA | 792.0776 | Not Found | N/A |
| y24* | (−2 Da) | 791.4058 | 791.4065 | −0.95 |
| | INVIAGVKEPIRAYGCSAAAANDA* | | | |
| y23 | NVIAGVKEPIRAYGCSAAAANDA | 754.3829 | Not Found | N/A |
| y23* | (−2 Da) | 753.7111 | 753.7101 | 1.26 |
| | NVIAGVKEPIRAYGCSAAAANDA* | | | |
| y22 | VIAGVKEPIRAYGCSAAAANDA | 1074.0493 | 1074.0521 | −2.60 |
| y22* | (−2 Da) | 1073.0415 | 1073.0378 | 3.47 |
| | VIAGVKEPIRAYGCSAAAANDA* | | | |
| y21 | IAGVKEPIRAYGCSAAAANDA | 1024.5151 | 1024.5168 | −1.62 |
| y21* | (−2 Da) IAGVKEPIRAYGCSAAAANDA* | 1023.5073 | 1023.5096 | −2.22 |
| y20 | AGVKEPIRAYGCSAAAANDA | 967.9731 | 967.9736 | −0.53 |
| y20* | (−2 Da) AGVKEPIRAYGCSAAAANDA* | 966.9653 | 966.9630 | 2.41 |
| y19 | GVKEPIRAYGCSAAAANDA | 932.4545 | 932.4523 | 2.35 |
| y19* | (−2 Da) GVKEPIRAYGCSAAAANDA* | 931.4467 | 931.4495 | −3.03 |
| y18 | VKEPIRAYGCSAAAANDA | 903.9438 | 903.9457 | −2.05 |
| y18* | (−2 Da) VKEPIRAYGCSAAAANDA* | 902.9360 | 902.9370 | −1.07 |
| y17 | KEPIRAYGCSAAAANDA | 854.4096 | 854.4074 | 2.54 |
| y17* | (−2 Da) KEPIRAYGCSAAAANDA* | 853.4018 | 853.4036 | −2.05 |
| y16 | EPIRAYGCSAAAANDA | 1579.7169 | 1579.7105 | 4.03 |
| y16* | (−2 Da) EPIRAYGCSAAAANDA* | 1577.7013 | 1577.6947 | 4.16 |
| y15 | PIRAYGCSAAAANDA | 1450.6743 | 1450.6747 | −0.26 |
| y15* | (−2 Da) PIRAYGCSAAAANDA* | 1448.6587 | 1448.6651 | −4.43 |
| y14 | IRAYGCSAAAANDA | 1353.6216 | 1353.6247 | −2.33 |
| y14* | (−2 Da) IRAYGCSAAAANDA* | 1351.6059 | Not Found | N/A |
| y13 | RAYGCSAAAANDA | 1240.5375 | 1240.5425 | −4.06 |
| y13* | (−2 Da) RAYGCSAAAANDA* | 1238.5219 | 1238.5188 | 2.52 |
| y12 | AYGCSAAAANDA | 1084.4364 | 1084.4336 | 2.56 |
| y12* | (−2 Da) AYGCSAAAANDA* | 1082.4207 | Not Found | N/A |
| y11 | YGCSAAAANDA | 1013.3993 | 1013.3966 | 2.71 |
| y11* | (−2 Da) YGCSAAAANDA* | 1011.3836 | 1011.3830 | 0.55 |
| y10 | GCSAAAANDA | 850.3360 | 850.3363 | −0.38 |
| y10* | (−2 Da) GCSAAAANDA* | 848.3203 | 848.3194 | 1.11 |
| y9 | CSAAAANDA | 793.3145 | 793.3140 | 0.59 |
| y9* | (−2 Da) CSAAAANDA* | 791.2988 | 791.3010 | −2.76 |
| y8 | SAAAANDA | 690.3053 | 690.3043 | 1.39 |
| y8* | (−1 Da) SAAAANDA* | 689.2975 | Not Found | N/A |
| y7 | AAAANDA | 603.2733 | 603.2733 | 0.00 |
| y7* | (−1 Da) AAAANDA* | 602.2655 | Not Found | N/A |
| y6 | AAANDA | 532.2362 | 532.2355 | 1.28 |
| y6* | (−1 Da) AAANDA* | 531.2283 | Not Found | N/A |
| y5 | AANDA | 461.1991 | 461.1994 | −0.60 |
| y5* | (−1 Da) AANDA* | 460.1912 | Not Found | N/A |
| y4 | ANDA | 390.1619 | 390.1618 | 0.29 |
| y4* | (−1 Da) ANDA* | 389.1541 | Not Found | N/A |
| y3 | NDA | 319.1248 | 319.1252 | −1.17 |
| y3* | (−1 Da) NDA* | 318.1170 | Not Found | N/A |
| y2 | DA | 205.0819 | 205.0821 | −1.00 |
| y2* | (−1 Da) DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

The MS/MS data is consistent with the formation of thioether crosslinks in non-α positions. In mild CID conditions, sactipeptide (sulfur-to-α carbon thioether crosslinked peptides) MS/MS spectra generally produce fragments at each residue location but contain a 2 Da loss at the acceptor (non-Cys) residue (Rea, M. C., et al. Proc. Natl. Acad. Sci. U.S.A. 2010, 107 (20), 9352-9357 and Lohans, C. T. J. Antibiot. 2014, 67, 23-30). Conversely, Cβ- and Cγ-thioether crosslinked peptides do not produce fragments within the macrocycle in mild CID conditions (Hudson, G. A., et al. J. Am. Chem. Soc. 2019, 141, 8228-8238). Previous work by the Mitchell lab calculated the zero-point energy between Cα- and Cβ-thioethers and revealed the Cβ-linkage electronic energy to be 12 kcal/mol more stable than that of the Cα-linkage. This energy difference provides an explanation for the difference seen in MS/MS spectra for these classes of RiPPs. All MS/MS spectra of the msPapA thioether motif expansions and contractions demonstrate a stable macrocycle—i.e., no fragments are found between the Cys and Asp residues.

The reactions with CX₀D and CX₆D peptides did not go to completion; therefore, unmodified peptide fragments are also seen in these reactions revealing cleavage between the C and D residues in the unmodified portion of the isolated envelope, serving as internal controls (FIG. 4D).

b. PapB Tolerates Extensions from the Leader Peptide and Processes in-Line and Nested Crosslinks Independently

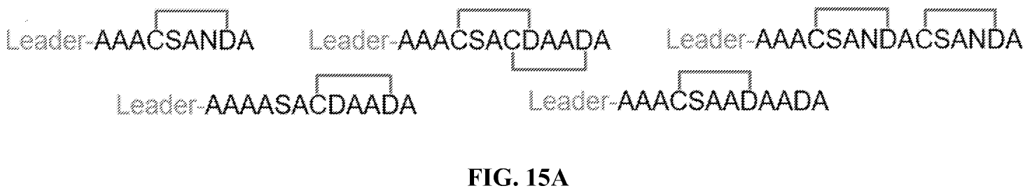

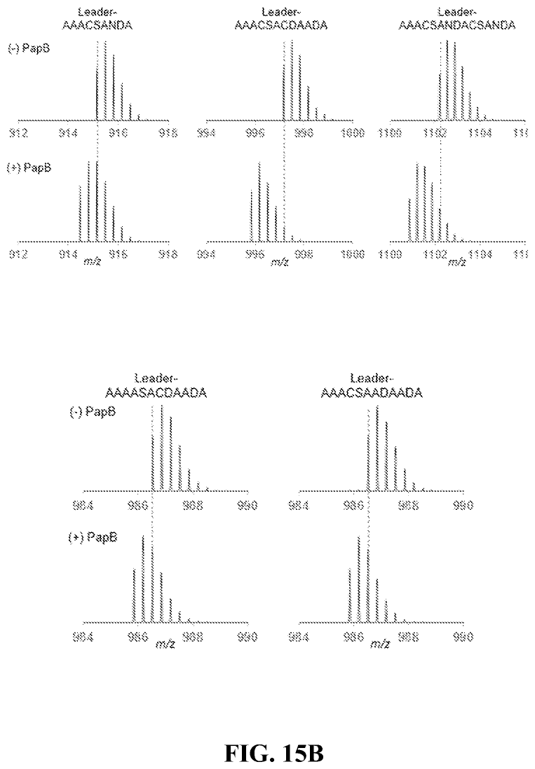

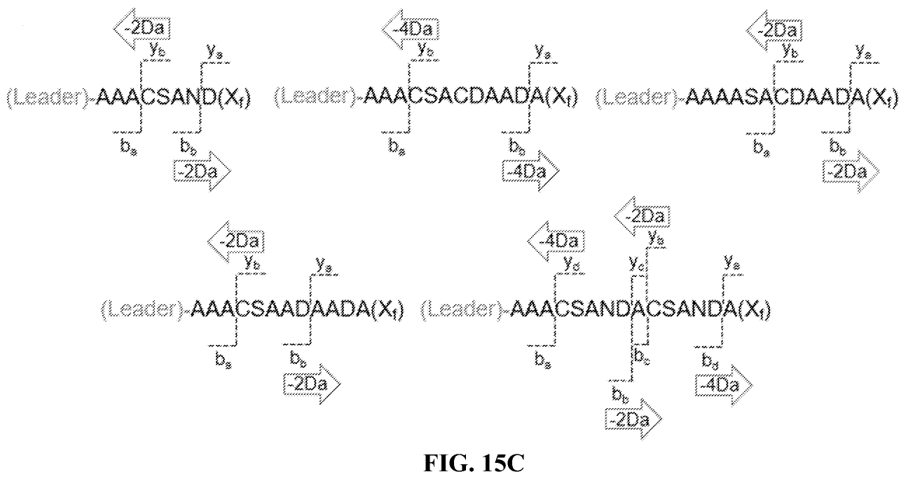

We next explored whether the sequence context of the CX₃D sequence in the natural peptide, and the specific amino acids within the motif are essential for recognition and crosslinking (FIG. 15A). We did not test an exhaustive number of modifications, as the addition of 3 or 4 Ala residues immediately adjacent to the recognition motif clearly did not impair cross linking activity. As FIG. 15B shows, all the peptides that were examined were efficiently crosslinked by the enzyme. FIG. 15C demonstrates that the crosslink occurs within the CX₃D sequence, even if an alternate D residue is available downstream.

The naturally occurring PapA peptide is processed by PapB to introduce six ranthionine linkages, which are either in line with the Cys and Asp residues within a CX₃D motif being crosslinked, or nested with the C residue occurring within one CX₃D motif crosslinking with an Asp residue located C-terminal to it (Precord, T. W., et al. ACS Chem. Biol. 2019, 14 (9), 1981-89). As FIG. 15 shows, both nested and in-line variants of the peptide were able to be crosslinked by simply repositioning the CX₃D element within the peptide. In the case of the in-line and nested crosslinks, the treatment with PapB results in the loss of 4 Da from the peptide (FIG. 15B). The observed monoisotopic masses for these species are <5 ppm of the expected monoisotopic masses (Table 9).

TABLE 9

| | Expected | Observed | |
|---|---|---|---|
| | | Monoiso- | |
| | Monoisotopic | topic | Ppm |
| Sequence | Mass | Mass | Error |
| Leader- | (−) PapB: 915.1568 | 915.1562 | −0.656 |
| AAACSANDA | (+) PapB (−2 Da): | 914.4824 | −2.843 |
| (z = 3) | 914.4850 | | |
| Leader-AAACS | (−) PapB: | 1102.2156 | −2.722 |
| ANDACSANDA | 1102.2186 | 1100.8722 | −2.362 |
| ( z= 3) | (+) PapB (−4 Da): | | |
| | 1100.8748 | | |
| Leader- | (−) PapB: 997.1793 | 997.1774 | −1.905 |
| AAACSACDAADA | (+) PapB (−4 Da): | 995.8310 | −4.519 |
| (z = 3) | 995.8355 | | |
| Leader- | (−) PapB: 986.5220 | 986.5232 | 1.216 |
| AAAASACDAADA | (+) PapB (−2 Da): | 985.8479 | −2.232 |
| (z = 3) | 985.8501 | | |
| Leader- | (−) PapB: 986.5220 | 986.5261 | 4.156 |
| AAACSAADAADA | (+) PapB (−2 Da): | 985.8527 | 2.637 |
| (z = 3) | 985.8501 | | |

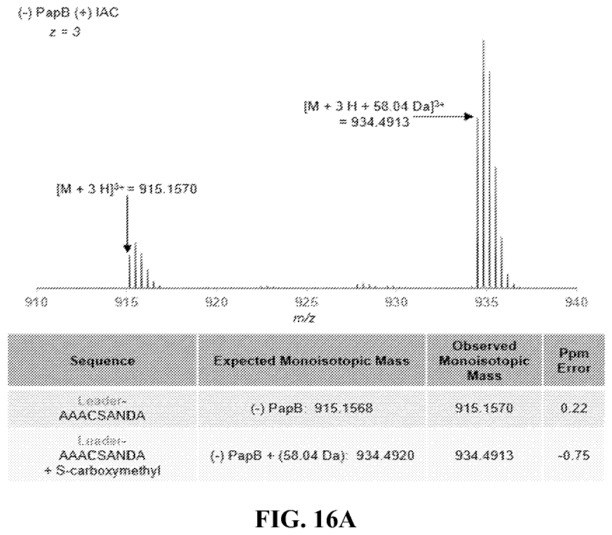

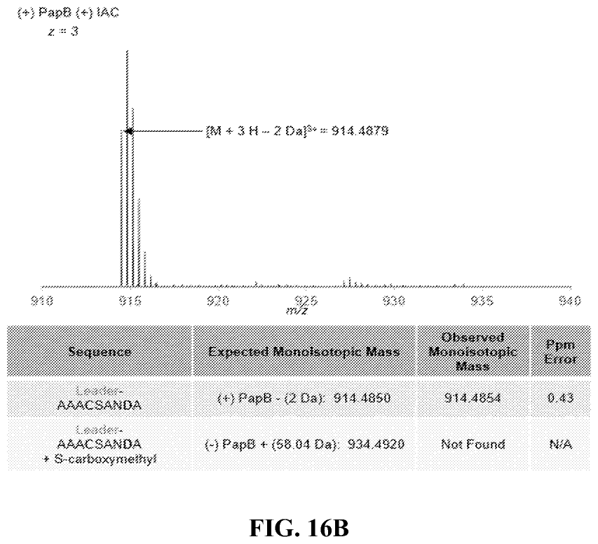

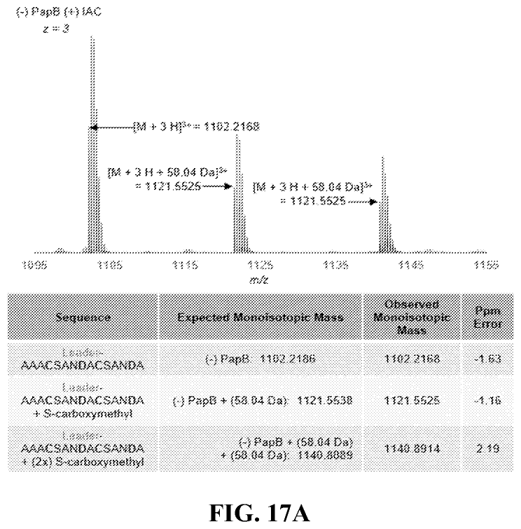

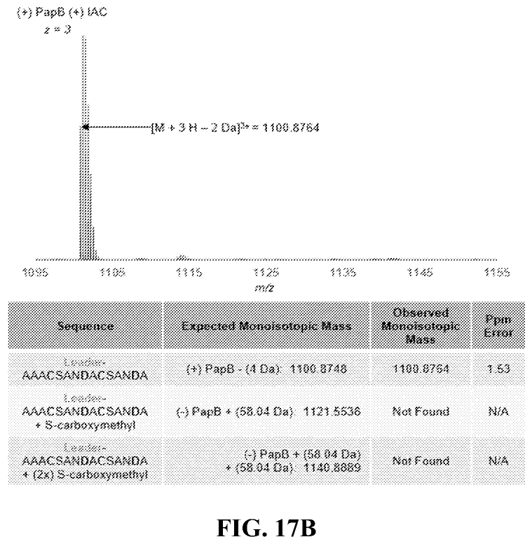

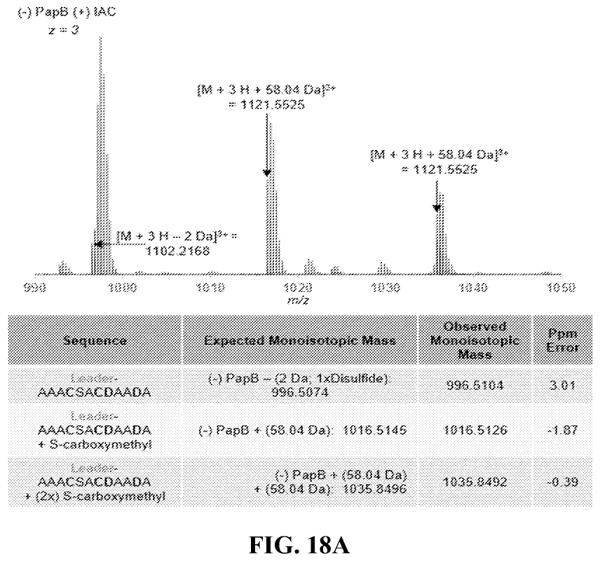

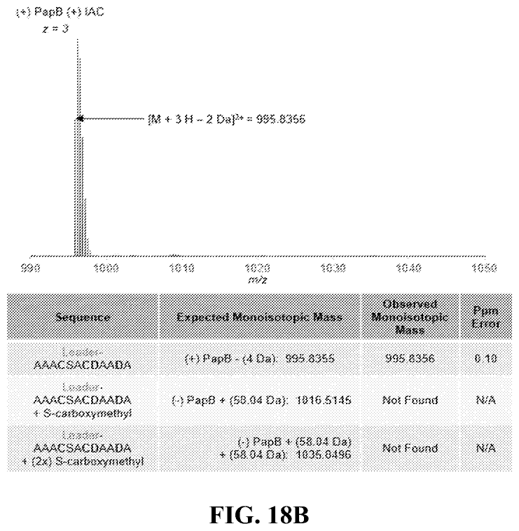

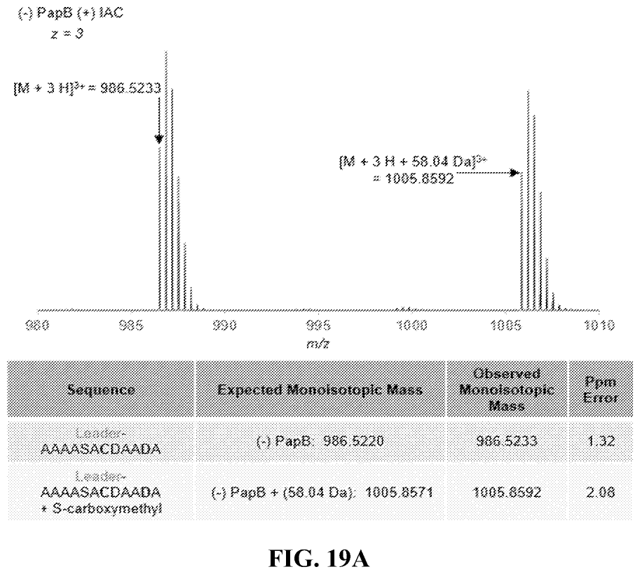

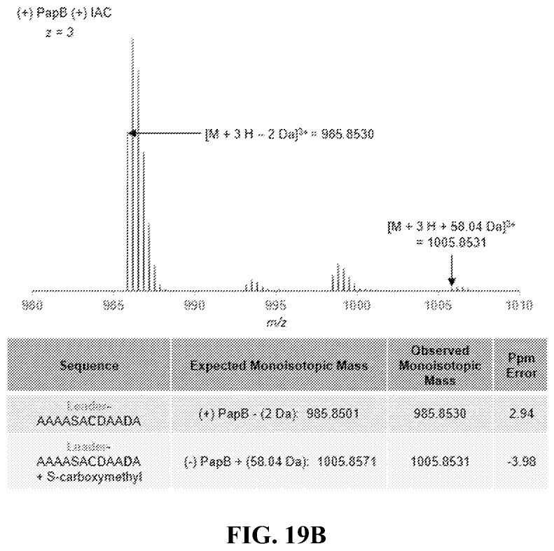

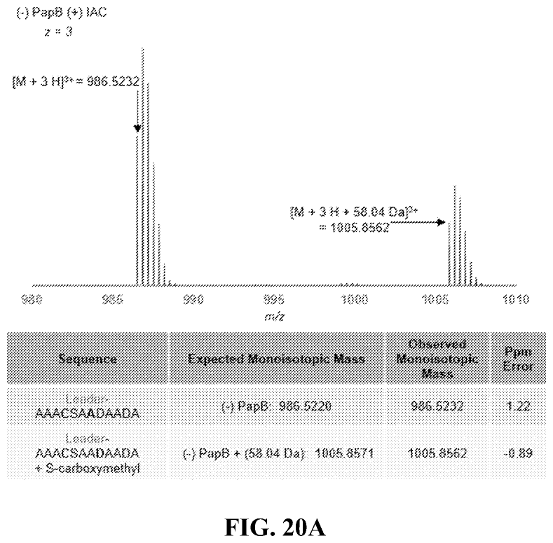

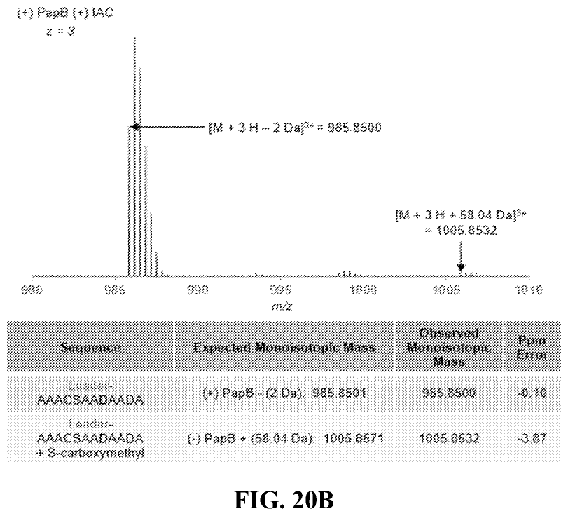

Tandem mass spectrometry reveals a similar pattern in the b and y fragments; a mass loss of 2 Da is seen in each b fragment after D and in each y fragment after C. The fragmentation data for all identifiable peaks are shown in Tables 10-14, a stable macrocycle is seen in each peptide. In all these cases, treatment with IAA resulted in no carboxymethylation of the modified peptide (FIG. 16-20).

TABLE 10
LKQINVIAGVKEPIRAYGAAACSANDA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1863 | −0.01 |
| b2* | LK* | 242.1863 | 242.1859 | 1.64 |
| b3 | LKQ | 370.2449 | 370.2448 | 0.38 |
| b3* | LKQ* | 370.2449 | 370.2441 | 2.20 |
| b4 | LKQI | 483.3289 | 483.3295 | −1.16 |
| b4* | LKQI* | 483.3289 | 483.3285 | 0.79 |
| b5 | LKQIN | 597.3719 | 597.3717 | 0.38 |
| b5* | LKQIN* | 597.3719 | 597.3704 | 2.45 |
| b6 | LKQINV | 696.4403 | 696.4410 | −1.06 |
| b6* | LKQINV* | 696.4403 | 696.4395 | 1.09 |
| b7 | LKQINVI | 809.5244 | 809.5236 | 1.02 |
| b7* | LKQINVI* | 809.5244 | 809.5218 | 3.27 |
| b8 | LKQINVIA | 880.5615 | 880.5588 | 3.12 |
| b8* | LKQINVIA* | 880.5615 | 880.5629 | −1.60 |
| b9 | LKQINVIAG | 937.5829 | 937.5810 | 2.02 |
| b9* | LKQINVIAG* | 937.5829 | 937.5856 | −2.88 |
| b10 | LKQINVIAGV | 1036.6513 | 1036.6480 | 3.19 |
| b10* | LKQINVIAGV* | 1036.6513 | 1036.6534 | −2.00 |
| b11 | LKQINVIAGVK | 1164.7463 | Not Found | N/A |
| b11* | LKQINVIAGVK* | 1164.7463 | 1164.7474 | −0.95 |
| b12 | LKQINVIAGVKE | 1293.7889 | 1293.7937 | −3.73 |
| b12* | LKQINVIAGVKE* | 1293.7889 | 1293.7903 | −1.09 |
| b13 | LKQINVIAGVKEP | 1390.8417 | Not Found | N/A |
| b13* | LKQINVIAGVKEP* | 1390.8417 | 1390.8400 | 1.22 |
| b14 | LKQINVIAGVKEPI | 1503.9257 | 1503.9298 | −2.73 |
| b14* | LKQINVIAGVKEPI* | 1503.9257 | 1503.9242 | 0.99 |
| b15 | LKQINVIAGVKEPIR | 1660.0268 | Not Found | N/A |
| b15* | LKQINVIAGVKEPIR* | 1660.0268 | 1659.9674 | 35.79 |
| b16 | LKQINVIAGVKEPIRA | 1731.0640 | 1731.0659 | −1.10 |
| b16* | LKQINVIAGVKEPIRA* | 1731.0640 | 1731.0593 | 2.74 |
| b17 | LKQINVIAGVKEPIRAY | 947.5673 | Not Found | N/A |
| b17* | LKQINVIAGVKEPIRAY* | 947.5673 | 947.5684 | −1.15 |
| b18 | LKQINVIAGVKEPIRAYG | 976.0780 | 976.0792 | −1.23 |
| b18* | LKQINVIAGVKEPIRAYG* | 976.0780 | 976.0803 | −2.33 |
| b19 | LKQINVIAGVKEPIRAYGA | 1011.5966 | Not Found | N/A |
| b19* | LKQINVIAGVKEPIRAYGA* | 1011.5966 | 1011.5958 | 0.83 |
| b20 | LKQINVIAGVKEPIRAYGAA | 1047.1151 | 1047.1161 | −0.96 |
| b20* | LKQINVIAGVKEPIRAYGAA* | 1047.1151 | 1047.1145 | 0.59 |
| b21 | LKQINVIAGVKEPIRAYGAAA | 1082.6337 | Not Found | N/A |
| b21* | LKQINVIAGVKEPIRAYGAAA* | 1082.6337 | 1082.6344 | −0.69 |
| b22 | LKQINVIAGVKEPIRAYGAAAC | 1134.1383 | 1134.1402 | −1.68 |
| b22* | (−1 Da) LKQINVIAGVKEPIRAYGAAAC* | 1133.6344 | Not Found | N/A |
| b23 | LKQINVIAGVKEPIRAYGAAACS | 1177.6543 | 1177.6577 | −2.89 |
| b23* | (−1 Da) LKQINVIAGVKEPIRAYGAAACS* | 1177.1504 | Not Found | N/A |
| b24 | LKQINVIAGVKEPIRAYGAAACSA | 809.1177 | 809.1198 | −2.60 |
| b24* | (−1 Da) LKQINVIAGVKEPIRAYGAAACSA* | 808.7817 | Not Found | N/A |
| b25 | LKQINVIAGVKEPIRAYGAAACSAN | 847.1320 | 847.1344 | −2.83 |
| b25* | (−1 Da) | 846.7960 | Not Found | N/A |
| | LKQINVIAGVKEPIRAYGAAACSAN* | | | |
| b26 | LKQINVIAGVKEPIRAYGAAACSAND | 885.4743 | Not Found | N/A |
| b26* | (−2 Da) | 884.8024 | 884.8020 | −0.45 |
| | LKQINVIAGVKEPIRAYGAAACSAND* | | | |
| [M] | LKQINVIAGVKEPIRAYGAAACSANDA | 915.1568 | 915.1577 | −0.98 |
| [M]* | (−2 Da) | 914.4850 | 914.4838 | 1.27 |
| | LKQINVIAGVKEPIRAYGAAACSANDA* | | | |
| y26 | KQINVIAGVKEPIRAYGAAACSANDA | 877.4621 | Not Found | N/A |
| y26* | (−2 Da) | 876.7903 | 876.7898 | 0.62 |
| | KQINVIAGVKEPIRAYGAAACSANDA* | | | |
| y25 | QINVIAGVKEPIRAYGAAACSANDA | 834.7638 | 834.7680 | −5.03 |
| y25* | (−2 Da) | 834.0919 | 834.0897 | 2.69 |
| | QINVIAGVKEPIRAYGAAACSANDA* | | | |
| y24 | INVIAGVKEPIRAYGAAACSANDA | 792.0776 | Not Found | N/A |
| y24* | (−2 Da) INVIAGVKEPIRAYGAAACSANDA* | 791.4058 | Not Found | N/A |
| y23 | NVIAGVKEPIRAYGAAACSANDA | 1131.0708 | 1131.0697 | 0.97 |
| y23* | (−2 Da) NVIAGVKEPIRAYGAAACSANDA* | 1130.0630 | 1130.0669 | −3.43 |
| y22 | VIAGVKEPIRAYGAAACSANDA | 1074.0493 | 1074.0493 | 0.00 |
| y22* | (−2 Da) VIAGVKEPIRAYGAAACSANDA* | 1073.0415 | 1073.0450 | −3.25 |
| y21 | IAGVKEPIRAYGAAACSANDA | 1024.5151 | Not Found | N/A |
| y21* | (−2 Da) IAGVKEPIRAYGAAACSANDA* | 1023.5073 | 1023.5087 | −1.35 |
| y20 | AGVKEPIRAYGAAACSANDA | 967.9731 | 967.9767 | −3.72 |
| y20* | (−2 Da) AGVKEPIRAYGAAACSANDA* | 966.9653 | 966.9623 | 3.09 |
| y19 | GVKEPIRAYGAAACSANDA | 932.4545 | 932.4587 | −4.50 |
| y19* | (−2 Da) GVKEPIRAYGAAACSANDA* | 931.4467 | 931.4490 | −2.47 |
| y18 | VKEPIRAYGAAACSANDA | 903.9438 | Not Found | N/A |
| y18* | (−2 Da) VKEPIRAYGAAACSANDA* | 902.9360 | 902.9366 | −0.61 |
| y17 | KEPIRAYGAAACSANDA | 854.4096 | Not Found | N/A |
| y17* | (−2 Da) KEPIRAYGAAACSANDA* | 853.4018 | 853.4033 | −1.77 |
| y16 | EPIRAYGAAACSANDA | 1579.7169 | 1579.7179 | −0.60 |
| y16* | (−2 Da) EPIRAYGAAACSANDA* | 1577.7013 | 1577.7056 | −2.75 |
| y15 | PIRAYGAAACSANDA | 1450.6743 | 1450.6682 | 4.20 |
| y15* | (−2 Da) PIRAYGAAACSANDA* | 1448.6587 | 1448.6620 | −2.27 |
| y14 | IRAYGAAACSANDA | 1353.6216 | 1353.6254 | −2.81 |
| y14* | (−2 Da) IRAYGAAACSANDA* | 1351.6059 | 1351.6104 | −3.30 |
| y13 | RAYGAAACSANDA | 1240.5375 | Not Found | N/A |
| y13* | (−2 Da) RAYGAAACSANDA* | 1238.5219 | 1238.5169 | 4.07 |
| y12 | AYGAAACSANDA | 1084.4364 | 1084.4388 | −2.21 |
| y12* | (−2 Da) AYGAAACSANDA* | 1082.4207 | 1082.4233 | −2.43 |
| y11 | YGAAACSANDA | 1013.3993 | 1013.4001 | −0.79 |
| y11* | (−2 Da) YGAAACSANDA* | 1011.3836 | 1011.3822 | 1.39 |
| y10 | GAAACSANDA | 850.3360 | 850.3392 | −3.76 |
| y10* | (−2 Da) GAAACSANDA* | 848.3203 | 848.3191 | 1.37 |
| y9 | AAACSANDA | 793.3145 | Not Found | N/A |
| y9* | (−2 Da) AAACSANDA* | 791.2988 | 791.3009 | −2.71 |
| y8 | AACSANDA | 722.2774 | 722.2773 | 0.08 |
| y8* | (−2 Da) AACSANDA* | 720.2617 | 720.2596 | 2.93 |
| y7 | ACSANDA | 651.2403 | 651.2393 | 1.60 |
| y7* | (−2 Da) ACSANDA* | 649.2246 | 649.2251 | −0.80 |
| y6 | CSANDA | 580.2032 | 580.2036 | −0.69 |
| y6* | (−2 Da) CSANDA* | 578.1875 | 578.1864 | 1.94 |
| y5 | SANDA | 477.1940 | 477.1935 | 1.09 |
| y5* | (−1 Da) SANDA* | 476.1861 | Not Found | N/A |
| y4 | ANDA | 390.1619 | 390.1614 | 1.20 |
| y4* | (−1 Da) ANDA* | 389.1541 | Not Found | N/A |
| y3 | NDA | 319.1248 | 319.1250 | −0.62 |
| y3* | (−1 Da) NDA* | 318.1170 | Not Found | N/A |
| y2 | DA | 205.0819 | 205.0821 | −1.13 |
| y2* | (−1 Da) DA* | 204.0741 | Not Found | N/A |

Unmodified indicates z = 1 charge state.
Underline indicates z = 2 charge state.
*Indicates the fragment originated from the modified peptide

TABLE 11
LKQINVIAGVKEPIRAYGAAACSANDACSANDA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1860 | 1.42 |
| b2* | LK* | 242.1863 | 242.1860 | 1.45 |
| b3 | LKQ | 370.2449 | 370.2443 | 1.62 |
| b3* | LKQ* | 370.2449 | 370.2441 | 2.14 |
| b4 | LKQI | 483.3289 | 483.3289 | −0.06 |
| b4* | LKQI* | 483.3289 | 483.3285 | 0.82 |
| b5 | LKQIN | 597.3719 | 597.3711 | 1.35 |
| b5* | LKQIN* | 597.3719 | 597.3704 | 2.57 |
| b6 | LKQINV | 696.4403 | 696.4404 | −0.20 |
| b6* | LKQINV* | 696.4403 | 696.4394 | 1.29 |
| b7 | LKQINVI | 809.5244 | 809.5230 | 1.77 |
| b7* | LKQINVI* | 809.5244 | 809.5270 | −3.21 |
| b8 | LKQINVIA | 880.5615 | 880.5643 | −3.22 |
| b8* | LKQINVIA* | 880.5615 | 880.5626 | −1.29 |
| b9 | LKQINVIAG | 937.5829 | 937.5804 | 2.66 |
| b9* | LKQINVIAG* | 937.5829 | 937.5853 | −2.53 |
| b10 | LKQINVIAGV | 1036.6513 | 1036.6474 | 3.75 |
| b10* | LKQINVIAGV* | 1036.6513 | 1036.6530 | −1.61 |
| b11 | LKQINVIAGVK | 1164.7463 | 1164.7498 | −3.03 |
| b11* | LKQINVIAGVK* | 1164.7463 | 1164.7469 | −0.49 |
| b12 | LKQINVIAGVKE | 1293.7889 | 1293.7933 | −3.36 |
| b12* | LKQINVIAGVKE* | 1293.7889 | 1293.7896 | −0.56 |
| b13 | LKQINVIAGVKEP | 1390.8417 | 1390.8433 | −1.19 |
| b13* | LKQINVIAGVKEP* | 1390.8417 | 1390.8146 | 19.48 |
| b14 | LKQINVIAGVKEPI | 1503.9257 | 1503.9281 | −1.57 |
| b14* | LKQINVIAGVKEPI* | 1503.9257 | 1503.9233 | 1.62 |
| b15 | LKQINVIAGVKEPIR | 1660.0268 | 1660.0201 | 4.04 |
| b15* | LKQINVIAGVKEPIR* | 1660.0268 | 1660.0304 | −2.15 |
| b16 | LKQINVIAGVKEPIRA | 1731.0640 | 1731.0642 | −0.12 |
| b16* | LKQINVIAGVKEPIRA* | 1731.0640 | 1731.0580 | 3.46 |
| b17 | LKQINVIAGVKEPIRAY | 947.5673 | 947.5700 | −2.88 |
| b17* | LKQINVIAGVKEPIRAY* | 947.5673 | 947.5681 | −0.80 |
| b18 | LKQINVIAGVKEPIRAYG | 976.0780 | 976.0748 | 3.30 |
| b18* | LKQINVIAGVKEPIRAYG* | 976.0780 | 976.0799 | −1.96 |
| b19 | LKQINVIAGVKEPIRAYGA | 1011.5966 | 1011.5976 | −1.01 |
| b19* | LKQINVIAGVKEPIRAYGA* | 1011.5966 | 1011.5954 | 1.22 |
| b20 | LKQINVIAGVKEPIRAYGAA | 1047.1151 | 1047.1165 | −1.31 |
| b20* | LKQINVIAGVKEPIRAYGAA* | 1047.1151 | 1047.1141 | 0.99 |
| b21 | LKQINVIAGVKEPIRAYGAAA | 1082.6337 | 1082.6366 | −2.64 |
| b21* | LKQINVIAGVKEPIRAYGAAA* | 1082.6337 | 1082.6340 | −0.27 |
| b22 | LKQINVIAGVKEPIRAYGAAAC | 1134.1383 | 1134.1423 | −3.53 |
| b22* | (−1 Da)LKQINVIAGVKEPIRAYGAAAC* | 1133.6344 | Not Found | N/A |
| b23 | LKQINVIAGVKEPIRAYGAAACS | 1177.6543 | 1177.6549 | −0.55 |
| b23* | (−1 Da) | 1177.1504 | Not Found | N/A |
| | LKQINVIAGVKEPIRAYGAAACS* | | | |
| b24 | LKQINVIAGVKEPIRAYGAAACSA | 1213.1728 | 1213.1729 | −0.07 |
| b24* | (−1 Da) | 1212.6689 | Not Found | N/A |
| | LKQINVIAGVKEPIRAYGAAACSA* | | | |
| b25 | LKQINVIAGVKEPIRAYGAAACSAN | 847.1320 | 847.1325 | −0.60 |
| b25* | (−1 Da) | 846.7960 | Not Found | N/A |
| | LKQINVIAGVKEPIRAYGAAACSAN* | | | |
| b26 | LKQINVIAGVKEPIRAYGAAACSAND | 885.4743 | 885.4729 | 1.62 |
| b26* | (−2 Da) | 884.8024 | 884.8031 | −0.77 |
| | LKQINVIAGVKEPIRAYGAAACSAND* | | | |
| b27 | LKQINVIAGVKEPIRAYGAAACSANDA | 909.1533 | 909.1520 | 1.45 |
| b27* | (−2 Da) | 908.4814 | 908.4811 | 0.34 |
| | LKQINVIAGVKEPIRAYGAAACSANDA* | | | |
| b28 | LKQINVIAGVKEPIRAYGAAACSANDA | 943.4897 | 943.4891 | 0.59 |
| | C | | | |
| b28* | (−3 Da) | 942.4819 | Not Found | N/A |
| | LKQINVIAGVKEPIRAYGAAACSANDA | | | |
| | C* | | | |
| b29 | LKQINVIAGVKEPIRAYGAAACSANDA | 972.5004 | 972.4985 | 1.96 |
| | CS | | | |
| b29* | LKQINVIAGVKEPIRAYGAAACSANDA | 971.4926 | Not Found | N/A |
| | CS* | | | |
| | (−3 Da) | | | |
| b30 | LKQINVIAGVKEPIRAYGAAACSANDA | 996.1794 | Not Found | N/A |
| | CSA | | | |
| b30* | LKQINVIAGVKEPIRAYGAAACSANDA | 995.1716 | Not Found | N/A |
| | CSA* | | | |
| | (−3 Da) | | | |
| b31 | LKQINVIAGVKEPIRAYGAAACSANDA | 1034.1937 | Not Found | N/A |
| | CSAN | | | |
| b31* | LKQINVIAGVKEPIRAYGAAACSANDA | 1033.1859 | Not Found | N/A |
| | CSAN* | | | |
| | (−3 Da) | | | |
| b32 | LKQINVIAGVKEPIRAYGAAACSANDA | 1072.5360 | 1072.5389 | −2.69 |
| | CSAND | | | |
| b32* | LKQINVIAGVKEPIRAYGAAACSANDA | 1071.1923 | 1071.1885 | 3.55 |
| | CSAND* | | | |
| | (−4 Da) | | | |
| [M] | LKQINVIAGVKEPIRAYGAAACSANDA | 1102.2186 | 1102.2185 | −0.09 |
| | CSANDA | | | |
| [M]* | LKQINVIAGVKEPIRAYGAAACSANDA | 1100.8748 | 1100.8764 | −1.42 |
| | CSANDA* | | | |
| | (−4 Da) | | | |
| y32 | KQINVIAGVKEPIRAYGAAACSANDACS | 1064.5239 | 1064.5226 | 1.24 |
| | ANDA | | | |
| y32* | KQINVIAGVKEPIRAYGAAACSANDAC | 1063.1802 | 1063.1791 | 1.04 |
| | SANDA* | | | |
| | (−4 Da) | | | |
| y31 | QINVIAGVKEPIRAYGAAACSANDACSA | 1021.8256 | Not Found | N/A |
| | NDA | | | |
| y31* | QINVIAGVKEPIRAYGAAACSANDACS | 1020.4818 | 1020.4795 | 2.27 |
| | ANDA* | | | |
| | (−4 Da) | | | |
| y30 | INVIAGVKEPIRAYGAAACSANDACSAN | 979.1394 | Not Found | N/A |
| | DA | | | |
| y30* | INVIAGVKEPIRAYGAAACSANDACSA | 977.7956 | Not Found | N/A |
| | NDA* | | | |
| | (−4 Da) | | | |
| y29 | NVIAGVKEPIRAYGAAACSANDACSAN | 941.4447 | 941.4449 | −0.19 |
| | DA | | | |
| y29* | NVIAGVKEPIRAYGAAACSANDACSAN | 940.1010 | 940.1020 | −1.07 |
| | DA* | | | |
| | (−4 Da) | | | |
| y28 | VIAGVKEPIRAYGAAACSANDACSAND | 903.4304 | 903.4336 | −3.53 |
| | A | | | |
| y28* | VIAGVKEPIRAYGAAACSANDACSAND | 902.0866 | 902.0877 | −1.24 |
| | A* | | | |
| | (−4 Da) | | | |
| y27 | IAGVKEPIRAYGAAACSANDACSANDA | 870.4076 | 870.4060 | 1.79 |
| y27* | (−4 Da) | 869.0638 | Not Found | N/A |
| | IAGVKEPIRAYGAAACSANDACSANDA | | | |
| | * | | | |
| y26 | AGVKEPIRAYGAAACSANDACSANDA | 832.7129 | Not Found | N/A |
| y26* | (−4 Da) | 831.3691 | Not Found | N/A |
| | AGVKEPIRAYGAAACSANDACSANDA* | | | |
| y25 | GVKEPIRAYGAAACSANDACSANDA | 809.0339 | 809.0317 | 2.71 |
| y25* | (−4 Da) | 807.6901 | Not Found | N/A |
| | GVKEPIRAYGAAACSANDACSANDA* | | | |
| y24 | VKEPIRAYGAAACSANDACSANDA | 1184.5364 | Not Found | N/A |
| y24* | (−4 Da) | 1182.5208 | Not Found | N/A |
| | VKEPIRAYGAAACSANDACSANDA* | | | |
| y23 | KEPIRAYGAAACSANDACSANDA | 1135.0022 | 1135.0031 | −0.78 |
| y23* | (−4 Da) | 1132.9866 | 1132.9903 | −3.25 |
| | KEPIRAYGAAACSANDACSANDA* | | | |
| y22 | EPIRAYGAAACSANDACSANDA | 1070.9548 | Not Found | N/A |
| y22* | (−4 Da) | 1068.9391 | 1068.9394 | −0.30 |
| | EPIRAYGAAACSANDACSANDA* | | | |
| y21 | PIRAYGAAACSANDACSANDA | 1006.4335 | 1006.4302 | 3.29 |
| y21* | (−4 Da)PIRAYGAAACSANDACSANDA* | 1004.4178 | 1004.4174 | 0.44 |
| y20 | IRAYGAAACSANDACSANDA | 957.9071 | Not Found | N/A |
| y20* | (−4 Da)IRAYGAAACSANDACSANDA* | 955.8914 | Not Found | N/A |
| y19 | RAYGAAACSANDACSANDA | 901.3650 | Not Found | N/A |
| y19* | (−4 Da)RAYGAAACSANDACSANDA* | 899.3494 | 899.3510 | −1.76 |
| y18 | AYGAAACSANDACSANDA | 823.3145 | 823.3163 | −2.16 |
| y18* | (−4 Da)AYGAAACSANDACSANDA* | 821.2988 | 821.2910 | 9.55 |
| y17 | YGAAACSANDACSANDA | 787.7959 | Not Found | N/A |
| y17* | (−4 Da)YGAAACSANDACSANDA* | 785.7803 | 785.7778 | 3.14 |
| y16 | GAAACSANDACSANDA | 1411.5213 | 1411.5230 | −1.18 |
| y16* | (−4 Da)GAAACSANDACSANDA* | 1407.4900 | 1407.4913 | −0.91 |
| y15 | AAACSANDACSANDA | 1354.4998 | Not Found | N/A |
| y15* | (−4 Da)AAACSANDACSANDA* | 1350.4685 | 1350.4675 | 0.72 |
| y14 | AACSANDACSANDA | 1283.4627 | 1283.4618 | 0.68 |
| y14* | (−4 Da)AACSANDACSANDA* | 1279.4314 | 1279.4341 | −2.12 |
| y13 | ACSANDACSANDA | 1212.4256 | 1212.4218 | 3.10 |
| y13* | (−4 Da)ACSANDACSANDA* | 1208.3943 | 1208.3950 | −0.55 |
| y12 | CSANDACSANDA | 1141.3885 | 1141.3852 | 2.92 |
| y12* | (−4 Da)CSANDACSANDA* | 1137.3572 | 1137.3611 | −3.43 |
| y11 | SANDACSANDA | 1038.3793 | 1038.3826 | −3.22 |
| y11* | (−3 Da)SANDACSANDA* | 1035.3558 | Not Found | N/A |
| y10 | ANDACSANDA | 951.3473 | 951.3504 | −3.24 |
| y10* | (−3 Da)ANDACSANDA* | 948.3238 | Not Found | N/A |
| y9 | NDACSANDA | 880.3101 | 880.3104 | −0.32 |
| y9* | (−3 Da)NDACSANDA* | 877.2867 | Not Found | N/A |
| y8 | DACSANDA | 766.2672 | 766.2684 | −1.61 |
| y8* | (−3 Da)DACSANDA* | 763.2437 | Not Found | N/A |
| y7 | ACSANDA | 651.2403 | 651.2387 | 2.52 |
| y7* | (−2 Da)ACSANDA* | 649.2246 | 649.2250 | −0.64 |
| y6 | CSANDA | 580.2032 | 580.2030 | 0.39 |
| y6* | (−2 Da)CSANDA* | 578.1875 | Not Found | N/A |
| y5 | SANDA | 477.1940 | 477.1930 | 2.20 |
| y5* | (−1 Da)SANDA* | 476.1861 | Not Found | N/A |
| y4 | ANDA | 390.1619 | 390.1628 | −2.27 |
| y4* | (−1 Da)ANDA* | 389.1541 | Not Found | N/A |
| y3 | NDA | 319.1248 | 319.1246 | 0.70 |
| y3* | (−1 Da)NDA* | 318.1170 | Not Found | N/A |
| y2 | DA | 205.0819 | 205.0818 | 0.37 |
| y2* | (−1 Da)DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 12
LKQINVIAGVKEPIRAYGAAACSACDAADA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1859 | 1.58 |
| b2* | LK* | 242.1863 | 242.1860 | 1.19 |
| b3 | LKQ | 370.2449 | 370.2452 | −0.82 |
| b3* | LKQ* | 370.2449 | 370.2455 | −1.52 |
| b4 | LKQI | 483.3289 | 483.3297 | −1.69 |
| b4* | LKQI* | 483.3289 | 483.3277 | 2.59 |
| b5 | LKQIN | 597.3719 | 597.3716 | 0.44 |
| b5* | LKQIN* | 597.3719 | 597.3723 | −0.70 |
| b6 | LKQINV | 696.4403 | 696.4407 | −0.54 |
| b6* | LKQINV* | 696.4403 | 696.4416 | −1.85 |
| b7 | LKQINVI | 809.5244 | 809.5228 | 2.03 |
| b7* | LKQINVI* | 809.5244 | 809.5240 | 0.55 |
| b8 | LKQINVIA | 880.5615 | 880.5638 | −2.61 |
| b8* | LKQINVIA* | 880.5615 | 880.5590 | 2.84 |
| b9 | LKQINVIAG | 937.5829 | 937.5796 | 3.55 |
| b9* | LKQINVIAG* | 937.5829 | 937.5811 | 1.89 |
| b10 | LKQINVIAGV | 1036.6513 | 1036.6539 | −2.54 |
| b10* | LKQINVIAGV* | 1036.6513 | 1036.6479 | 3.29 |
| b11 | LKQINVIAGVK | 1164.7463 | 1164.7476 | −1.14 |
| b11* | LKQINVIAGVK* | 1164.7463 | 1164.7499 | −3.10 |
| b12 | LKQINVIAGVKE | 1293.7889 | 1293.7901 | −0.94 |
| b12* | LKQINVIAGVKE* | 1293.7889 | 1293.7929 | −3.06 |
| b13 | LKQINVIAGVKEP | 1390.8417 | 1390.8395 | 1.62 |
| b13* | LKQINVIAGVKEP* | 1390.8417 | Not Found | N/A |
| b14 | LKQINVIAGVKEPI | 1503.9257 | 1503.9232 | 1.66 |
| b14* | LKQINVIAGVKEPI* | 1503.9257 | 1503.9268 | −0.70 |
| b15 | LKQINVIAGVKEPIR | 1660.0268 | 1660.0298 | −1.84 |
| b15* | LKQINVIAGVKEPIR* | 1660.0268 | Not Found | N/A |
| b16 | LKQINVIAGVKEPIRA | 1731.0640 | 1731.0572 | 3.90 |
| b16* | LKQINVIAGVKEPIRA* | 1731.0640 | Not Found | N/A |
| b17 | LKQINVIAGVKEPIRAY | 947.5673 | 947.5691 | −1.95 |
| b17* | LKQINVIAGVKEPIRAY* | 947.5673 | 947.5707 | −3.63 |
| b18 | LKQINVIAGVKEPIRAYG | 976.0780 | 976.0810 | −3.04 |
| b18* | LKQINVIAGVKEPIRAYG* | 976.0780 | 976.0754 | 2.65 |
| b19 | LKQINVIAGVKEPIRAYGA | 1011.5966 | 1011.5964 | 0.22 |
| b19* | LKQINVIAGVKEPIRAYGA* | 1011.5966 | 1011.5982 | −1.55 |
| b20 | LKQINVIAGVKEPIRAYGAA | 1047.1151 | 1047.1150 | 0.08 |
| b20* | LKQINVIAGVKEPIRAYGAA* | 1047.1151 | 1047.1169 | −1.73 |
| b2 | LKQINVIAGVKEPIRAYGAAA | 1082.6337 | 1082.6349 | −1.10 |
| b21* | LKQINVIAGVKEPIRAYGAAA* | 1082.6337 | 1082.6369 | −2.96 |
| b22 | LKQINVIAGVKEPIRAYGAAAC | 1134.1383 | 1134.1403 | −1.76 |
| b22* | (−1 Da)LKQINVIAGVKEPIRAYGAAAC* | 1133.6344 | Not Found | N/A |
| b23 | LKQINVIAGVKEPIRAYGAAACS | 1177.6543 | 1177.6526 | 1.41 |
| b23* | (−1 Da)LKQINVIAGVKEPIRAYGAAACS* | 1177.1504 | Not Found | N/A |
| b24 | LKQINVIAGVKEPIRAYGAAACSA | 1213.1728 | 1213.1703 | 2.03 |
| b24* | (−1 Da)LKQINVIAGVKEPIRAYGAAACSA* | 1212.6689 | Not Found | N/A |
| b25 | LKQINVIAGVKEPIRAYGAAACSAC | 843.4540 | 843.4554 | −1.68 |
| b25* | (−2 Da)LKQINVIAGVKEPIRAYGAAACSAC* | 842.7822 | Not Found | N/A |
| b26 | LKQINVIAGVKEPIRAYGAAACSACD | 881.7964 | 881.7980 | −1.76 |
| b26* | LKQINVIAGVKEPIRAYGAAACSACD* | 880.7885 | Not Found | N/A |
| | (−3 Da) | | | |
| b27 | LKQINVIAGVKEPIRAYGAAACSACDA | 905.4754 | 905.4773 | −2.10 |
| b27* | LKQINVIAGVKEPIRAYGAAACSACDA* | 904.4676 | Not Found | N/A |
| | (−3 Da) | | | |
| b28 | LKQINVIAGVKEPIRAYGAAACSACDAA | 929.1544 | 929.1557 | −1.40 |
| b28* | LKQINVIAGVKEPIRAYGAAACSACDAA* | 928.1466 | Not Found | N/A |
| | (−3 Da) | | | |
| b29 | LKQINVIAGVKEPIRAYGAAACSACDAAD | 967.4968 | 967.4979 | −1.17 |
| b29* | LKQINVIAGVKEPIRAYGAAACSACDAAD* | 966.1530 | 966.1525 | 0.55 |
| | (−4 Da) | | | |
| [M] | LKQINVIAGVKEPIRAYGAAACSACDAADA | 997.1793 | 997.1821 | −2.82 |
| [M]* | LKQINVIAGVKEPIRAYGAAACSACDAADA* | 995.8355 | 995.8339 | 1.61 |
| | (−4 Da) | | | |
| y29 | KQINVIAGVKEPIRAYGAAACSACDAADA | 959.4846 | 959.4855 | −0.92 |
| y29* | KQINVIAGVKEPIRAYGAAACSACDAADA* | 958.1409 | 958.1426 | −1.80 |
| | (−4 Da) | | | |
| y28 | QINVIAGVKEPIRAYGAAACSACDAADA | 916.7863 | 916.7838 | 2.69 |
| y28* | QINVIAGVKEPIRAYGAAACSACDAADA* | 915.4425 | 915.4442 | −1.85 |
| | (−4 Da) | | | |
| y27 | INVIAGVKEPIRAYGAAACSACDAADA | 874.1001 | Not Found | N/A |
| y27* | INVIAGVKEPIRAYGAAACSACDAADA* | 872.7563 | 872.7557 | 0.70 |
| | (−4 Da) | | | |
| y26 | NVIAGVKEPIRAYGAAACSACDAADA | 836.4054 | 836.4028 | 3.10 |
| y26* | NVIAGVKEPIRAYGAAACSACDAADA* | 835.0617 | 835.0637 | −2.36 |
| | (−4 Da) | | | |
| y25 | VIAGVKEPIRAYGAAACSACDAADA | 798.3911 | 798.3895 | 2.01 |
| y25* | VIAGVKEPIRAYGAAACSACDAADA* | 797.0473 | 797.0499 | −3.20 |
| | (−4 Da) | | | |
| y24 | IAGVKEPIRAYGAAACSACDAADA | 765.3683 | 765.3690 | −0.90 |
| y24* | (−4 Da)IAGVKEPIRAYGAAACSACDAADA* | 764.0245 | Not Found | N/A |
| y23 | AGVKEPIRAYGAAACSACDAADA | 1091.0068 | 1091.0108 | −3.65 |
| y23* | (−4 Da)AGVKEPIRAYGAAACSACDAADA* | 1088.9911 | 1088.9907 | 0.37 |
| y22 | GVKEPIRAYGAAACSACDAADA | 1055.4882 | Not Found | N/A |
| y22* | (−4 Da)GVKEPIRAYGAAACSACDAADA* | 1053.4726 | 1053.4763 | −3.47 |
| y21 | VKEPIRAYGAAACSACDAADA | 1026.9775 | Not Found | N/A |
| y21* | (−4 Da)VKEPIRAYGAAACSACDAADA* | 1024.9619 | 1024.9644 | −2.46 |
| y20 | KEPIRAYGAAACSACDAADA | 977.4433 | 977.4417 | 1.67 |
| y20* | (−4 Da)KEPIRAYGAAACSACDAADA* | 975.4276 | 975.4250 | 2.63 |
| y19 | EPIRAYGAAACSACDAADA | 913.3958 | Not Found | N/A |
| y19* | (−4 Da)EPIRAYGAAACSACDAADA* | 911.3802 | 911.3797 | 0.59 |
| y18 | PIRAYGAAACSACDAADA | 848.8745 | 848.8769 | −2.80 |
| y18* | (−4 Da)PIRAYGAAACSACDAADA* | 846.8589 | 846.8587 | 0.19 |
| y17 | IRAYGAAACSACDAADA | 800.3481 | Not Found | N/A |
| y17* | (−4 Da)IRAYGAAACSACDAADA* | 798.3325 | Not Found | N/A |
| y16 | RAYGAAACSACDAADA | 1486.6049 | 1486.6094 | −3.01 |
| y16* | (−4 Da)RAYGAAACSACDAADA* | 1482.5736 | 1482.5713 | 1.55 |
| y15 | AYGAAACSACDAADA | 1330.5038 | Not Found | N/A |
| y15* | (−4 Da)AYGAAACSACDAADA* | 1326.4725 | 1326.4740 | −1.13 |
| y14 | YGAAACSACDAADA | 1259.4667 | Not Found | N/A |
| y14* | (−4 Da)YGAAACSACDAADA* | 1255.4354 | 1255.4320 | 2.72 |
| y13 | GAAACSACDAADA | 1096.4034 | 1096.4049 | −1.38 |
| y13* | (−4 Da)GAAACSACDAADA* | 1092.3721 | 1092.3726 | −0.46 |
| y12 | AAACSACDAADA | 1039.3819 | Not Found | N/A |
| y12* | (−4 Da)AAACSACDAADA* | 1035.3506 | 1035.3513 | −0.67 |
| y11 | AACSACDAADA | 968.3448 | Not Found | N/A |
| y11* | (−4 Da)AACSACDAADA* | 964.3135 | 964.3110 | 2.60 |
| y10 | ACSACDAADA | 897.3077 | 897.3093 | −1.82 |
| y10* | (−4 Da)ACSACDAADA* | 893.2764 | 893.2777 | −1.41 |
| y9 | CSACDAADA | 826.2706 | 826.2701 | 0.63 |
| y9* | (−4 Da)CSACDAADA* | 822.2393 | 822.2373 | 2.43 |
| y8 | SACDAADA | 723.2614 | 723.2628 | −1.91 |
| y8* | (−3 Da)SACDAADA* | 720.2379 | Not Found | N/A |
| y7 | ACDAADA | 636.2294 | 636.2309 | −2.38 |
| y7* | (−3 Da)ACDAADA* | 633.2059 | Not Found | N/A |
| y6 | CDAADA | 565.1923 | 565.1928 | −0.87 |
| y6* | (−3 Da)CDAADA* | 562.1688 | Not Found | N/A |
| y5 | DAADA | 462.1831 | 462.1823 | 1.82 |
| y5* | (−2 Da)DAADA* | 460.1674 | Not Found | N/A |
| y4 | AADA | 347.1561 | 347.1562 | −0.21 |
| y4* | (−1 Da)AADA* | 346.1483 | Not Found | N/A |
| y3 | ADA | 276.1190 | 276.1186 | 1.50 |
| y3* | (−1 Da)ADA* | 275.1112 | Not Found | N/A |
| y2 | DA | 205.0819 | 205.0819 | −0.14 |
| y2* | (−1 Da)DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 13
LKQINVIAGVKEPIRAYGAAAASACDAADA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1861 | 0.69 |
| b2* | LK* | 242.1863 | 242.1862 | 0.54 |
| b3 | LKQ | 370.2449 | 370.2444 | 1.30 |
| b3* | LKQ* | 370.2449 | 370.2446 | 0.91 |
| b4 | LKQI | 483.3289 | 483.3289 | −0.09 |
| b4* | LKQI* | 483.3289 | 483.3292 | −0.65 |
| b5 | LKQIN | 597.3719 | 597.3709 | 1.59 |
| b5* | LKQIN* | 597.3719 | 597.3714 | 0.88 |
| b6 | LKQINV | 696.4403 | 696.4401 | 0.26 |
| b6* | LKQINV* | 696.4403 | 696.4407 | −0.57 |
| b7 | LKQINVI | 809.5244 | 809.5224 | 2.45 |
| b7* | LKQINVI* | 809.5244 | 809.5232 | 1.50 |
| b8 | LKQINVIA | 880.5615 | 880.5636 | −2.41 |
| b8* | LKQINVIA* | 880.5615 | 880.5645 | −3.44 |
| b9 | LKQINVIAG | 937.5829 | 937.5795 | 3.58 |
| b9* | LKQINVIAG* | 937.5829 | 937.5806 | 2.50 |
| b10 | LKQINVIAGV | 1036.6513 | 1036.6542 | −2.79 |
| b10* | LKQINVIAGV* | 1036.6513 | 1036.6475 | 3.66 |
| b11 | LKQINVIAGVK | 1164.7463 | 1164.7483 | −1.73 |
| b11* | LKQINVIAGVK* | 1164.7463 | 1164.7498 | −3.03 |
| b12 | LKQINVIAGVKE | 1293.7889 | 1293.7913 | −1.86 |
| b12* | LKQINVIAGVKE* | 1293.7889 | 1293.7931 | −3.26 |
| b13 | LKQINVIAGVKEP | 1390.8417 | Not Found | N/A |
| b13* | LKQINVIAGVKEP* | 1390.8417 | Not Found | N/A |
| b14 | LKQINVIAGVKEPI | 1503.9257 | 1503.9253 | 0.24 |
| b14* | LKQINVIAGVKEPI* | 1503.9257 | 1503.9277 | −1.33 |
| b15 | LKQINVIAGVKEPIR | 1660.0268 | 1660.0328 | −3.59 |
| b15* | LKQINVIAGVKEPIR* | 1660.0268 | 1660.0195 | 4.39 |
| b16 | LKQINVIAGVKEPIRA | 866.0356 | 866.0382 | −3.00 |
| b16* | LKQINVIAGVKEPIRA* | 866.0356 | 866.0330 | 2.96 |
| b17 | LKQINVIAGVKEPIRAY | 947.5673 | 947.5691 | −1.95 |
| b17* | LKQINVIAGVKEPIRAY* | 947.5673 | 947.5702 | −3.04 |
| b18 | LKQINVIAGVKEPIRAYG | 976.0780 | 976.0810 | −3.12 |
| b18* | LKQINVIAGVKEPIRAYG* | 976.0780 | 976.0749 | 3.16 |
| b19 | LKQINVIAGVKEPIRAYGA | 1011.5966 | 1011.5966 | 0.04 |
| b19* | LKQINVIAGVKEPIRAYGA* | 1011.5966 | 1011.5977 | −1.12 |
| b20 | LKQINVIAGVKEPIRAYGAA | 1047.1151 | 1047.1153 | −0.20 |
| b20* | LKQINVIAGVKEPIRAYGAA* | 1047.1151 | 1047.1166 | −1.39 |
| b21 | LKQINVIAGVKEPIRAYGAAA | 1082.6337 | 1082.6353 | −1.47 |
| b21* | LKQINVIAGVKEPIRAYGAAA* | 1082.6337 | 1082.6366 | −2.70 |
| b22 | LKQINVIAGVKEPIRAYGAAAA | 1118.1522 | 1118.1565 | −3.82 |
| b22* | LKQINVIAGVKEPIRAYGAAAA* | 1118.1522 | 1118.1490 | 2.85 |
| b23 | LKQINVIAGVKEPIRAYGAAAAS | 774.7813 | 774.7822 | −1.12 |
| b23* | LKQINVIAGVKEPIRAYGAAAAS* | 774.7813 | 774.7829 | −2.04 |
| b24 | LKQINVIAGVKEPIRAYGAAAASA | 798.4603 | 798.4587 | 2.04 |
| b24* | LKQINVIAGVKEPIRAYGAAAASA* | 798.4603 | 798.4594 | 1.10 |
| b25 | LKQINVIAGVKEPIRAYGAAAASAC | 832.7967 | 832.7954 | 1.61 |
| b25* | (−1 Da)LKQINVIAGVKEPIRAYGAAAASAC* | 832.4608 | Not Found | N/A |
| b26 | LKQINVIAGVKEPIRAYGAAAASACD | 871.1390 | 871.1364 | 2.97 |
| b26* | LKQINVIAGVKEPIRAYGAAAASACD* | 870.8031 | Not Found | N/A |
| | (−1 Da) | | | |
| b27 | LKQINVIAGVKEPIRAYGAAAASACDA | 894.8180 | 894.8162 | 1.96 |
| b27* | LKQINVIAGVKEPIRAYGAAAASACDA* | 894.4821 | Not Found | N/A |
| | (−1 Da) | | | |
| b28 | LKQINVIAGVKEPIRAYGAAAASACDAA | 918.4971 | 918.4973 | −0.24 |
| b28* | LKQINVIAGVKEPIRAYGAAAASACDAA* | 918.1611 | Not Found | N/A |
| | (−1 Da) | | | |
| b29 | LKQINVIAGVKEPIRAYGAAAASACDAAD | 956.8394 | Not Found | N/A |
| b29* | LKQINVIAGVKEPIRAYGAAAASACDAAD* | 956.1675 | 956.1692 | −1.78 |
| | (−2 Da) | | | |
| [M] | LKQINVIAGVKEPIRAYGAAAASACDAADA | 986.5220 | 986.5250 | 3.03 |
| [M]* | LKQINVIAGVKEPIRAYGAAAASACDAADA* | 985.8501 | 985.8506 | −0.48 |
| | (−2 Da) | | | |
| y29 | KQINVIAGVKEPIRAYGAAAASACDAADA | 948.8273 | Not Found | N/A |
| y29* | KQINVIAGVKEPIRAYGAAAASACDAADA* | 948.1554 | 948.1583 | −3.05 |
| | (−2 Da) | | | |
| y28 | QINVIAGVKEPIRAYGAAAASACDAADA | 906.1289 | Not Found | N/A |
| y28* | QINVIAGVKEPIRAYGAAAASACDAADA* | 905.4571 | Not Found | N/A |
| | (−2 Da) | | | |
| y27 | INVIAGVKEPIRAYGAAAASACDAADA | 863.4428 | 863.4457 | −3.36 |
| y27* | INVIAGVKEPIRAYGAAAASACDAADA* | 862.7709 | 862.7732 | −2.70 |
| | (−2 Da) | | | |
| y26 | NVIAGVKEPIRAYGAAAASACDAADA | 825.7481 | Not Found | N/A |
| y26* | NVIAGVKEPIRAYGAAAASACDAADA* | 825.0762 | Not Found | N/A |
| | (−2 Da) | | | |
| y25 | VIAGVKEPIRAYGAAAASACDAADA | 787.7338 | Not Found | N/A |
| y25* | VIAGVKEPIRAYGAAAASACDAADA* | 787.0619 | Not Found | N/A |
| | (−2 Da) | | | |
| y24 | IAGVKEPIRAYGAAAASACDAADA | 754.7110 | 754.7127 | −2.21 |
| y24* | (−2 Da)IAGVKEPIRAYGAAAASACDAADA* | 754.0391 | Not Found | N/A |
| y23 | AGVKEPIRAYGAAAASACDAADA | 1075.0208 | 1075.0237 | −2.70 |
| y23* | (−2 Da)AGVKEPIRAYGAAAASACDAADA* | 1074.0129 | 1074.0145 | −1.52 |
| y22 | GVKEPIRAYGAAAASACDAADA | 1039.5022 | Not Found | N/A |
| y22* | (−2 Da)GVKEPIRAYGAAAASACDAADA* | 1038.4944 | 1038.4938 | 0.57 |
| y21 | VKEPIRAYGAAAASACDAADA | 1010.9915 | 1010.9942 | −2.66 |
| y21* | (−2 Da)VKEPIRAYGAAAASACDAADA* | 1009.9836 | 1009.9824 | 1.14 |
| y20 | KEPIRAYGAAAASACDAADA | 961.4573 | 961.4545 | 2.88 |
| y20* | (−2 Da)KEPIRAYGAAAASACDAADA* | 960.4494 | 960.4527 | −3.42 |
| y19 | EPIRAYGAAAASACDAADA | 897.4098 | Not Found | N/A |
| y19* | (−2 Da)EPIRAYGAAAASACDAADA* | 896.4020 | 896.3995 | 2.78 |
| y18 | PIRAYGAAAASACDAADA | 832.8885 | 832.8865 | 2.36 |
| y18* | (−2 Da)PIRAYGAAAASACDAADA* | 831.8807 | 831.8795 | 1.39 |
| y17 | IRAYGAAAASACDAADA | 784.3621 | Not Found | N/A |
| y17* | (−2 Da)IRAYGAAAASACDAADA* | 783.3543 | Not Found | N/A |
| y16 | RAYGAAAASACDAADA | 727.8201 | Not Found | N/A |
| y16* | (−2 Da)RAYGAAAASACDAADA* | 726.8122 | 726.8131 | −1.26 |
| y15 | AYGAAAASACDAADA | 1298.5318 | Not Found | N/A |
| y15* | (−2 Da)AYGAAAASACDAADA* | 1296.5161 | 1296.5117 | 3.41 |
| y14 | YGAAAASACDAADA | 1227.4946 | Not Found | N/A |
| y14* | (−2 Da)YGAAAASACDAADA* | 1225.4790 | 1225.4783 | 0.56 |
| y13 | GAAAASACDAADA | 1064.4313 | 1064.4308 | 0.48 |
| y13* | (−2 Da)GAAAASACDAADA* | 1062.4157 | 1062.4177 | −1.85 |
| y12 | AAAASACDAADA | 1007.4099 | Not Found | N/A |
| y12* | (−2 Da)AAAASACDAADA* | 1005.3942 | 1005.3940 | 0.19 |
| y11 | AAASACDAADA | 936.3727 | 936.3759 | −3.44 |
| y11* | (−2 Da)AAASACDAADA* | 934.3571 | 934.3557 | 1.46 |
| y10 | AASACDAADA | 865.3356 | 865.3377 | −2.40 |
| y10* | (−2 Da)AASACDAADA* | 863.3200 | 863.3203 | −0.30 |
| y9 | ASACDAADA | 794.2985 | 794.2972 | 1.66 |
| y9* | (−2 Da)ASACDAADA* | 792.2829 | 792.2849 | −2.50 |
| y8 | SACDAADA | 723.2614 | 723.2623 | −1.20 |
| y8* | (−2 Da)SACDAADA* | 721.2457 | 721.2472 | −2.04 |
| y7 | ACDAADA | 636.2294 | 636.2303 | −1.37 |
| y7* | (−2 Da)ACDAADA* | 634.2137 | 634.2152 | −2.30 |
| y6 | CDAADA | 565.1923 | 565.1921 | 0.41 |
| y6* | (−2 Da)CDAADA* | 563.1766 | 563.1781 | −2.61 |
| y5 | DAADA | 462.1831 | 462.1838 | −1.59 |
| y5* | (−1 Da)DAADA* | 461.1752 | Not Found | N/A |
| y4 | AADA | 347.1561 | 347.1554 | 2.02 |
| y4* | (−1 Da)AADA* | 346.1483 | Not Found | N/A |
| y3 | ADA | 276.1190 | 276.1190 | 0.16 |
| y3* | (−1 Da)ADA* | 275.1112 | Not Found | N/A |
| y2 | DA | 205.0819 | 205.0820 | −0.49 |
| y2* | (−1 Da)DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 14
LKQINVIAGVKEPIRAYGAAACSAADAAADA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1860 | 1.18 |
| b2* | LK* | 242.1863 | 242.1861 | 0.71 |
| b3 | LKQ | 370.2449 | 370.2442 | 1.89 |
| b3* | LKQ* | 370.2449 | 370.2444 | 1.35 |
| b4 | LKQI | 483.3289 | 483.3286 | 0.59 |
| b4* | LKQI* | 483.3289 | 483.3289 | 0.00 |
| b5 | LKQIN | 597.3719 | 597.3705 | 2.35 |
| b5* | LKQIN | 597.3719 | 597.3709 | 1.71 |
| b6 | LKQINV | 696.4403 | 696.4396 | 1.07 |
| b6* | LKQINV* | 696.4403 | 696.4400 | 0.40 |
| b7 | LKQINVI | 809.5244 | 809.5217 | 3.33 |
| b7* | LKQINVI* | 809.5244 | 809.5223 | 2.63 |
| b8 | LKQINVIA | 880.5615 | 880.5628 | −1.49 |
| b8* | LKQINVIA* | 880.5615 | 880.5635 | −2.21 |
| b9 | LKQINVIAG | 937.5829 | 937.5855 | −2.73 |
| b9* | LKQINVIAG* | 937.5829 | 937.5862 | −3.47 |
| b10 | LKQINVIAGV | 1036.6513 | 1036.6532 | −1.80 |
| b10* | LKQINVIAGV* | 1036.6513 | 1036.6540 | −2.56 |
| b11 | LKQINVIAGVK | 1164.7463 | 1164.7471 | −0.68 |
| b11* | LKQINVIAGVK* | 1164.7463 | 1164.7480 | −1.47 |
| b12 | LKQINVIAGVKE | 1293.7889 | 1293.7899 | −0.75 |
| b12* | LKQINVIAGVKE* | 1293.7889 | 1293.7909 | −1.57 |
| b13 | LKQINVIAGVKEP | 1390.8417 | 1390.8395 | 1.61 |
| b13* | LKQINVIAGVKEP* | 1390.8417 | 1390.8406 | 0.78 |
| b14 | LKQINVIAGVKEPI | 1503.9257 | 1503.9235 | 1.44 |
| b14* | LKQINVIAGVKEPI* | 1503.9257 | 1503.9248 | 0.58 |
| b15 | LKQINVIAGVKEPIR | 1660.0268 | 1660.0307 | −2.33 |
| b15* | LKQINVIAGVKEPIR* | 1660.0268 | 1660.0321 | −3.22 |
| b16 | LKQINVIAGVKEPIRA | 866.0356 | 866.0374 | −2.09 |
| b16* | LKQINVIAGVKEPIRA* | 866.0356 | 866.0380 | −2.81 |
| b17 | LKQINVIAGVKEPIRAY | 947.5673 | 947.5682 | −1.00 |
| b17* | LKQINVIAGVKEPIRAY* | 947.5673 | 947.5689 | −1.74 |
| b18 | LKQINVIAGVKEPIRAYG | 976.0780 | 976.0801 | −2.16 |
| b18* | LKQINVIAGVKEPIRAYG* | 976.0780 | 976.0808 | −2.91 |
| b19 | LKQINVIAGVKEPIRAYGA | 1011.5966 | 1011.5956 | 1.02 |
| b19* | LKQINVIAGVKEPIRAYGA* | 1011.5966 | 1011.5963 | 0.26 |
| b20 | LKQINVIAGVKEPIRAYGAA | 1047.1151 | 1047.1143 | 0.80 |
| b20* | LKQINVIAGVKEPIRAYGAA* | 1047.1151 | 1047.1151 | 0.03 |
| b21 | LKQINVIAGVKEPIRAYGAAA | 1082.6337 | 1082.6342 | −0.46 |
| b21* | LKQINVIAGVKEPIRAYGAAA* | 1082.6337 | 1082.6350 | −1.23 |
| b22 | LKQINVIAGVKEPIRAYGAAAC | 1134.1383 | 1134.1397 | −1.23 |
| b22* | (−1 Da)LKQINVIAGVKEPIRAYGAAAC* | 1133.6344 | Not Found | N/A |
| b23 | LKQINVIAGVKEPIRAYGAAACS | 1177.6543 | 1177.6521 | 1.84 |
| b23* | (−1 Da)LKQINVIAGVKEPIRAYGAAACS* | 1177.1504 | Not Found | N/A |
| b24 | LKQINVIAGVKEPIRAYGAAACSA | 809.1177 | 809.1177 | −0.06 |
| b24* | (−1 Da)LKQINVIAGVKEPIRAYGAAACSA* | 808.7817 | Not Found | N/A |
| b25 | LKQINVIAGVKEPIRAYGAAACSAA | 832.7967 | 832.7946 | 2.50 |
| b25* | (−1 Da)LKQINVIAGVKEPIRAYGAAACSAA* | 832.4608 | Not Found | N/A |
| b26 | LKQINVIAGVKEPIRAYGAAACSAAD | 871.1390 | 871.1417 | −3.12 |
| b26* | (−2 Da) | 870.4671 | 870.4661 | 1.19 |
| | LKQINVIAGVKEPIRAYGAAACSAAD* | | | |
| b27 | LKQINVIAGVKEPIRAYGAAACSAADA | 894.8180 | 894.8154 | 2.88 |
| b27* | (−2 Da) | 894.1462 | 894.1437 | 2.75 |
| | LKQINVIAGVKEPIRAYGAAACSAADA* | | | |
| b28 | LKQINVIAGVKEPIRAYGAAACSAADAA | 918.4971 | 918.4965 | 0.69 |
| b28* | LKQINVIAGVKEPIRAYGAAACSAADAA* | 917.8252 | Not Found | N/A |
| | (−2 Da) | | | |
| b29 | LKQINVIAGVKEPIRAYGAAACSAADAAD | 956.8394 | Not Found | N/A |
| b29* | LKQINVIAGVKEPIRAYGAAACSAADAAD* | 956.1675 | 956.1679 | −0.47 |
| | (−2 Da) | | | |
| [M] | LKQINVIAGVKEPIRAYGAAACSAADAADA | 986.5220 | 986.5240 | −2.06 |
| [M]* | LKQINVIAGVKEPIRAYGAAACSAADAADA* | 985.8501 | Not Found | N/A |
| | (−2 Da) | | | |
| y29 | KQINVIAGVKEPIRAYGAAACSAADAADA | 948.8273 | Not Found | N/A |
| y29* | KQINVIAGVKEPIRAYGAAACSAADAADA* | 948.1554 | Not Found | N/A |
| | (−2 Da) | | | |
| y28 | QINVIAGVKEPIRAYGAAACSAADAADA | 906.1289 | Not Found | N/A |
| y28* | QINVIAGVKEPIRAYGAAACSAADAADA* | 905.4571 | 905.4576 | −0.58 |
| | (−2 Da) | | | |
| y27 | INVIAGVKEPIRAYGAAACSAADAADA | 863.4428 | Not Found | N/A |
| y27* | (−2 Da) | 862.7709 | 862.7722 | −1.50 |
| | INVIAGVKEPIRAYGAAACSAADAADA* | | | |
| y26 | NVIAGVKEPIRAYGAAACSAADAADA | 825.7481 | Not Found | N/A |
| y26* | (−2 Da) | 825.0762 | 825.0771 | −1.09 |
| | NVIAGVKEPIRAYGAAACSAADAADA* | | | |
| y25 | VIAGVKEPIRAYGAAACSAADAADA | 787.7338 | 787.7348 | −1.30 |
| y25* | (−2 Da) | 787.0619 | 787.0596 | 2.93 |
| | VIAGVKEPIRAYGAAACSAADAADA* | | | |
| y24 | IAGVKEPIRAYGAAACSAADAADA | 754.7110 | Not Found | N/A |
| y24* | (−2 Da)IAGVKEPIRAYGAAACSAADAADA* | 754.0391 | 754.0395 | −0.57 |
| y23 | AGVKEPIRAYGAAACSAADAADA | 1075.0208 | 1075.0226 | −1.69 |
| y23* | (−2 Da)AGVKEPIRAYGAAACSAADAADA* | 1074.0129 | 1074.0130 | −0.06 |
| y22 | GVKEPIRAYGAAACSAADAADA | 1039.5022 | 1039.5000 | 2.13 |
| y22* | (−2 Da)GVKEPIRAYGAAACSAADAADA* | 1038.4944 | Not Found | N/A |
| y21 | VKEPIRAYGAAACSAADAADA | 1010.9915 | Not Found | N/A |
| y21* | (−2 Da)VKEPIRAYGAAACSAADAADA* | 1009.9836 | Not Found | N/A |
| y20 | KEPIRAYGAAACSAADAADA | 961.4573 | 961.4607 | −3.52 |
| y20* | (−2 Da)KEPIRAYGAAACSAADAADA* | 960.4494 | 960.4514 | −2.10 |
| y19 | EPIRAYGAAACSAADAADA | 897.4098 | Not Found | N/A |
| y19* | (−2 Da)EPIRAYGAAACSAADAADA* | 896.4020 | Not Found | N/A |
| y18 | PIRAYGAAACSAADAADA | 832.8885 | Not Found | N/A |
| y18* | (−2 Da)PIRAYGAAACSAADAADA* | 831.8807 | 831.8786 | 2.55 |
| y17 | IRAYGAAACSAADAADA | 1567.7169 | Not Found | N/A |
| y17* | (−2 Da)IRAYGAAACSAADAADA* | 1565.7013 | 1565.6957 | 3.59 |
| y16 | RAYGAAACSAADAADA | 1454.6329 | 1454.6279 | 3.41 |
| y16* | (−2 Da)RAYGAAACSAADAADA* | 1452.6172 | 1452.6187 | −1.05 |
| y15 | AYGAAACSAADAADA | 1298.5318 | 1298.5363 | −3.47 |
| y15* | (−2 Da)AYGAAACSAADAADA* | 1296.5161 | Not Found | N/A |
| y14 | YGAAACSAADAADA | 1227.4946 | Not Found | N/A |
| y14* | (−2 Da)YGAAACSAADAADA* | 1225.4790 | 1225.4763 | 2.19 |
| y13 | GAAACSAADAADA | 1064.4313 | Not Found | N/A |
| y13* | (−2 Da)GAAACSAADAADA* | 1062.4157 | 1062.4161 | −0.41 |
| y12 | AAACSAADAADA | 1007.4099 | Not Found | N/A |
| y12* | (−2 Da)AAACSAADAADA* | 1005.3942 | Not Found | N/A |
| y11 | AACSAADAADA | 936.3727 | 936.3750 | −2.50 |
| y11* | (−2 Da)AACSAADAADA* | 934.3571 | 934.3545 | 2.75 |
| y10 | ACSAADAADA | 865.3356 | 865.3369 | −1.49 |
| y10* | (−2 Da)ACSAADAADA* | 863.3200 | 863.3192 | 0.90 |
| y9 | CSAADAADA | 794.2985 | 794.2965 | 2.53 |
| y9* | (−2 Da)CSAADAADA* | 792.2829 | 792.2817 | −1.51 |
| y8 | SAADAADA | 691.2893 | 691.2872 | 3.10 |
| y8* | (−1 Da)SAADAADA* | 690.2815 | Not Found | N/A |
| y7 | AADAADA | 604.2573 | 604.2559 | 2.37 |
| y7* | (−1 Da)AADAADA* | 603.2495 | Not Found | N/A |
| y6 | ADAADA | 533.2202 | 533.2191 | 2.12 |
| y6* | (−1 Da)ADAADA* | 532.2124 | Not Found | N/A |
| y5 | DAADA | 462.1831 | 462.1835 | −0.92 |
| y5* | DAADA* | 461.1752 | Not Found | N/A |
| y4 | AADA | 347.1561 | 347.1567 | −1.82 |
| y4* | AADA* | 347.1561 | 347.1554 | 2.07 |
| y3 | ADA | 276.1190 | 276.1188 | 0.67 |
| y3* | ADA* | 276.1190 | 276.1189 | 0.18 |
| y2 | DA | 205.0819 | 205.0819 | −0.05 |
| y2* | DA* | 205.0819 | 205.0820 | −0.49 |

Unmodified indicates z = 1 charge state.
Underline indicates z = 2 charge state.
Italics indicates z = 3 charge state.
*Indicates the fragment originated from the modified peptide

The results with expansions of the CXₙD motif in the previous section demonstrate a lack of defined specificity in the recognition sequence, beyond the preference for Cys and Asp. The data with the nested crosslinks above extend this to include distance from the leader peptide recognition sequence, as well as the individual amino acids within the processed peptide. These observations suggest that the only elements that guide binding and crosslinking activity is the presence of proximal Cys and Asp residues, and the leader sequence, which is presumed to be required for RiPP maturases (Precord, T. W., et al. ACS Chem. Biol. 2019, 14 (9), 1981-89). These observations support the notion that PapB may be able to be used widely to introduce thioether crosslinks in peptides that are completely unrelated to the naturally occurring PapA substrate.

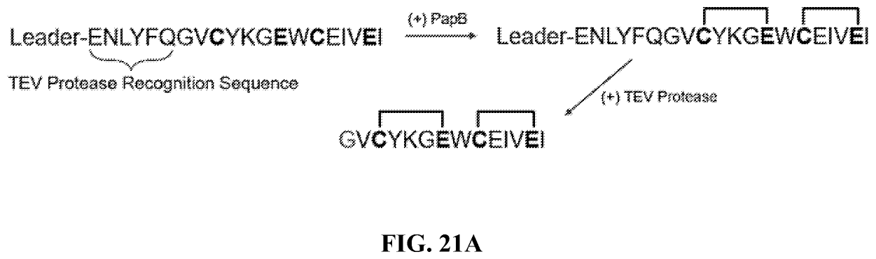

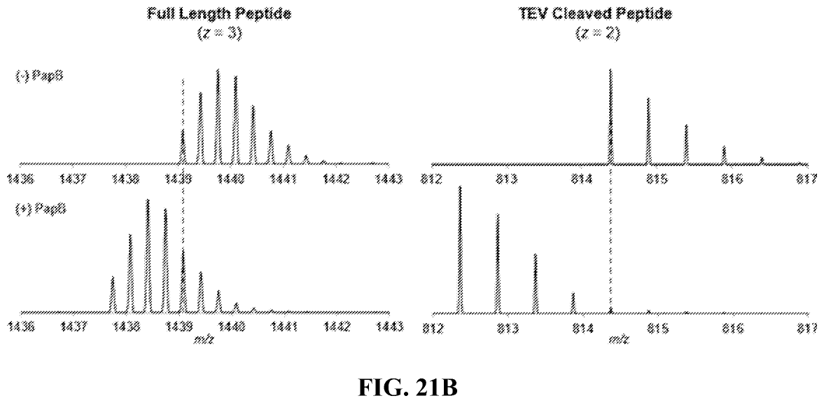

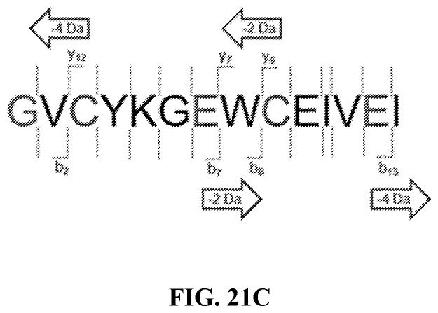

Indeed, PapB has been used recently to prepare peptide products that are capable of binding single protein targets, such as the SARS-COV-2 spike receptor binding domain (King, A. M., et al. Nat. Commun. 2021, 12, 6343). The peptide in that design contained a leader sequence, which through a TEV protease recognition sequence is connected to a minimal substrate containing two CX₃E motifs. The initial report on PapB had demonstrated that both Asp and Glu are crosslinked by the enzyme (Precord, T. W., et al. ACS Chem. Biol. 2019, 14 (9), 1981-89). In the more recent article, however, while the peptide contained two potential crosslinking motifs, only a single crosslink was observed. Considering the in vitro data with highly active protein shows that the topology of the modification can be essentially directed, this result was revisited to determine if the absence of the second crosslinks reflects the in vivo system employed rather than inherent to PapB. A synthetic peptide that is identical to the unnatural peptide used to target the SARS-COV-2 spike receptor-binding domain was synthesized and treated with the protein as described above (FIG. 21A). As the mass spectrum of the peptide shows, the enzyme installs two crosslinks, as evidenced by the loss of 4 Da in the modified peptide (FIG. 21B). Next, TEV cleavage of the resulting product was carried out to release mature peptide and as the MS shows, it also exhibits a 4 Da loss, localizing the modification to the peptide. The observed monoisotopic masses for both the full length and the TEV cleaved peptide are <3 ppm of the expected values (Table 15).

TABLE 15

| | Expected | Observed | |
|---|---|---|---|
| | Monoisotopic | Monoisotopic | Ppm |
| Sequence | Mass | Mass | Error |
| Leader- | (−)PapB: | 1439.0844 | 2.154 |
| ENLYFQGVCY | 1439.0813 | 1437.7386 | 0.765 |
| KGEWCEIVEI | (+)PapB | | |
| (z = 3) | (−4 Da): | | |
| | 1437.7375 | | |
| GVCYKGEWCE | (−)PapB: | 814.3784 | 1.228 |
| IVEI | 814.3774 | 812.3649 | 2.708 |
| | (+)PapB | | |
| | (−4 Da): | | |
| | 812.3627 | | |

Tandem mass spectrometry shows a fragmentation pattern that is indicative of two thioether events occurring; one between Cys3 and Glu7, and the other between Cys9 and Glu13 (FIG. 21C, Table 16). Therefore, the presence of a single cross link in the reported peptide was likely due to the in vivo conditions employed.

TABLE 16
GVCYKGEWCEIVEI

| | | Expected | Observed | |
|---|---|---|---|---|
| | | Mono- | Mono- | |
| | | isotopic | isotopic | Ppm |
| Ion | Sequence | Mass | Mass | Error |
| b2 | GV | 157.0972 | 157.0972 | −0.01 |
| b2* | GV* | 157.0972 | 157.0972 | −0.03 |
| b3 | GVC | 260.1063 | 260.1058 | 1.75 |
| b3* | GVC* | 259.0985 | Not Found | N/A |
| b4 | GVCK | 388.2013 | 388.2022 | −2.29 |
| b4* | GVCK* | 387.1935 | Not Found | N/A |
| b5 | GVCKY | 551.2646 | 551.2648 | −0.35 |
| b5* | GVCKY* | 550.2568 | Not Found | N/A |
| b6 | GVCKYG | 608.2861 | 608.2868 | −1.12 |
| b6* | GVCKYG* | 607.2783 | Not Found | N/A |
| b7 | GVCKYGE | 737.3287 | 737.3305 | −2.40 |
| b7* | GVCKYGE* | 735.3130 | 735.3131 | −0.07 |
| b8 | GVCKYGEW | 923.4080 | 923.4054 | 2.84 |
| b8* | GVCKYGEW* | 921.3923 | 921.3938 | −1.63 |
| b9 | GVCKYGEWC | 1026.4172 | 1026.4167 | 0.53 |
| b9* | GVCKYGEWC* | 1023.3937 | Not Found | N/A |
| b10 | GVCKYGEWCE | 1155.4598 | 1155.4610 | −1.03 |
| b10* | GVCKYGEWCE* | 1152.4363 | Not Found | N/A |
| b11 | GVCKYGEWCEI | 1268.5438 | 1268.5428 | 0.75 |
| b11* | GVCKYGEWCEI* | 1265.5204 | Not Found | N/A |
| b12 | GVCKYGEWCEIV | 1367.6123 | 1367.6174 | −3.71 |
| b12* | GVCKYGEWCEIV* | 1364.5888 | Not Found | N/A |
| b13 | GVCKYGEWCEIVE | 1496.6548 | 1496.6590 | −2.79 |
| b13* | GVCKYGEWCEIVE* | 1492.6235 | 1492.6197 | 2.57 |
| [M] | GVCKYGEWCEIVEI | 1627.7495 | 1627.7434 | 3.72 |
| [M]* | GVCKYGEWCEIVEI* | 1623.7182 | 1623.7208 | −1.58 |
| y13 | VCKYGEWCEIVEI | 1570.7280 | 1570.7260 | 1.26 |
| y13* | VCKYGEWCEIVEI* | 1566.6967 | Not Found | N/A |
| y12 | CKYGEWCEIVEI | 1471.6596 | 1471.6622 | −1.73 |
| y12* | CKYGEWCEIVEI* | 1467.6283 | 1467.6302 | −1.31 |
| y11 | KYGEWCEIVEI | 1368.6504 | 1368.6492 | 0.87 |
| y11* | KYGEWCEIVEI* | 1365.6269 | Not Found | N/A |
| y10 | YGEWCEIVEI | 1240.5555 | 1240.5524 | 2.50 |
| y10* | YGEWCEIVEI* | 1237.5320 | Not Found | N/A |
| y9 | GEWCEIVEI | 1077.4921 | 1077.4904 | 1.57 |
| y9* | GEWCEIVEI* | 1074.4686 | Not Found | N/A |
| y8 | EWCEIVEI | 1020.4707 | 1020.4704 | 0.29 |
| y8* | EWCEIVEI* | 1017.4472 | Not Found | N/A |
| y7 | WCEIVEI | 891.4281 | 891.4287 | −0.64 |
| y7* | WCEIVEI* | 889.4124 | 889.4137 | −1.44 |
| y6 | CEIVEI | 705.3488 | 705.3476 | 1.64 |
| y6* | CEIVEI* | 703.3331 | 703.3317 | 2.01 |
| y5 | EIVEI | 602.3396 | 602.3408 | −1.94 |
| y5* | EIVEI* | 601.3317 | Not Found | N/A |
| y4 | IVEI | 473.2970 | 473.2968 | 0.49 |
| y4* | IVEI* | 472.2892 | Not Found | N/A |
| y3 | VEI | 360.2129 | 360.2135 | −1.77 |
| y3* | VEI* | 359.2051 | Not Found | N/A |
| y2 | EI | 261.1445 | 261.1443 | 0.72 |
| y2* | EI* | 260.1367 | Not Found | N/A |

Unmodified indicates z = 1 charge state.
Underline indicates z = 2 charge state.
Italics indicates z = 3 charge state.
*Indicates the fragment originated from the modified peptide

c. D-Amino Acids are Processed by PapB

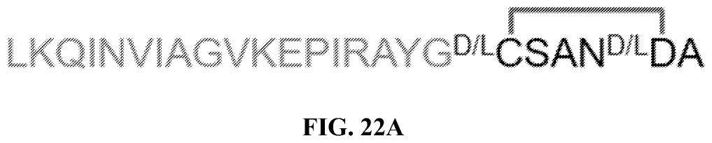

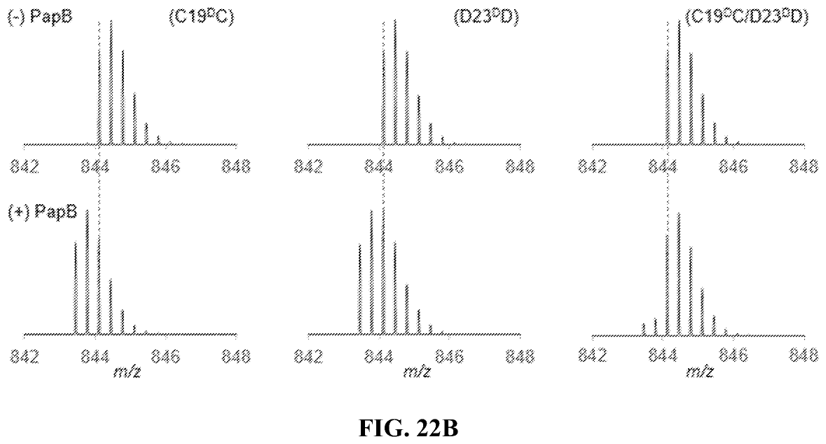

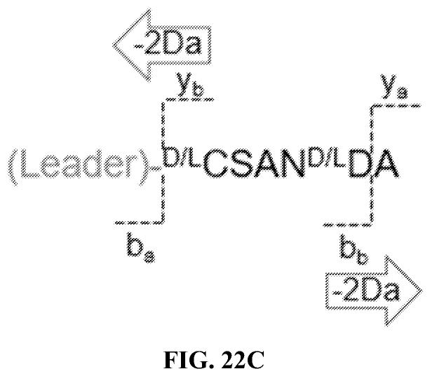

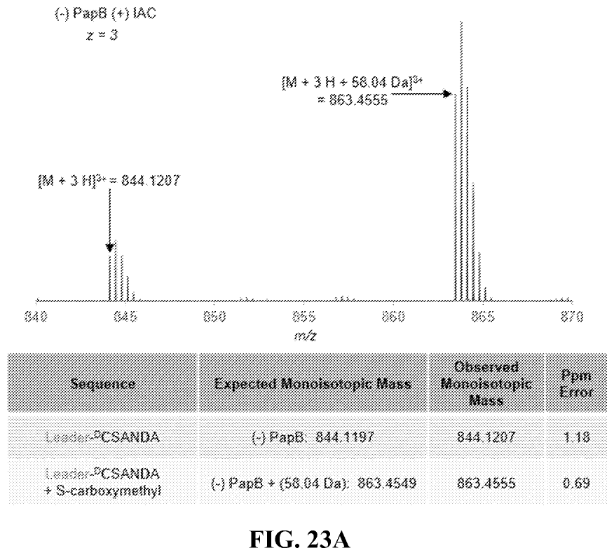

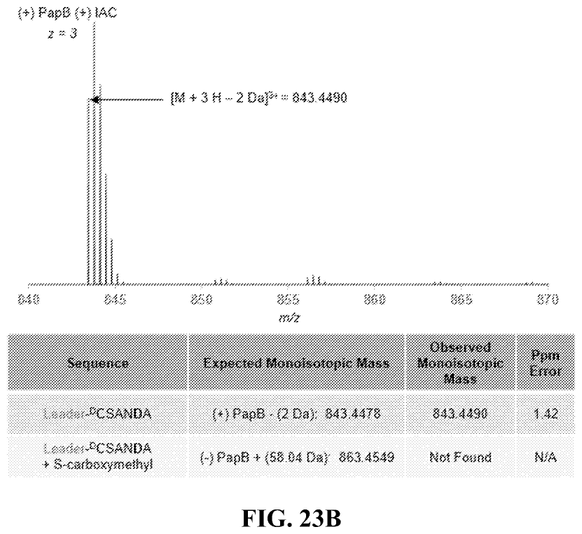

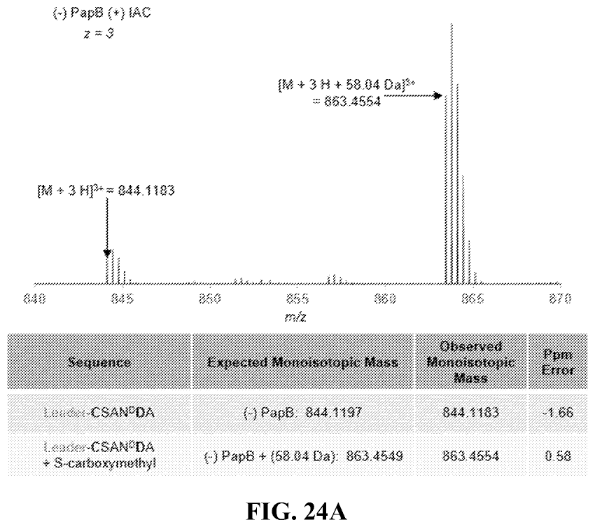

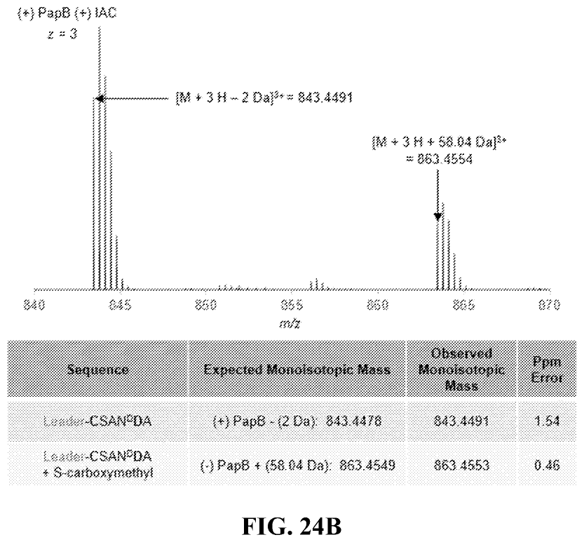

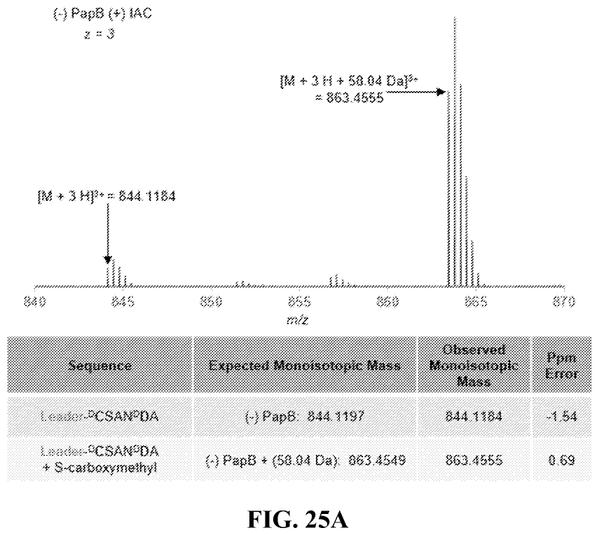

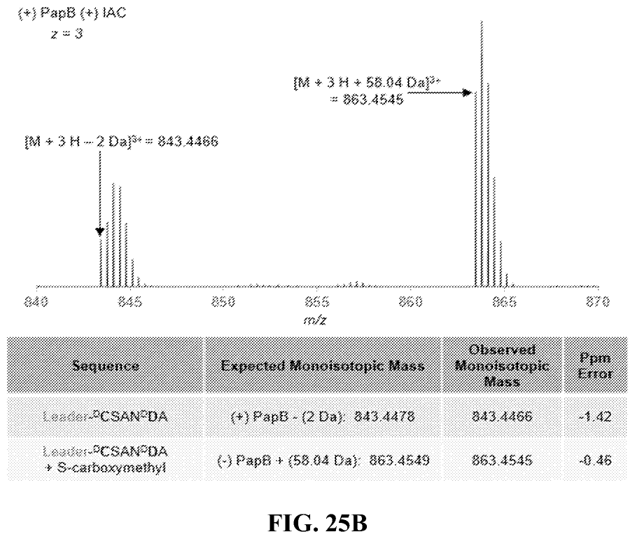

In initial experiments with PapA/PapB the CX₃D spacing was intriguing, which suggested that the enzyme recognizes the Cys and Asp residues as part of a helical fragment, since Cys and Asp sidechains would be expected to be located on the same face of an alpha helix. However, the expansions and contractions of the motif clearly demonstrated that the spacing is immaterial. The expansion and contraction results suggest that only the identity of the amino acid or specific chemical moieties is important. Therefore, whether PapB can process msPapA when Cys and Asp are replaced with their dextrorotatory enantiomers was explored (FIG. 22A). With the leader-^{D}CSANDA peptide, full conversion to the crosslinked peptide is seen, as evidenced by the loss of 2 Da (FIG. 22B). With the leader-CSAN^{D}DA peptide, significant substrate turnover is observed as well, but the conversion is not complete (FIG. 22B). The leader-^{D}CSAN^{D}DA is processed inefficiently under these conditions, though some product is clearly observed in the MS. While it is possible to suggest that the small amount of product observed with this peptide is due to contaminating L-amino acids in the commercially available sources, that impurity would only account to 1-2% of product turnover. Based on the MS data, at least ˜15% of the substrate is converted to product, arguing that the modification represents a bona fide ^{D}Cys to ^{D}Asp thioether cross link. Finally, CID MS/MS spectrometry shows a loss of 2 Da in each y-fragment after the C residue and in the single b-fragment after the D residue in all three D-peptide scenarios (FIG. 22C, Tables 17-19) showing a stable macrocycle. Control experiments show that treatment with IAA results in no carboxymethylation in the C19^{D}C peptide (FIG. 23). In the case of the D23^{D}D and C19^{D}C/D23^{D}D peptides, carboxymethylation is present upon IAA treatment due to incomplete turnover (FIG. 23-25). However, the carboxymethylated species does not show any evidence of a 2 Da loss, which gives evidence that the Cys thiol is participating in the newly installed bond in these unnatural peptides.

TABLE 17
LKQINVIAGVKEPIRAYG^{D}CSANDA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1864 | −0.27 |
| b2* | LK* | 242.1863 | 242.1866 | −1.33 |
| b3 | LKQ | 370.2449 | 370.2447 | 0.52 |
| b3* | LKQ* | 370.2449 | 370.2450 | −0.27 |
| b4 | LKQI | 483.3289 | 483.3292 | −0.72 |
| b4* | LKQI* | 483.3289 | 483.3295 | −1.32 |
| b5 | LKQIN | 597.3719 | 597.3712 | 1.09 |
| b5* | LKQIN* | 597.3719 | 597.3715 | 0.67 |
| b6 | LKQINV | 696.4403 | 696.4404 | −0.14 |
| b6* | LKQINV* | 696.4403 | 696.4406 | −0.43 |
| b7 | LKQINVI | 809.5244 | 809.5226 | 2.16 |
| b7* | LKQINVI* | 809.5244 | 809.5228 | 2.03 |
| b8 | LKQINVIA | 880.5615 | 880.5638 | −2.63 |
| b8* | LKQINVIA* | 880.5615 | 880.5639 | −2.68 |
| b9 | LKQINVIAG | 937.5829 | 937.5797 | 3.41 |
| b9* | LKQINVIAG* | 937.5829 | 937.5797 | 3.43 |
| b10 | LKQINVIAGV | 1036.6513 | 1036.6543 | −2.89 |
| b10* | LKQINVIAGV* | 1036.6513 | 1036.6542 | −2.75 |
| b11 | LKQINVIAGVK | 1164.7463 | 1164.7483 | −1.72 |
| b11* | LKQINVIAGVK* | 1164.7463 | 1164.7480 | −1.44 |
| b12 | LKQINVIAGVKE | 1293.7889 | 1293.7912 | −1.75 |
| b12* | LKQINVIAGVKE* | 1293.7889 | 1293.7906 | −1.34 |
| b13 | LKQINVIAGVKEP | 1390.8417 | 1390.8408 | 0.64 |
| b13* | LKQINVIAGVKEP* | 1390.8417 | 1390.8401 | 1.15 |
| b14 | LKQINVIAGVKEPI | 1503.9257 | 1503.9250 | 0.50 |
| b14* | LKQINVIAGVKEPI* | 1503.9257 | 1503.9240 | 1.12 |
| b15 | LKQINVIAGVKEPIR | 1660.0268 | 1660.0322 | −3.23 |
| b15* | LKQINVIAGVKEPIR* | 1660.0268 | Not Found | N/A |
| b16 | LKQINVIAGVKEPIRA | 866.0356 | 866.0384 | −3.24 |
| b16* | LKQINVIAGVKEPIRA* | 866.0356 | 866.0385 | −3.31 |
| b17 | LKQINVIAGVKEPIRAY | 947.5673 | 947.5693 | −2.11 |
| b17* | LKQINVIAGVKEPIRAY* | 947.5673 | 947.5693 | −2.08 |
| b18 | LKQINVIAGVKEPIRAYG | 976.0780 | 976.0812 | −3.27 |
| b18* | LKQINVIAGVKEPIRAYG* | 976.0780 | 976.0811 | −3.20 |
| b19 | LKQINVIAGVKEPIRAYG^{D}C | 1027.5826 | 1027.5820 | 0.56 |
| b19* | (−1 Da)LKQINVIAGVKEPIRAYG^{D}C* | 1027.0787 | Not Found | N/A |
| b20 | LKQINVIAGVKEPIRAYG^{D}CS | 1071.0986 | 1071.0984 | 0.16 |
| b20* | (−1 Da)LKQINVIAGVKEPIRAYG^{D}CS* | 1070.5947 | Not Found | N/A |
| b21 | LKQINVIAGVKEPIRAYG^{D}CSA | 1106.6172 | 1106.6158 | 1.23 |
| b21* | (−1 Da)LKQINVIAGVKEPIRAYG^{D}CSA* | 1106.1133 | Not Found | N/A |
| b22 | LKQINVIAGVKEPIRAYG^{D}CSAN | 776.0948 | 776.0930 | 2.26 |
| b22* | (−1 Da)LKQINVIAGVKEPIRAYG^{D}CSAN* | 775.7589 | Not Found | N/A |
| b23 | LKQINVIAGVKEPIRAYGDG^{D}CSAND | 814.4372 | 814.4379 | −0.84 |
| b23* | (−2 Da)LKQINVIAGVKEPIRAYG^{D}CSAND* | 813.7653 | 813.7661 | −0.98 |
| [M] | LKQINVIAGVKEPIRAYG^{D}CSANDA | 844.1197 | 844.1179 | 2.19 |
| [M]* | (−2 Da) | 843.4478 | 843.4496 | −2.18 |
| | LKQINVIAGVKEPIRAYG^{D}CSANDA* | | | |
| y23 | KQINVIAGVKEPIRAYG^{D}CSANDA | 806.4250 | 806.4243 | 0.84 |
| y23* | (−2 Da) | 805.7532 | 805.7516 | 2.00 |
| | KQINVIAGVKEPIRAYG^{D}CSANDA* | | | |
| y22 | QINVIAGVKEPIRAYG^{D}CSANDA | 763.7267 | 763.7248 | 2.46 |
| y22* | (−2 Da)QINVIAGVKEPIRAYG^{D}CSANDA* | 763.0548 | 763.0549 | −0.08 |
| y21 | INVIAGVKEPIRAYG^{D}CSANDA | 721.0405 | 721.0402 | 0.40 |
| y21* | (−2 Da)INVIAGVKEPIRAYGPCSANDA* | 720.3686 | 720.3706 | −2.84 |
| y20 | NVIAGVKEPIRAYG^{D}CSANDA | 1024.5151 | 1024.5118 | 3.20 |
| y20* | (−2 Da)NVIAGVKEPIRAYG^{D}CSANDA* | 1023.5073 | 1023.5095 | −2.12 |
| y19 | VIAGVKEPIRAYG^{D}CSANDA | 967.4936 | 967.4910 | 2.69 |
| y19* | (−2 Da)VIAGVKEPIRAYG^{D}CSANDA* | 966.4858 | 966.4857 | 0.10 |
| y18 | IAGVKEPIRAYGPCSANDA | 917.9594 | 917.9565 | 3.11 |
| y18* | (−2 Da)IAGVKEPIRAYG^{D}CSANDA* | 916.9516 | 916.9485 | 3.39 |
| y17 | AGVKEPIRAYGPCSANDA | 861.4174 | 861.4162 | 1.34 |
| y17* | (−2 Da)AGVKEPIRAYG^{D}CSANDA* | 860.4096 | 860.4101 | −0.64 |
| y16 | GVKEPIRAYGPCSANDA | 1650.7904 | 1650.7861 | 2.61 |
| y16* | (−2 Da)GVKEPIRAYG^{D}CSANDA* | 1648.7748 | 1648.7671 | 4.66 |
| y15 | VKEPIRAYGPCSANDA | 1593.7690 | 1593.7707 | −1.04 |
| y15* | (−2 Da)VKEPIRAYG^{D}CSANDA* | 1591.7533 | 1591.7500 | 2.08 |
| y14 | KEPIRAYGPCSANDA | 1494.7006 | 1494.7028 | −1.46 |
| y14* | (−2 Da)KEPIRAYG^{D}CSANDA* | 1492.6849 | 1492.6898 | −3.31 |
| y13 | EPIRAYGPCSANDA | 1366.6056 | 1366.6030 | 1.93 |
| y13* | (−2 Da)EPIRAYG^{D}CSANDA* | 1364.5899 | 1364.5922 | −1.70 |
| y12 | PIRAYGPCSANDA | 1237.5630 | 1237.5675 | −3.66 |
| y12* | (−2 Da)PIRAYG^{D}CSANDA* | 1235.5473 | 1235.5464 | 0.72 |
| y11 | IRAYGPCSANDA | 1140.5102 | 1140.5066 | 3.20 |
| y11* | (−2 Da)IRAYG^{D}CSANDA* | 1138.4946 | 1138.4908 | 3.37 |
| y10 | RAYGPCSANDA | 1027.4262 | 1027.4258 | 0.34 |
| y10* | (−2 Da)RAYG^{D}CSANDA* | 1025.4105 | 1025.4142 | −3.59 |
| y9 | AYGPCSANDA | 871.3251 | 871.3256 | −0.60 |
| y9* | (−2 Da)AYG^{D}CSANDA* | 869.3094 | 869.3107 | −1.53 |
| y8 | YGPCSANDA | 800.2879 | 800.2862 | 2.18 |
| y8* | (−2 Da)YG^{D}CSANDA* | 798.2723 | 798.2718 | 0.58 |
| y7 | GPCSANDA | 637.2246 | 637.2248 | −0.32 |
| y7* | (−2 Da)G^{D}CSANDA* | 635.2090 | 635.2085 | 0.75 |
| y6 | DCSANDA | 580.2032 | 580.2032 | 0.07 |
| y6* | (−2 Da)DCSANDA* | 578.1875 | 578.1874 | 0.10 |
| y5 | SANDA | 477.1940 | 477.1933 | 1.52 |
| y5* | (−1 Da)SANDA* | 476.1861 | Not Found | N/A |
| y4 | ANDA | 390.1619 | 390.1614 | 1.40 |
| y4* | (−1 Da)ANDA* | 389.1541 | Not Found | N/A |
| y3 | NDA | 319.1248 | 319.1250 | −0.63 |
| y3* | (−1 Da)NDA* | 318.1170 | Not Found | N/A |
| y2 | DA | 205.0819 | 205.0822 | −1.53 |
| y2* | (−1 Da)DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

TABLE 18
LKQINVIAGVKEPIRAYGCSAN^{D}DA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1866 | −1.06 |
| b2* | LK* | 242.1863 | 242.1860 | 1.20 |
| b3 | LKQ | 370.2449 | 370.2452 | −0.85 |
| b3* | LKQ* | 370.2449 | 370.2456 | −1.85 |
| b4 | LKQI | 483.3289 | 483.3301 | −2.53 |
| b4* | LKQI* | 483.3289 | 483.3279 | 2.02 |
| b5 | LKQIN | 597.3719 | 597.3726 | −1.11 |
| b5* | LKQIN* | 597.3719 | 597.3728 | −1.48 |
| b6 | LKQINV | 696.4403 | 696.4422 | −2.66 |
| b6* | LKQINV* | 696.4403 | 696.4422 | −2.79 |
| b7 | LKQINVI | 809.5244 | 809.5250 | −0.68 |
| b7* | LKQINVI* | 809.5244 | 809.5249 | −0.56 |
| b8 | LKQINVIA | 880.5615 | 880.5603 | 1.36 |
| b8* | LKQINVIA* | 880.5615 | 880.5601 | 1.63 |
| b9 | LKQINVIAG | 937.5829 | 937.5827 | 0.21 |
| b9* | LKQINVIAG* | 937.5829 | 937.5823 | 0.60 |
| b10 | LKQINVIAGV | 1036.6513 | 1036.6500 | 1.30 |
| b10* | LKQINVIAGV* | 1036.6513 | 1036.6494 | 1.88 |
| b11 | LKQINVIAGVK | 1164.7463 | 1164.7433 | 2.61 |
| b11* | LKQINVIAGVK* | 1164.7463 | 1164.7423 | 3.43 |
| b12 | LKQINVIAGVKE | 1293.7889 | 1293.7854 | 2.73 |
| b12* | LKQINVIAGVKE* | 1293.7889 | 1293.7840 | 3.77 |
| b13 | LKQINVIAGVKEP | 1390.8417 | 1390.8467 | −3.62 |
| b13* | LKQINVIAGVKEP* | 1390.8417 | 1390.8451 | −2.41 |
| b14 | LKQINVIAGVKEPI | 1503.9257 | 1503.9317 | −4.00 |
| b14* | LKQINVIAGVKEPI* | 1503.9257 | 1503.9296 | −2.61 |
| b15 | LKQINVIAGVKEPIR | 1660.0268 | 1660.0241 | 1.61 |
| b15* | LKQINVIAGVKEPIR* | 1660.0268 | Not Found | N/A |
| b16 | LKQINVIAGVKEPIRA | 866.0356 | 866.0350 | 0.73 |
| b16* | LKQINVIAGVKEPIRA* | 866.0356 | Not Found | N/A |
| b17 | LKQINVIAGVKEPIRAY | 947.5673 | 947.5654 | 1.96 |
| b17* | LKQINVIAGVKEPIRAY* | 947.5673 | 947.5651 | 2.37 |
| b18 | LKQINVIAGVKEPIRAYG | 976.0780 | 976.0772 | 0.85 |
| b18* | LKQINVIAGVKEPIRAYG* | 976.0780 | 976.0767 | 1.31 |
| b19 | LKQINVIAGVKEPIRAYGC | 1027.5826 | 1027.5855 | −2.87 |
| b19* | (−1 Da)LKQINVIAGVKEPIRAYGC* | 1027.0787 | Not Found | N/A |
| b20 | LKQINVIAGVKEPIRAYGCS | 1071.0986 | 1071.1022 | −3.37 |
| b20* | (−1 Da)LKQINVIAGVKEPIRAYGCS* | 1070.5947 | Not Found | N/A |
| b21 | LKQINVIAGVKEPIRAYGCSA | 1106.6172 | 1106.6198 | −2.38 |
| b21* | (−1 Da)LKQINVIAGVKEPIRAYGCSA* | 1106.1133 | Not Found | N/A |
| b22 | LKQINVIAGVKEPIRAYGCSAN | 776.0948 | 776.0952 | −0.49 |
| b22* | (−1 Da)LKQINVIAGVKEPIRAYGCSAN* | 775.7589 | Not Found | N/A |
| b23 | LKQINVIAGVKEPIRAYGCSAN^{D}D | 814.4372 | 814.4347 | 3.07 |
| b23* | (−2 Da)LKQINVIAGVKEPIRAYGCSAN^{D}D* | 813.7653 | 813.7627 | 3.19 |
| [M] | LKQINVIAGVKEPIRAYGCSAN^{D}DA | 844.1197 | 844.1190 | 0.83 |
| [M]* | (−2 Da) | 843.4478 | 843.4461 | 2.05 |
| | LKQINVIAGVKEPIRAYGCSAN^{D}DA* | | | |
| y23 | KQINVIAGVKEPIRAYGCSAN^{D}DA | 806.4250 | 806.4266 | −2.00 |
| y23* | (−2 Da)KQINVIAGVKEPIRAYGCSAN^{D}DA* | 805.7532 | 805.7537 | −0.58 |
| y22 | QINVIAGVKEPIRAYGCSAN^{D}DA | 763.7267 | Not Found | N/A |
| y22* | (−2 Da)QINVIAGVKEPIRAYGCSAN^{D}DA* | 763.0548 | 763.0568 | −2.58 |
| y21 | INVIAGVKEPIRAYGCSAN^{D}DA | 721.0405 | Not Found | N/A |
| y21* | (−2 Da)INVIAGVKEPIRAYGCSAN^{D}DA* | 720.3686 | 720.3678 | 1.11 |
| y20 | NVIAGVKEPIRAYGCSAN^{D}DA | 1024.5151 | 1024.5153 | −0.22 |
| y20* | (−2 Da)NVIAGVKEPIRAYGCSAN^{D}DA* | 1023.5073 | 1023.5048 | 2.49 |
| y19 | VIAGVKEPIRAYGCSAN^{D}DA | 967.4936 | 967.4942 | −0.58 |
| y19* | (−2 Da)VIAGVKEPIRAYGCSAN^{D}DA* | 966.4858 | 966.4885 | −2.78 |
| y18 | IAGVKEPIRAYGCSAN^{D}DA | 917.9594 | 917.9594 | −0.04 |
| y18* | (−2 Da)IAGVKEPIRAYGCSAN^{D}DA* | 916.9516 | 916.9511 | 0.60 |
| y17 | AGVKEPIRAYGCSAN^{D}DA | 861.4174 | 861.4188 | −1.65 |
| y17* | (−2 Da)AGVKEPIRAYGCSAN^{D}DA* | 860.4096 | Not Found | N/A |
| y16 | GVKEPIRAYGCSAN^{D}DA | 1650.7904 | 1650.7940 | −2.19 |
| y16* | (−2 Da)GVKEPIRAYGCSAN^{D}DA* | 1648.7748 | 1648.7736 | 0.74 |
| y15 | VKEPIRAYGCSAN^{D}DA | 1593.7690 | 1593.7630 | 3.74 |
| y15* | (−2 Da)VKEPIRAYGCSAN^{D}DA* | 1591.7533 | 1591.7561 | −1.77 |
| y14 | KEPIRAYGCSAN^{D}DA | 1494.7006 | 1494.6958 | 3.23 |
| y14* | (−2 Da)KEPIRAYGCSAN^{D}DA* | 1492.6849 | 1492.6817 | 2.14 |
| y13 | EPIRAYGCSAN^{D}DA | 1366.6056 | 1366.6087 | −2.29 |
| y13* | (−2 Da)EPIRAYGCSAN^{D}DA* | 1364.5899 | 1364.5851 | 3.54 |
| y12 | PIRAYGCSAN^{D}DA | 1237.5630 | 1237.5621 | 0.75 |
| y12* | (−2 Da)PIRAYGCSAN^{D}DA* | 1235.5473 | 1235.5505 | −2.61 |
| y11 | IRAYGCSAN^{D}DA | 1140.5102 | 1140.5108 | −0.50 |
| y11* | (−2 Da)IRAYGCSAN^{D}DA* | 1138.4946 | 1138.4944 | 0.20 |
| y10 | RAYGCSAN^{D}DA | 1027.4262 | 1027.4294 | −3.08 |
| y10* | (−2 Da)RAYGCSAN^{D}DA* | 1025.4105 | 1025.4095 | 1.02 |
| y9 | AYGCSAN^{D}DA | 871.3251 | 871.3222 | 3.38 |
| y9* | (−2 Da)AYGCSAN^{D}DA* | 869.3094 | 869.3070 | 2.76 |
| y8 | YGCSAN^{D}DA | 800.2879 | 800.2884 | −0.65 |
| y8* | (−2 Da)YGCSAN^{D}DA* | 798.2723 | 798.2739 | −1.99 |
| y7 | GCSAN^{D}DA | 637.2246 | 637.2263 | −2.66 |
| y7* | (−2 Da)GCSAN^{D}DA* | 635.2090 | 635.2099 | −1.48 |
| y6 | CSAN^{D}DA | 580.2032 | 580.2044 | −2.08 |
| y6* | (−2 Da)CSAN^{D}DA* | 578.1875 | 578.1887 | −2.00 |
| y5 | SAN^{D}DA | 477.1940 | 477.1941 | −0.27 |
| y5* | (−1 Da)SAN^{D}DA* | 476.1861 | Not Found | N/A |
| y4 | AN^{D}DA | 390.1619 | 390.1619 | −0.06 |
| y4* | (−1 Da)AN^{D}DA* | 389.1541 | Not Found | N/A |
| y3 | N^{D}DA | 319.1248 | 319.1254 | −1.78 |
| y3* | (−1 Da)N^{D}DA* | 318.1170 | Not Found | N/A |
| y2 | ^{D}DA | 205.0819 | 205.0816 | 1.28 |
| y2* | (−1 Da)D^{D}A* | 204.0741 | 204.0739 | 0.79 |

Unmodified indicates z = 1 charge state.
Underline indicates z = 2 charge state.
Italics indicates z = 3 charge state.
*Indicates the fragment originated from the modified peptide

TABLE 19
LKQINVIAGVKEPIRAYGPCSAN^{D}DA

| | | Expected | Observed | |
|---|---|---|---|---|
| | | Monoisotopic | Monoisotopic | Ppm |
| Ion | Sequence | Mass | Mass | Error |
| b2 | LK | 242.1863 | 242.1865 | −0.83 |
| b2* | LK* | 242.1863 | 242.1867 | −1.51 |
| b3 | LKQ | 370.2449 | 370.2450 | −0.40 |
| b3* | LKQ* | 370.2449 | 370.2455 | −1.67 |
| b4 | LKQI | 483.3289 | 483.3298 | −1.91 |
| b4* | LKQI* | 483.3289 | 483.3281 | 1.59 |
| b5 | LKQIN | 597.3719 | 597.3721 | −0.34 |
| b5* | LKQIN* | 597.3719 | 597.3734 | −2.45 |
| b6 | LKQINV | 696.4403 | 696.4415 | −1.76 |
| b6* | LKQINV* | 696.4403 | 696.4389 | 2.07 |
| b7 | LKQINVI | 809.5244 | 809.5241 | 0.34 |
| b7* | LKQINVI* | 809.5244 | 809.5263 | −2.41 |
| b8 | LKQINVIA | 880.5615 | 880.5593 | 2.46 |
| b8* | LKQINVIA* | 880.5615 | 880.5619 | −0.48 |
| b9 | LKQINVIAG | 937.5829 | 937.5816 | 1.37 |
| b9* | LKQINVIAG* | 937.5829 | 937.5845 | −1.72 |
| b10 | LKQINVIAGV | 1036.6513 | 1036.6486 | 2.56 |
| b10* | LKQINVIAGV* | 1036.6513 | 1036.6521 | −0.78 |
| b11 | LKQINVIAGVK | 1164.7463 | 1164.7416 | 3.99 |
| b11* | LKQINVIAGVK* | 1164.7463 | 1164.7459 | 0.34 |
| b12 | LKQINVIAGVKE | 1293.7889 | 1293.7834 | 4.22 |
| b12* | LKQINVIAGVKE* | 1293.7889 | 1293.7885 | 0.28 |
| b13 | LKQINVIAGVKEP | 1390.8417 | Not Found | N/A |
| b13* | LKQINVIAGVKEP* | 1390.8417 | 1390.8380 | 2.65 |
| b14 | LKQINVIAGVKEPI | 1503.9257 | 1503.9292 | −2.33 |
| b14* | LKQINVIAGVKEPI* | 1503.9257 | 1503.9220 | 2.48 |
| b15 | LKQINVIAGVKEPIR | 1660.0268 | 1660.0211 | 3.40 |
| b15* | LKQINVIAGVKEPIR* | 1660.0268 | Not Found | N/A |
| b16 | LKQINVIAGVKEPIRA | 866.0356 | 866.0340 | 1.82 |
| b16* | LKQINVIAGVKEPIRA* | 866.0356 | 866.0365 | −1.08 |
| b17 | LKQINVIAGVKEPIRAY | 947.5673 | 947.5643 | 3.13 |
| b17* | LKQINVIAGVKEPIRAY* | 947.5673 | 947.5673 | 0.01 |
| b18 | LKQINVIAGVKEPIRAYG | 976.0780 | 976.0760 | 2.05 |
| b18* | LKQINVIAGVKEPIRAYG* | 976.0780 | 976.0791 | −1.15 |
| b19 | LKQINVIAGVKEPIRAYG^{D}C | 1027.5826 | 1027.5843 | −1.62 |
| b19* | (−1 Da)LKQINVIAGVKEPI | 1027.0787 | 1027.0802 | −1.48 |
| | RAYG^{D}C* | | | |
| b20 | LKQINVIAGVKEPIRAYG^{D}CS | 1071.0986 | 1071.1008 | −2.08 |
| b20* | (−1 Da)LKQINVIAGVKEPIRA | 1070.5947 | 1070.5977 | −2.79 |
| | YG^{D}CS* | | | |
| b21 | LKQINVIAGVKEPIRAYG^{D}CSA | 1106.6172 | 1106.6184 | −1.06 |
| b21* | (−1 Da)LKQINVIAGVKEPIRAY | 1106.1133 | 1106.1162 | −2.62 |
| | G^{D}CSA* | | | |
| b22 | LKQINVIAGVKEPIRAYG^{D}CSAN | 776.0948 | 776.0944 | 0.50 |
| b22* | (−1 Da)LKQINVIAGVKEPIRAY | 775.7589 | 775.7583 | 0.83 |
| | G^{D}CSAN* | | | |
| b23 | LKQINVIAGVKEPIRAYG^{D}CSAN^{D}D | 814.4372 | 814.4394 | −2.67 |
| b23* | (−2 Da) | 813.7653 | 813.7642 | 1.32 |
| | LKQINVIAGVKEPIRAYG^{D}CSAN^{D}D* | | | |
| [M] | LKQINVIAGVKEPIRAYG^{D}CSAN^{D}DA | 844.1197 | 844.1194 | 0.31 |
| [M]* | (−2 Da) | 843.4478 | 843.4477 | 0.07 |
| | LKQINVIAGVKEPIRAYG^{D}CSAN^{D}DA* | | | |
| y23 | KQINVIAGVKEPIRAYG^{D}CSAN^{D}DA | 806.4250 | 806.4258 | −0.97 |
| y23* | (−2 Da) | 805.7532 | 805.7551 | −2.41 |
| | KQINVIAGVKEPIRAYG^{D}CSAN^{D}DA* | | | |
| y22 | QINVIAGVKEPIRAYG^{D}CSAN^{D}DA | 763.7267 | Not Found | N/A |
| y22* | (−2 Da)QINVIAGVKEPIRAYG^{D} | 763.0548 | 763.0530 | 2.31 |
| | CSAN^{D}DA* | | | |
| y21 | INVIAGVKEPIRAYG^{D}CSAN^{D}DA | 721.0405 | Not Found | N/A |
| y21* | (−2 Da)INVIAGVKEPIRAYG^{D} | 720.3686 | Not Found | N/A |
| | CSAN^{D}DA* | | | |
| y20 | NVIAGVKEPIRAYG^{D}CSAN^{D}DA | 1024.5151 | 1024.5140 | 1.03 |
| y20* | (−2 Da)NVIAGVKEPIRAYG^{D} | 1023.5073 | 1023.5074 | −0.14 |
| | CSAN^{D}DA* | | | |
| y19 | VIAGVKEPIRAYG^{D}CSAN^{D}DA | 967.4936 | 967.4930 | 0.61 |
| y19* | (−2 Da)VIAGVKEPIRAYG^{D} | 966.4858 | 966.4837 | 2.17 |
| | CSAN^{D}DA* | | | |
| y18 | IAGVKEPIRAYGPCSAN^{D}DA | 917.9594 | 917.9584 | 1.10 |
| y18* | (−2 Da)IAGVKEPIRAYG^{D} | 916.9516 | 916.9531 | −1.65 |
| | CSAN^{D}DA* | | | |
| y17 | AGVKEPIRAYG^{D}CSAN^{D}DA | 861.4174 | 861.4179 | −0.57 |
| y17* | (−2 Da)AGVKEPIRAYG^{D} | 860.4096 | 860.4082 | 1.59 |
| | CSAN^{D}DA* | | | |
| y16 | GVKEPIRAYG^{D}CSAN^{D}DA | 1650.7904 | 1650.7911 | −0.41 |
| y16* | (−2 Da)GVKEPIRAYG^{D} | 1648.7748 | 1648.7810 | −3.77 |
| | CSAN^{D}DA* | | | |
| y15 | VKEPIRAYG^{D}CSAN^{D}DA | 1593.7690 | 1593.7753 | −3.98 |
| y15* | (−2 Da)VKEPIRAYG^{D}CSAN^{D}DA* | 1591.7533 | 1591.7480 | 3.33 |
| y14 | KEPIRAYGPCSAN^{D}DA | 1494.7006 | 1494.7070 | −4.28 |
| y14* | (−2 Da)KEPIRAYG^{D}CSAN^{D}DA* | 1492.6849 | 1492.6878 | −1.94 |
| y13 | EPIRAYG^{D}CSAN^{D}DA | 1366.6056 | 1366.6066 | −0.73 |
| y13* | (−2 Da)EPIRAYG^{D}CSAN^{D}DA* | 1364.5899 | 1364.5901 | −0.17 |
| y12 | PIRAYGPCSAN^{D}DA | 1237.5630 | 1237.5603 | 2.20 |
| y12* | (−2 Da)PIRAYG^{D}CSAN^{D}DA* | 1235.5473 | 1235.5443 | 2.41 |
| y11 | IRAYGPCSAN^{D}DA | 1140.5102 | 1140.5092 | 0.86 |
| y11* | (−2 Da)IRAYG^{D}CSAN^{D}DA* | 1138.4946 | 1138.4978 | −2.80 |
| y10 | RAYGDCSAN^{D}DA | 1027.4262 | 1027.4281 | −1.83 |
| y10* | (−2 Da)RAYG^{D}CSAN^{D}DA* | 1025.4105 | 1025.4122 | −1.61 |
| y9 | AYGPCSAN^{D}DA | 871.3251 | 871.3273 | −2.53 |
| y9* | (−2 Da)AYG^{D}CSAN^{D}DA* | 869.3094 | 869.3088 | 0.69 |
| y8 | YGPCSAN^{D}DA | 800.2879 | 800.2876 | 0.37 |
| y8* | (−2 Da)YG^{D}CSAN^{D}DA* | 798.2723 | 798.2700 | 2.91 |
| y7 | GPCSAN^{D}DA | 637.2246 | 637.2258 | −1.83 |
| y7* | (−2 Da)G^{D}CSAN^{D}DA* | 635.2090 | 635.2107 | −2.61 |
| y6 | DCSAN^{D}DA | 580.2032 | 580.2040 | −1.32 |
| y6* | (−2 Da)DCSAN^{D}DA* | 578.1875 | 578.1859 | 2.82 |
| y5 | SAN^{D}DA | 477.1940 | 477.1938 | 0.34 |
| y5* | (−1 Da)SAN^{D}DA* | 476.1861 | Not Found | N/A |
| y4 | AN^{D}DA | 390.1619 | 390.1617 | 0.43 |
| y4* | (−1 Da)AN^{D}DA* | 389.1541 | Not Found | N/A |
| y3 | N^{D}DA | 319.1248 | 319.1253 | −1.41 |
| y3* | (−1 Da)N^{D}DA* | 318.1170 | 318.1170 | 0.14 |
| y2 | ^{D}DA | 205.0819 | 205.0816 | 1.43 |
| y2* | (−1 Da)^{D}DA* | 204.0741 | Not Found | N/A |

Unmodified indicates z = 1 charge state.
Underline indicates z = 2 charge state.
Italics indicates z = 3 charge state.
*Indicates the fragment originated from the modified peptide

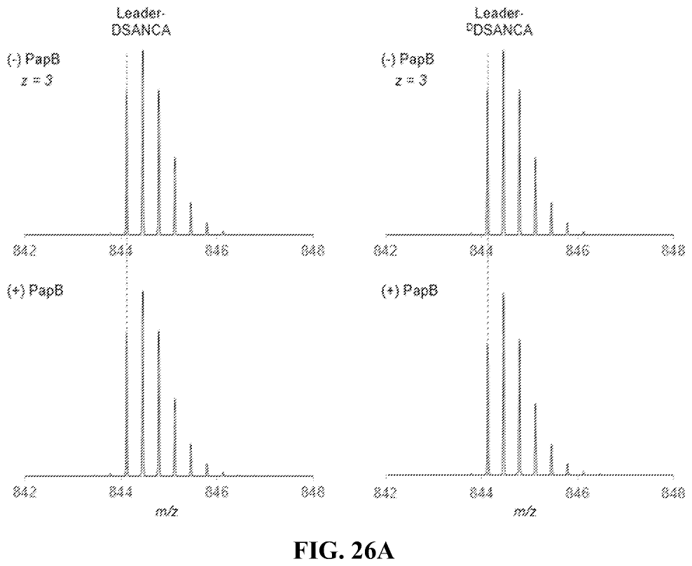

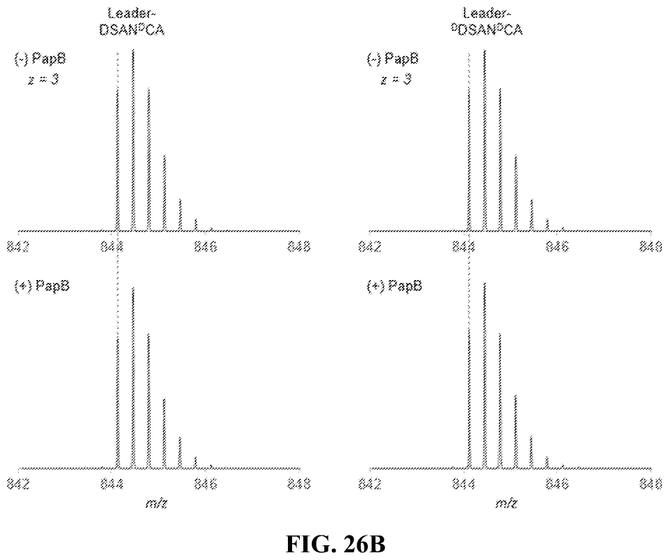

Next, it was attempted to transpose the Cys and Asp residues by using a DSANCA motif attached to the leader. However, we were unable to observe any crosslinked product with the transposed peptides, either with L- or D-amino acids (FIG. 26). These data support the notion that the active site has substantial flexibility with regards to the Cys, but that the interaction with Asp limit the range of available productive conformations. Previous studies have shown that mutation of a conserved PapB Arg residue, which may be near the PapA peptide Asp binding site, to an Ala abolishes activity. Inversion of the sidechain would similarly eliminate the interaction leading to no crosslinking.

d. PapB Processes Sequences Unrelated to the Wild Type Peptide Sequence

The results presented in the previous sections highlight the remarkable lack of sequence specificity in PapB, suggesting that the enzyme may be able to cross link virtually any sequence that is tethered to the leader sequence, so long as a Cys and a downstream Asp/Glu residue is present in the peptide. As a proof of concept, the use of PapB to generate an analog of octreotide was explored. Octreotide is an FDA-approved drug used to treat excessive human growth hormone production, to control symptoms in several types of cancers, and to treat gastrointestinal bleeding (Lamberts, S. W. J., et al. Eur. J. Endocrinol. 2019, 181, R173-R183). Octreotide has two D-amino acids, making it less susceptible to protease degradation in vivo (Muttenthaler, M., et al. Nat. Rev. Drug Discov. 2021, 20, 309-325).

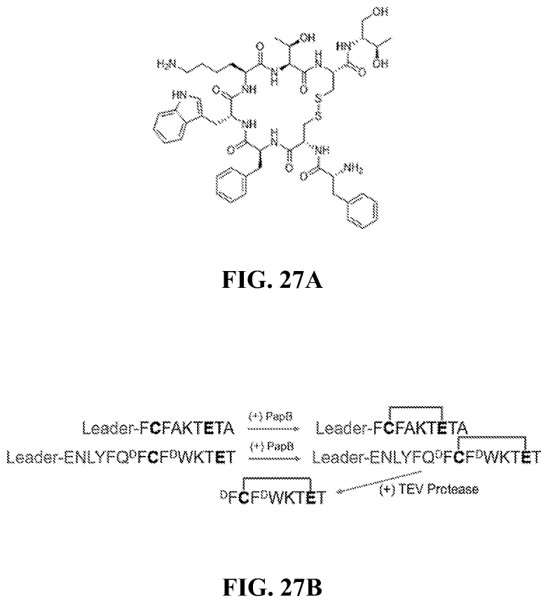

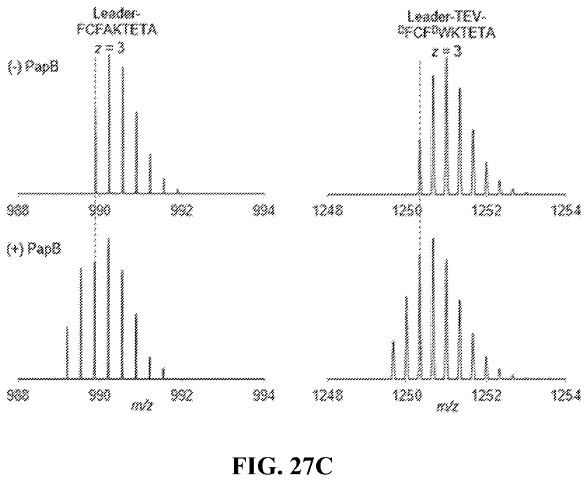

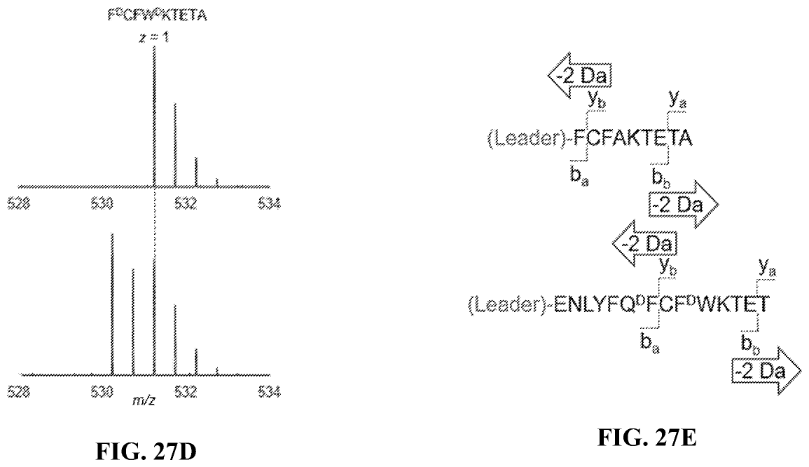

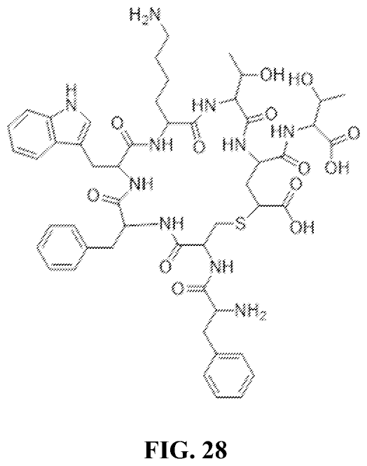

Octreotide is an 8-mer peptide with the sequence ^{D}FCF^{D}WKTCT, with D-amino acids at the first and fourth positions. The two C residues form a disulfide-linked macrocycle. It has been noted that WT-PapA contains positively charged, nonpolar, polar uncharged, and bulky side-chain residues between the six donor and acceptor residue motifs (Precord, T. W., et al. ACS Chem. Biol. 2019, 14 (9), 1981-89). This observation, in combination with the successful crosslinking in an expanded motif, suggested that PapB may be able to introduce disulfide mimetic bonds via a thioether in a variety of peptide substrates so long as a thiol and carboxylate moiety are present. As a proof of concept, two octreotide analogs were synthesized. Both designs dispensed with the C-terminal Cys in favor of a Glu, which was used to cross link to the Cys with PapB. In the first design attempt, the sequence was further simplified by replacing ^{D}W4 with Ala (FIG. 27A). The octreotide analog sequence was covalently attached to the PapA leader peptide by solid-phase peptide synthesis (SPPS). The second design contained only the C7E replacement, but to facilitate removal of the leader peptide, an ENLYFQ sequence was incorporated between the leader and the peptide to provide a convenient site for TEV cleavage. The incubation of either of the designed octreotide analogs with PapB leads to formation of a new product. In each case, the product is 2 Da lighter than the starting material, consistent with the formation of a crosslink (FIG. 27B). An intrapeptide disulfide can be eliminated as the source of this loss because the peptide only contains one Cys residue. It is noted that the reaction is ˜75% complete with this analog, as assessed from the isotopic envelope. However, the observed monoisotopic masses for each peptide products species are in good agreement with the expected monoisotopic masses for a single cross link (<3 ppm, FIG. 27B and FIG. 27C, Table 20). A structure of the synthesized octreotide analog can be found in FIG. 28. Subsequent MS/MS analysis corroborates the initial mass spectrometry data; the fragmentation pattern of the peptide depicts small fragments between the crosslinked Cys and Glu due to incomplete crosslinking. There is a clear 2 Da loss pattern in every y-fragment after the Cys and a 2 Da loss in every b-fragment after the C-terminal Glu with the modified peptide (Tables 21-22).

TABLE 20

| | Expected | Observed | |
|---|---|---|---|
| | Mono- | Mono- | |
| | isotopic | isotopic | Ppm |
| Sequence | Mass | Mass | Error |
| Leader- | (−)PapB: 989.8757 | 989.8746 | −1.111 |
| FCFAKTETA | (+)PapB (−2 Da): | 989.2042 | 0.404 |
| (z = 3) | 989.2038 | | |
| Leader-ENLYFQ | (−)PapB: 1250.3235 | 1250.3226 | −0.720 |
| DFCFDWTET | (+)PapB (−2 Da): | 1249.6507 | −0.720 |
| (z = 3) | 1249.6516 | | |
| DFCFWDKTET | (−)PapB: 531.2417 | 531.2427 | 1.882 |
| | (+)PapB (−2 Da): | 530.2354 | 2.829 |
| | 530.2339 | | |

TABLE 21
LKQINVIAGVKEPIRAYGFCFAKTETA

| | | Expected | Observed | |
|---|---|---|---|---|
| | | Monoisotopic | Monoisotopic | Ppm |
| Ion | Sequence | Mass | Mass | Error |
| b2* | LK* | 242.1863 | 242.1862 | 0.40 |
| b3* | LKQ* | 370.2449 | 370.2444 | 1.26 |
| b4* | LKQI* | 483.3289 | 483.3289 | 0.07 |
| b5* | LKQIN* | 597.3719 | 597.3708 | 1.93 |
| b6* | LKQINV* | 696.4403 | 696.4398 | 0.73 |
| b7* | LKQINVI* | 809.5244 | 809.5219 | 3.08 |
| b8* | LKQINVIA* | 880.5615 | 880.5630 | −1.70 |
| b9* | LKQINVIAG* | 937.5829 | 937.5856 | −2.89 |
| b10* | LKQINVIAGV* | 1036.6513 | 1036.6533 | −1.89 |
| b11* | LKQINVIAGVK* | 1164.7463 | 1164.7471 | −0.69 |
| b12* | LKQINVIAGVKE* | 1293.7889 | 1293.7898 | −0.68 |
| b13* | LKQINVIAGVKEP* | 1390.8417 | 1390.8393 | 1.74 |
| b14* | LKQINVIAGVKEPI* | 1503.9257 | 1503.9232 | 1.63 |
| b15* | LKQINVIAGVKEPIR* | 1660.0268 | 1660.0302 | −2.05 |
| b16* | LKQINVIAGVKEPIRA* | 866.0356 | 866.0376 | −2.31 |
| b17* | LKQINVIAGVKEPIRAY* | 947.5673 | Not Found | N/A |
| b18* | LKQINVIAGVKEPIRAYG* | 976.0780 | 976.0802 | −2.29 |
| b19* | LKQINVIAGVKEPIRAYGF* | 1049.6122 | Not Found | N/A |
| b20* | LKQINVIAGVKEPIRAYGFC* | 1100.6129 | Not Found | N/A |
| b21* | LKQINVIAGVKEPIRAYGFCF* | 783.1005 | Not Found | N/A |
| b22* | LKQINVIAGVKEPIRAYGFCFA* | 806.7795 | Not Found | N/A |
| b23* | LKQINVIAGVKEPIRAYGFCFAK* | 849.4779 | Not Found | N/A |
| b24* | LKQINVIAGVKEPIRAYGFCFAKT* | 883.1604 | Not Found | N/A |
| b25* | LKQINVIAGVKEPIRAYGFCFAKTE* | 925.8387 | 925.8365 | 2.38 |
| b26* | LKQINVIAGVKEPIRAYGFCFAKTET* | 959.5212 | 959.5200 | 1.28 |
| [M]* | LKQINVIAGVKEPIRAYGFCFAKTETA* | 989.2038 | 989.2037 | 0.06 |
| y26* | KQINVIAGVKEPIRAYGFCFAKTETA* | 951.5091 | 951.5088 | 0.36 |
| y25* | QINVIAGVKEPIRAYGFCFAKTETA* | 908.8108 | 908.8126 | −2.02 |
| y24* | INVIAGVKEPIRAYGFCFAKTETA* | 866.1246 | 866.1222 | 2.77 |
| y23* | NVIAGVKEPIRAYGFCFAKTETA* | 828.4299 | 828.4302 | −0.40 |
| y22* | VIAGVKEPIRAYGFCFAKTETA* | 790.4156 | 790.4152 | 0.46 |
| y21* | IAGVKEPIRAYGFCFAKTETA* | 757.3928 | 757.3936 | −1.00 |
| y20* | AGVKEPIRAYGFCFAKTETA* | 1079.0435 | 1079.0453 | −1.65 |
| y19* | GVKEPIRAYGFCFAKTETA* | 1043.5249 | Not Found | N/A |
| y18* | VKEPIRAYGFCFAKTETA* | 1015.0142 | 1015.0142 | 0.00 |
| y17* | KEPIRAYGFCFAKTETA* | 965.4800 | 965.4811 | −1.18 |
| y16* | EPIRAYGFCFAKTETA* | 901.4325 | 901.4332 | −0.77 |
| y15* | PIRAYGFCFAKTETA* | 1672.8152 | 1672.8202 | −2.99 |
| y14* | IRAYGFCFAKTETA* | 1575.7624 | 1575.7599 | 1.58 |
| y13* | RAYGFCFAKTETA* | 1462.6784 | 1462.6848 | −4.38 |
| y12* | AYGFCFAKTETA* | 1306.5773 | 1306.5724 | 3.78 |
| y11* | YGFCFAKTETA | 1235.5401 | 1235.5352 | 3.93 |
| y10* | GFCFAKTETA* | 1072.4768 | 1072.4783 | −1.44 |
| y9* | FCFAKTETA* | 1015.4553 | 1015.4590 | −3.68 |
| y8* | CFAKTETA* | 868.3869 | 868.3869 | 0 |
| y7* | FAKTETA* | 766.3856 | Not Found | N/A |
| y6* | AKTETA* | 619.3172 | Not Found | N/A |
| y5* | KTETA* | 548.2800 | Not Found | N/A |
| y4* | TETA* | 420.1851 | Not Found | N/A |
| y3* | ETA* | 319.1374 | Not Found | N/A |
| y2* | TA* | 191.1026 | 191.1023 | 1.53 |

Unmodified indicates z = 1 charge state.
Underline indicates z = 2 charge state.
Italics indicates z = 3 charge state.
* Indicates the fragment originated from the modified peptide

TABLE 22
LKQINVIAGVKEPIRAYGENLYFQ^{D}FCF^{D}WKTET

| | | Expected | Observed | |
|---|---|---|---|---|
| | | Mono- | Mono- | |
| | | isotopic | isotopic | Ppm |
| Ion | Sequence | Mass | Mass | Error |
| b2* | LK* | 242.1863 | 242.1863 | 0.16 |
| b3* | LKQ* | 370.2449 | 370.2442 | 1.82 |
| b4* | LKQI* | 483.3289 | 483.3283 | 1.24 |
| b5* | LKQIN* | 597.3719 | 597.3732 | −2.16 |
| b6* | LKQINV* | 696.4403 | 696.4383 | 2.86 |
| b7* | LKQINVI* | 809.5244 | 809.5253 | −1.08 |
| b8* | LKQINVIA* | 880.5615 | 880.5605 | 1.16 |
| b9* | LKQINVIAG* | 937.5829 | 937.5827 | 0.17 |
| b10* | LKQINVIAGV* | 1036.6513 | 1036.6497 | 1.51 |
| b11* | LKQINVIAGVK* | 1164.7463 | 1164.7426 | 3.14 |
| b12* | LKQINVIAGVKE* | 1293.7889 | 1293.7843 | 3.56 |
| b13* | LKQINVIAGVKEP* | 1390.8417 | Not Found | N/A |
| b14* | LKQINVIAGVKEPI* | 1503.9257 | 1503.9298 | −2.71 |
| b15* | LKQINVIAGVKEPIR* | 830.5171 | 830.5180 | −1.14 |
| b16* | LKQINVIAGVKEPIRA* | 866.0356 | 866.0352 | 0.49 |
| b17* | LKQINVIAGVKEPIRAY* | 947.5673 | 947.5655 | 1.94 |
| b18* | LKQINVIAGVKEPIRAYE* | 1012.0886 | 1012.0883 | 0.25 |
| b19* | LKQINVIAGVKEPIRAYEN* | 1069.1100 | 1069.1100 | 0.03 |
| b20* | LKQINVIAGVKEPIRAYENL* | 1125.6521 | 1125.6530 | −0.79 |
| b21* | LKQINVIAGVKEPIRAYENLY* | 1207.1837 | 1207.1862 | −2.06 |
| b22* | LKQINVIAGVKEPIRAYENLYF* | 854.1477 | 854.1463 | 1.61 |
| b23* | LKQINVIAGVKEPIRAYENLYFQ* | 896.8339 | 896.8345 | −0.69 |
| b24* | LKQINVIAGVKEPIRAYENLYFQF* | 945.8567 | 945.8596 | −3.06 |
| b25* | LKQINVIAGVKEPIRAYENLYFQFC* | 979.8572 | Not Found | N/A |
| b26* | LKQINVIAGVKEPIRAYENLYFQFCF* | 1028.8800 | Not Found | N/A |
| b27* | LKQINVIAGVKEPIRAYENLYFQFCFW* | 1090.9064 | Not Found | N/A |
| b28* | LKQINVIAGVKEPIRAYENLYFQFCFWK* | 1133.6047 | Not Found | N/A |
| b29* | LKQINVIAGVKEPIRAYENLYFQFCFWKT* | 1167.2873 | Not Found | N/A |
| b30* | LKQINVIAGVKEPIRAYENLYFQFCFWKTE* | 1209.9655 | 1209.9655 | 0.01 |
| [M]* | LKQINVIAGVKEPIRAYENLYFQFCFWKTET* | 1249.6516 | 1249.6538 | −1.75 |
| y30* | KQINVIAGVKEPIRAYENLYFQFCFWKTET* | 1211.9569 | 1211.9537 | 2.62 |
| y29* | QINVIAGVKEPIRAYENLYFQFCFWKTET* | 1169.2586 | 1169.2607 | −1.82 |
| y28* | INVIAGVKEPIRAYENLYFQFCFWKTET* | 1126.5724 | 1126.5758 | −3.06 |
| y27* | NVIAGVKEPIRAYENLYFQFCFWKTET* | 1088.8777 | 1088.8819 | −3.86 |
| y26* | VIAGVKEPIRAYENLYFQFCFWKTET* | 1050.8634 | 1050.8645 | −1.01 |
| y25* | IAGVKEPIRAYENLYFQFCFWKTET* | 1017.8406 | 1017.8382 | 2.39 |
| y24* | AGVKEPIRAYENLYFQFCFWKTET* | 980.1459 | Not Found | N/A |
| y23* | GVKEPIRAYENLYFQFCFWKTET* | 956.4669 | 956.4659 | 1.06 |
| y22* | VKEPIRAYENLYFQFCFWKTET* | 1405.6860 | 1405.6838 | 1.54 |
| y21* | KEPIRAYENLYFQFCFWKTET* | 1356.1518 | 1356.1510 | 0.58 |
| y20* | EPIRAYENLYFQFCFWKTET* | 1292.1043 | 1292.1088 | −3.51 |
| y19* | PIRAYENLYFQFCFWKTET* | 1227.5830 | 1227.5785 | 3.64 |
| y18* | IRAYENLYFQFCFWKTET* | 1179.0566 | 1179.0574 | −0.64 |
| y17* | RAYENLYFQFCFWKTET* | 1122.5146 | 1122.5166 | −1.81 |
| y16* | AYENLYFQFCFWKTET* | 1044.4640 | 1044.4647 | −0.66 |
| y15* | YENLYFQFCFWKTET* | 1008.9455 | 1008.9429 | 2.59 |
| y14* | ENLYFQFCFWKTET* | 927.4138 | Not Found | N/A |
| y13* | NLYFQFCFWKTET* | 862.8925 | Not Found | N/A |
| y12* | LYFQFCFWKTET* | 1610.7348 | 1610.7303 | 2.81 |
| y11* | YFQFCFWKTET* | 1497.6507 | 1497.6439 | 4.52 |
| y10* | FQFCFWKTET* | 1334.5874 | 1334.5889 | −1.11 |
| y9* | QFCFWKTET* | 1187.5190 | 1187.5209 | −1.61 |
| y8* | FCFWKTET* | 1059.4604 | 1059.4621 | −1.64 |
| y7* | CFWKTET* | 912.3920 | 912.3932 | −1.29 |
| y6* | FWKTET* | 810.3907 | Not Found | N/A |
| y5* | WKTET* | 663.3222 | Not Found | N/A |
| y4 | KTET* | 477.2429 | Not Found | N/A |
| y3* | TET* | 349.1480 | Not Found | N/A |
| y2* | ET* | 248.1003 | Not Found | N/A |

Unmodified indicates z = 1 charge state.
Underline indicates z = 2 charge state.
Italics indicates z = 3 charge state.
*Indicates the fragment originated from the modified peptide

Next, it was attempted to use TEV protease to release the modified peptide to show the feasibility of the use of this method to generate a novel octreotide analog. Other than Pro, the TEV protease can accommodate other amino acids at the P1′ position (Kapust, R. B., et al. Biochem. Biophy. Res. Commun. 2002, 294 (5), 949-955), but G or S are preferred. Residues other than G or S are acceptable at the P1′ position, though they result in diminished enzymatic efficacy. A crystal structure of a catalytically inactive form of TEV protease that was co-crystallized with an oligopeptide substrate revealed that the side chain of the residue at the P1′ position is partially exposed to solvent (Phan, J., et al. J. Biol. Chem. 2002, 277 (52), 50564-60672). D-amino acids have likely not been tested at the P1′ position. When treated with TEV protease, the peptide containing the TEV cleavage site undergoes cleavage to release the C-terminal fragment (FIG. 27C). The results highlight that DF is tolerated the P1′ position. This proof-of-concept experiment demonstrates that PapB and TEV protease can be used together to generate therapeutic analogs from synthetic peptide substrates that contain both Cys and Asp/Glu residues, where PapB installs thioether bond(s) between Cys and Asp/Glu to replace disulfide bridges.

These findings support the notion that PapB can modify peptides with large spacing between the thiol and carboxylate moieties as well as sequences unrelated to PapA. These initial results indicate that PapB has utility as a bimoiety-dependent thioether installation tool; (1) it is tolerant of a variety of sidechains spanning the peptide between the donor and acceptor Cys and Asp/Glu residues, (2) the orientation as well as spacing of the carboxylate and thiol moieties is flexible, and (3) TEV recognition sequences can be introduced to allow for modified peptides to be isolated from the leader sequence.

e. Discussion

In the 20 years since Sofia and coworkers established the RS superfamily (Sofia, H. J., et al. Nucleic Acids Res. 2001, 29 (5), 1097-1106), there has been an explosion of complex transformations that are attributable to RS enzymes. RS enzymes vastly expand the biochemical reaction repertoire because of their ability to activate C—H bonds for a variety of transformations, which can range from epimerizations to bonds to other carbon atoms or to heteroatoms. PapB catalyzes one such transformation, which entails activation of the carbon adjacent to a carboxylate moiety to crosslink to the thiol sidechain of Cys (Precord, T. W., et al. ACS Chem. Biol. 2019, 14 (9), 1981-89). The mechanistic details of thioether crosslink formation remain to be elucidated. However, these results highlights hitherto unknown promiscuity in PapA/B that will have implications in mechanism of substrate recognition.

Based on all available structural and biochemical data on RiPP maturase proteins, one would expect that the leader sequence binds to the RiPP recognition element (RRE) domain and directs the peptide to the active site of the protein to be modified. Implicit in this is the assumption that the specificity in the substrate selection is governed by the binding energy of interactions with the leader sequence to the RRE domain. A conserved Asn sidechain in the leader sequence has been proposed previously as being required for the peptide-RRE interaction. The results that show PapB can accept substrates with Cys-to-Asp separation ranging from 0-6 amino acids, perhaps is evidence for this in that the binding energy for interactions with the leader sequence is leveraged towards reactivity. However, when these studies initially began, it was assumed that the 3 amino acid separation likely meant that the peptide has helical structure, as has been proposed previously (King, A. M., et al. Nat. Commun. 2021, 12, 6343), which would place the sidechains of the Cys and Asp residues near one another in three-dimensional space. The observation that the enzyme can accept substrate with variable Cys-to-Asp spacing, however, suggests that the recognition relies on the specific sidechain and not the secondary structure. In other words, the enzyme specifically recognizes the Cys and Asp/Glu sidechains. Since there are no structural data on this enzyme, it is difficult to know how this may be accomplished, but it is noted that it has been proposed that in the thioether crosslinking enzymes, the thiolate of the Cys can interact with one of the auxiliary Fe—S clusters. One can imagine that the recognition of the Asp/Glu may involve a hydrophilic or positively charged patch of residues. An Arg residue in PapB (Arg372) has previously been implicated by sequence alignments, mutation of which abolished crosslinking activity. Therefore, the model for recognition that best fits the data is one where the peptide to be modified has only two albeit very specific interactions with the enzymes, outside of the leader sequence.

The observation that crosslinking efficiency is decreased when the separation is zero or six likely results from either constraint on the degrees of freedom in the shorter span, or the presence of too many possible conformations in the longer separation, both of which would lead to fewer productive interactions between the residues to be crosslinked and the specific locations in which they bind. Additional evidence for the absence of significant sequence dependence, other than the identity of the Cys and Asp/Glu is the fact that D-amino acids are tolerated. Outside of the leader sequence, it is proposed that there are no specific interactions between the enzyme and the rest of the peptide other than the binding of the thiolate and carboxylate. As with other RS enzymes, one can anticipate that the binding occurs to place the 5′-position of SAM within or near van Der Waals radius of the H-atom to be abstracted, which is in turn, within close proximity of the crosslinking Cys sulfur.

While there are several examples of promiscuity by rSAM RiPP maturases including hybrid RiPPs produced using chimeric leader peptides (Burkhart, B. J, et al. ACS Cent. Sci. 2017, 3 (6), 629-638), RiPPs with acceptor residue alterations (Himes, P. M., et al. ACS Chem. Biol. 2016, 11 (6), 1737-1744), truncated (i.e., leaderless) peptides being accepted by epimerases (Himes, P. M., et al. ACS Chem. Biol. 2016, 11 (6), 1737-1744), changes in the macrocycle core being tolerated (King, A. M, et al. Nat. Commun. 2021, 12, 6343), and residue-epimerization shifts based on core sequence changes (Korneli, M., et al. ACS Synth. Biol. 2021, 10 (2), 236-242), this work demonstrates a highly predictable pattern for crosslink formation.

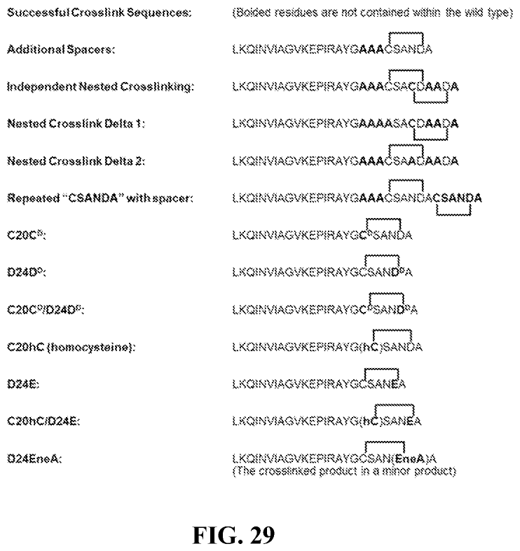

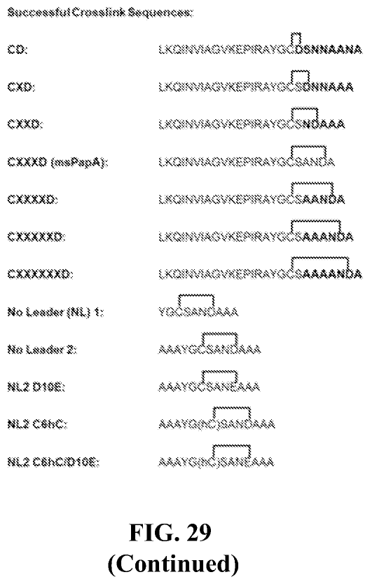

FIG. 29 provides a brief summary of successful PapB-mediated thioether crosslinks in tested peptide sequences.

### 4. The Leader Peptide Sequence is not Required

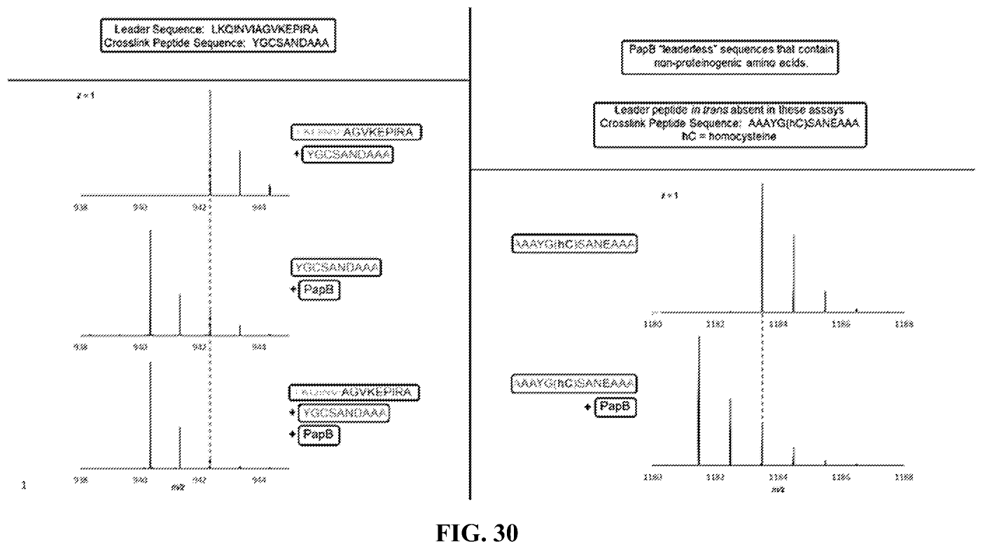

As shown in FIG. 30, the leader peptide sequence is not required for modification via PapB. PapB “leaderless” sequences that contain non-proteinogenic amino acids still demonstrated thioether linkages via mass spectrometry.

### 5. Interpeptide Crosslink Studies

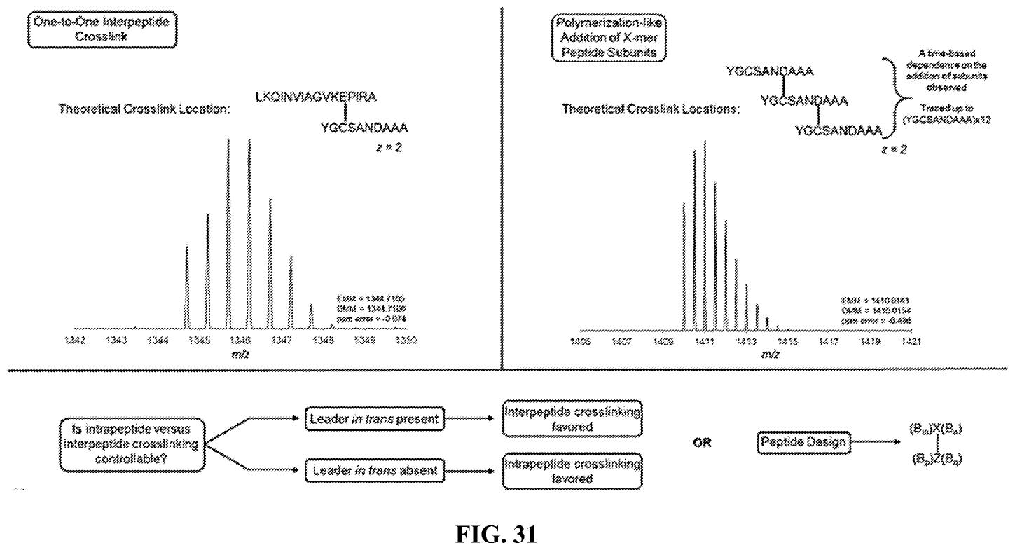

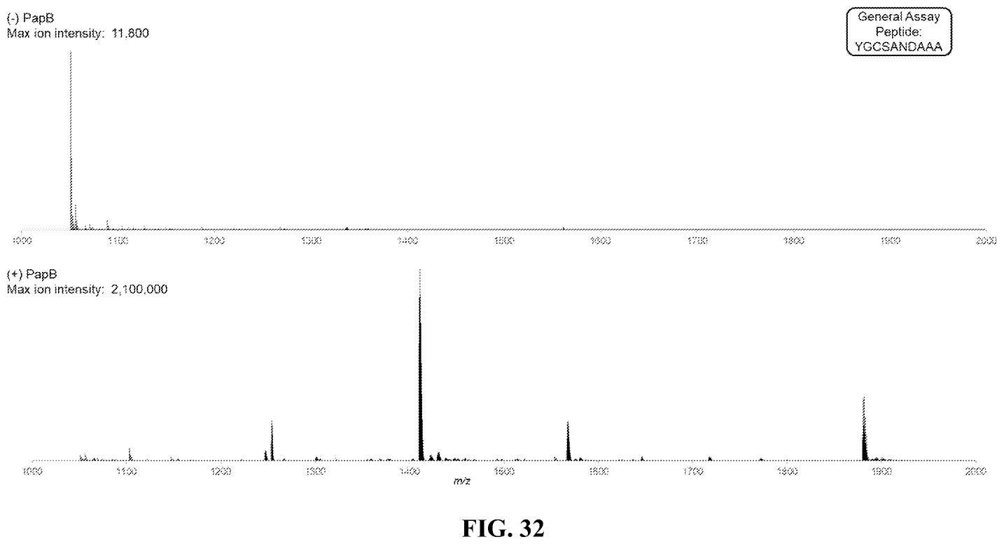

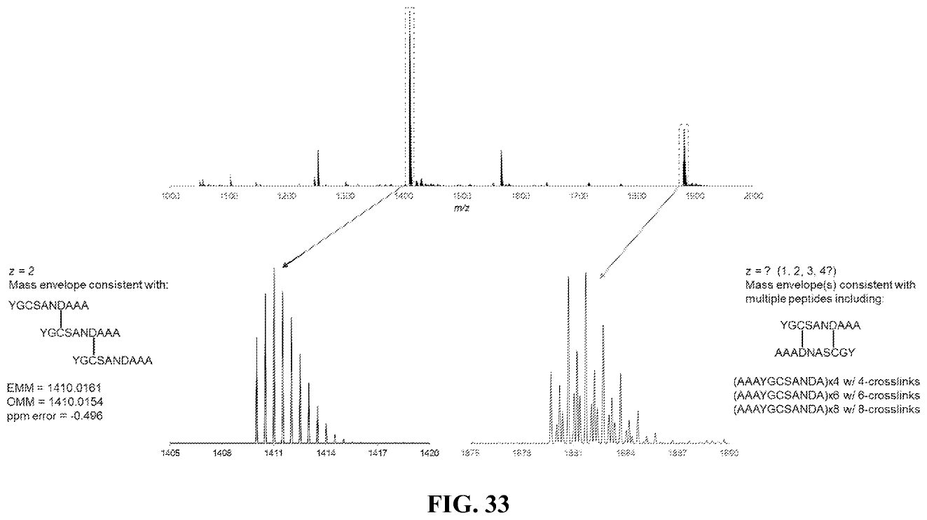

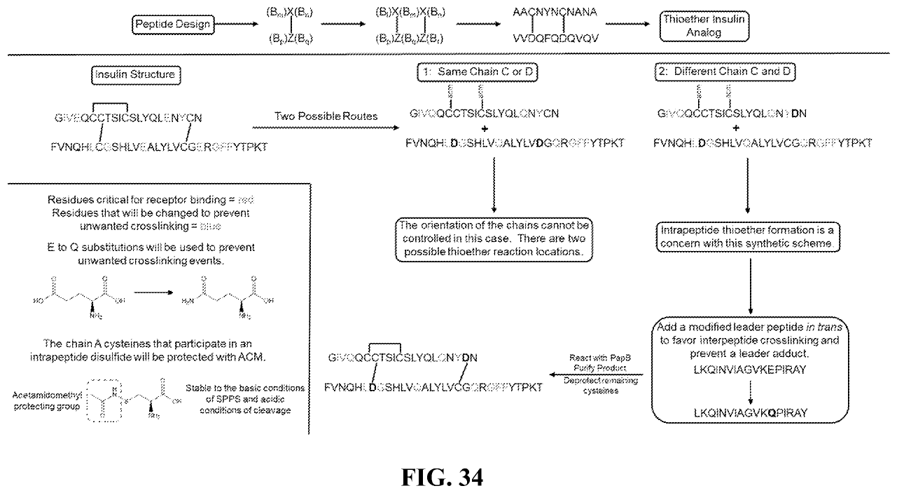

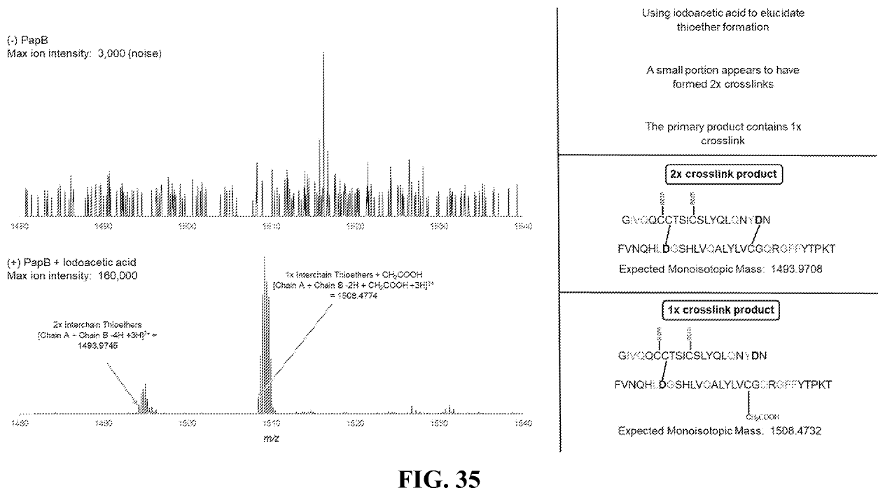

As shown in FIG. 31-FIG. 35, mass spectrometry results reveal evidence that interpeptide crosslinking can also be achieved with PapB. FIG. 31 shows mass spectrometry data for a one-to-one interpeptide crosslink as well as polymerization-like addition of X-mer peptide subunits. FIG. 32 shows results for a general assay peptide before and after PapB, demonstrating the presence of interpeptide products. FIG. 33 shows mass spectrometry results showing evidence of simple and complex mass envelopes. As a proof-of-concept for interpeptide crosslinking using PapB, thioether insulin analogs were synthesized. The results showing the crosslinked products can be found in FIG. 34 and FIG. 35.

### 6. Additional Studies

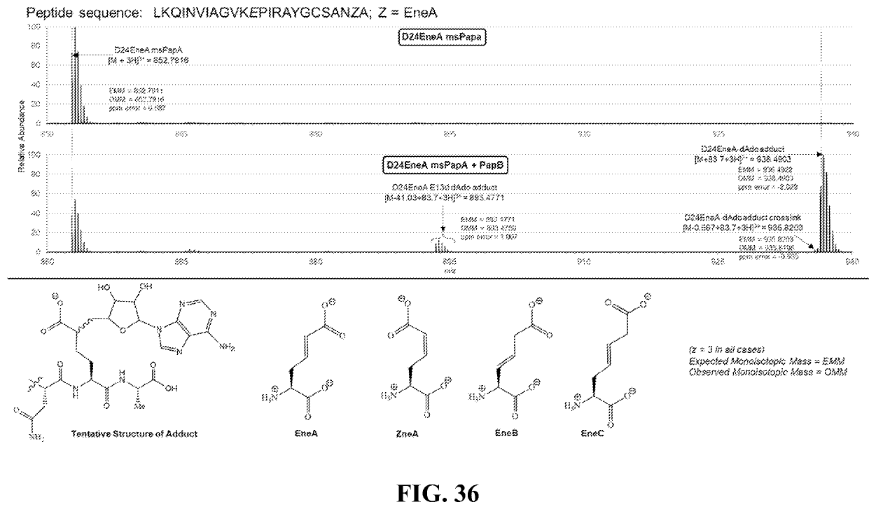

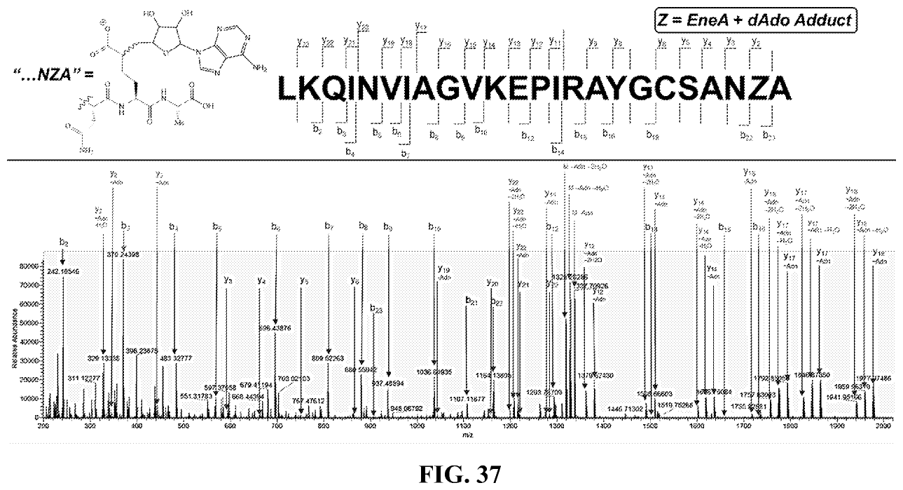

Results showing the crosslinking that occurs in studies where the peptide sequence contains (S,E)-5-aminohex-2-enedioic acid are shown in FIG. 36. Tandem mass spectrometry results of dADo+msPapA adduct are shown in FIG. 37.

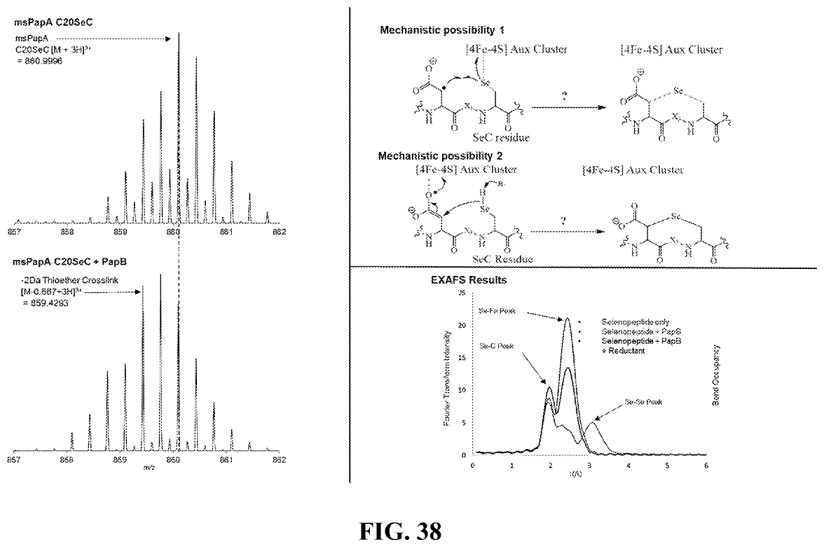

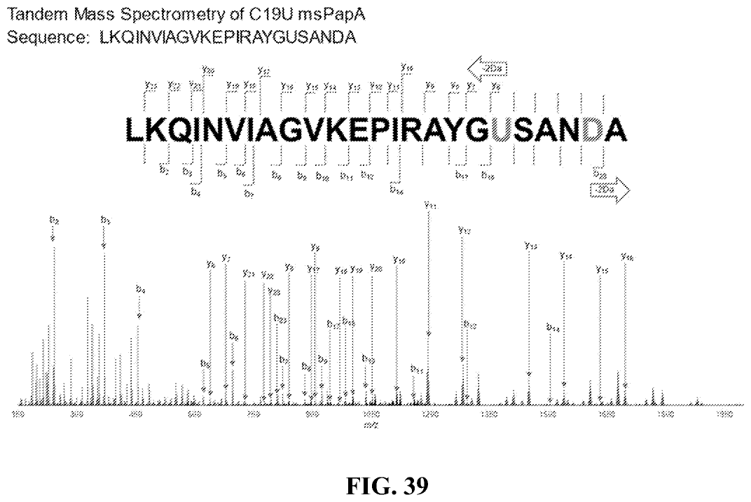

Studies demonstrating that thioether crosslinking occurs via PapB in peptides that contain a selenopeptide sequence were performed, and the results (mass spectrometry and EXAFS) are shown in FIG. 38. The tandem mass spectrometry results of C19U msPapA is shown in FIG. 39.

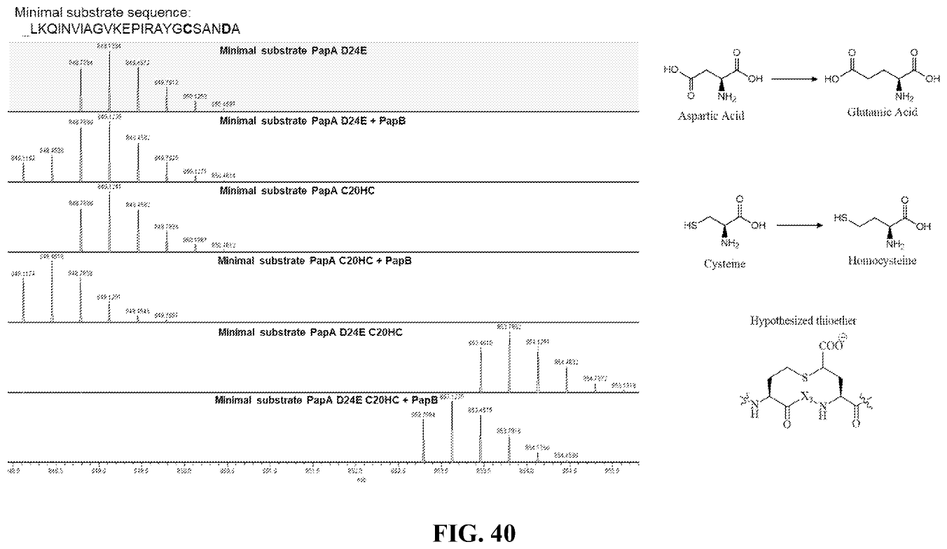

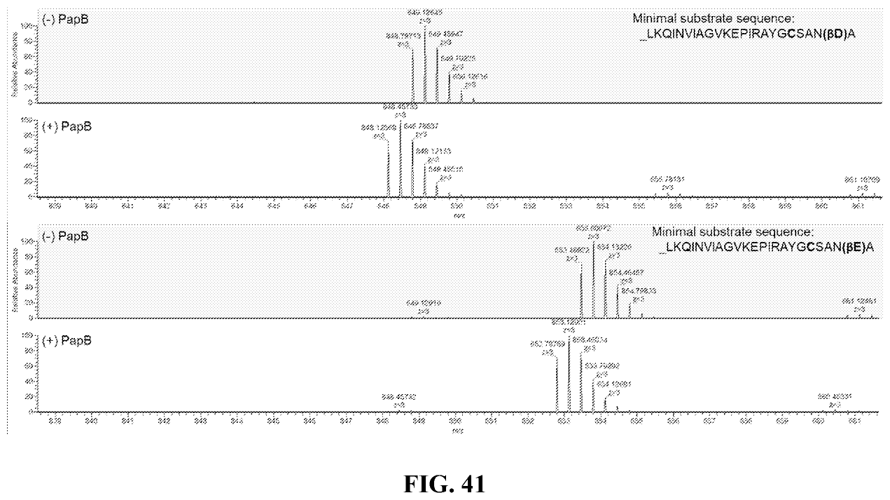

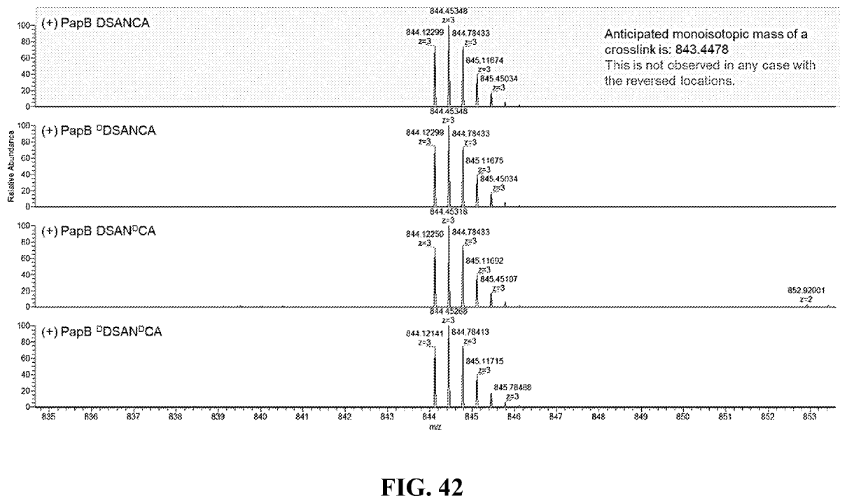

Donor and acceptor substitutions were explored, and the successful crosslinking results are shown in FIG. 40 and FIG. 41. However, it was found that interchanging the position of the model system C and D residues resulted in no crosslinking (FIG. 42).

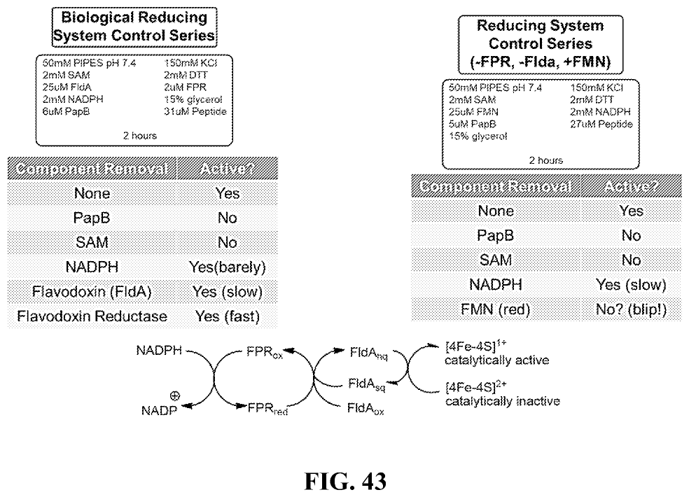

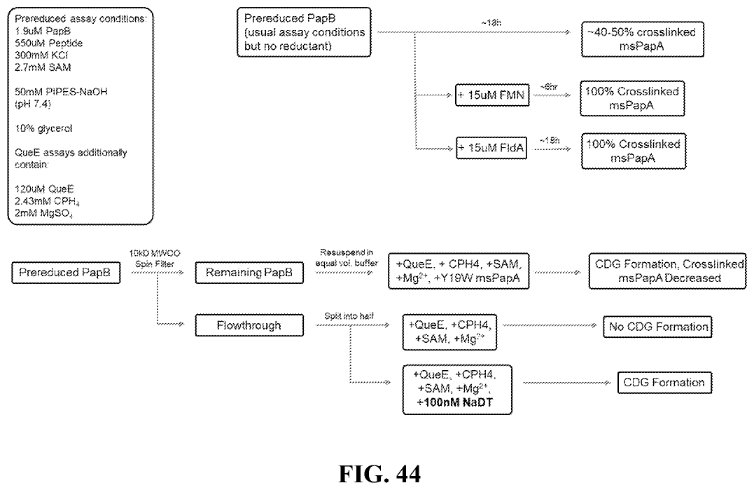

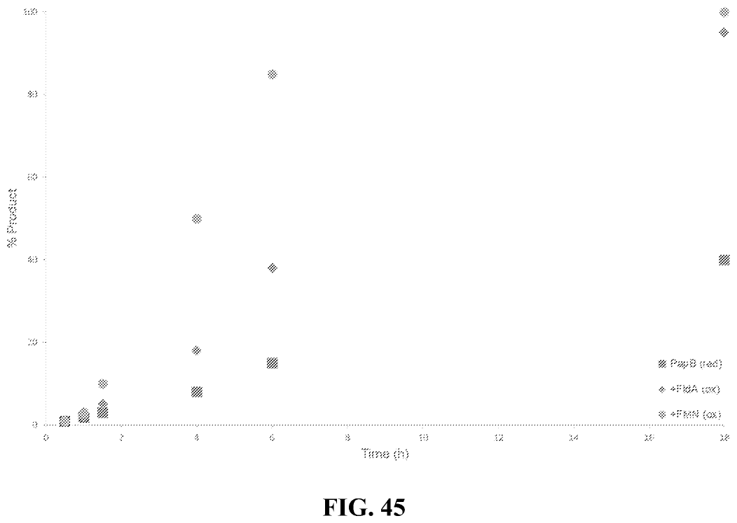

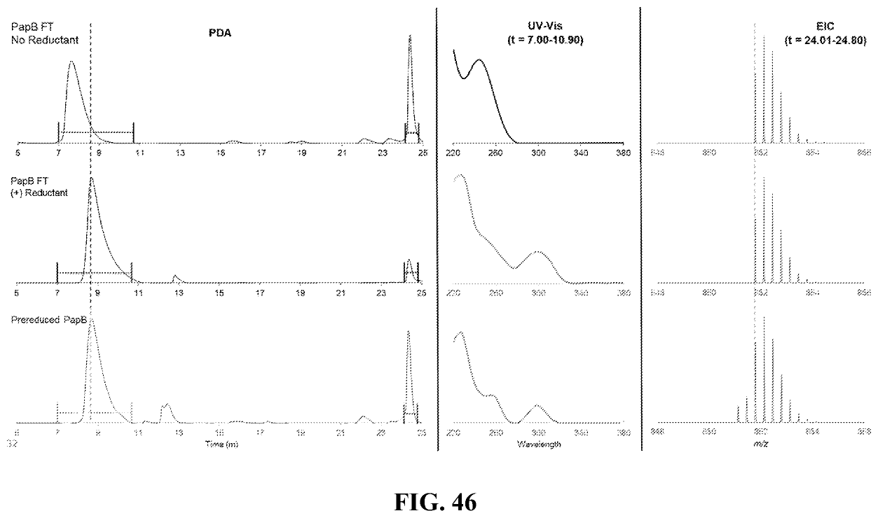

Studies were then conducted to explore the nature of the electron transfer reaction occurring during modification. The results are shown in FIG. 43, and demonstrate that the system is active in both tested reduction systems, with only loss of activity when flavodoxin mononucleotide is removed from the reducing system control series. Following these results, “prereduced” PapB studies were carried out to characterize the turnover in the absence of reductant. These results are shown in FIG. 44-FIG. 46.

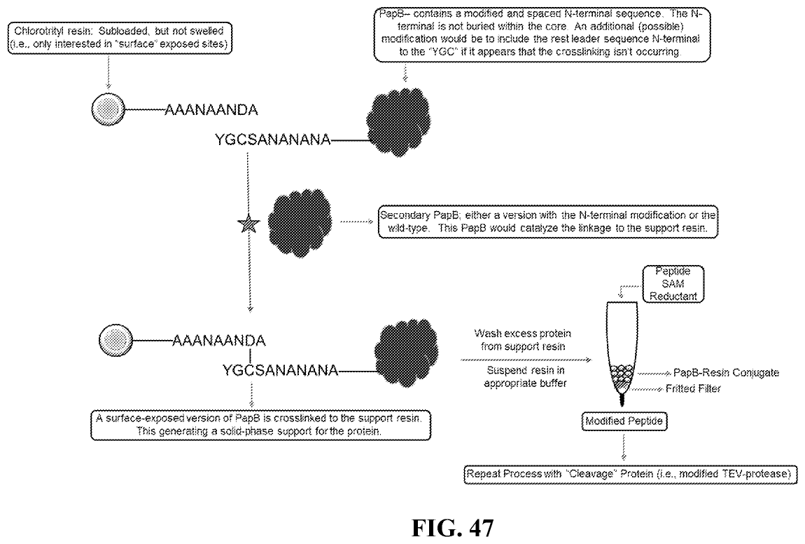

A concept schematic for a bioreactor setup for peptide modification via PapB is shown in FIG. 47.

### 7. PapB Tolerates C-Terminal Glycine

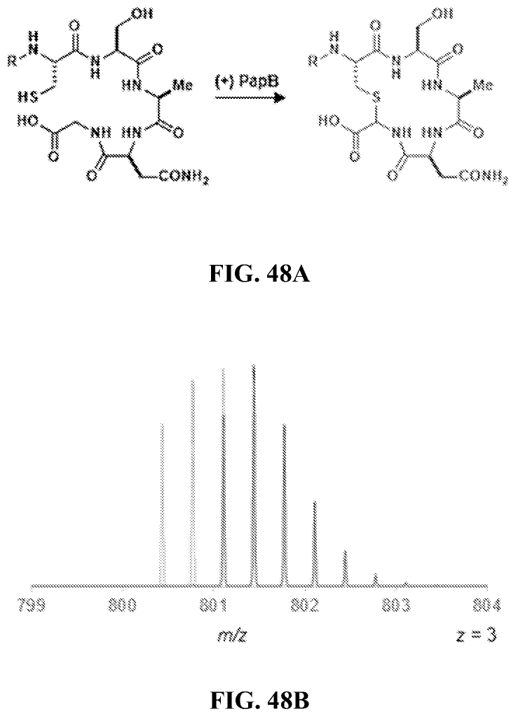

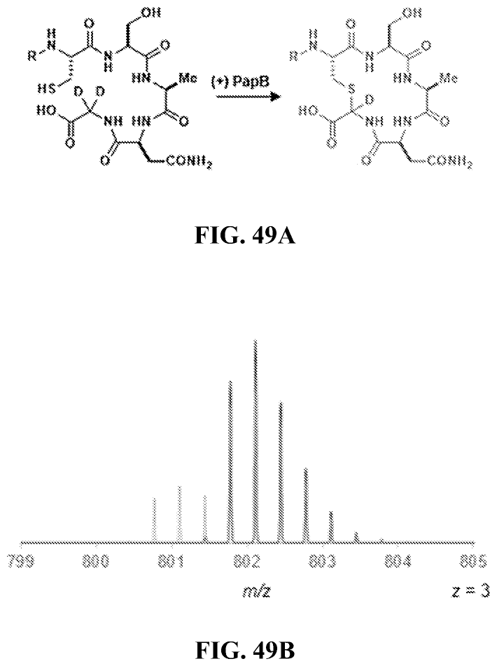

The sequence of leader-CX₃G for recognition and crosslinking was explored (FIG. 48A-B). Labeling experiments with deuterated glycine show the crosslinked peptide with a corresponding loss of 3 Da indicating that the thioether crosslink occurred on the carbon adjacent to the carboxylic acid (FIG. 49A-B).

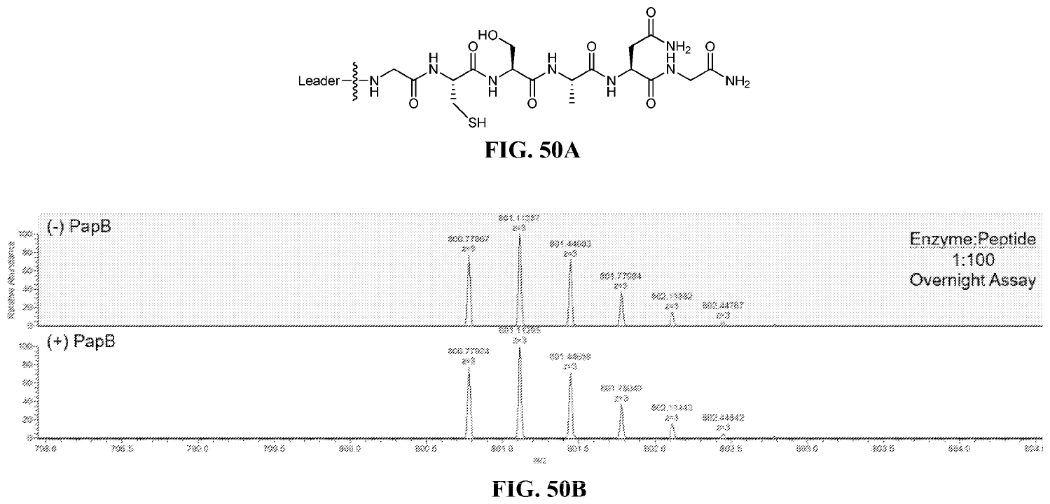

However, the sequence of leader-CX₃(CH₂)C(O)NH₂ did not demonstrate thioether crosslinking (FIG. 50A-B). The loading, synthesis, and cleavage of the C-terminal glycine carboxamide peptide, which differs from that described earlier as to all alternative C-terminal carboxylate peptides, is detailed below.

Briefly, 150 mg of Rink Amide Resin was swelled in 5 mL of DMF for 1 h in a polyprep chromatography column. Nitrogen gas was then used to remove DMF from the column. The Fmoc protecting group on the resin was deprotected by adding 10 mL of 20% (v/v) of piperidine in DMF and rocking the resin for 1 h. Nitrogen gas was used to remove the piperidine mixture from the resin. The resin was then washed three times with 5 mL of DMF. Next, 0.03 mmole of Fmoc-Gly was weighed added to 0.15 mmole HATU and 0.15 mmole HOAt in a glass scintillation vial. 10 mL of 20% N-methylmorpholine in DMF was added to the vial and gently shaken to dissolve the materials. The mixture was then added to the deprotected resin and rocked at room temperature for 4 h. The amino acid solution was then pushed out of the column with nitrogen gas and washed three times with 5 mL of DMF. The resin was then capped with a 3:2 ratio of acetic anhydride and pyridine. 10 mL of the mixture was added to the column and the resin mixture was shaken for 30 m at room temperature. The capping solution was pushed out of the column with nitrogen gas and washed 3× with 5 mL of DMF. All Fmoc-amino acids in the peptide synthesis (0.15 mmol, 6 equivalents) were coupled by in situ activation with N-[(dimethylamino)-1H-1,2,3-triazo[4,5-b] pyridin-1-ylmethylene]-N-methylmethanaminium hexafluorophosphate-N-oxide (HATU) (0.15 mmol, 6 equivalents; ChemPep) in 0.4 M N-methylmorpholine. After synthesis completion, the peptide was cleaved from the resin with 10 mL of 18:1:1 TFA:H₂O:triisopropylsilane. The solution was rocked at room temperature for 1 h. The cleavage reaction was filtered into 30 mL of ice-cold diethyl ether to precipitate the peptide. The solution was poured over a Büchner funnel filter and vacuumed to collect the peptide precipitate. The peptide dried on the vacuum for 15 min before being washed with 80 mL of ice-cold diethyl ether. After drying for an additional hour, the peptide was resuspended in 20 mL of water and sonified for 15 min to aid in the dissolution of the peptide. The solution was then flash frozen in liquid nitrogen and lyophilized.

After lyophilization, the peptide was resuspended in 0.05 M PIPES·NaOH (pH 7.4), 2 mM DTT, 300 mM KCl, and 15% glycerol buffer solution. The peptide was then assayed with PapB using the following parameters: 6.1 μM PapB, 100 μM msPapA C-terminal Gly carboxamide, 2 mM DTT, 2.1 mM SAM, and 15% glycerol in 100 uL total volume. The negative control (no PapB) and overnight complete assay (+PapB) were quenched by adding 11 μL of 30% (w/v) TCA to the mixture. The quenched assays were spun at 16,000×g for 10 m to pellet any precipitated enzyme or PIPES. The assays were analyzed using a Vanquish UHPLC with a diode-array detector connected to a Q-Exactive mass spectrometer operated in positive ion mode, the FT analyzer was set to 70,000 resolution, 1 microscan, and 200 ms maximum injection time. Xcalibur software was used to analyze data. A 20 μL aliquot was injected onto a Hypersil GOLD C18 column (msPapA) (2.1 mm×150 mm, 1.9 μm particle size) (Thermo Fisher) pre-equilibrated in 0.1% (v/v) LC-MS Optima TFA (Fisher) in LC-MS Optima water (Fisher). Chromatographic steps were carried out at 0.2 mL/min with buffer A containing 0.1% (v/v) TFA in Optima water and buffer B containing Optima grade acetonitrile with 0.1% (v/v) TFA. The separation consisted of washing with 100% A from 0 to 3 min, followed by a linear gradient from 100% to 0% A from 3 to 6 min, washing with 0% A from 6 to 10 min, and reequilibration with 100% A from 10 to 14 min.

### 8. PapB Tolerates C-Terminal β-Amino Acids

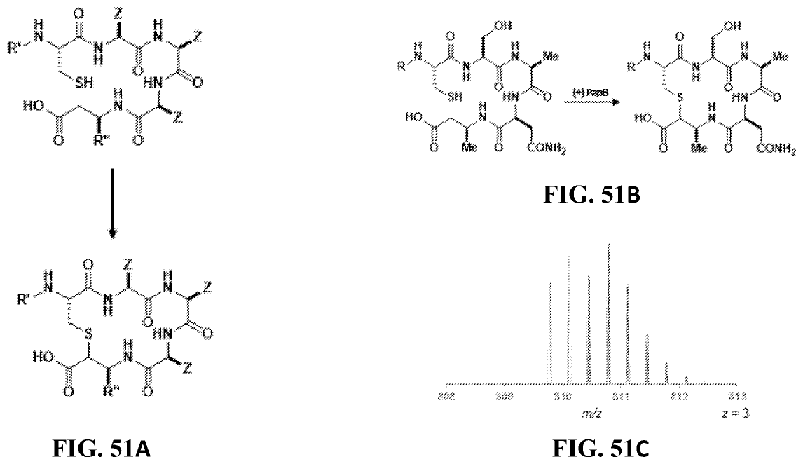

The sequence of leader-CX₃(β-amino acids) for recognition and crosslinking was explored (FIG. 51A). The sequence of leader-CX₃(L-3-aminobutyric acid) was probed and treatment with PapB resulted in loss of 2 Da indicating the thioether crosslinked product (FIG. 51B-C)

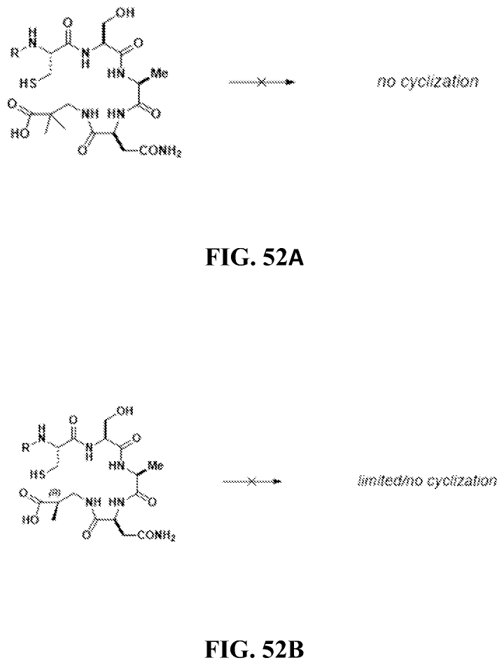

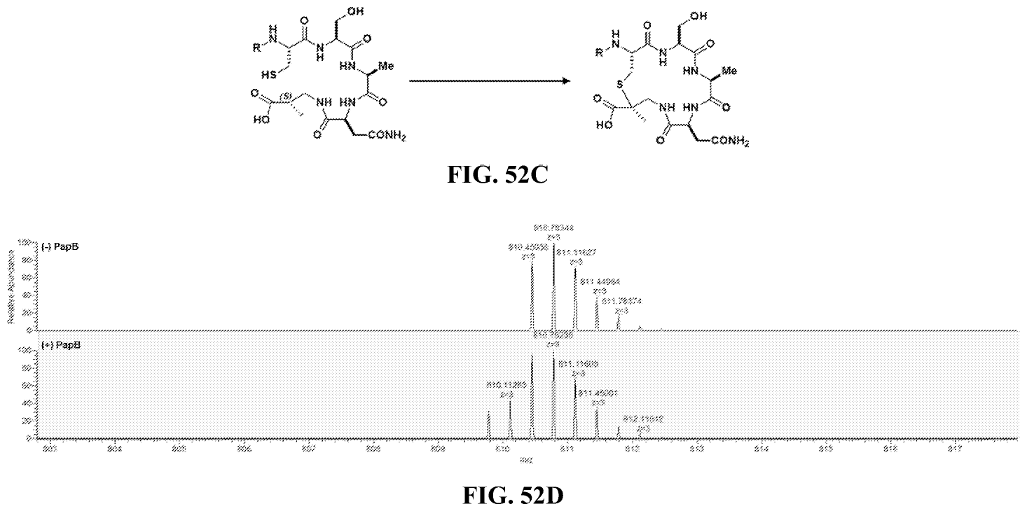

Alternative β-amino acids were explored to probe the sequence requirements for recognition and crosslinking. The sequences of leader-CX₃(3-amino-2,2-dimethylbutanoicacid) (FIG. 52A) and leader-CX₃((R)-3-amino-2-methylpropanoic acid) (FIG. 52B) did not show evidence of a 2 Da loss, which indicated that the thioether crosslinked product was not formed.

The sequence of leader-CX₃((S)-3-amino-2-methylpropanoic acid) (FIG. 52C) did show evidence of a 2 Da loss (FIG. 52D), which gives indication that the thioether crosslinked product was formed. Without wishing to be bound by theory, the culmination of these selective methylations on a C-terminal β-amino acid demonstrates that only one position is not amenable to substitution. The position alpha to the carboxylate must contain an H-atom in the pro-R position. The reference point to the pro-R position described is a singly methylated alpha-to-the-carboxylate moiety. In any scenario where the absolute stereochemistry priority may shift due to a substituent priority change, the scenario of the sidechains must be compared to that singly methylated case.

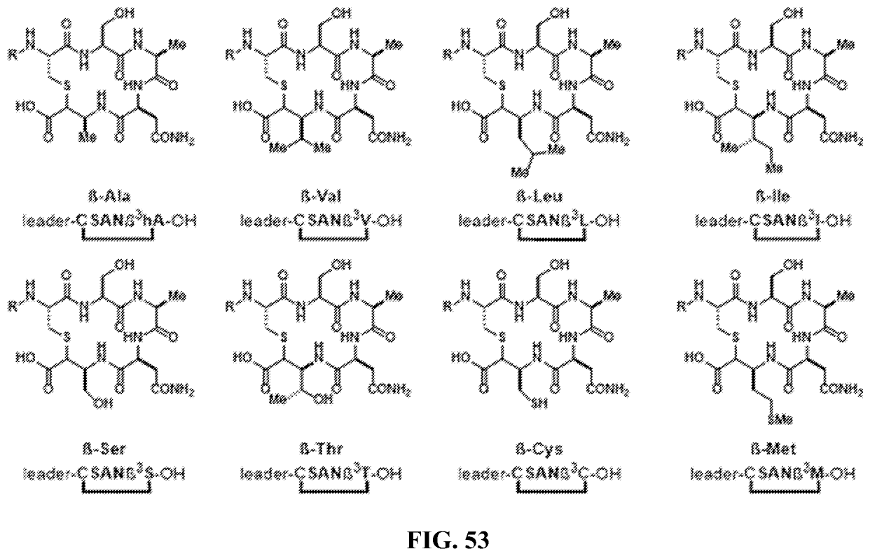

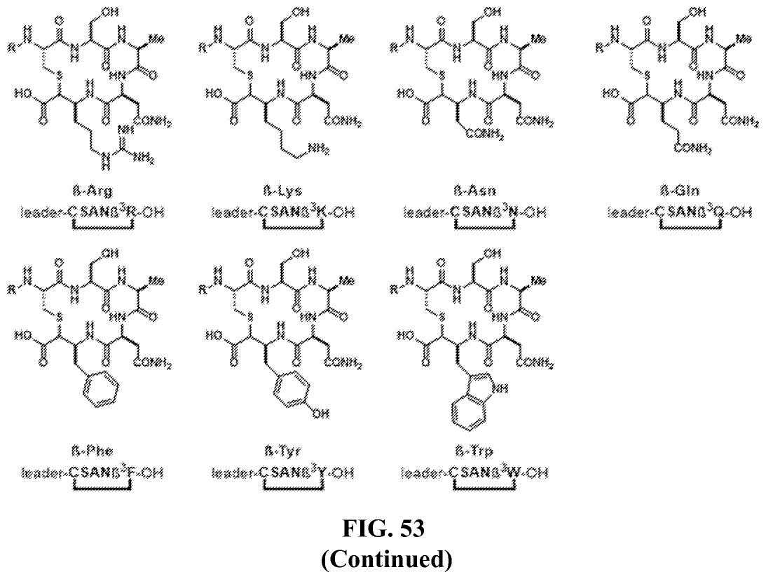

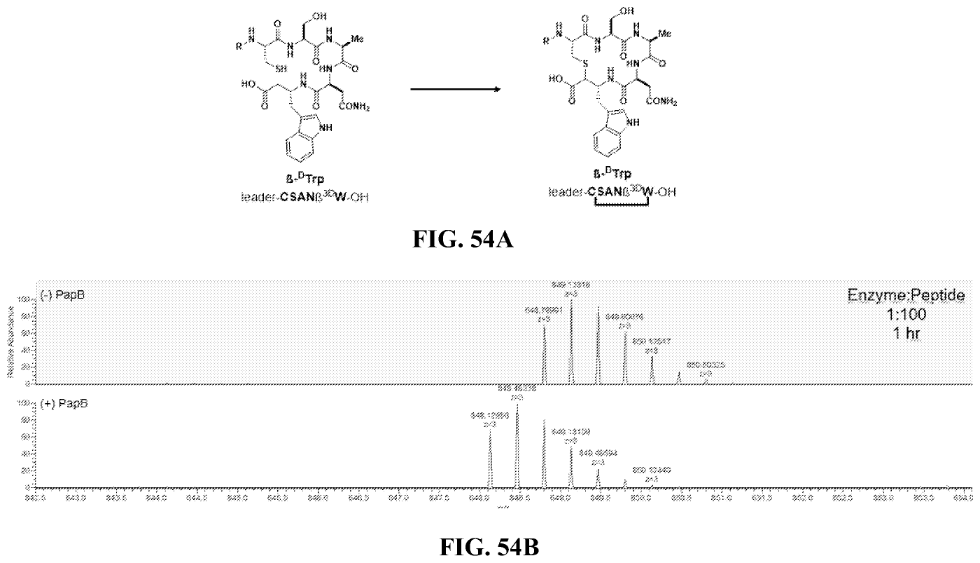

The sequence of leader-CX₃(β-amino acids) was further explored utilizing analogs representative of natural amino acids (FIG. 53). A representative analog with B-tryptophan (FIG. 54A) did show evidence of a 2 Da loss (FIG. 54B), which gives indication that the thioether crosslinked product was formed.

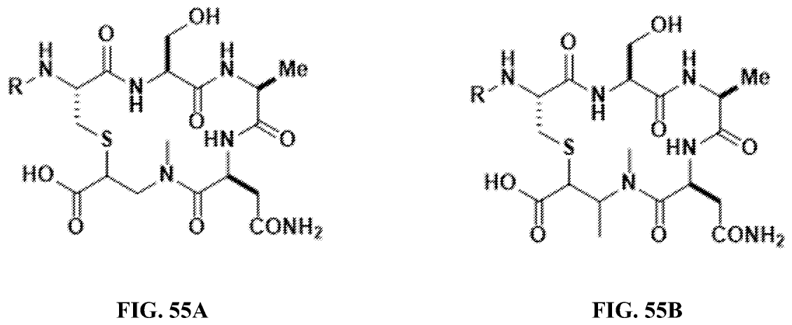

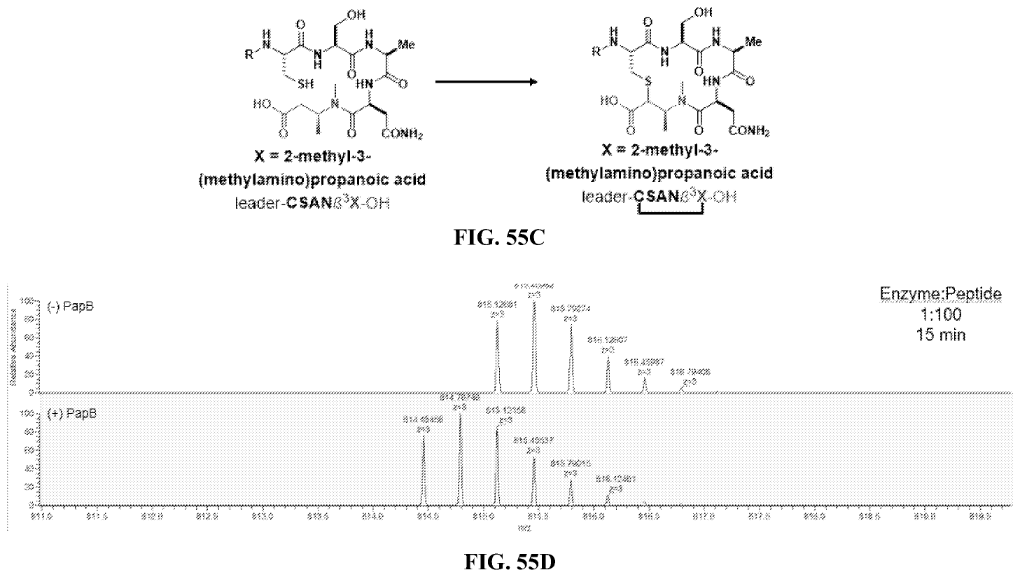

The sequence of leader-CX₃(β-amino acids) was further explored by probing the reaction with N-methylated β-amino acids (FIG. 55A-B). A representative analog with sequence of leader-CX₃(2-methyl-3-(methylamino) propanoic acid) was treated under reaction conditions with PapB (FIG. 55C). The reaction showed evidence of a 2 Da loss (FIG. 55D), which gives indication that the thioether crosslinked product was formed.

### 9. PapB Tolerates C-Terminal D-Amino Acids

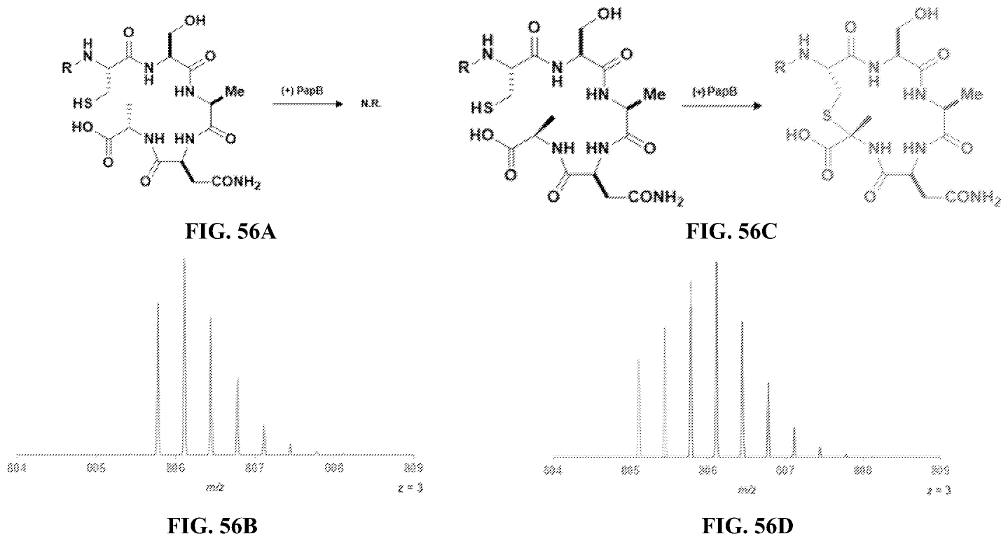

To assess the requirements of the C-terminal carboxylic acid D and L variants, Leader-CSAD^{L}A and Leader-CSAD^{D}A were prepared and incubated with PapB (FIG. 56A-D) In the case of L-alanine (FIG. 56A) a loss of 2 Da was not observed indicating that thioether crosslinked product was not formed (FIG. 56B). In the case of D-alanine (FIG. 56C) a loss of 2 Da was observed indicating that thioether crosslinked product was formed (FIG. 56D).

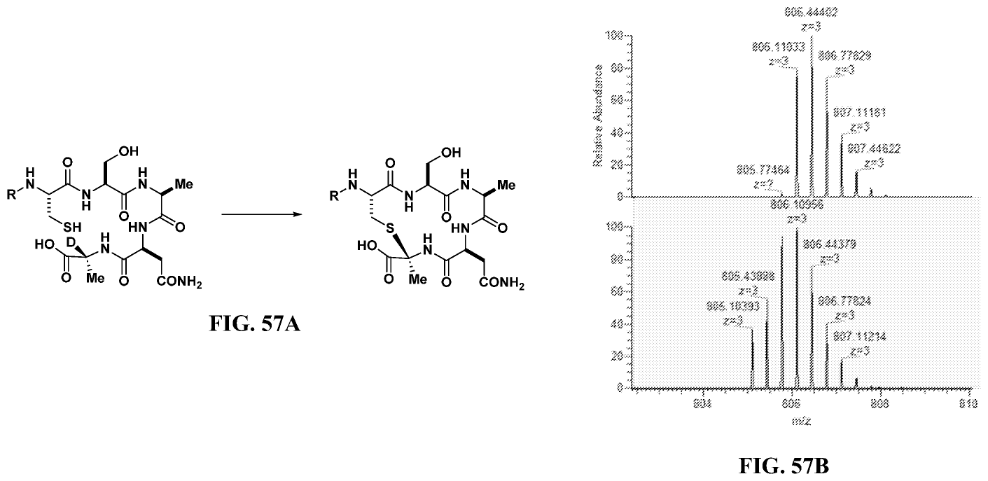

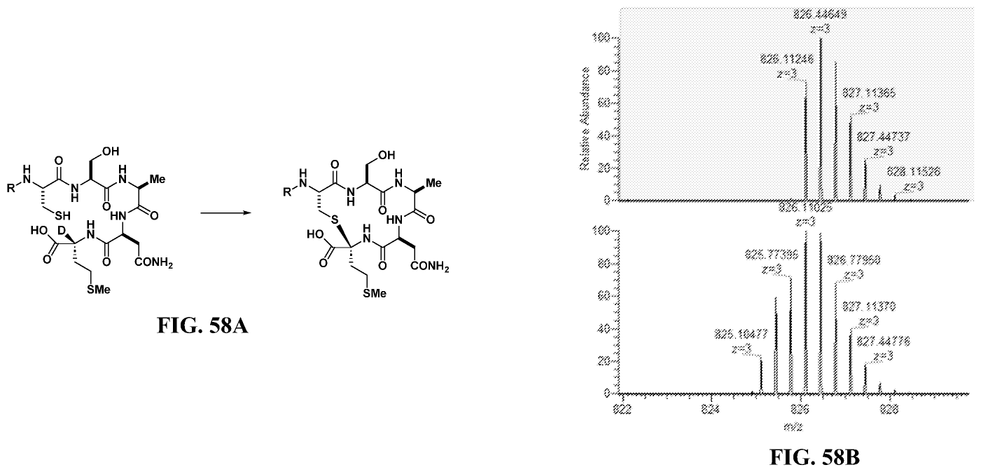

Further exploration with deuterated D-alanine at the carbon adjacent to the carboxylic acid incorporated into the leader-CSAD^{D}A sequence (FIG. 57A) showed evidence of a 3 Da loss, which indicated that the thioether crosslinked product was formed adjacent to the carboxylic acid (FIG. 57B). Similarly, deuterated D-methionine at the carbon adjacent to the carboxylic acid leader-CSAD^{D}M sequence (FIG. 58A) showed evidence of a 3 Da loss, which indicated that the thioether crosslinked product was formed adjacent to the carboxylic acid (FIG. 58B).

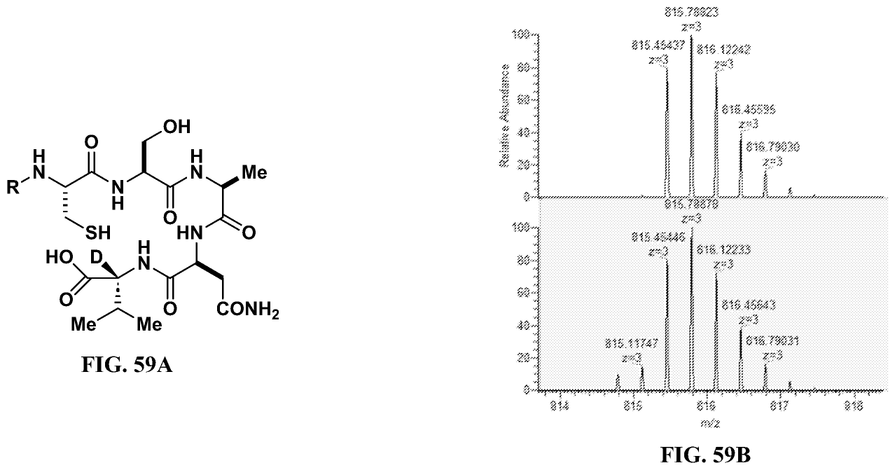

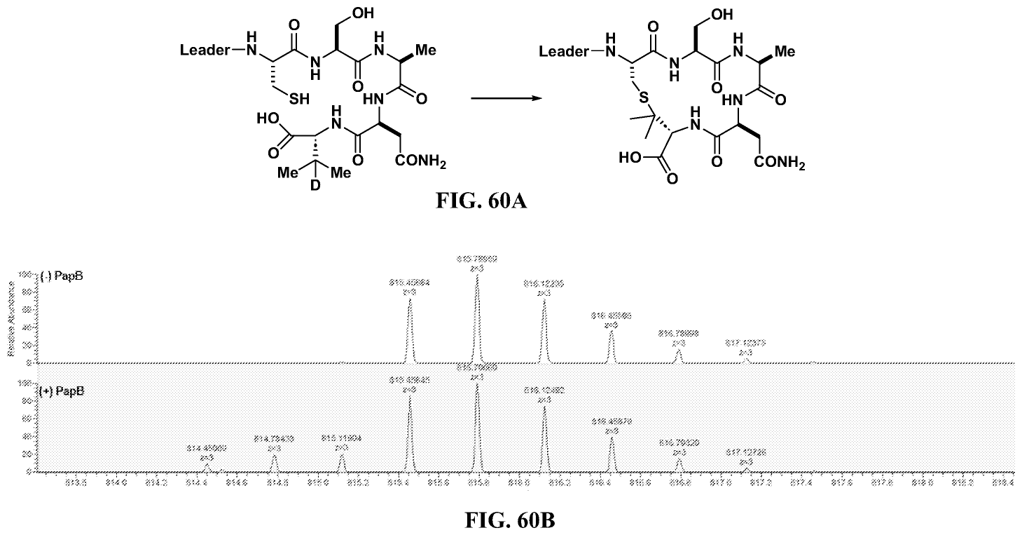

However, deuterated D-valine at the carbon adjacent to the carboxylic acid leader-CSAD^{D}V sequence (FIG. 59A) showed evidence of a 2 Da loss, which indicated that the thioether crosslinked product was formed (FIG. 59B) but the thioether crosslinked product was not at the carbon adjacent to the carboxylic acid. A deuterated D-valine at the tertiary sidechain carbon leader-CSAD^{D}V sequence (FIG. 60A) showed evidence of a 3 Da loss (FIG. 60B), which indicated that the thioether crosslinked product was formed at the tertiary carbon

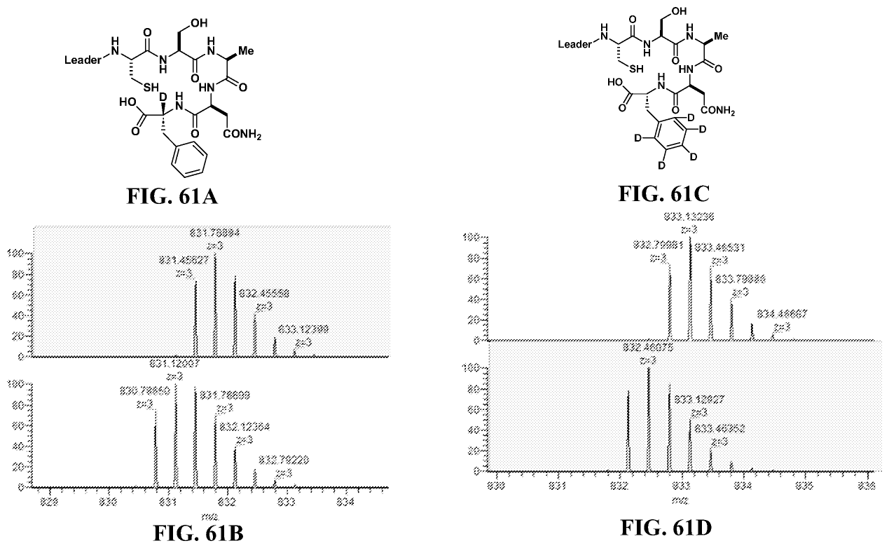

Deuterium labeled phenyl alanine at the carbon adjacent to the carboxylic acid was incorporated into leader-CSAD^{D}F sequence (FIG. 61A) and incubated with PapB. The product showed evidence of a 2 Da loss (FIG. 61B). This is again indicative of successful thioether crosslinked product but the thioether crosslinked product was not at the carbon adjacent to the carboxylic acid. Similarly, phenyl alanine d5 was incorporated into leader-CSAD^{D}F sequence (FIG. 61C) and incubated with PapB. The product showed evidence of a 2 Da loss (FIG. 61D). This is again indicative of successful thioether crosslinked product but the thioether crosslinked product was not formed with the aromatic group.

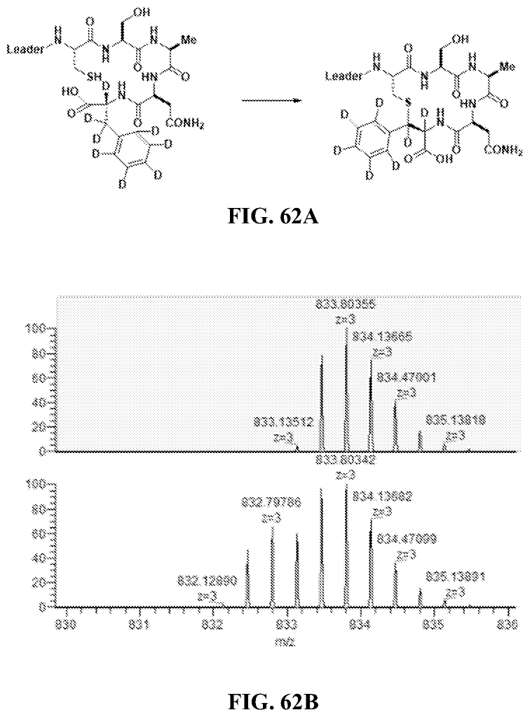

Phenyl alanine d8 was incorporated into leader-CSAD^{D}F sequence (FIG. 62A) and incubated with PapB. The product showed evidence of a 3 Da loss (FIG. 62B). This is indicative of successful thioether crosslinked product to the methylene of the phenylalanine side chain.

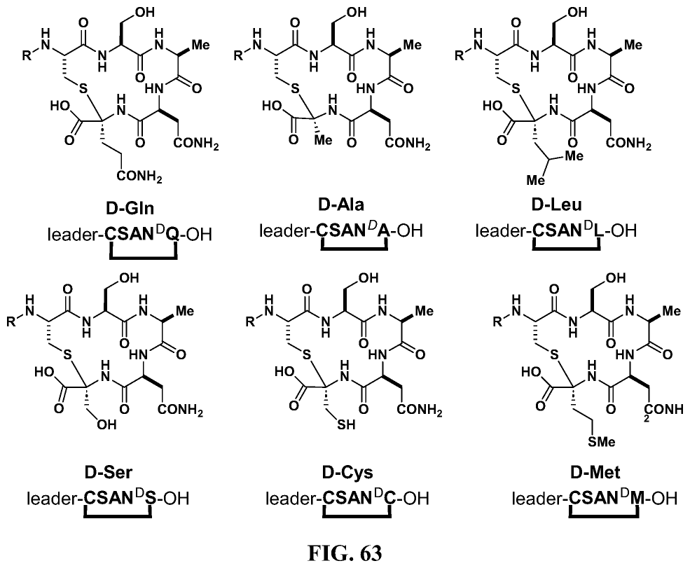

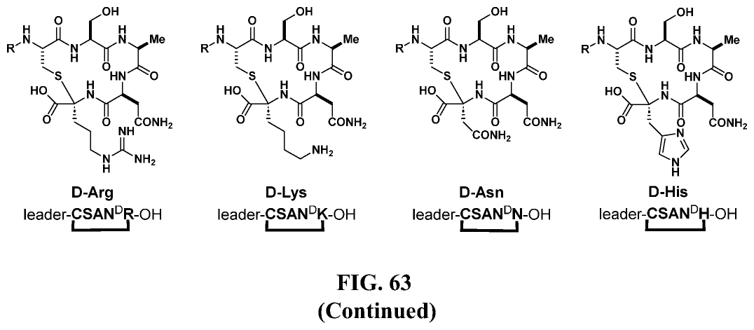

The sequence of CX₃(C-terminal D-amino acids) was further explored utilizing analogs representative of natural amino acids to form sactipeptides (FIG. 63).

The sequence of CX₃(C-terminal D-amino acids) of certain amino acids provided ranthipeptides (FIG. 64).

### 10. PapB Tolerates Non Peptide Analogs

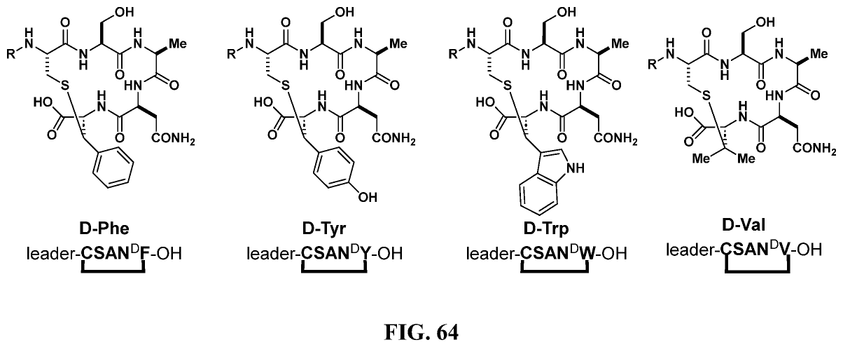

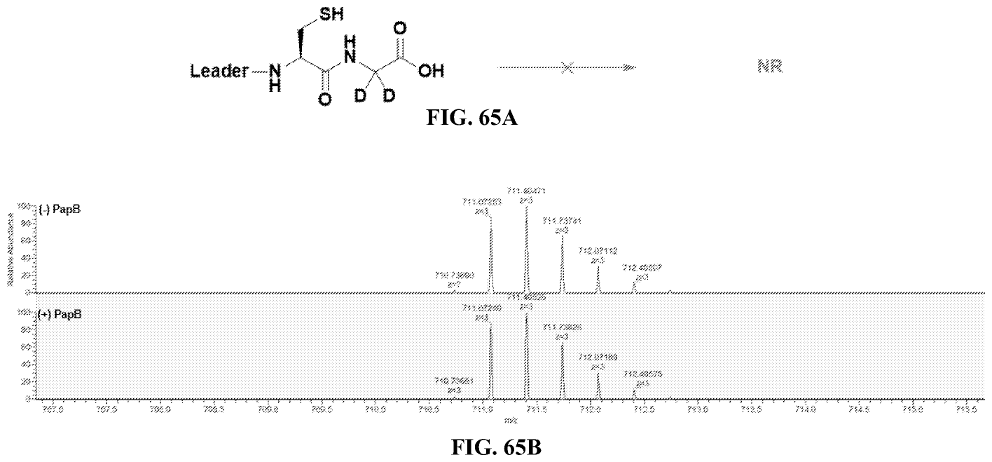

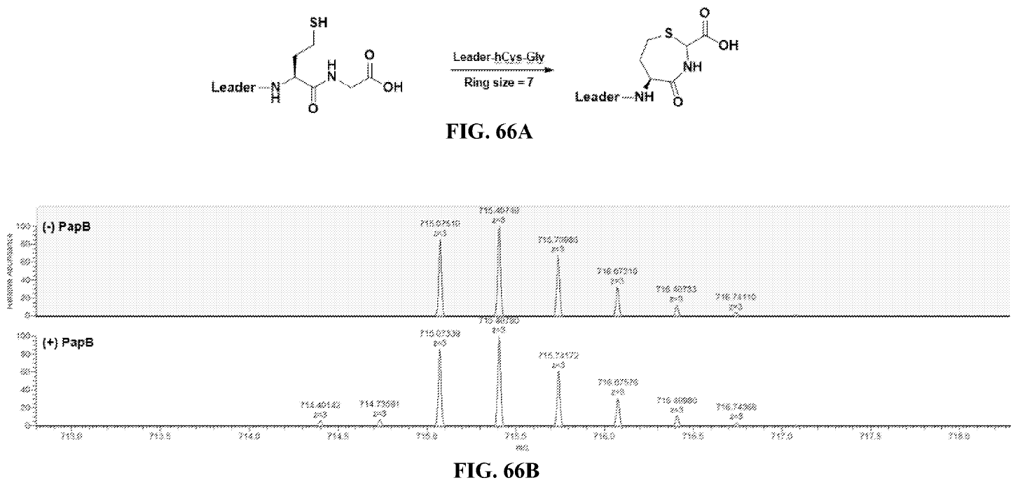

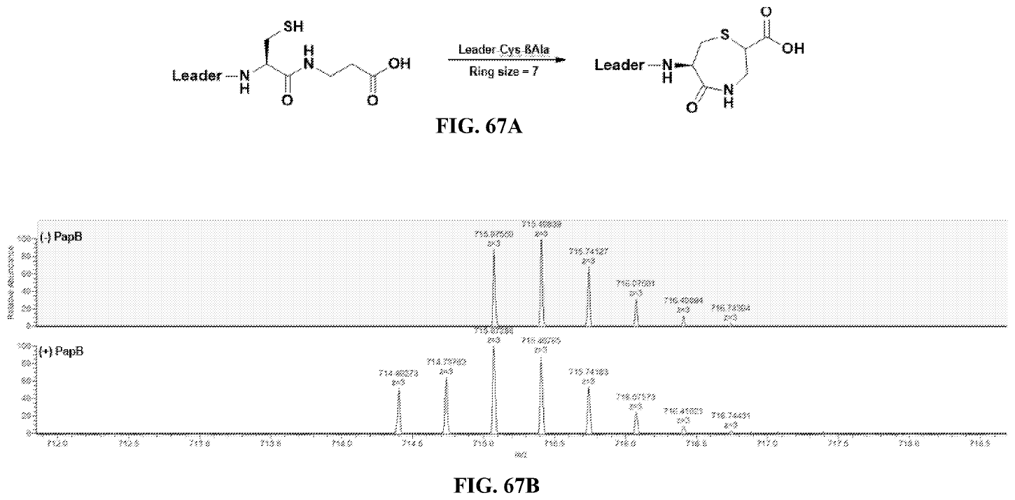

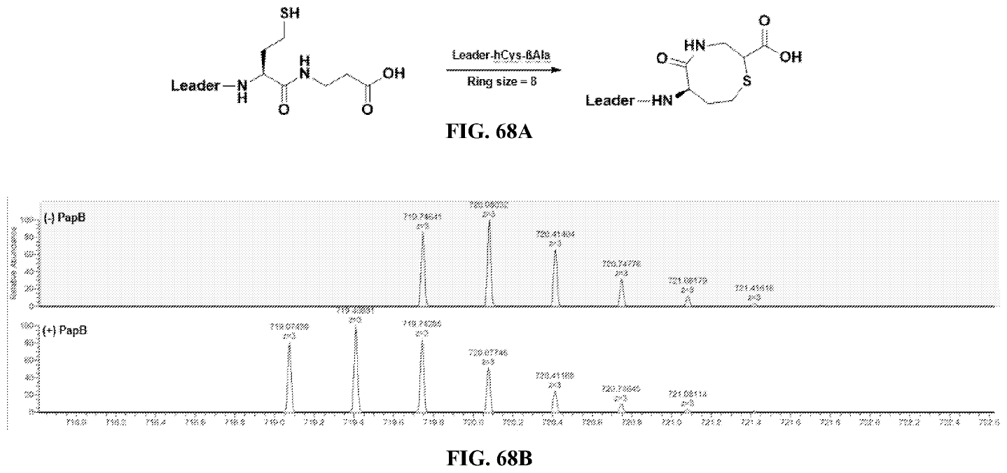

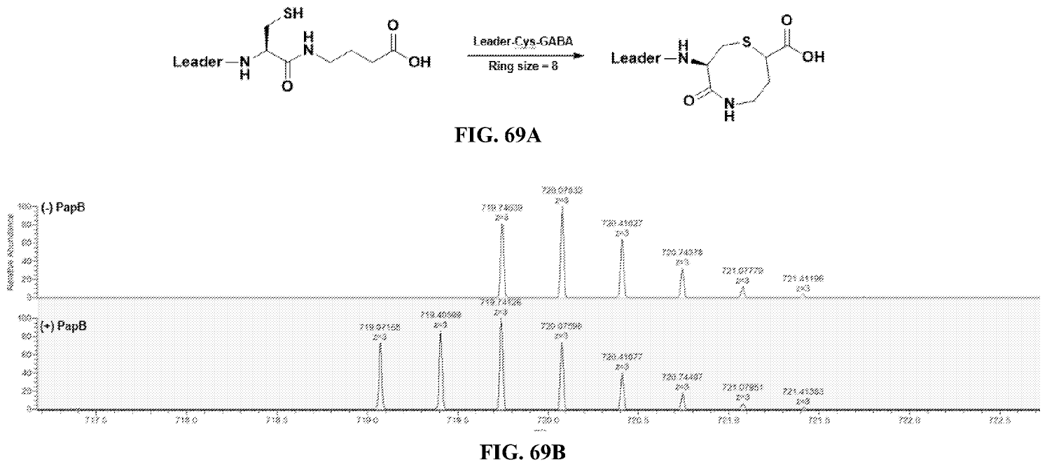

The rings size of non-peptide sequences was explored for carbon linked analogs (FIG. 65-70). In the case of Leader-CG, no crosslinking is observed (FIG. 65A). This places a lower limit on amenable crosslink formation (FIG. 65B). In the case of Leader-(hCys)-(Gly) (FIG. 66A), a 2 Da loss is observed in the mass spectrum upon reaction with PapB (FIG. 67B). Similarly, with Leader-(Cys)-(β-Ala) (FIG. 67A), thioether crosslinking is observed upon addition of PapB (67B). The smallest ring observed is a 7-membered ring. Extensions of a single CH₂ group on either the thiol- or carboxylate-containing moiety from the baseline Leader-CG scenario resulted in crosslinking. Further, the ring size can be expanded with additional CH₂ groups. See FIG. 68A (Leader-hCys-βAla) for the sequence and FIG. 68B for the representative mass spectra illustrating a 2 Da loss, FIG. 69A (Leader-Cys-gamma amino butyric acid) for the sequence and FIG. 69B for the representative mass spectra illustrating a 2 Da loss, and FIG. 70A (Leader-hCys-gamma amino butyric acid) for the sequence and FIG. 70B for the representative mass spectra illustrating a 2 Da loss.

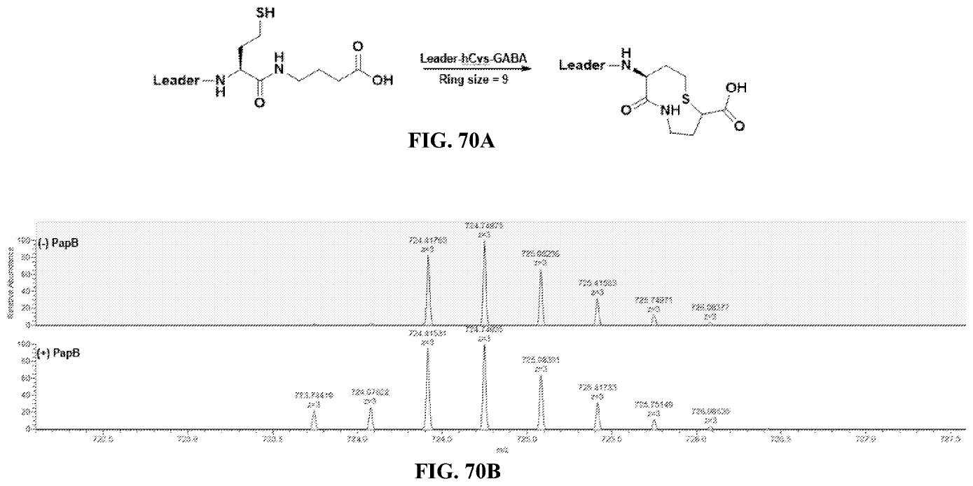

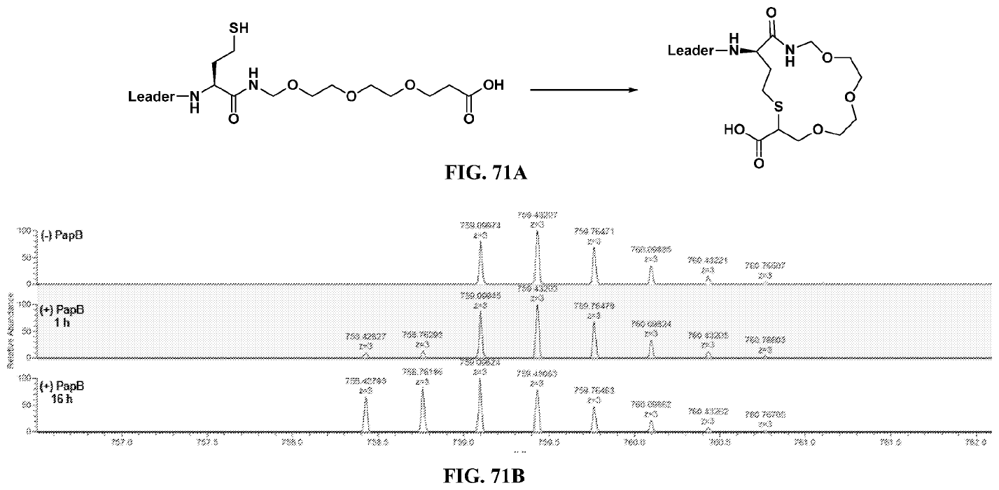

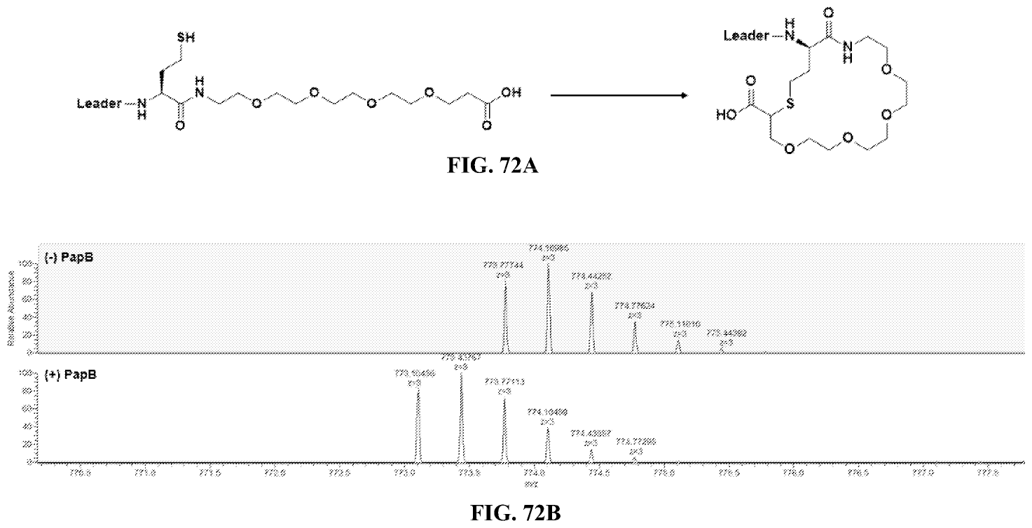

The maximum ring size of non-peptide sequences was explored using PEG-based moieties terminating in a carboxylate (FIG. 71-72). In the case of 3× PEGylation (FIG. 71A), thioether crosslinking is observed upon addition of PapB (FIG. 71B). 4× PEGylation (FIG. 72A) shows thioether formation upon addition of PapB (FIG. 72B), thioether formation is observed.

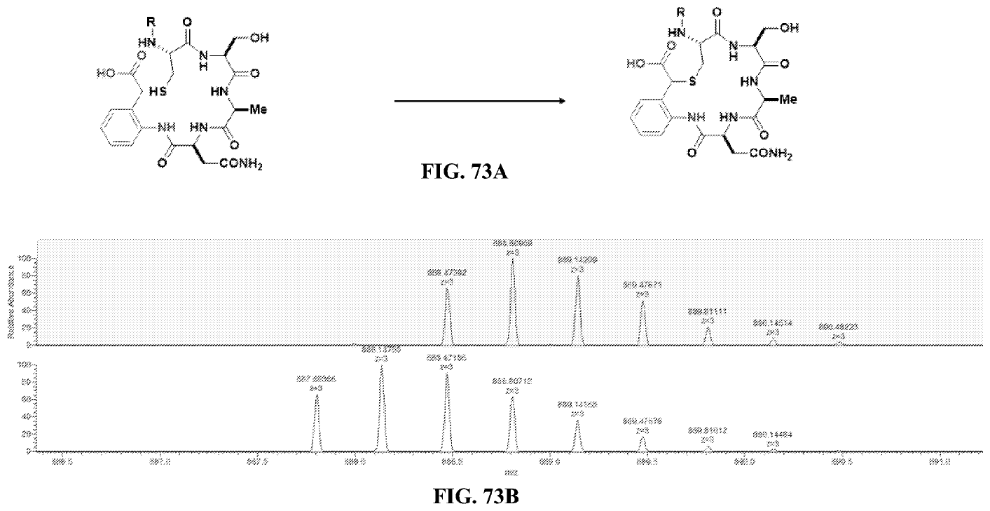

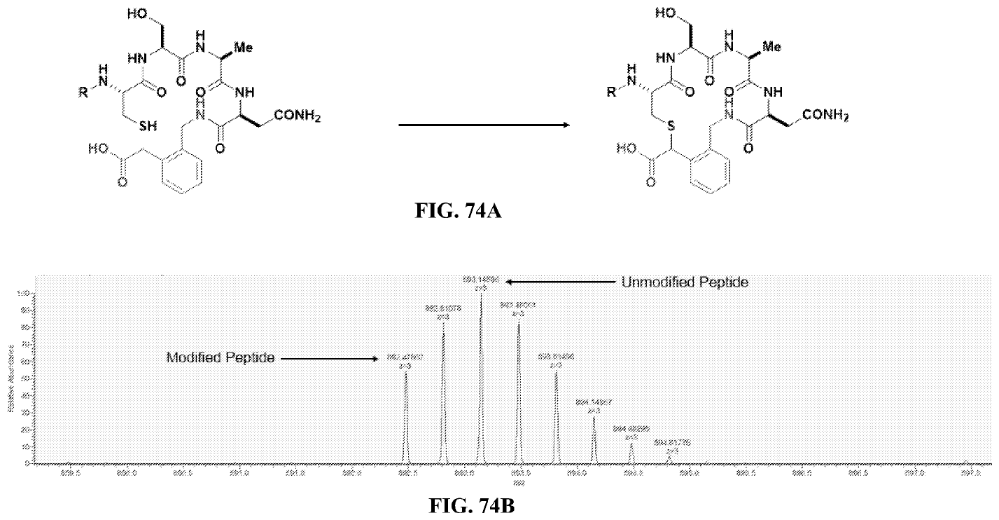

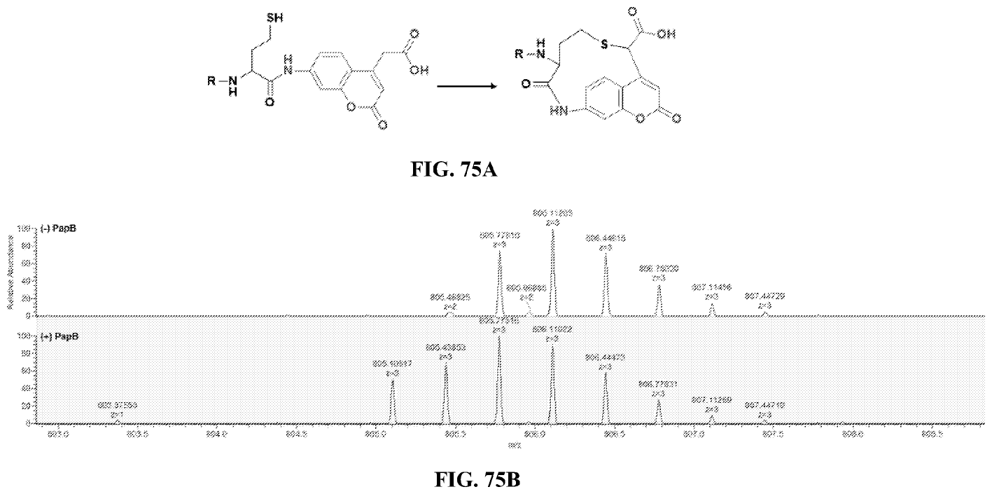

Alternate scaffolds were also explored (FIG. 73-75) to establish that both aromatics and heterocycles can be included in the thioether macrocycle. FIG. 73A demonstrates the structures of a peptide chain containing a substituted aniline in the peptide backbone and the reacted thioether macrocycle. FIG. 73B shows the mass spectra of the unreacted (top, without PapB) and reacted (bottom, with PapB) aniline-containing peptide chain. FIG. 74A demonstrates the structures of a peptide chain containing a substituted benzylamine in the peptide and the reacted thioether macrocycle. FIG. 74B shows the mass spectra of the reacted benzylamine-containing peptide chain. FIG. 75A show the structures of a modified courmarin-containing peptide. The coumarin-like moiety is the most C-terminal aspect of the peptide. FIG. 75B demonstrates the mass spectra of the coumarin-containing peptide both unreacted (top, without PapB) and reacted (bottom, with PapB).

### 11. PapB Utilized to Prepare Thioether Crosslinked Peptidomimetics

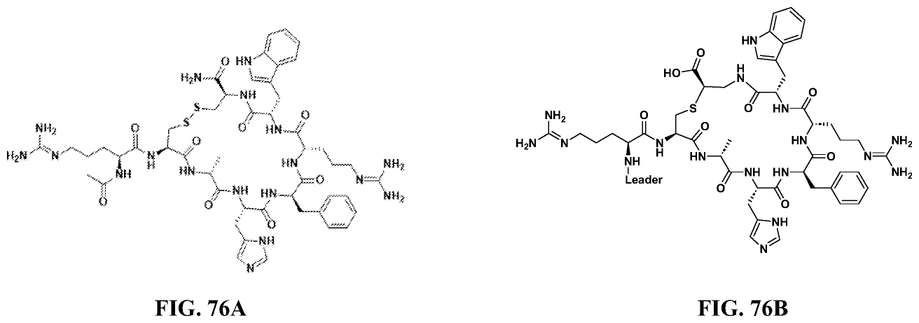

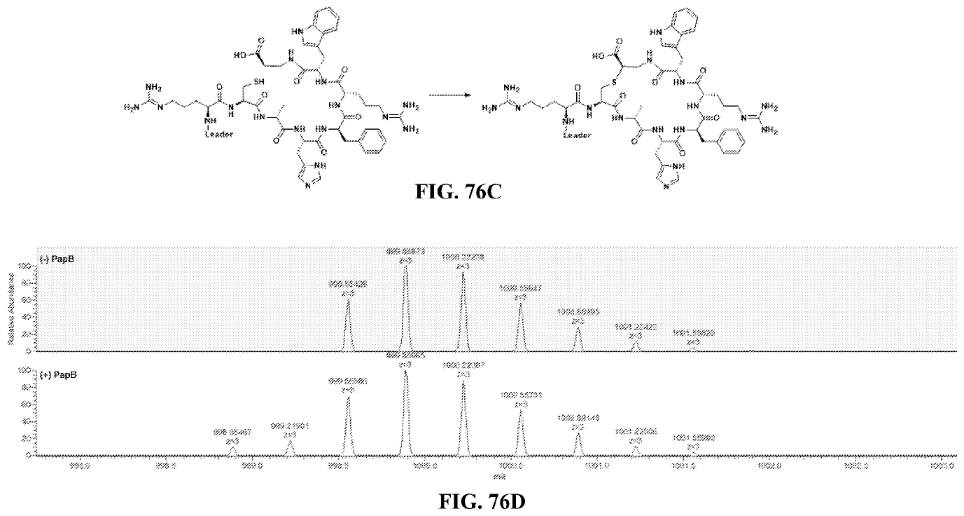

Setmalanotide, an MC4R agonist, is an FDA-approved drug indicated for chronic weight management (FIG. 76A). A similar peptidomimetic analog based on the PapB reaction to form thioether crosslinked products was envisioned (FIG. 76B). The sequence of Leader-CD^{D}AHD^{D}FRWX (FIG. 76C, where X=β-Ala) was explored. The reaction showed evidence of a 2 Da loss (FIG. 76D), which gives indication that the thioether crosslinked product was formed.

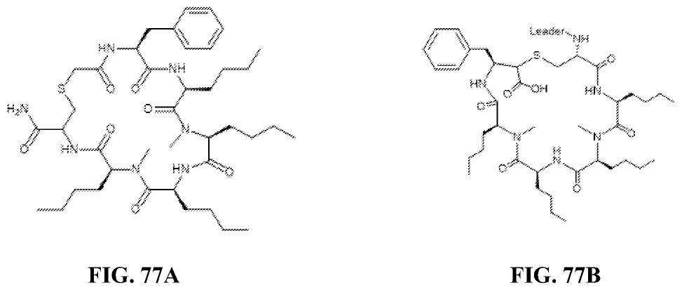

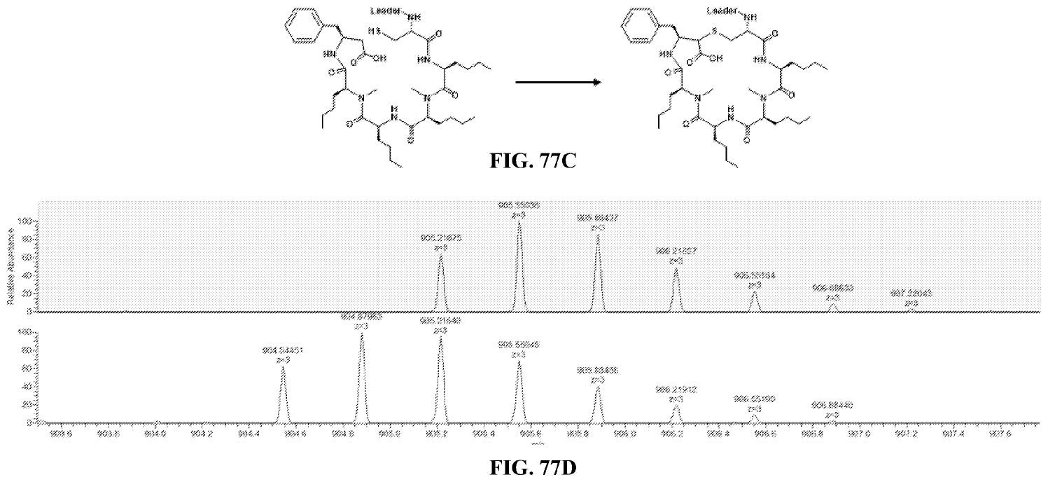

An orally available crosslinked thioether peptidiomimetic was recently disclosed (J. Med. Chem. 2021, 64, 5, 2622-2633) (FIG. 77A) A similar peptidomimetic analog based on the disclosed PapB reaction to formed thioether crosslinked products was envisioned (FIG. 77B). The sequence of Leader-CBXBXF (FIG. 77C, where B=norleucine, X═N-methyl norleucine) was explored. The reaction showed evidence of a 2 Da loss (FIG. 77D), which gives indication that the thioether crosslinked product was formed.

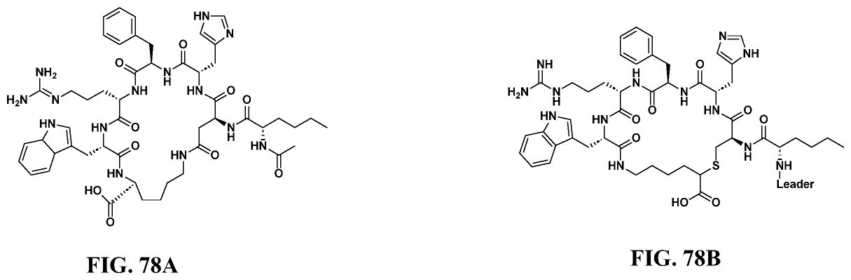

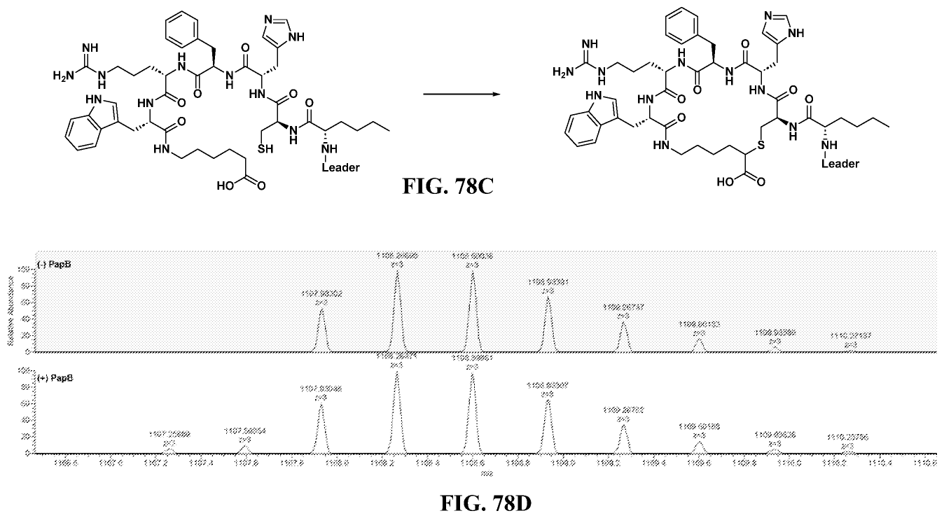

Bremelanotide, an agonist of MC1R, MC4R, MC3R, MC5R, and MC2R, is an FDA-approved drug that treats hypoactive sexual desire in premenopausal women. A similar peptidomimetic analog with the sequence Leader-BC^{D}FRWZ (where B=norleucine, and Z=ε-ACP) was generated (FIG. 78A). The envisioned thioether crosslinked analog is shown in FIG. 78B. The PapB transformation is shown in FIG. 78C. The mass spectra showing the reaction both in the absence (top) and presence (bottom) of PapB is shown in FIG. 78D. The proposed thioether analog is shown in FIG. 78B.

### 12. PapB Crosslinks Extended Sidechains of Thiol- and Carboxylate-Containing Residues

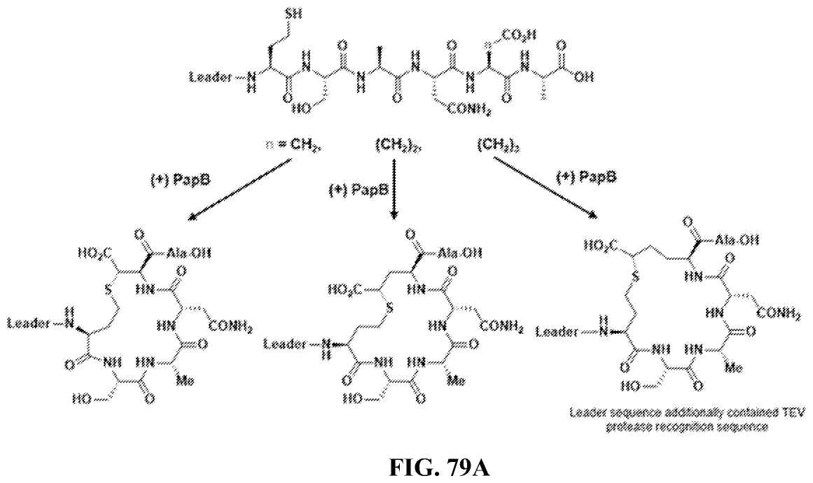

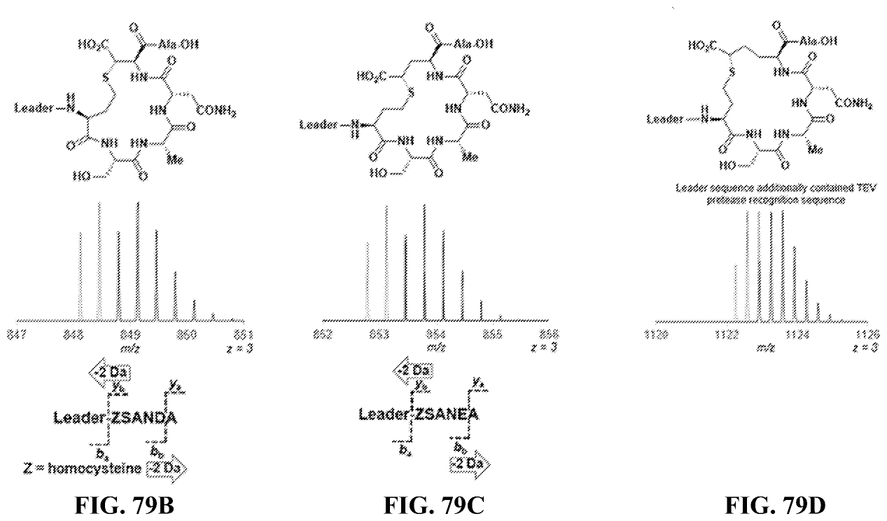

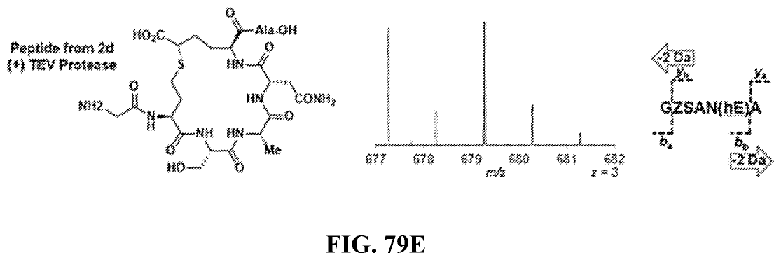

Previous studies have demonstrated that PapB can tolerate extended sidechains of the acidic residue, as both CX₃D and CX₃E sequences are crosslinked. PapB forms crosslinks in msPapA with a homocysteine (hCys) substitution at position 19 and an Asp in position 23, C19hCys and D23E, and C19hCys and homoglutamate (hGlu) at position 23 (FIG. 79A). MS analysis confirmed that PapB catalyzes formation of a crosslink in peptides containing hCys at position 19 and Asp (FIG. 79B), hCys at position 19 and Glu (FIG. 79C), or hCys at position 19 and hGlu at position 23 (FIG. 79D). Previous results demonstrated that changing either the donor or acceptor residue was amenable, however in the case of D-amino acids, little crosslinking was seen when both residues were altered simultaneously. Changing both the donor and acceptor residue and observing efficient substrate conversion is uncommon in RiPP maturation. The MS analysis of the reaction products show that each substrate undergoes crosslinking, as evidenced by a 2 Da loss relative to the substrate due to the loss of one H from the hCys thiol and a second H from the sidechain of the carboxylate-containing sidechain at residue 23. As a proof of concept for the use of PapB in generating macrocyclized peptides, in the case of C19hCys/D23hGlu variant, the leader sequence has been shown to be cleaved to generate the macrocyclized core peptide (FIG. 79E).

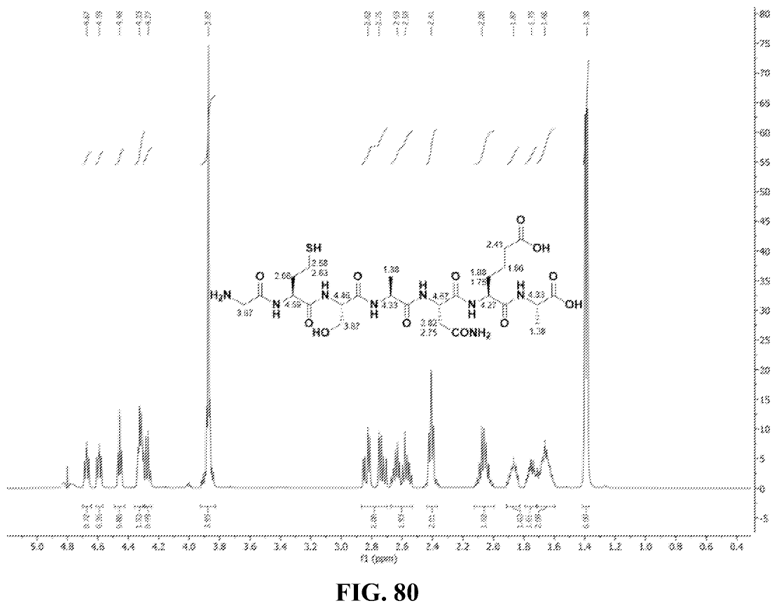

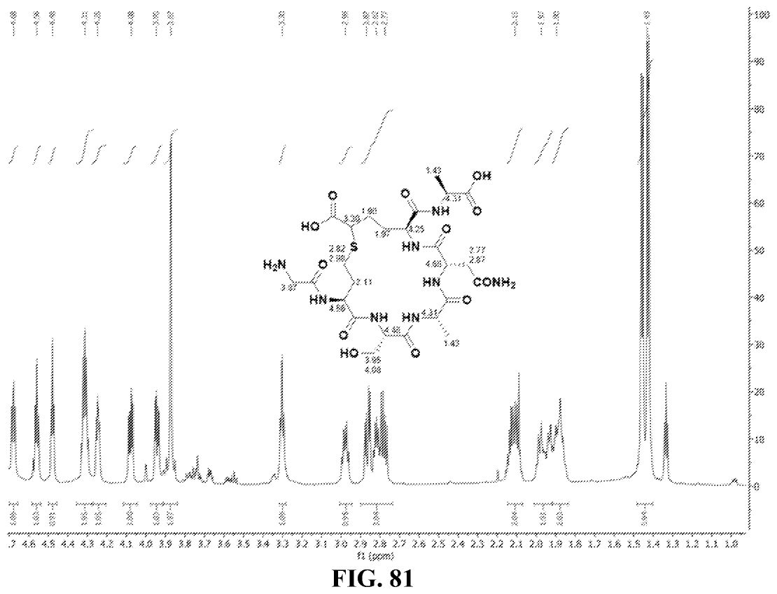

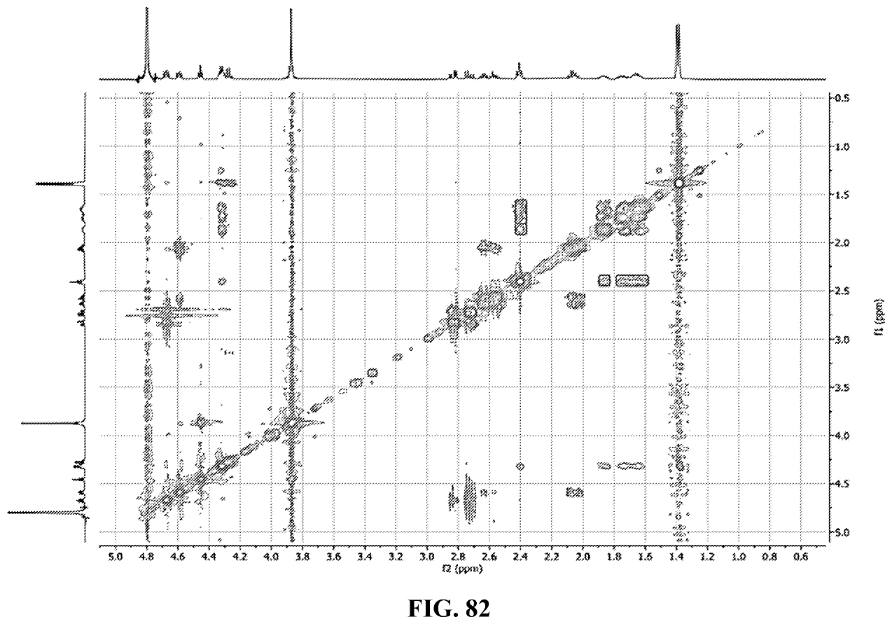

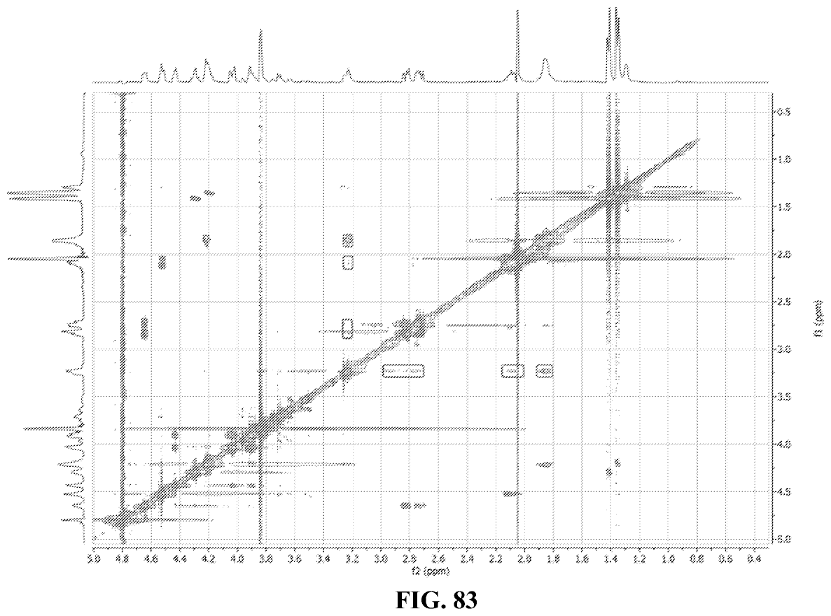

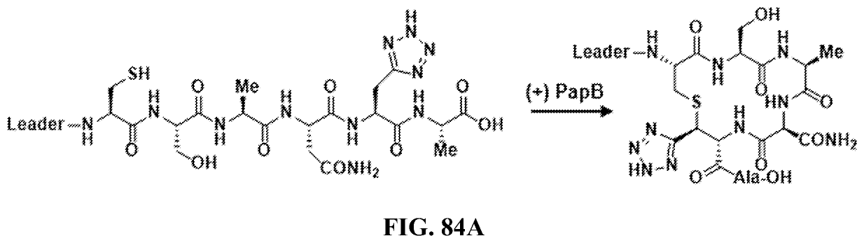

The observation of a 2 Da shift, when taken together with the MS/MS data and the loss of the IAM-sensitivity clearly shows that a thioether crosslink has formed. However, further confirmation that as with the wildtype substrate, the crosslink was formed at the carbon atom that is alpha to the carboxylate of the sidechain was required. To this end, either unmodified or crosslinked C19hCys/D23hGlu was treated with TEV protease to liberate the modified core from the leader peptide, purified by HPLC, and subjected to both one- and two-dimensional NMR analysis. The 1D NMR spectrum of the peptide prior to the treatment with PapB reveals a resonance at 2.41 ppm, which is composed of a doublet of triplets integrating to two protons (FIG. 80). This feature can reasonably be assigned to He of hGlu. In the NMR spectrum of the treated peptide (FIG. 81), this resonance is absent and a new triplet at 3.34 ppm integrating to a single hydrogen is observed. This new resonance is consistent with thioether installation at the position alpha to the carboxylate in the hGlu sidechain. To further correlate the positions, the modified and unmodified peptides were subjected to ROESY analysis to establish through-space correlations of protons. In the unmodified peptide, the resonance at 2.41 ppm is coupled to resonances at 1.66, 1.75, and 1.88 ppm, corresponding to through-space coupling of He in the hGlu sidechain to Hγ and Hβ of hGlu, respectively (see FIG. 82). In the modified peptide, the new resonance at 3.25 ppm is coupled to resonances at 1.8-1.9 ppm (see FIG. 83). The comparison of the linear and the cyclized peptide 1D spectra show a ppm shift of ˜0.2 in the resonances between 1.6 and 2.0, which would make the coupled resonances in the modified peptide ROESY spectra consistent with a through-space coupling of Hα of the hGlu sidechain to Hβ and Hγ of hGlu. Additionally, a weak coupling between 3.25 to 2.10 and 2.79-2.98 is observed. These cross peaks are consistent with Hα of the hGlu sidechain coupling with HB (2.10 ppm) and Hγ (2.79-2.98) of the hCys sidechain. Without wishing to be bound by theory, these results demonstrate that PapB can crosslink extended sidechains of thiol- and carboxylate-containing residues.

While various levels of promiscuity have been reported in the rSAM RiPP field, changing both the identity of both the donor and acceptor residues and forming a chemically consistent product is unprecedented. These findings expand the understanding of the capabilities of PapB and have implications for the biosynthesis of RiPPs that contain such extended sidechains.

### 13. Crosslinking with a Tetrazole Moiety: Implications for Peptide-Based Therapies

While the data with hCys, hGlu, and D-amino acid containing peptides all suggest a high level of tolerance in PapB for various substrates, the examples shown are limited by the fact that they all contain a thiol- and carboxylate-containing amino acid. It has been demonstrated herein that selenocysteine peptides are processed by PapB, but no examples of an isostere of a carboxylate moiety being processed by an rSAM RiPP maturase currently exist. Tetrazole moieties are commonly used as a bioisostere of carboxylic acids in small-molecule drug development. Tetrazoles improve the bioavailability of drugs, increase their lipophilicity, and reduce side-effects when compared to carboxylate-containing compounds. This is due to the metabolic stability of tetrazole moieties-metabolic transformations of carboxylic acids are driven in part by microsomes of the liver, many of which are evaded by using a tetrazole isostere. The tetrazole pharmacophore has been used in a variety of drug classes, including nonsteroidal anti-inflammatory drugs, angiotensin receptor blockers, and proton pump inhibitors.

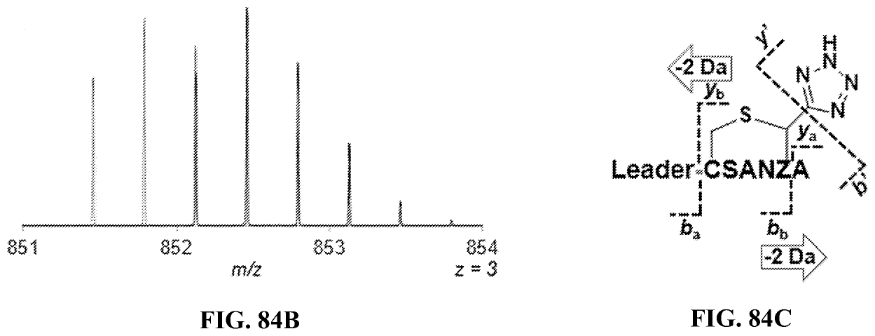

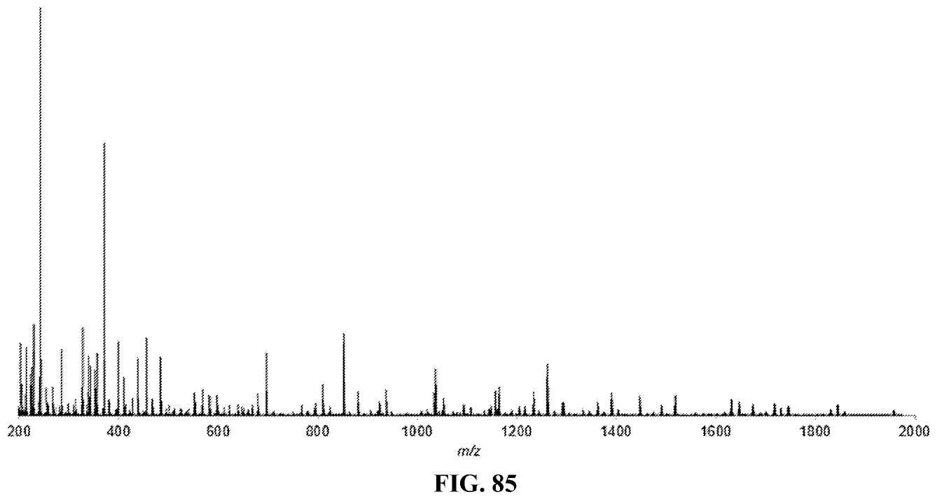

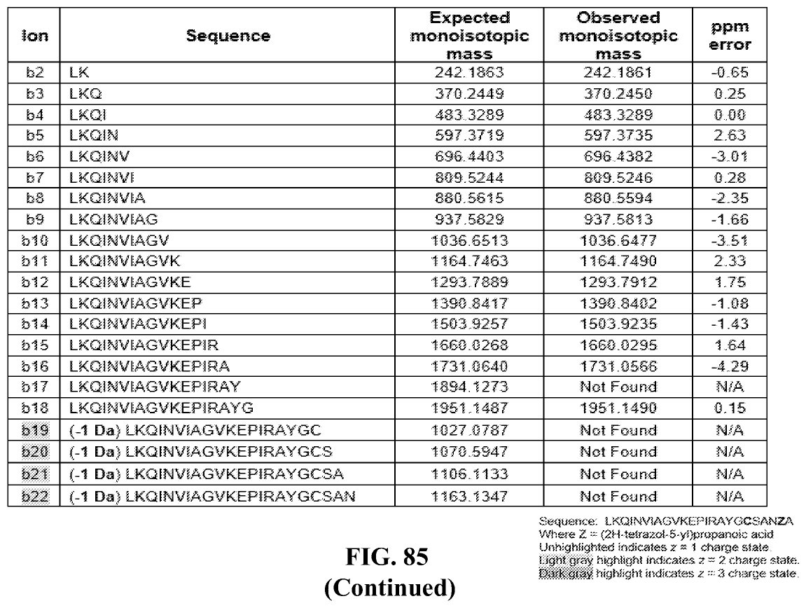

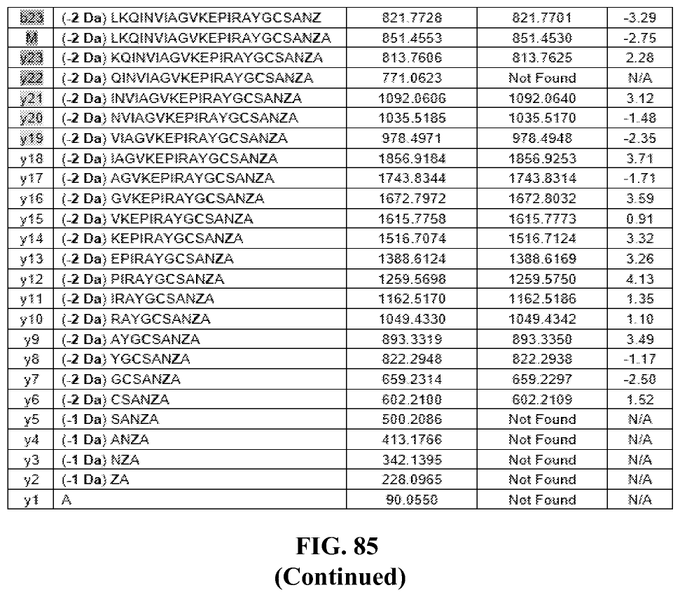

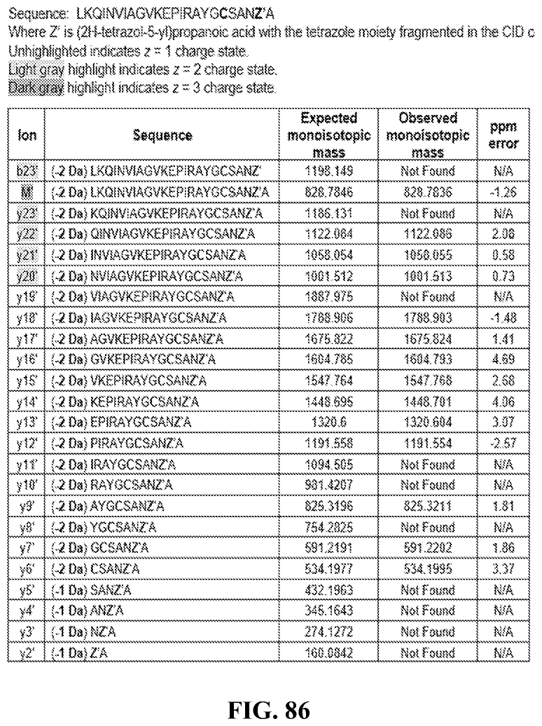

(2H-tetrazol-5-yl) propanoic acid (T4Az) was incorporated into msPapA (D23T4Az) by SPPS and incubated it with PapB to determine if the carboxylate-containing amino acid of msPapA can be replaced with the isosteric tetrazole-containing amino acid. The structures of the linear and cyclized peptides are shown in FIG. 84A. Upon reaction with PapB, a 2 Da shift is clearly observed (FIG. 84B), suggesting formation of a crosslink. The MS/MS spectra of D23T4Az msPapA reveals no fragmentation between the Cys and T4Az residue (FIG. 84C), but the anticipated 2 Da loss is observed in the b-ions after T4Az, and in the y-ions after the Cys residue (see FIG. 85 for the MS/MS spectra and all found fragments). The regiochemistry for crosslink is unconfirmed, however, several unusual peaks are present in the MS/MS that are potentially informative. For example, peaks are observed (FIG. 84C, y′ and b′) that are consistent with loss of the tetrazole sidechain moiety (see also FIG. 86 for additional y′- and b′-fragments). Without wishing to be bound by theory, these fragments suggest that the thioether forms alpha to the tetrazole.

This data with the tetrazole analog is the first-demonstrated ability of rSAM RiPP maturases to crosslink with a tetrazole moiety, opening new avenues for the development of peptide-based therapeutics. By using the tetrazole moiety in place of a carboxylate, the metabolic stability and pharmacokinetic properties of potential peptide therapeutics can be improved. This finding greatly expands the scope of rSAM RiPP maturases.

### 14. REFERENCES

(1) Van der Donk, W. A.; Bindman, N. A. Nat. Prod.: Discourse, Delivery, and Design, John Wiley & Sons: Oxford, 2014; pp 197-218.
(2) Sberro, H.; Fremin, B. J.; Zlitni, S.; Edfors, F.; Greenfield, N.; Snyder, M. P.; Pavlopoulos, G. A.; Kyrpides, N. C.; Bhatt, A. S. Large-Scale Analyses of Human Microbiomes Reveal Thousands of Small, Novel Genes. Cell. 2019, 178 (5), 1245-1259.
(3) Hetrick, K. J.; van der Donk, W. A. Ribosomally synthesized and post-translationally modified peptide natural product discovery in the genomic era. Curr. Opin. Chem. Biol. 2017, 38, 36-44.
(4) Süssmuth, R. D.; Mainz, A. Nonribosomal Peptide Synthesis-Principles and Prospects Angew. Chem. Int. Ed. Engl. 2017, 56, 3770.
(5) deGruyter, J. N.; Malins, L. R.; Baran, P. S. Residue-Specific Peptide Modifications: A Chemist's Guide. Biochem. 2017, 56 (30), 3863-3873.
(6) Benjdia, A.; Balty, C.; Berteau, O. Radical SAM Enzymes in the Biosynthesis of Ribosomally Synthesized and Post-translationally Modified Peptides (RiPPs). Front. Chem. 2017, 5 (87).
(7) Sofia, H. J.; Chen, G.; Hetzler, B. G.; Reyes-Spindola, J. F., Miller, N. E. Radical SAM, a novel protein superfamily linking unresolved steps in familiar biosynthetic pathways with radical mechanisms: functional characterization using new analysis and information visualization methods. Nucleic Acids Res. 2001, 29 (5), 1097-1106.
(8) Duffus, B. J.; Duschene, K. S.; Shepard, E. M.; Broderick, J. B. Radical S-Adenosylmethionine Enzymes. Chem. Rev. 2014, 114 (8), 4229-4317.
(9) Wecksler, S. R.; Stoll, S.; Tran, H.; Magnusson, O. T.; Wu, S. P.; King, D.; et al. Pyrroloquinoline quinone biogenesis: demonstration that PqqE from Klebsiella pneumoniae is a radical S-adenosyl-L-methionine enzyme. Biochem. 2009, 48, 10151-10161.
(10) Schramma, K. R.; Bushin, L. B.; and Seyedsayamdost, M. R. Structure and biosynthesis of a macrocyclic peptide containing an unprecedented lysine-to-tryptophan crosslink. Nat. Chem. 2015, 7, 431-437.
(11) Khaliullin, B.; Aggarwal, P.; Bubas, M.; Eaton, G. R.; Eaton, S. S.; Latham, J. A. Mycofactocin biosynthesis: modification of the peptide MftA by the radical S-adenosylmethionine protein MftC. FEBS Lett. 2016, 590, 2538-2548.
(12) Bushin, L. B.; Clark, K. A.; Pelczer, I.; and Seyedsayamdost, M. R. Charting an Unexplored Streptococcal Biosynthetic Landscape Reveals a Unique Peptide Cyclization Motif. J. Am. Chem. Soc. 2018, 140, 17674-17684.
(13) Imai, Y.; Meyer, K. J.; Iinishi, A.; Favre-Godal, Q.; Green, R.; Manuse, S.; et al. A New Antibiotic Selectively Kills Gram-Negative Pathogens. Nature 2019, 576, 459-464.
(14) Clark, K. A.; Bushin, L. B.; and Seyedsayamdost, M. R. Aliphatic Ether Bond Formation Expands the Scope of Radical SAM Enzymes in Natural Product Biosynthesis. J. Am. Chem. Soc. 2019, 141, 10610-10615.
(15) Flühe, L.; Burghaus, O.; Wieckowski, B. M.; Giessen, T. W.; Linne, U.; and Marahiel, M. A. (2013). Two [4Fe-4S] Clusters Containing Radical SAM Enzyme SkfB Catalyze Thioether Bond Formation during the Maturation of the Sporulation Killing Factor. J. Am. Chem. Soc. 2013, 135, 959-962.
(16) Balty, C.; Guillot, A.; Fradale, L.; Brewee, C.; Boulay, M.; Kubiak, X.; et al. Ruminococcin C, an Anti-clostridial Sactipeptide Produced by a Prominent Member of the Human Microbiota Ruminococcus Gnavus. J. Biol. Chem. 2019, 294, 14512-14525.
(17) Caruso, A., Bushin, L. B., Clark, K. A., Martinie, R. J., and Seyedsayamdost, M. R. Radical Approach to Enzymatic β-Thioether Bond Formation. J. Am. Chem. Soc. 2019, 141, 990-997.
(18) Hudson, G. A.; Burkhart, B. J.; DiCaprio, A. J.; Schwalen, C. J.; Kille, B.; Pogorelev, T. V.; Mitchell, D. A. Bioinformatic Mapping of S-Adenosylmethionine-Dependent Ribosomally Synthesized and Post-Translationally Modified Peptides Identifies new Cα, Cβ, or Cγ-linked Thioether-Containing Peptides. J. Am. Chem. Soc. 2019, 141, 8228-8238.
(19) Bruender, N. A.; Wilcoxen, J.; Britt, R. D.; and Bandarian, V. Biochemical and Spectroscopic Characterization of a Radical S-Adenosyl-L-Methionine Enzyme Involved in the Formation of a Peptide Thioether Cross-Link. Biochem. 2016, 55, 2122-2134.
(20) Freeman, M. F.; Gurgui, C.; Helf, M. J.; Morinaka, B. I.; Uria, A. R.; Oldham, N. J.; et al. Metagenome Mining Reveals Polytheonamides as Posttranslationally Modified Ribosomal Peptides. Science 2012, 338, 387-390.
(21) Vagstad, A. L.; Kuranaga, T.; Püntener, S.; Pattabiraman, V. R.; Bode, J. W.; and Piel, J. (2019). Introduction of D-Amino Acids in Minimalistic Peptide Substrates by an S-Adenosyl-L-Methionine Radical Epimerase. Angew. Chem., Int. Ed. 2019, 58, 2246-2250.
(22) Popp, P. F.; Friebel, A. L.; Benjdia, A.; Guillot, A.; Berteau, O.; and Mascher, T. (2021). The Epipeptide Biosynthesis Locus epeXEPAB Is Widely Distributed in Firmicutes and Triggers Intrinsic Cell Envelope Stress. Microb. Physiol. 2021, 1-12.
(23) Lewis, J. K.; Jochimsen, A. S.; Lefave, S. J.; Young, A. P.; Kincannon, W. M.; Roberts, A. G.; Kieber-Emmons, M. T.; Bandarian V. New Role for Radical SAM Enzymes in the Biosynthesis of Thio(seleno)oxazole RiPP Natural Products. Biochem. 2021, 60 (45), 3347-3361.
(24) Precord, T. W.; Mahanata, N.; Mitchell, D. A. Reconstitution and Substrate Specificity of the Thioether-Forming Radical S-Adenosylmethionine Enzyme in Freyrasin Biosynthesis. ACS Chem. Biol. 2019, 14 (9), 1981-89.
(25) Outten, F. W.; Djaman, O.; Storz, G. A suf operon requirement for Fe—S cluster assembly during iron starvation in Escherichia coli. Mol. Microbio. 2004, 52, 861-872.
(26) Wollers, S.; Layer, G.; Garcia-Serres, R.; Signor, L.; Clemancey, M.; Latour, J. M.; Fontecave, M.; Ollagnier de Choudens, S. Iron-sulfur (Fe—S) cluster assembly: the SufBCD complex is a new type of Fe—S scaffold with a flavin redox cofactor. J. Biol. Chem. 2010, 285 (30), 23331-23341.
(27) Rea, M. C.; Sit, C. S.; Clayton, E.; O'Connor, P. M.; Whittal, R. M.; Zheng, J.; Vederas, J. C.; Ross, R. P.; Hill, C. Thuricin CD, a posttranslationally modified bacteriocin with a narrow spectrum of activity against Clostridium difficile. Proc. Natl. Acad. Sci. U.S.A 2010, 107 (20), 9352-9357.
(28) Lohans, C. T.; Vederas, J. C. Structural characterization of thioether-bridged bacteriocins. J. Antibiot. 2014, 67, 23-30.
(29) King, A. M.; Anderson, D. A.; Glassey, E.; Segall-Shapiro, T. H.; Zhang, Z.; Niquille, D. L.; Embree, A. C.; Pratt, K.; Williams, T. L.; Gordon, D. B.; Voigt, C. A. Selection for constrained peptides that bind to a single target protein. Nat. Commun. 2021, 12, 6343.
(30) Lamberts, S. W. J.; Hofland, L. J. Octreotide, 40 years later. Eur. J. Endocrinol. 2019, 181, R173-R183.
(31) Muttenthaler, M.; King, G. F.; Adams, D. J.; Alewood, P. F. Trends in peptide drug discovery. Nat. Rev. Drug Discov. 2021, 20, 309-325.
(32) Kapust, R. B.; Tozser, J.; Copeland, T. D.; Waugh, D. S. The P1′ specificity of tobacco etch virus protease. Biochem. Biophy. Res. Commun. 2002, 294 (5), 949-955.
(33) Phan, J.; Zdanov, A.; Evdokimov, A. G.; Tropea, J. E.; Peters, H. K.; Kapust, R. B.; Li, M.; Wlodawer, A.; Waugh, D. S. Structural basis for the substrate specificity of tobacco etch virus protease. J. Biol. Chem. 2002, 277 (52), 50564-60672.
(34) Grove, T. L.; Himes, P. M.; Hwang, S.; Yumerefendi, H.; Bonanno, J. B.; Kuhlman, B.; Almo, S. C.; Bowers, A. B. Structural Insights into Thioether Bond Formation in the Biosynthesis of Sactipeptides. J. Am. Chem. Soc. 2017, 139 (34), 11734-11744.
(35) Lanz, N. D. Booker, S. J. Identification and function of auxiliary iron-sulfur clusters in radical SAM enzymes. Biochim. Biophys. Acta. 2012, 1824 (11), 1196-1212.
(36) Grell, T. A. J., Goldman, P. J., Drennan, C. L. SPASM and Twitch Domains in S-Adenosylmethionine (SAM) Radical Enzymes. J. Biol. Chem. 2015, 290 (7), 3964-3971.
(37) Haft, D. H., Basu, M. K. Biological systems discovery in silico: radical S-adenosylmethionine protein families and their target peptides for posttranslational modification. J. Bacteriol. 2011, 193 (11), 2745-2755.
(38) Grell, T. A. J., Kincannon, W. M., Bruender, N. A., Blaesi, E. J., Krebs, C., Bandarian, V., Drennan, C. L. Structural and spectroscopic analyses of the sporulation killing factor biosynthetic enzyme SkfB, a bacterial AdoMet radical sactisynthase. J. Biol. Chem. 2018, 293 (45),17349-17361.
(39) Burkhart, B. J.; Kakkar, N.; Hudson, G. A.; van der Donk, W. A.; Mitchell, D. A. Chimeric Leader Peptides for the Generation of Non-Natural Hybrid RiPP Products. ACS Cent. Sci. 2017, 3 (6), 629-638.
(40) Himes, P. M.; Allen, S. E.; Hwang, S.; Bowers, A. A. Production of Sactipeptides in Escherichia coli: Probing the Substrate Promiscuity of Subtilosin A Biosynthesis. ACS Chem. Biol. 2016, 11 (6), 1737-1744.
(41) Vagstad, A. L.; Kuranaga, T.; Püntener, S.; Pattabiraman, V. R.; Bode, J. W.; Piel, J. Introduction of D-Amino Acids in Minimalistic Peptide Substrates by an S-Adenosyl-L-Methionine Radical Epimerase. Angew. Chem., Int. Ed. 2018, 58, 2246-2250.
(42) Korneli, M.; Fuchs, S. W.; Felder, K.; Ernst, C.; Zinsli, L. V.; Piel, J. Promiscuous Installation of D-Amino Acids in Gene-Encoded Peptides. ACS Synth. Biol. 2021, 10 (2), 236-242.
(43) Chen, D.; Ruzicka, F. J.; Frey, P. A. A novel lysine 2,3-aminomutase encoded by the yodO gene of Bacillus subtilis: characterization and the observation of organic radical intermediates. Biochem. J. 2000, 348, 539-549.
(44) Crain, A. V.; Broderick, J. B. Pyruvate Formate-lyase and Its Activation by Pyruvate Formate-lyase Activating Enzyme. J. Biol. Chem. 2014, 289 (9), 5723-5729.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of chemically modifying a compound to install a thioether linkage, the method comprising reacting the compound with PapB, wherein the compound has a structure represented by a formula: 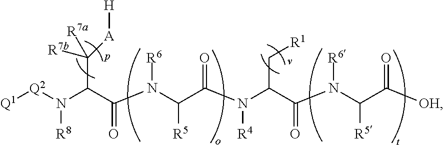
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein A is S or Se;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 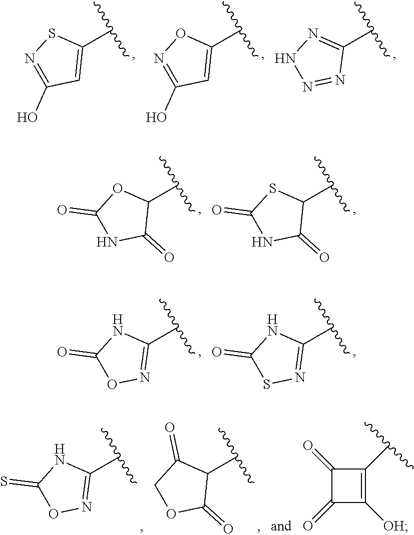
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl,
provided that the compound is not PapA.

2. The method of claim 1, wherein o is independently 0, 1, 2, 3, 4, 5, 6, or 7.

3. The method of claim 1, wherein t is 0.

4. The method of claim 1, wherein v is 1 or 2.

5. The method of claim 1, wherein R¹ is —CO₂H or a structure:

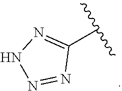 [SMILES: *c1nn[nH]n1]

6. The method of claim 1, wherein R¹ is —CO₂H.

7. The method of claim 1, wherein the cleavable moiety is —CO₂—(C4-C8 alkylene)-OC(O)—.

8. The method of claim 1, wherein the cleavable moiety is —CO₂CH₂CH═CHCH₂OC(O)—.

9. The method of claim 1, wherein the cleavable moiety is a protease recognition sequence.

10-13. (canceled)

14. The method of claim 1, wherein PapB installs a single thioether linkage in the compound.

15. The method of claim 1, wherein PapB installs two or more thioether linkages in the compound.

16. The method of claim 1, wherein the compound has a structure represented by a formula:

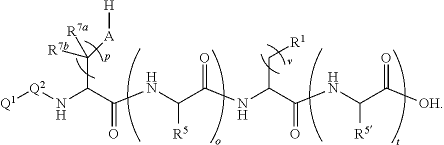

17. The method of claim 1, wherein the compound has a structure represented by a formula:

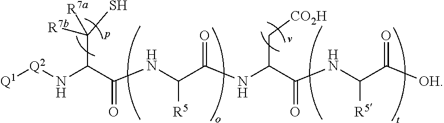

18. The method of claim 1, wherein the method produces a thioether compound having a structure represented by a formula: 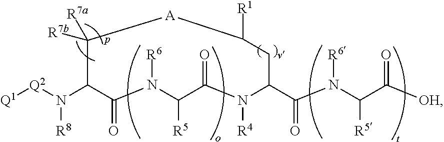
wherein v′ is 0, 1, 2, or 3.

19. The method of claim 1, wherein the method further comprises addition of a reducing agent.

20. The method of claim 19, wherein the method further comprises addition of a protease.

21. (canceled)

22. The method of claim 20, wherein the thioether compound is selected from:

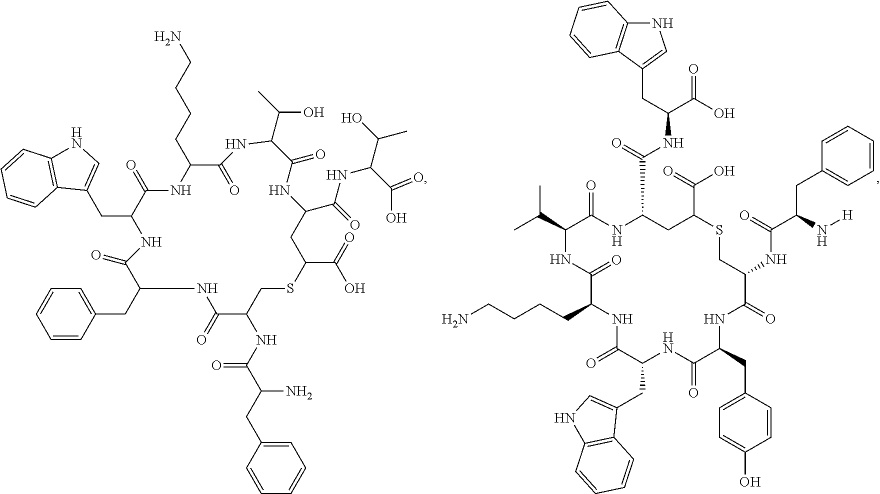 [SMILES: CC(O)C(NC(=O)C1CC(C(=O)O)SCC(NC(=O)C(N)Cc2ccccc2)C(=O)NC(Cc2ccccc2)C(=O)NC(Cc2c[nH]c3ccccc23)C(=O)NC(CCCCN)C(=O)NC(C(C)O)C(=O)N1)C(=O)O ; CC(C)[C@@H]1NC(=O)[C@H](CCCCN)NC(=O)[C@@H](Cc2c[nH]c3ccccc23)NC(=O)[C@H](Cc2ccc(O)cc2)NC(=O)[C@@H](NC(=O)[C@H](N)Cc2ccccc2)CSC(C(=O)O)C[C@@H](C(=O)N[C@@H](Cc2c[nH]c3ccccc23)C(=O)O)NC1=O]

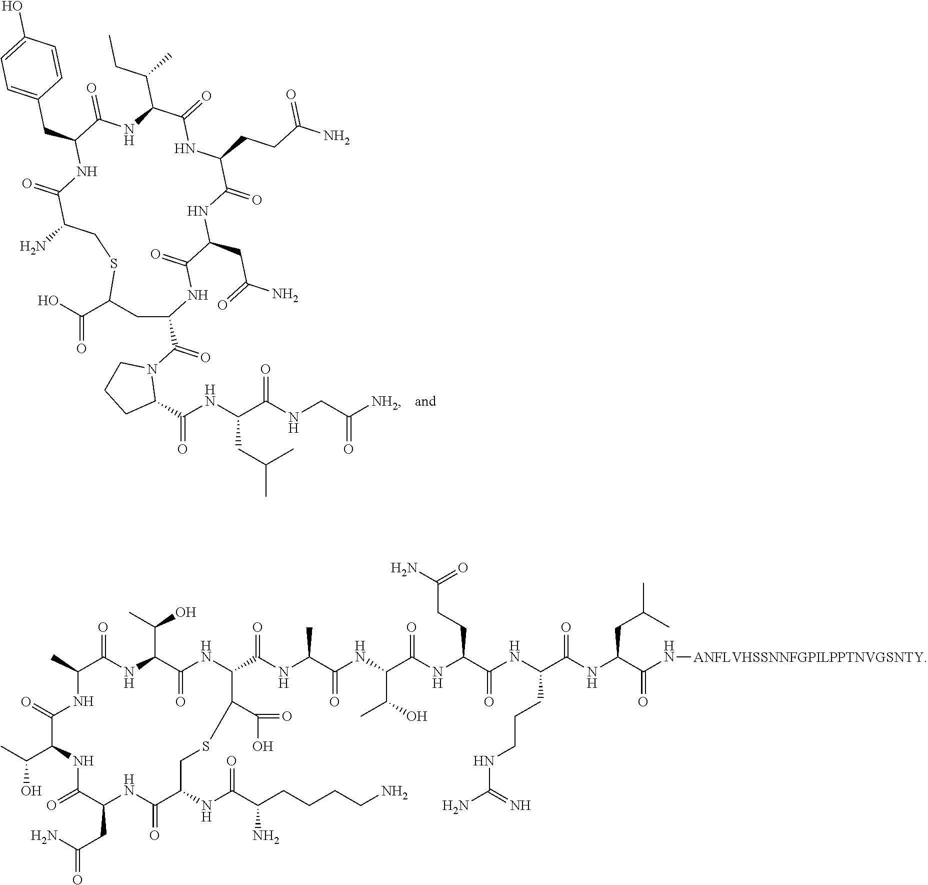

23-24. (canceled)

25. A method of chemically modifying a compound to install a thioether linkage, the method comprising reacting the compound with PapB, wherein the compound has a structure represented by a formula: 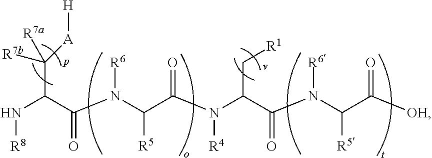
wherein o is 0, 1, 2, 3, 4, 5, 6, 7, 8, or 9;
wherein p is 1 or 2;
wherein t is an integer from 0 to 500;
wherein v is 1, 2, 3, 4, or 5;
wherein A is S or Se;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 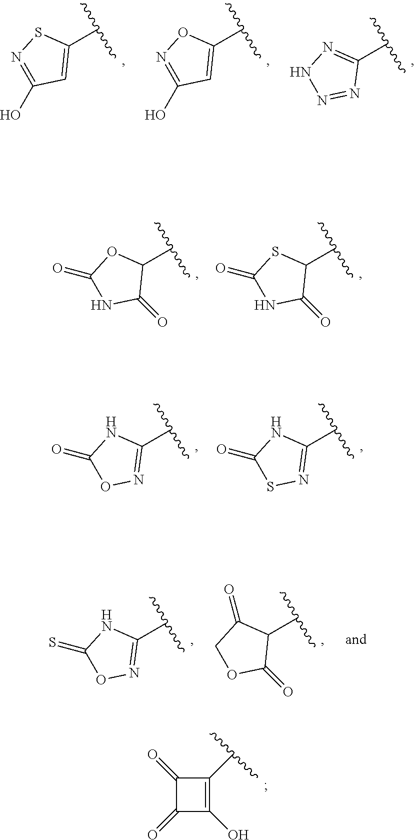
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl; and
wherein R⁸ is selected from hydrogen and methyl,
provided that the compound is not PapA.

26. A method of chemically modifying a compound to install a thioether linkage, the method comprising reacting the compound with PapB, wherein the compound has a structure represented by a formula: 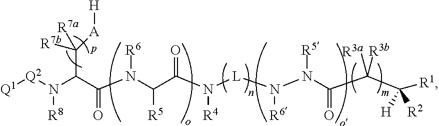
wherein m is 0, 1, 2, 3, or 4;
wherein n is 0 or 1;
wherein each of o and o′ is independently 0, 1, 2, 3, 4, 5, 6, 7, 8, or 9;
wherein p is 1 or 2;
wherein A is S or Se;
wherein L, when present, is selected from C2-C4 alkyl, —(C1-C4 alkyl) (OCH₂CH₂)_{q}, and a structure selected from: 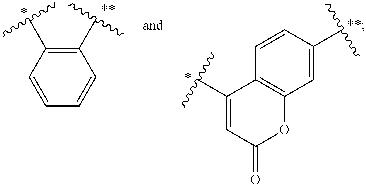
wherein q is 1, 2, 3, or 4;
wherein Q¹ is a leader sequence;
wherein Q² is a cleavable moiety;
wherein R¹ is selected from —CO₂H, —C(O)NHOH, —SO₂NH₂, —SO₂NHC(O)CH₃, —SO₃H, —NHC(O)NHSO₂CH₃, —P(O)(OH)₂, and a structure selected from: 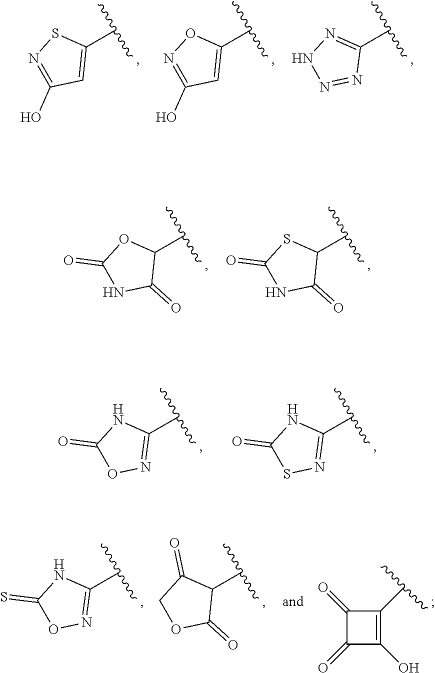
wherein R² is a residue of a side chain of amino acid, provided that the amino acid is not isoleucine or threonine;
wherein each of R^{3a} and R^{3b}, when present, is independently selected from C2-C5 alkynyl, C1-C5 azido, and a residue of a side chain of an amino acid;
wherein R⁴ is selected from hydrogen and methyl;
wherein each occurrence of R⁵ and R^{5′}, when present, is independently a residue of a side chain of amino acid;
wherein each occurrence of R⁶ and R^{6′}, when present, is independently selected from hydrogen and methyl, or wherein R⁶ or R^{6′} is covalently bonded to R⁵ or R^{5′}, respectively, and, together with the intermediate atoms, comprise an unsubstituted 5-membered heterocycle;
wherein each of R^{7a} and R^{7b}, when present, is independently selected from hydrogen and C1-C4 alkyl,
provided that the compound is not PapA.

27-77. (canceled)

78. The method of claim 0, wherein the method produces a thioether compound having a structure represented by a formula:

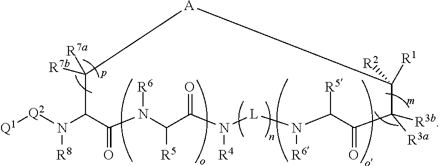

79-186. (canceled)
